(19) United States
(12) Patent Application Publication
(10) Pub. No.: US 2018/0171408 A1
(43) Pub. Date: Jun. 21, 2018

(54) Characterizing Gastro-Intestinal Disease

(71) Applicant: Vanderbilt University, Nashville, TN (US)

(72) Inventors: Thomas M. Aune, Nashville, TN (US); Philip S. Crooke, Nashville, TN (US); Nancy J. Olsen, Dallas, TX (US); John T. Tossberg, Nashville, TN (US)

(21) Appl. No.: 15/835,268

(22) Filed: Dec. 7, 2017

Related U.S. Application Data
(63) Continuation of application No. 14/089,102, filed on Nov. 25, 2013, now abandoned.
(60) Provisional application No. 61/731,265, filed on Nov. 29, 2012.

(51) Int. Cl. C12Q 1/6883

(52) U.S. Cl. CPC C12Q 1/6883; C12Q 2600/158

(57) ABSTRACT

A method for characterizing a gastro-intestinal disease in a subject involves comparing ratios of expression levels of genes in a biological sample from a subject to references, wherein the gastro-intestinal disease is characterized based on a difference in the ratios of the expression values of genes in the biological sample from the subject as compared to the references.

### RELATED APPLICATIONS

This application claims priority from U.S. patent application Ser. No. 14/089,102 filed Nov. 25, 2013, which claims priority from U.S. Provisional Application No. 61/731,265 filed Nov. 29, 2012, the entire disclosures of which are incorporated herein by this reference.

### GOVERNMENT INTEREST

This invention was made with government support under AI53984 and A1044924 awarded by the National Institutes of Health. The government has certain rights in the invention.

### TECHNICAL FIELD

The presently-disclosed subject matter relates to the characterization of gastro-intestinal (GI) diseases in a subject, including diagnosis of GI diseases and exclusion of a diagnosis of GI diseases.

### INTRODUCTION

Inflammatory bowel diseases (IBD), Crohn's disease (CD), Celiac's disease (CeD), and ulcerative colitis (UC) are chronic relapsing remitting inflammatory conditions affecting the gastrointestinal tract, primarily the small intestine and colon [1]. CD is most frequently diagnosed in patients in their 20s and UC in their 30s; however, the diagnosis can be made at any age [2]. IBD diagnosis is often straightforward, as disease can be seen by endoscopy or imaging modalities. However, diagnosis can be difficult as patients may experience symptoms consistent with IBD but ultimately have other diagnoses including functional gastrointestinal disorders such as irritable bowel syndrome (IBS) [3-6]. Patients with IBS can have symptoms very similar to those with IBD. IBD can be limited to difficult to evaluate areas of the GI tract such as isolated small bowel disease. Also, within IBD, differentiating between CD and UC can be difficult, especially within patients with severe inflammatory activity, often termed indeterminate colitis [7]. When the clinical presentation is severe and an operation including colectomy is indicated, differentiating CD and UC is imperative, as ileal pouch-anal anastomosis (IPAA) is generally contraindicated in CD due to high morbidity [8].

Developing biomarkers that can be easily obtained and allow for the correct diagnosis early into evaluation can avoid costly interventions that expose patients to multiple unnecessary procedures. Blood markers for both IBD and IBS have been sought for decades. For IBD, perinuclear antineutrophil cytoplasmic antibody (p-ANCA) and anti-Saccharomyces cerevisiae antibody (ASCA) have been reported to be markers for UC and CD, respectively. However, p-ANCA is also detected in 10-40% of patients with CD and ASCA is detected in 6-14% of patients with UC [1]. Other markers increased in subjects with CD include antibodies to (a) Escherichia coli outer membrane porin C (Omp-C), (b) protein from Pseudomonas fluorescens [9] and (c) flagellin c-BIR1 (anti-CBIR1) [10], but these markers remain insensitive. In patients with indeterminate colitis, those with one or more positive antibodies, including ANCA, ASCA, 12 (antibody to Pseudomonas fluorescens), and Omp-C, have significantly higher post-operative complications [11]. Other inflammatory biomarkers such as C-reactive protein, fecal calprotectin, and fecal lactoferrin differentiate IBD from other gastrointestinal disorders such as IBS [5], but tests do not differentiate among various types of inflammatory colitides [12].

Therefore, improved tests that can effectively, efficiently, and noninvasively characterize GI diseases are needed, including tests to diagnose GI diseases and/or to exclude a diagnosis of a GI disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 includes gene-expression profiles in multiple gastrointestinal disorders. Expression levels of 44 target genes were determined by quantitative RT-PCR and normalized to expression of GAPDH. Expression levels of 25 genes are shown; expression levels of the remainder were not statistically different between CTRL and any disease cohort. Results are expressed as transcript levels of individual genes relative to transcript levels of GAPDH using the formula: 2^{(GAPDH Ct-target gene Ct)}. Genes are identified that showed statistically significant (p-value<0.05 after Bonferroni's correction) increased or decreased expression in individual disease cohorts relative to CTRL subjects.

FIG. 2 includes a discrimination of IBD from CTRL and IBS from CTRL using the ratioscore system. (A) Ability of a single ratio, PGK1/POU6F1, to discriminate IBD and CTRL subjects. (B) The most discriminatory 25 gene-expression ratios were identified to segregate IBD and CTRL subjects. The ratioscore system was applied to combine ratio performance into a single discriminator. (C) Ability of a single ratio, PGK1/POU6F1, to discriminate IBS and CTRL subjects. (D) The most discriminatory 19 gene-expression ratios were identified to segregate IBS and CTRL subjects. The ratioscore system was applied to combine ratio performance into a single discriminator * indicates ratios found in both IBD:CTRL and IBS:CTRL comparisons.

FIG. 3 includes a discrimination of IBD from IBS using the ratioscore system. (A) Ability of a single ratio, HRAS/TBP, to discriminate IBD and IBS subjects. (B) The most discriminatory 25 gene-expression ratios were identified to segregate IBD and IBS subjects. The ratioscore system was applied to combine ratio performance into a single discriminator.

FIG. 4 includes a discrimination of UC from CD using the ratioscore system. (A) Ability of a single ratio, POU6F1/ANAPC1, to discriminate UC and CD subjects. (B) The most discriminatory 20 gene-expression ratios were identified to segregate UC and CD subjects. The ratioscore system was applied to combine ratio performance into a single discriminator.

FIG. 5 includes ROC curves derived from SVM #2 method, wherein sensitivity, specificity, and AUC were determined using the Mathematica program for the following comparisons: IBD:CTRL, IBS:CTRL, IBD:IBS, and CD:UC.

FIG. 6 includes proposed tiered analyses to discriminate subjects with IBD or IBS and, if positive for IBD, to discriminate between CD and UC.

FIG. 7 is a flow chart of the processing of the data and creation of the classifiers.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods, devices, and kits useful for characterizing an auto-immune disease in a subject and, more particularly, for characterizing gastro-intestinal (GI) diseases in a subject. In some embodiments, the method involves providing a biological sample from the subject; determining expression values of at least two genes in the biological sample; calculating one or more ratios of the expression values of the at least two genes; and comparing each ratios to a reference, wherein the GI disease(s) is characterized based on a difference in the ratios of the expression values of the at least two genes in the biological sample from the subject as compared to the references. In some embodiments, the biological sample is blood obtained from the subject or another biological sample containing a cell obtained from the subject, e.g., a subject suspected of having a GI disease. The method can be used, in some embodiments, to diagnose the subject with a GI disease. In some embodiments, the method can be used to exclude the subject from a diagnosis of GI disease.

The method can be used, in some embodiments, to diagnose the subject with a GI disease that is either an inflammatory bowel disease (IBD) or inflammatory bowel syndrome (IBS). In some embodiments, the method can be used to exclude the subject from a diagnosis of an IBD. In some embodiments, the method can be used to exclude the subject from a diagnosis of an IBD and to diagnose the subject with IBS. In some embodiments, the method can be used to exclude the subject from a diagnosis of IBS. In some embodiments, the method can be used to exclude the subject from a diagnosis of IBS and to diagnose the subject with an IBD.

The method can be used, in some embodiments, to diagnose the subject with a GI disease that is either Crohn's disease (CD) or ulcerative colitis (UC). In some embodiments, the subject is one who has received a diagnosis of IBD. In some embodiments, the method can be used to exclude the subject from a diagnosis of CD. In some embodiments, the method can be used to exclude the subject from a diagnosis of CD and to diagnose the subject with UC. In some embodiments, the method can be used to exclude the subject from a diagnosis of UC. In some embodiments, the method can be used to exclude the subject from a diagnosis of UC and to diagnose the subject with CD.

Methods of the presently-disclosed methods include determining expression values of genes in biological samples. As such, nucleic acid molecules or nucleotides are relevant to the disclosed subject matter. Nucleotides or genes, the expression of which is desired to be determined for characterizing an auto-immune disease, include, but are not limited to those identified in Table A, the isolated nucleic acid molecules of any one of SEQ ID NOs: 1-47, fragments of the isolated nucleic acid molecules of any one of SEQ ID NOs: 1-47 where detection of such fragments are indicative of expression of an associated gene, e.g., as identified in Table A, complementary nucleic acid molecules, isolated nucleic acid molecules capable of hybridizing to any one of the SEQ ID NOs: 1-47 under conditions disclosed herein, and corresponding RNA and/or DNA molecules.

As used herein, “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. The term “isolated”, when used in the context of an isolated DNA molecule or an isolated polypeptide, is a DNA molecule or polypeptide that, by the hand of man, exists apart from its native environment and is therefore not a product of nature.

Unless otherwise indicated, a particular nucleotide sequence also implicitly encompasses complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism. In one embodiment, a nucleic acid is derived from a biological sample isolated from a subject.

The terms “complementary” and “complementary sequences”, as used herein, refer to two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth herein, or is defined as being capable of hybridizing to the nucleic acid segment in question under conditions such as those described herein. In one embodiment, a complementary sequence is at least 80% complementary to the nucleotide sequence with which is it capable of pairing. In another embodiment, a complementary sequence is at least 85% complementary to the nucleotide sequence with which is it capable of pairing. In another embodiment, a complementary sequence is at least 90% complementary to the nucleotide sequence with which is it capable of pairing. In another embodiment, a complementary sequence is at least 95% complementary to the nucleotide sequence with which is it capable of pairing. In another embodiment, a complementary sequence is at least 98% complementary to the nucleotide sequence with which is it capable of pairing. In another embodiment, a complementary sequence is at least 99% complementary to the nucleotide sequence with which is it capable of pairing. In still another embodiment, a complementary sequence is at 100% complementary to the nucleotide sequence with which is it capable of pairing. A particular example of a complementary nucleic acid segment is an antisense oligonucleotide.

“Stringent hybridization conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences. An extensive guide to the hybridization of nucleic acids is found in Tijssen 1993, which is incorporated herein by this reference. In general, a signal to noise ratio of 2-fold (or higher) than that observed for a negative control probe in a same hybridization assay indicates detection of specific or substantial hybridization.

It is understood that in order to determine a gene expression level by hybridization, a full-length cDNA need not be employed. To determine the expression level of a gene represented by one of SEQ ID NOs: 1-47, any representative fragment or subsequence of the sequences set forth in SEQ ID NOs: 1-47 can be employed in conjunction with the hybridization conditions disclosed herein. As a result, a nucleic acid sequence used to assay a gene expression level can comprise sequences corresponding to the open reading frame (or a portion thereof), the 5′ untranslated region, and/or the 3′ untranslated region. It is understood that any nucleic acid sequence that allows the expression level of a reference gene to be specifically determined can be employed with the methods and compositions of the presently disclosed subject matter.

As used herein, the terms “corresponding to” and “representing”, “represented by” and grammatical derivatives thereof, when used in the context of a nucleic acid sequence corresponding to or representing a gene, refers to a nucleic acid sequence that results from transcription, reverse transcription, or replication from a particular genetic locus, gene, or gene product (for example, an mRNA). In other words, a partial cDNA, or full-length cDNA corresponding to a particular reference gene is a nucleic acid sequence that one of ordinary skill in the art would recognize as being a product of either transcription or replication of that reference gene (for example, a product produced by transcription of the reference gene). One of ordinary skill in the art would understand that the partial cDNA, or full- length cDNA itself is produced by in vitro manipulation to convert the mRNA into a cDNA, for example by reverse transcription of an isolated RNA molecule that was transcribed from the reference gene. One of ordinary skill in the art will also understand that the product of a reverse transcription is a double-stranded DNA molecule, and that a given strand of that double-stranded molecule can embody either the coding strand or the non-coding strand of the gene. The sequences presented in the Sequence Listing are single-stranded, however, and it is to be understood that the presently claimed subject matter is intended to encompass the genes represented by the sequences presented in SEQ ID NOs: 1-47, including the specific sequences set forth as well as the reverse/complement of each of these sequences.

The term “gene expression” generally refers to the cellular processes by which a biologically active polypeptide is produced from a DNA sequence. Generally, gene expression comprises the processes of transcription and translation, along with those modifications that normally occur in the cell to modify the newly translated protein to an active form and to direct it to its proper subcellular or extracellular location.

The terms “gene expression level” and “expression level” as used herein refer to an amount of gene-specific RNA or polypeptide that is present in a biological sample. When used in relation to an RNA molecule, the term “abundance” can be used interchangeably with the terms “gene expression level” and “expression level”.

Determination of expression levels of genes of interest can be achieved using any technique known the skilled artisan. For example, in some embodiments, RNA can be purified from the biological sample, converted to the more-stable complementary DNA (cDNA), before the gene expression products of genes of interest are detected. As will be recognized by the skilled artisan, where amplification of the sample is desired, polymerase chain reaction amplification can be employed. Determining the expression levels can be achieved, for example, using reverse transcription-polymerase chain reaction (RT-PCR), microarray analysis, or other techniques known to the skilled artisan.

In some embodiments, determining the expression levels of genes in the biological sample includes determining the expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 genes represented by SEQ ID NOs: 1-47. In some embodiments, determining the expression levels of genes in the biological sample includes determining the expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A.

TABLE A
Genes

| | | | | SEQ |
|---|---|---|---|---|
| Gene | | | ABI Assay | ID |
| Abbreviation | Gene | NCBI Ref. No. | Number: | NO: |
| ABR | active BCR-related gene, transcript | NM_001159746.1 | Hs00254300_m1 | 1 |
| | variant 3 | | | |
| ACTB | actin, beta | NM_001101.3 | Hs99999903_m1 | 2 |
| ACTR1A | ARP1 actin-related protein 1 | NM_005736.3 | Hs00194913_m1 | 3 |
| | homolog A, centractin alpha (yeast) | | | |
| ADAMTSL4 | ADAMTS-like 4 (ADAMTSL4), | NM_019032.4 | Hs00296775_m1 | 4 |
| | transcript variant 1 | | | |
| ANAPC1 | anaphase promoting complex | NM_022662.2 | Hs00224096_m1 | 5 |
| | subunit 1 | | | |
| APOBEC3F | apolipoprotein B mRNA editing | NM_145298.5 | Hs00272529_m1 | 6 |
| | enzyme, catalytic polypeptide-like | | | |
| | 3F | | | |
| ASL | argininosuccinate lyase | NM_001024943.1 | Hs00163695_m1 | 7 |
| B2M | beta-2-microglobulin | NM_004048.2 | Hs99999907_m1 | 8 |
| BRCA1 | breast cancer 1, early onset | NR_027676.1 | Hs00173237_m1 | 9 |
| | (BRCA1), transcript variant 6, non- | | | |
| | coding RNA | | | |
| CD55 | CD55 molecule, decay accelerating | NM_000574.3 | Hs00167090_m1 | 10 |
| | factor for complement (Cromer | | | |
| | blood group), transcript variant 1 | | | |
| CDH1 | cadherin 1, type 1, E-cadherin | NM_004360.3 | Hs00170423_m1 | 11 |
| | (epithelial) | | | |
| CDKN1B | cyclin-dependent kinase inhibitor | NM_004064.3 | Hs00153277_m1 | 12 |
| | 1B (p27, Kip1) | | | |
| CHEK2 | checkpoint kinase 2 (CHEK2), | NM_001005735.1 | Hs00200485_m1 | 13 |
| | transcript variant 3 | | | |
| CSF3R | colony stimulating factor 3 receptor | NM_156039.3 | Hs00167918_m1 | 14 |
| | (granulocyte), transcript variant 3 | | | |
| CTSS | cathepsin S, transcript variant 1 | NM_004079.4 | Hs00175403_m1 | 15 |
| EPHX2 | epoxide hydrolase 2, cytoplasmic | NM_001979.4 | Hs00157403_m1 | 16 |
| EXT2 | exostosin 2, transcript variant 2 | NM_207122.1 | Hs00181158_m1 | 17 |
| FOS | FBJ murine osteosarcoma viral | NM_005252.3 | Hs00170630_m1 | 18 |
| | oncogene homolog | | | |
| FOSL1 | FOS-like antigen 1 | NM_005438.3 | Hs00759776_s1 | 19 |
| FOXN3 | forkhead box N3, transcript variant 1 | NM_001085471.1 | Hs00231993_m1 | 20 |
| GAPDH-1 | glyceraldehyde-3-phosphate | NM_002046.3 | Hs99999905_m1 | 21 |
| | dehydrogenase | | | |
| GAPDH-2 | glyceraldehyde-3-phosphate | NM_002046.3 | Hs99999905_m1 | 22 |
| | dehydrogenase | | | |
| GATA3 | GATA binding protein 3 | NM_001002295.1 | Hs00231122_m1 | 23 |
| GNB5 | guanine nucleotide binding protein | NM_006578.3 | Hs00275095_m1 | 24 |
| | (G protein), beta 5, transcript | | and | |
| | variant 1 | | Hs01034253_m1 | |
| GSTM4 | glutathione S-transferase mu 4, | NM_147148.2 | Hs00426432_m1 | 25 |
| | transcript variant 2 | | | |
| HLA-DRA | major histocompatibility complex, | NM_019111.4 | Hs00219575_m1 | 26 |
| | class II, DR alpha | | | |
| HRAS | v-Ha-ras Harvey rat sarcoma viral | NM_001130442.1 | Hs00610483_m1 | 27 |
| | oncogene homolog (HRAS), | | | |
| | transcript variant 3 | | | |
| IFI27 | interferon, alpha-inducible protein | NM_001130080.1 | Hs00271467_m1 | 28 |
| | 27 (IFI27), transcript variant 1 | | | |
| IL11RA | interleukin 11 receptor, alpha, | NM_001142784.1 | Hs00234415_m1 | 29 |
| | transcript variant 3 | | | |
| JUN | jun proto-oncogene | NM_002228.3 | Hs00277190_s1 | 30 |
| KRAS | v-Ki-ras2 Kirsten rat sarcoma viral | NM_004985.3 | Hs00270666_m1 | 31 |
| | oncogene homolog, transcript | | | |
| | variant b | | | |
| LEPREL4 | leprecan-like 4 | NM_006455.2 | Hs00197668_m1 | 32 |
| LLGL2 | lethal giant larvae homolog 2 | NM_001015002.1 | Hs00189729_m1 | 33 |
| | (Drosophila), transcript variant 2 | | | |
| NRAS | neuroblastoma RAS viral (v-ras) | NM_002524.4 | Hs00180035_m1 | 34 |
| | oncogene homolog | | | |
| OAS1 | 2′-5′-oligoadenylate synthetase 1, | NM_001032409.1 | Hs00242943_m1 | 35 |
| | 40/46 kDa, transcript variant 3, | | | |
| ORC1 | origin recognition complex, subunit | NM_001190819.1 | Hs00172751_m1 | 36 |
| | 1 (ORC1), transcript variant 3 | | | |
| PGK1 | phosphoglycerate kinase 1 | NM_000291.3 | Hs99999906_m1 | 37 |
| PMAIP1 | phorbol-12-myristate-13-acetate- | NM_021127.2 | Hs00560402_m1 | 38 |
| | induced protein 1 | | | |
| POU6F1 | POU class 6 homeobox 1, | NR_026893.1 | Hs00231276_m1 | 39 |
| | transcript variant 2 | | | |
| RANGAP1 | Ran GTPase activating protein 1 | NM_002883.2 | Hs00610049_m1 | 40 |
| SPIB | Spi-B transcription factor (Spi- | NM_003121.3 | Hs00162150_m1 | 41 |
| | 1/PU.1 related) | | | |
| TAF11 | TAF11 RNA polymerase II, TATA | NM_005643.2 | Hs00194573_m1 | 42 |
| | box binding protein (TBP)- | | | |
| | associated factor, 28 kDa | | | |
| TBP | TATA box binding protein, | NM_001172085.1 | Hs00427620_m1 | 43 |
| | transcript variant 2 | | | |
| TGFBR2 | transforming growth factor, beta | NM_001024847.2 | Hs00559661_m1 | 44 |
| | receptor II (70/80 kDa), transcript | | | |
| | variant 1 | | | |
| TP53 | tumor protein p53 (TP53), | NM_001126113.1 | Hs00153340_m1 | 45 |
| | transcript variant 4 | | | |
| TP53-2 | tumor protein p53 (TP53), | NM_001126112.1 | Hs01034253_m1 | 46 |
| | transcript variant 2 | | | |
| TXK | TXK tyrosine kinase | NM_003328.2 | Hs00177433_m1 | 47 |
| IL11R1 | | | | |

In some embodiments, determining the expression levels of genes in the biological sample includes determining the expression levels of the genes corresponding to ABR, ACTB, ACTR1A, EXT2, KRAS, LLGL2, NRAS, PGK1, and POU6F1.

In some embodiments, determining the expression levels of genes in the biological sample includes determining the expression levels of the genes corresponding to ACTR1A, CD55, HRAS, IL11RA, JUN, PGK1, POU6F1, TAF11, TBP, and TP53.

In some embodiments, determining the expression levels of genes in the biological sample includes determining the expression levels of the genes corresponding to ABR, CD55, CTSS, GAPDH, HLA-DRA, HRAS, JUN, OAS1, ORC1L, and TBP.

In some embodiments, determining the expression levels of genes in the biological sample includes determining the expression levels of the genes corresponding to ANAPC1, CDH1, EXT2, GAPDH, GNB5, NRAS, ORC1L, POU6F1, TBP, and TP53.

In some embodiments, determining the expression levels of genes in the biological sample includes determining the expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

As used herein, a “ratio” or “expression ratio” is the expression value of a first biomarker (numerator) divided by the expression value of a second biomarker (denominator), e.g., Gene A/Gene B. As such, once the expression levels of at least two genes are determined, a ratio can be calculated. Ratios can be calculated using expression levels of genes in a biological sample obtained from a subject. In some embodiments, a reference can be a ratio calculated using expression levels of genes from another source. As such, the term “subject ratio” can used herein to refer to a ratio calculated using expression values of a gene pair in a biological sample obtained from a subject, while the term “reference ratio” can be used to refer to a ratio of the same biomarker pair in a reference sample, which serves as a reference to which the subject ratio is compared.

TABLE B
Ratios

| IBD vs. CTRL | IBS vs. Control | IBD vs. IBS | CD vs. UC |
|---|---|---|---|
| Expression Ratios | Expression Ratios | Expression Ratios | Expression Ratios |
| Numerator/ | Numerator/ | Numerator/ | Numerator/ |
| Denominator | Denominator | Denominator | Denominator |
| PGK1/POU6F1 | PGK1/POU6F1* | HRAS/GAPDH | POU6F1/ANAPC1 |
| PGK1/EXT2 | PGK1/ACTR1A* | HRAS/TBP | POU6F1/GAPDH |
| PGK1/ACTR1A | PGK1/TBP | HRAS/HLA-DRA | POU6F1/TBP |
| PGK1/NRAS | JUN/TBP* | HRAS/ORC1L | POU6F1/GNB5 |
| ABR/LLGL2 | JUN/CD55 | ABR/OAS1 | POU6F1/ORC1L |
| KRAS/LLGL2 | IL11RA/TBP* | ABR/JUN | POU6F1/TP53 |
| ACTB/LLGL2 | TAF11/TP53 | ABR/CTSS | GAPDH/CDH1 |
| NRAS/LLGL2 | HRAS/TP53 | ABR/CD55 | NRAS/EXT2 |
| GAPDH/ANAPC1 | ORC1L/TP53 | CDH1/PGK1 | ORC1L/APOBEC3F |
| GAPDH/TP53 | KRAS/APOBEC3F | CDH1/CTSS | SC65/ORC1L |
| GAPDH/GSTM4 | KRAS/ADAMTSL4 | PGK1/TBP | GATA3/TP53 |
| B2M/TP53 | ASL/ANAPC1 | ACTR1A/ORC1L | ASL/LLGL2 |
| B2M/APOBEC3F | GSTM4/TBP | TP53/SPIB | JUN/GAPDH |
| IL11RA/TBP | ABR/ANAPC1 | TP53/EXT2 | ADAMTSL4/KRAS |
| IL11RA/FOS | LLGL2/IL11RA | APOBEC3F/TAF11 | APOBEC3F/GAPDH |
| KRAS/ANAPC1 | KRAS/TBP | ADAMTSL4/ORC1L | CHEK2/GNB5 |
| KRAS/CHEK2 | CSF3R/TGFBR2 | CDKN1B/PMAIP1 | CDH1/GAPDH |
| JUN/TBP | GSTM4/OAS1 | IL11RA/TBP | LLGL2/CDH1 |
| JUN/SPIB | TXK/NRAS | JUN/TBP | IL11RA/PMAIP1 |
| NRAS/SC65 | | SC65/PGK1 | OAS1/IFI27 |
| ABR/CDH1 | | CSF3R/HLA-DRA | |
| HLA-DRA/ASL | | ACTB/FOS | |
| EPHX2/OAS1 | | ASL/GAPDH | |
| GSTM4/TP53 | | GATA3/TP53 | |
| LLGL2/CDH1 | | GNB5/JUN | |

In embodiments of the presently-disclosed subject matter, the method involves calculating one or more ratios of expression levels of genes corresponding to those set forth in Table A, wherein each ratio is calculated by dividing the expression level of a first gene in Table A by the expression level of a second gene in Table A.

In embodiments of the presently-disclosed subject matter, the method involves calculating one or more ratios set forth in Table B. In some embodiments, the method includes calculating 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, or 89 ratios set forth in Table B.

In embodiments of the presently-disclosed subject matter, the method involves calculating one or more ratios set forth in Column 1 (IBD vs. CTRL) of Table B. In some embodiments, the method includes calculating 1, 2, 3, 4, 5, 6, 7, 8, 9, 10, 11, 12, 13, 14, 15, 16, 17, 18, 19, 20, 21, 22, 23, 24, or 25 ratios set forth in Column 1 (IBD vs. Control) of Table B.

In embodiments of the presently-disclosed subject matter, the method involves calculating one or more ratios set forth in Column 2 (IBS v. Control) of Table B. In some embodiments, the method includes calculating 1, 2, 3, 4, 5, 6, 7, 8, 9, 10, 11, 12, 13, 14, 15, 16, 17, 18, or 19 ratios set forth in Column 2 (IBS v. Control) of Table B.

In embodiments of the presently-disclosed subject matter, the method involves calculating one or more ratios set forth in Column 3 (IBD vs. IBS) of Table B. In some embodiments, the method includes calculating 1, 2, 3, 4, 5, 6, 7, 8, 9, 10, 11, 12, 13, 14, 15, 16, 17, 18, 19, 20, 21, 22, 23, 24, or 25 ratios set forth in Column 3 (IBD vs. IBS) of Table B.

In embodiments of the presently-disclosed subject matter, the method involves calculating one or more ratios set forth in Column 4 (CD vs. UC) of Table B. In some embodiments, the method includes calculating 1, 2, 3, 4, 5, 6, 7, 8, 9, 10, 11, 12, 13, 14, 15, 16, 17, 18, 19, or 20 ratios set forth in Column 4 (CD vs. UC) of Table B.

Various references are appropriate for use in connection with the presently-disclosed subject matter, with non-limiting examples described herein. In some embodiments, the reference comprises a reference ratio calculated using of the expression level of two genes in a biological sample taken from one or more individuals, which two genes are the same two genes used to calculate the subject ratio. The expression levels of genes in biological samples from one or more individuals can be a expression levels from a reference group or comparator group.

In some embodiments, a “comparator group” or “reference group” includes individuals having a common characterization, for example, healthy control individuals, individuals who have been diagnosed with a condition often confused with an auto-immune disease of interest in the context of clinical diagnosis, individuals who have been diagnosed with an auto-immune disease of interest, or individuals who have another common characterization of interest. Expression values of biomarkers obtained from biological samples of individuals in a comparator group can be used to calculate reference ratios. Data associated with one or more comparator groups can be stored, for example, in a database that can be accessed when practicing a method in accordance with the presently-disclosed subject matter.

With reference to Table B, for example, ratios-of-interest are provided for use with a healthy control comparator group (CTRL, column 1 and column 2) or a comparator group of individuals having IBS or IBD (column 3), or having CD or UC (column 4). Examples of comparator groups relevant to characterization of a GI disease include, but are not limited to: healthy control (CTRL), irritable bowel syndrome (IBS), inflammatory bowel diseases (IBD), Crohn's disease (CD), Celiac's disease (CeD), and ulcerative colitis (UC). Because a comparator group can include data from multiple individuals, as will be recognized by one of ordinary skill in the art, it is expected that the expression values of biomarkers in biological samples obtained from different individuals in the same comparator group might differ. As such, identification of a reference ratio for a particular gene pair can be made with reference to a “threshold reference ratio” for the gene pair within the comparator group. In some embodiments, for example, the threshold expression ratio could be a median, an average, a value based on statistical analysis of the distribution of ratios of expression levels of the gene pair within the comparator group, or another threshold value, e.g., top value in the group, second highest value in the group, third highest value in the group, etc.

In some embodiments, the reference comprises a reference ratio calculated using a standard sample containing standard biomarker amounts, which can be analyzed in the same manner or even concurrently with the biological sample. In some embodiments, the reference comprises ratio values, such as standard threshold values. Such values can be published in a format useful for the practitioner, such as in a list, table, database, or incorporated into a software or system for use in connection with the presently-disclosed subject matter. Such values can in some cases be based, for example, on information obtained from a comparator group.

Ratios of interest, or ratios of gene pairs that are useful for characterizing GI diseases, have the ability to distinguish groups, e.g., IBD group and health control group, IBS group and health control group, IBD group and IBS group, CD group and UC group. Table B includes examples of ratios of interest for IBD vs. healthy control (CTRL), IBS vs. healthy control, IBD vs. IBS, and CD vs. UC. In this regard, an auto-immune disease can be characterized based on a difference in the ratios of the expression values of at least two genes in a biological sample from the subject as compared to a reference ratio.

In some embodiments, it can be useful to compare one or more subject ratios to one or more first reference ratios, e.g., from a first comparator group, and also to compare the one or more subject ratios to one or more second reference ratios, e.g., from a second comparator group. Such a multi-tiered approach can improve the efficacy of the characterization of GI diseases, as will be explained further in the Examples section.

Characterizing can include providing a diagnosis, prognosis, and/or theragnosis of an auto-immune disease in a subject.

“Making a diagnosis” or “diagnosing,” as used herein, are further inclusive of making a prognosis, which can provide for predicting a clinical outcome (with or without medical treatment), selecting an appropriate treatment (or whether treatment would be effective), or monitoring a potential auto-immune disease, based on calculated ratios of expression levels of genes. Diagnostic testing that involves treatment, such as treatment monitoring or decision making can be referred to as “theranosis.” Further, in some embodiments of the presently disclosed subject matter, multiple determinations of ratios of expression levels of genes over time can be made to facilitate diagnosis (including prognosis), evaluating treatment efficacy, and/or progression of a potential auto-immune disease or auto-immune disease. A temporal change in one or more ratios can be used to predict a clinical outcome, monitor the progression of the condition, and/or efficacy of administered therapies. In such an embodiment for example, one could observe a change in a particular ratio in a biological sample over time during the progression of a condition and/or during the course of a therapy.

The presently disclosed subject matter further provides in some embodiments a method for theranostic testing, such as evaluating progression of a condition and/or treatment efficacy in a subject. In some embodiments, the method comprises providing a series of biological samples over a time period from the subject; determining expression values of at least two genes in each of the biological samples; calculating one or more ratios of the expression values of the at least two genes for each of the biological samples; and determining any measurable change in the ratios in each of the biological samples from the series to thereby evaluate progression of the condition and/or treatment efficacy.

Any changes in the ratios, and changes in the ratios relative to references, over the time period can be used to make a diagnosis, predict clinical outcome, determine whether to initiate or continue the therapy, and whether a current therapy is effectively.

The phrase “determining the prognosis” as used herein refers to methods by which the skilled artisan can predict the course or outcome of a condition in a subject. The term “prognosis” can refer to the ability to predict the course or outcome of a condition with up to 100% accuracy, or predict that a given course or outcome is more or less likely to occur based on the ratios of expression values of genes of interest. The term “prognosis” can also refer to an increased probability that a certain course or outcome will occur; that is, that a course or outcome is more likely to occur in a subject when compared to individuals in a comparator group. For example, in individuals exhibiting subject ratios-of-interest that are higher than reference ratio-of-interest, the chance of a given outcome (e.g., a GI disease diagnosis) may be very high. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome can be performed using statistical analysis. For example, subject ratios that are higher than reference ratios in some embodiments can signal that a subject is more likely to suffer from an auto-immune disease than subjects with ratios that are substantially equal to reference ratios, as determined by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001. When performing multiple statistical tests, p values can be corrected for multiple comparisons using techniques known in the art.

Further with respect to the methods of the presently disclosed subject matter, a preferred subject is a vertebrate subject. A preferred vertebrate is warm-blooded; a preferred warm-blooded vertebrate is a mammal. A mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter further includes kits and devices useful for detecting and/or determining expression levels of at least two genes in a biological sample.

The kits of the presently-disclosed subject matter can include primer pairs for determining expression levels of at least two genes, which can be useful for calculating ratios as disclosed herein. In some embodiments, the kit includes primer pairs for determining expression levels of at least two genes represented by SEQ ID NOs: 1-47. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes represented by SEQ ID NOs: 1-47. In some embodiments, the kit includes primer pairs for determining expression levels of at least two genes corresponding to those set forth in Table A. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A.

In some embodiments, the kit includes primer pairs for determining expression levels of the genes corresponding to ABR, ACTB, ACTR1A, EXT2, KRAS, LLGL2, NRAS, PGK1, and POU6F1. In some embodiments, the kit includes primer pairs for determining expression levels of the genes corresponding to ACTR1A, CD55, HRAS, IL11RA, JUN, PGK1, POU6F1, TAF11, TBP, and TP53. In some embodiments, the kit includes primer pairs for determining expression levels of the genes corresponding to ABR, CD55, CTSS, GAPDH, HLA-DRA, HRAS, JUN, OAS1, ORC1L, and TBP. In some embodiments, the kit includes primer pairs for determining expression levels of the genes corresponding to ANAPC1, CDH1, EXT2, GAPDH, GNB5, NRAS, ORC1L, POU6F1, TBP, and TP53. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

The devices of the presently-disclosed subject matter can include a probe for selectively binding each of at least two gene expression products to detect at least two genes, which can be useful for determining expression levels of the genes and for calculating ratios as disclosed herein. Such probes can selectively bind the gene products, for example, by hybridization of the probe and a nucleotide gene product. In some embodiments, the device includes probes for detecting each of at least two genes represented by SEQ ID NOs: 1-47. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes represented by SEQ ID NOs: 1-47. In some embodiments, the device includes probes for detecting each of at least two genes corresponding to those set forth in Table A. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A.

In some embodiments, the device includes probes for detecting each of the genes corresponding to ABR, ACTB, ACTR1A, EXT2, KRAS, LLGL2, NRAS, PGK1, and POU6F1. In some embodiments, the device includes probes for detecting each of the genes corresponding to ACTR1A, CD55, HRAS, IL11RA, JUN, PGK1, POU6F1, TAF11, TBP, and TP53. In some embodiments, the device includes probes for detecting each of the genes corresponding to ABR, CD55, CTSS, GAPDH, HLA-DRA, HRAS, JUN, OAS1, ORC1L, and TBP. In some embodiments, the device includes probes for detecting each of the genes corresponding to ANAPC1, CDH1, EXT2, GAPDH, GNB5, NRAS, ORC1L, POU6F1, TBP, and TP53. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

Some of the gene sequences disclosed herein are cross-referenced to GENBANK® accession numbers. The sequences cross-referenced in the GENBANK® database are expressly incorporated by reference as are equivalent and related sequences present in GENBANK® or other public databases. Also expressly incorporated herein by reference are all annotations present in the GENBANK® database associated with the sequences disclosed herein. Unless otherwise indicated or apparent, the references to the GENBANK® database are references to the most recent version of the database, as of the filing date of this Application.

While the terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

Inflammatory bowel diseases, ulcerative colitis and Crohn's disease are considered to be of autoimmune origin, but the etiology of irritable bowel syndrome remains elusive. Furthermore, classifying patients into irritable bowel syndrome and inflammatory bowel diseases can be difficult without invasive testing and holds important treatment implications. Our aim was to assess the ability of gene expression profiling in blood to differentiate among these subject groups.

It is generally thought that different profiles of biomarkers could provide useful information to guide clinical decision-making; from diagnosis to choice of optimal therapies and in some cases these biomarker profiles are being implemented in clinical practice [3,12-24]. Searches for optimal biomarker profiles can be achieved using clustering methods e.g., heirarchical clustering, K-means clustering, which depend upon the general ability to find common features across a sample population or forms of linear discriminate analysis, which depend upon the ability to find linear combinations of features that have the ability to separate two or more classes. The former method is a common method to analyze large numbers of features, such as microarray data whereas the latter is a more common method for analysis of smaller numbers of features. Both methods are suitable for further analyses using machine learning methods such as support vector machines, logistic regression, principal components analysis or prediction analysis for microarrays. Using a form of linear discriminant analysis, we have attempted to employ mRNA transcript profiles to distinguish between subjects with multiple sclerosis and other comparator groups [25,26]. Our results clearly demonstrate that mRNA transcript profiling has the capacity to distinguish between MS, even early in the disease process, and homogeneous comparator groups, such as healthy subjects (CTRL), or subjects with clinically related diseases such as neuromyelitis optica or transverse myelitis. Thus, these binary comparisons can produce a test of exclusion of multiple sclerosis. Here, we applied this approach to IBD and IBS. Our results demonstrate that distinct mRNA profiles accurately discriminate IBD from CTRL, IBS from CTRL, IBD from IBS, and CD from UC with high degrees of sensitivity and specificity. We propose these approaches may provide useful guides for clinical decision-making.

Methods

Transcript levels of a total of 45 genes in blood were determined by quantitative real-time polymerase chain reaction (RT-PCR). We applied three separate analytic approaches; one utilized a scoring system derived from combinations of ratios of expression levels of two genes and two different support vector machines.

Human Subjects

Blood samples collected in PAXgene tubes were obtained from CTRL, IBS, CeD, CD or UC subjects. Diagnosis of IBD, both CD and UC, was made by colonoscopy or sigmoidoscopy and tissue biopsy to localize inflammation to all layers of the intestinal wall (CD) or only the inner lining layer (UC). Diagnosis of IBS was made by the absence of pathologic damage in the colon after examination by colonoscopy or sigmoidoscopy. Inclusion criteria were diagnosis by a gastro-intestinal specialist using these methods. Age, race and gender were not statistically different among the different study groups. Time of blood draw, for example, morning/afternoon clinics, was also not statistically significant among the different study groups. Relevant institutional review board approval was obtained from all participating sites.

MRNA Transcript Determination

Total RNA was purified using Qiagen's RNA isolation kits using standard protocols and was reverse-transcribed using poly-A primers using Superscript III (Invitrogen, Carlsbad, Calif., USA). A TaqMan Low Density Array (TLDA) was designed to analyze expression levels of 44 target genes and of four housekeeping genes in 300 ng cDNA. The gene probes on the TLDA plate were: ABR, ACTB, ACTR1A, ADAMTSL4, ANAPC1, APOBEC3F, ASL, B2M, BRCA1, CD55, CDH1, CDKN1B, CHEK2, CSF3R, CTSS, EPHX2, EXT2, FOS, FOSL1, GAPDH, GATA3, GNB5-1, GNB5-1, GSTM4, HLA-DRA, HRAS, IFI27, IL11RA, JUN, KRAS, LEPREL4, LLGL2, NRAS, OAS1, ORC1L, PGK1, PMAIP1, POU6F1, RANGAP1, SC65, SPIB, TAF11, TBP, TGFBR2, TP53-1 TP53-2, TXK. GNB5-1 and -2 and TP53-1 and -2 interrogate different exon-intron junctions. [26]. Inclusion of the specific gene targets was based upon the following criteria: (a) previous studies demonstrating differential expression among control and multiple autoimmune diseases, (b) protein products possess known inflammatory functions, (c) expression levels change in response to pro-inflammatory stimuli (cytokines), and/or (d) protein products have known roles in cell cycle progression and/or apoptosis. Patient diagnosis was blinded for all experimental procedures. Relative expression levels were determined directly from the observed threshold cycle (CT). Linear expression levels were determined using the formula, 240-CT.

Ratioscore and Support Vector Machine (SVM) Methods

Principal Component Analysis (PCA) was applied directly to the normalized gene expression data using MATLAB's Bioinformatics Toolkit (The MathWorks, Inc.) and other techniques to identify a lower dimensional space of gene expressions that could be used to classify controls from cases. The results were disappointing and we concluded that looking at ratios of the gene expression data may be a more productive approach. The computational algorithm and permutation testing strategy employed to identify discriminatory combinations of ratios to create the ratioscore (our terminology) have been previously described [26]. For completeness, we summarize the algorithm used in the Ratioscore Method below. Let D denote the set of gene-expression levels associated with the disease group and C denote the set of gene-expression levels associated with the control group. The algorithm searches for the “best” set of gene ratios that partitions D and C:
80% of the control and disease groups are randomly selected. Gene-expression level ratios are formed for elements in D and C. For each ratio, the number of elements in the disease group that are larger than the largest ratio in the control group is computed. The top 500 ratios that separate elements in D and C are saved. This calculation is repeated 200 times resulting in a set of 200 subsets of ratios (each subset having 500 ratios).
The 500 subsets are then processed looking for the smallest number of ratios, R={r₁, r₂, . . . , rₙ}, that produce the maximum of separation of D and C. Associate with each of the ratios in R, there are threshold values, T={t₁, t₂, . . . , tₙ}, which correspond to the highest value in the control group for each of the ratios in R .
For each member of the disease group D, the ratios in R are computed, {a₁, a₂, . . . , aₙ}. If aᵢ≥tᵢ, then we assign the ratio a 1; otherwise, it is assigned a 0. In this way, we generate an n-tuple of 1's and 0's for each member of D. For example, if n=6, then a typical 6-tuple would be {1, 1, 0, 0, 1, 0}. This would mean that this individual in the disease group would have 3 ratios that exceed the corresponding ratios in the control group.
Lastly, the percentage of members in the disease group that have nonzero n-tuples is calculated. The larger the percentage, the better the separation of D and C.

The algorithm allows one to identify the smallest number of ratios that partitions the case and control groups.

Two support vector machines (SVM) were independently created and trained using ratios identified by the Ratioscore Method. The first SVM was coded in Mathematica (Wolfram Research, Inc.) and the second SVM employed LS-SVMLab software (http://www.esat.kuleuven.be/sista/lssvmab). We decided to use the two independently developed SVM since the choice of kernels, optimization algorithms, and the training algorithms can produce differing results. There was little difference in the performance of the two machines when classifying the different case-control combinations. To confirm the results of the Ratioscore Method and the SVM approaches, logistic regression was employed to separate to the case and control sets using the gene ratios. Its performance was in line with the other two approaches and hence, we have chosen not to report these results.

Statistical Analysis

The Welch's-corrected T-test not assuming equal variances was employed to calculate p-values in two-way comparisons. Fisher's exact test was employed to calculate p-values in 2 by 2 comparisons. The Bonferroni's method was employed to correct for multiple testing [27].

Results

All methods discriminated different subject cohorts, irritable bowel syndrome from control, inflammatory bowel disease from control, irritable bowel syndrome from inflammatory bowel disease, and ulcerative colitis from Crohn's disease, with high degrees of sensitivity and specificity.

Gene-Expression Patterns in Distinct Gastrointestinal Diseases

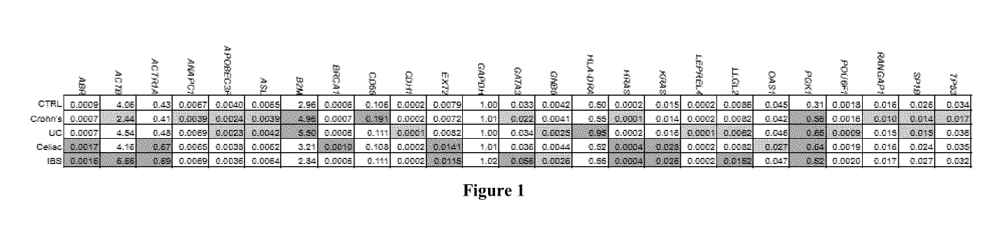

CTRL, IBD (CD and UC), IBS subjects were recruited from multiple sites within the United States. Demographic characteristics of the different gastrointestinal disease cohorts were not statistically different from the CTRL cohort (Table 1). We measured expression patterns of a common set of genes assayed using a common platform in CTRL and subjects with different gastrointestinal conditions, CD and UC, IBS, and CeD. Genes for analysis were selected from prior microarray studies [20,26]. Gene transcript levels were determined by quantitative RT-PCR and normalized to GAPDH transcript levels. We employed a heatmap to depict those genes differentially expressed in individual subject cohorts relative to the CTRL cohort, p-value<0.05 (after Bonferroni correction for multiple testing; see FIG. 1 with red=over-expressed gene, green=under-expressed gene). Ratios of transcript levels of individual genes in the indicated disease cohorts relative to GAPDH were calculated and depicted within each box. Each disease exhibited an underlying unique pattern of gene-expression. However, these profiles were sufficiently overlapping to prohibit accurate discrimination of one disease from another disease using the expression profile alone. For example, while PGK1 was over-expressed in all four conditions, ABR, ACTR1A, EXT2, HRAS, and KRAS were over-expressed in CeD and IBS but not CD and UC. Similarly, APOBEC3F, ASL, and SPIB were under-expressed in CD and UC, but not CeD and IBS. Other genes, ANAPC1, RANGAP1, and TP53, were only under-expressed in CD. Certain genes, e.g., APOBEC3F, ASL, GNB5, SPIB, were only under-expressed relative to the CTRL cohort, while other genes, e.g., ACTB, GATA3, HRAS, and LLGL2, were under-expressed in specific disease cohorts relative to CTRL but over-expressed in other disease cohorts relative to CTRL. Thus, each gene was differentially expressed in at least one disease cohort relative to CTRL. However, each individual disease cohort did not possess a unique expression profile distinguishing it from all other disease cohorts. For these reasons, we decided to look at other separation techniques.

TABLE 1
Demographic characteristics of the different subject populations

| | | AGE | | GENDER | | ETHNICITY | |
|---|---|---|---|---|---|---|---|
| | # | yrs | P* | (% F) | P | (% C/AA/As/H) | P |
| IBD | 97 | 40 ± 9 | NS | 62 | NS | 92/5/0/1 | NS |
| CD | 46 | 38 ± 10 | NS | 63 | NS | 91/4/0/0 | NS |
| UC | 40 | 41 ± 8 | NS | 59 | NS | 93/5/0/2 | NS |
| IBS | 44 | 43 ± 10 | NS | 79 | NS | 90/7/0/3 | NS |
| CeD | 16 | 44 ± 12 | NS | 69 | NS | 100/0/0/0 | NS |
| CTRL | 113 | 41 ± 11 | | 67 | | 89/9/0/2 | |

*P calculated by Student T-test (Age) or Fisher's exact test, NS: p-value > 0.05
^{†}C, Caucasian; AA, African American; As, Asian; H, Hispanic

Discrimination of IBD or IBS from CTRL Based Upon Gene-Expression Ratios

Initially, we employed standard methods of microarray analyses including unsupervised heirarchical clustering, supervised heirarchical clustering, and principal components analysis using the TIGR microarray software Multiexperiment Viewer to segregate patient groups. After normalization to GAPDH, gene expression data from IBD samples or IBS samples and CTRL samples were analyzed using unsupervised and supervised heirarchical clustering using all genes or only those genes whose expression was statistically significant using the supervised T-test. We found that unsupervised heirarchical clustering segregated 72% of IBD samples in one major branch and 28% of IBD samples in the second major branch. Similarly, 36% of CTRL samples were segregated into the branch with most of the IBD samples while 64% of CTRL samples were segregated into the alternate branch. Comparison of IBS and CTRL using unsupervised heirarchical clustering also did not produce the desired level of discrimination between case and control cohorts. Supervised heirarchical clustering and principal components analysis produced a similar low level of overall accuracy.

For these reasons, we turned to a type of linear discriminant analysis classifier

(Ratioscore Method) that we employed previously to discriminate subjects with multiple sclerosis from different control cohorts. We employed a search algorithm to identify those ratios of gene-expression levels in which the greatest number of subjects in the test group possessed a ratio value greater than the highest ratio value in the comparator group. We employed a second algorithm to perform permutation testing of one subject group to identify the optimum set of discriminatory ratios. CeD was excluded from this analysis due to the low number of cases in this cohort. Examination of expression levels of ratios of genes rather than individual genes offered the following advantages. First, ratios normalized for differences in mRNA or cDNA template quantity and quality among different samples. Second, ratios obviated the need for inclusion of a housekeeping genes in the analysis and the assumption that expression levels of housekeeping genes did not vary among different subject populations. Third, comparisons of ratios or combinations of ratios may more accurately identify cellular phenotypes that may contribute to disease. For example, a ratio containing one gene in the numerator that is over-expressed in the case cohort relative to the control cohort and one gene in the denominator that is under-expressed in the case cohort relative to the control cohort should produce a greater ratio value difference between individuals in the two cohorts than a single expression value. Fourth, ANAPC1, RANGAP1, and LEPREL4 genes encode unique proteins and each participates in mitosis [28-33]. Thus, a defect in expression of any one of these genes could produce a common cellular phenotype; a defect in mitosis, and for example, one subject with a given disease may exhibit a deficiency in expression of ANAPC1 while a second individual with the same disease may exhibit a deficiency in expression of RANGAP1 and a third with the same disease may exhibit a defect in LEPREL4 expression levels. Any of these defects has the potential to produce a common cellular phenotype. Our approach makes it possible to capture each subject as positive for a given disease. We refer to this as the Ratioscore Method.

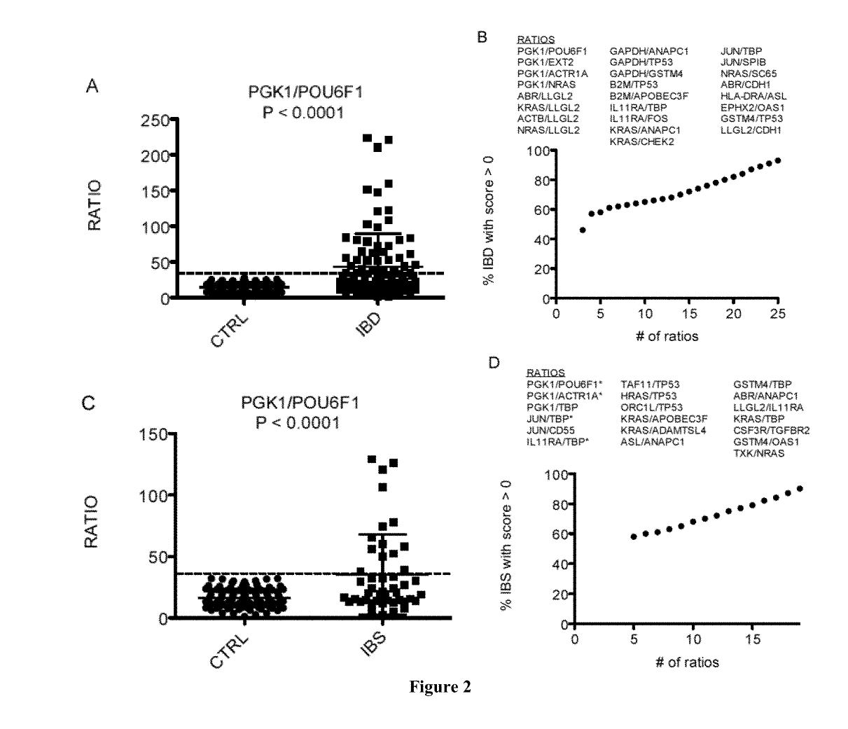

We applied this approach to determine how accurately it would distinguish subjects with IBD or IBS from CTRL. First, we identified ratios capable of discriminating IBD subjects from CTRL. Second, we applied a re-sampling permutation testing strategy to identify ratios that consistently displayed high discriminatory power. Third, we identified the smallest number of ratios producing the greatest discrimination between two comparator groups. The single ratio with the greatest discriminatory power was PGK1/POU6F1 (FIG. 2A). Using this ratio, 30% of IBD subjects achieved a ratioscore value higher than all CTRL subjects and were awarded one point. A combination of 25 ratios produced a scoring panel where 100% of CTRL subjects achieved a score of 0 and 94% of IBD subjects achieved a ratio≥1 (FIG. 2B). Thus, we conclude that gene-expression ratios we identified accurately distinguished IBD subjects from CTRL.

We continued our analysis to determine how well IBS and CTRL cohorts were differentiated. Interestingly, the optimum ratio that distinguished the IBD cohort from the CTRL cohort, PGK1/POU6F 1, was also the optimum ratio that distinguished the IBS cohort from the CTRL cohort (FIG. 2C). We identified a total of 19 ratios that, in combination, produced a point system whereby 100% of CTRL subjects achieved a score of 0 and 90% of IBS subjects achieved a ratio≥1 (FIG. 2D). Thus, even though IBS is generally considered not to be an inflammatory disease, we conclude our approach accurately distinguishes these subjects from the CTRL group.

IBS-IBD Discrimination Based Upon the Ratioscore Method

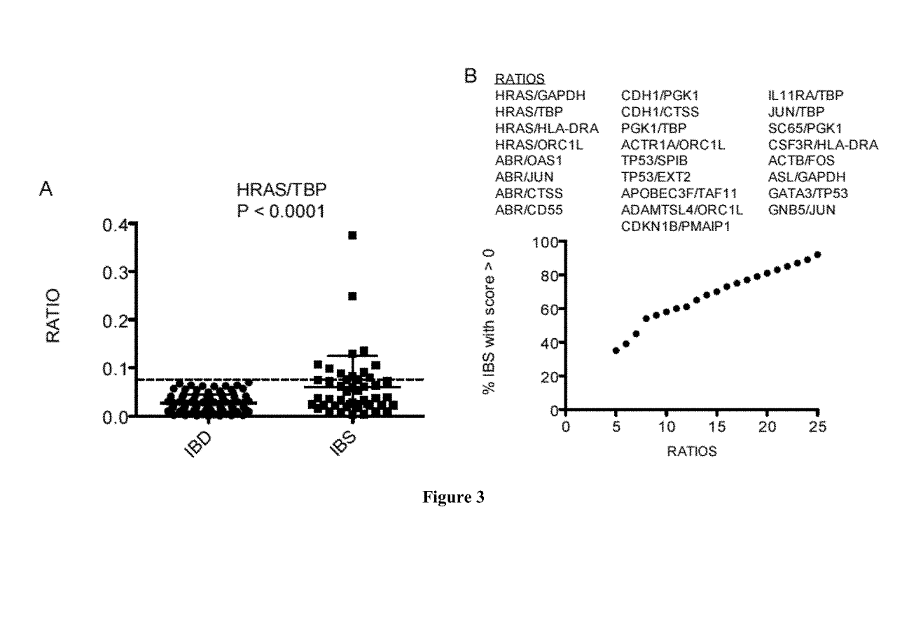

Next, we assessed our ability to distinguish IBS and IBD cohorts. The optimum ratio we identified was HRAS/TBP, p-value<0.0001 (FIG. 3A). We identified a total of 25 ratios that, combined, produced a ratioscore whereby 100% of IBD subjects achieved a score of 0 and 92% of IBS subjects were awarded a ratio≥1 (FIG. 3B). Thus, we conclude that the ratioscore method was capable of discriminating between subjects with IBD and subjects with IBS.

UC-CD Discrimination Disease Based Upon the Ratioscore Method

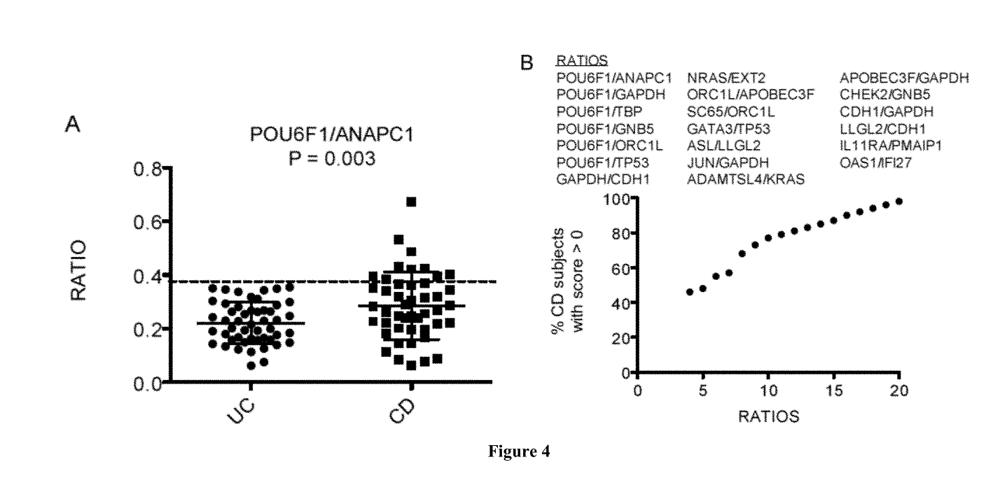

Finally, we determined if our approach accurately discriminated between the two inflammatory bowel diseases, UC and CD. The optimum ratio was POU6F1/ANAPC1, p-value=0.003 (FIG. 4A). We identified a total of 20 ratios that, in combination, produced a point system that awarded 100% of UC subjects a score of 0 and 98% of subjects with CD a ratio≥1 (FIG. 4B). Thus, the Ratioscore Method accurately discriminated between the two major subclasses: IBD:UC and IBD:CD.

Disease Discrimination Based Upon the SVM Method

Support Vector Machines (SVM) were also employed to classify the data into two distinct groups. The inputs for the SVM were the same ratios used to calculate the ratioscores. For example, when separating IBS patients from CTRL subjects, the same 19 ratios of normalized gene-expression ratios employed to compute the ratioscore were used as input to the SVM. In the SVM calculations, we chose the radial basis kernel (RBK) to perform the kernel trick. This kernel contains a fitting parameter β. We also used the “soft margin” approach to the fitting of the hyper-surface that separates the two groups (cases and controls). This introduced a second fitting parameter C. Programs written in Mathematica (Wolfram Research, Inc.) were created and random training subsets of the two groups were chosen to find the parameters, β and C. Each training subset consisted of 60% of the total dataset. The values of the two fitting parameters that produced the smallest number of incorrect cases and controls were used to define the SVM. This SVM analysis also accurately discriminated the different subject groups: (i) IBD and CTRL, (ii) IBS and CTRL, (iii) IBD and IBS, and (iv) CD and UC (Table 2).

TABLE 2
Case/Control discrimination by support vector machines (SVM #1)

| | | Training set | Case | | CTRL | |
|---|---|---|---|---|---|---|
| Comparison | Total # | % of total | TP # | FN # | TN # | FP # |
| IBD* vs. CTRL | 209 | 60 | 95 | 1 | 100 | 13 |
| IBD* vs. CTRL | 160 | 60 | 47 | 0 | 96 | 17 |
| IBD* vs. IBS | 143 | 60 | 45 | 2 | 86 | 10 |
| CD* vs. UC | 85 | 60 | 45 | 2 | 31 | 7 |

*Case cohort
^{†}TP = true positive, FN = false negative, TN = true negative, FP = false positive

A second SVM was also employed using LS-SVMLab software (http://www.esat.kuleuven.ac.be/sista/lssvmlab) to validate the SVM created with Mathematica. The procedure for training the SVM followed the following algorithm:
X(X=50%, 60%, and 80%) was randomly selected from the total set of data and used to train the SVM.
On the selected training set, L-fold cross-validation was performed. In this type of training a certain fraction of the training set was omitted from training and the remaining portion of the partial training set was used to estimate the parameters of the SVM. This was repeated L times. We used L=10. At the completion of the training, a composite estimate for the parameters was obtained.
Once the SVM was trained on X % of the total data, the SVM was applied to the total data set.

Numbers of correct and incorrect classifications were tabulated for total sets (training and validation), training sets and validation sets (Table 3). Overall accuracy in the training sets was greater than overall accuracy of the validation sets. The different training sessions did not produce much variation in the overall accuracy of the corresponding validation sets. Using the above algorithm, two different kernels, a polynomial kernel and Radial Basis Function (RBF) kernel, were used to create different machines. Overall, the SVM with the RBF kernels performed somewhat better than the polynomial kernels.

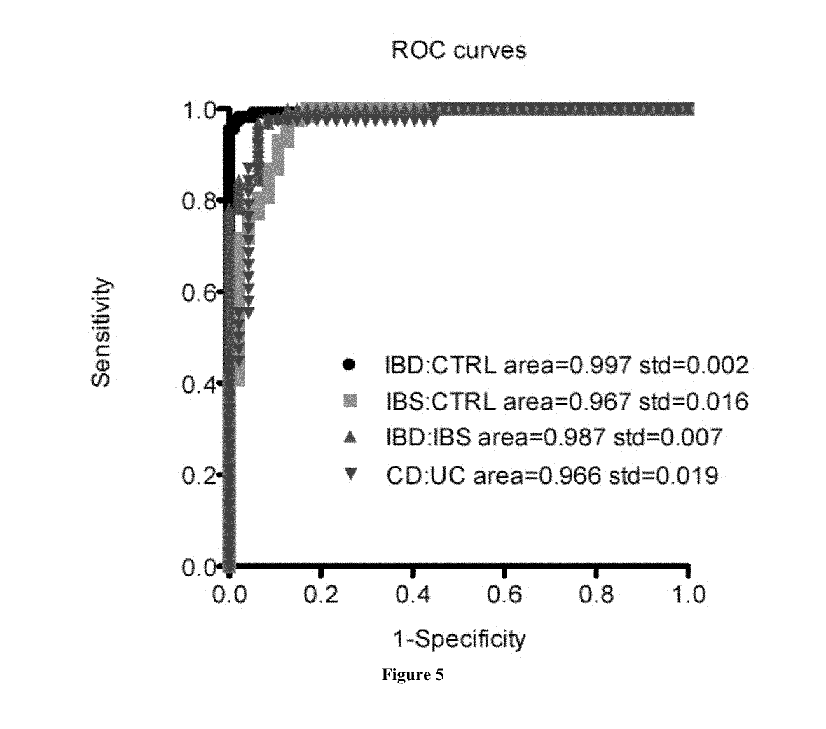

This second SVM was used to discriminate between the different subject groups, IBD and CTRL, IBS and CTRL, IBD and IBS, and CD and UC producing levels of sensitivity and specificity comparable to the Ratioscore Method or the first SVM method (Table 4). We determined receiver operating characteristic (ROC) curves from data produced by the second SVM method. The area-under-the-curve (AUC) for each comparison exceeded 0.96 (FIG. 5).

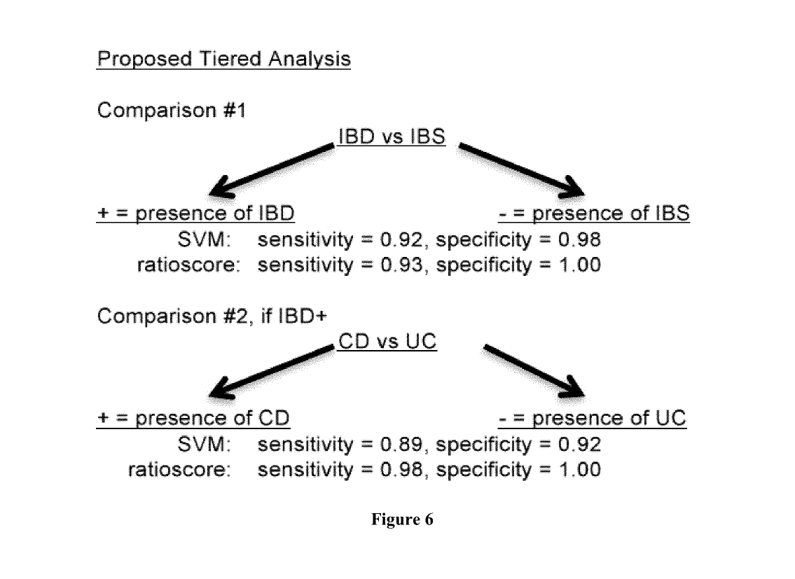

The IBD:CTRL comparison produced the greatest overall accuracy (AUC of 0.997). Thus, a tiered approach, using either ratioscore or SVM analysis, can be employed to segregate between IBD and IBS, first, followed by segregation between CD and UC if a subject is IBD positive. This approach produced high levels of sensitivity and specificity at both tiers of the analysis (FIG. 6).

TABLE 4
Sensitivity and specificity produced
by Ratioscore and two SVM methods

| | Method | | | | | |
|---|---|---|---|---|---|---|
| | Ratioscore | | SVM#1* | | SVM#2* | |
| | sensi- | speci- | sensi- | speci- | sensi- | speci- |
| | tivity | ficity | tivity | ficity | tivity | ficity |
| IBD vs. CTRL | 0.94 | 1.00 | 0.97 | 0.94 | 0.99 | 0.97 |
| IBS vs. CTRL | 0.91 | 1.00 | 1.00 | 0.68 | 0.85 | 0.99 |
| IBD vs. IBS | 0.93 | 1.00 | 0.97 | 0.91 | 0.92 | 0.98 |
| CD vs. UC | 0.98 | 1.00 | 0.94 | 0.85 | 0.89 | 0.92 |

*Training set = 80% of total,
**Training set = 60% of total
Sensitivity = # true positives/(# true positives + # false negatives)
Specificity = # true negatives/(# true negatives + # false positives)

In the above discussion, two support vector machines were independently created and trained using the ratios identified by the Ratioscore Method. There was little difference in the performance of the two machines when used to classify the different case-control combinations. One advantage of the SVM-based approach is that it can be used to classify more than two groups. As an example of classification into three groups, we considered data for UC (N=40), CD (N=46), and CTRL (N=113). Using gene ratios determined by comparing CTRL (controls) to UC+CD (cases), the SVM identified 99.8% of CTRL, 72.5% of UC, and 56.5% of the CD. Hence, the performance of the tertiary classification was not as accurate as the binary classifications. However, the tertiary classification was improved by using a different set of gene ratios, e.g., the union of the set from CTRL vs. CD, CTRL vs. UC, and CD vs. UC. In this case, the SVM identified 99.1% of CTRL, 100% of UC, and 84.8% of CD. One factor that may contribute to this increased accuracy is that the number of gene ratios used in the training of the SVM was increased from 23 ratios to 49 thus introducing additional parameters into the SVM structure.

Discussion

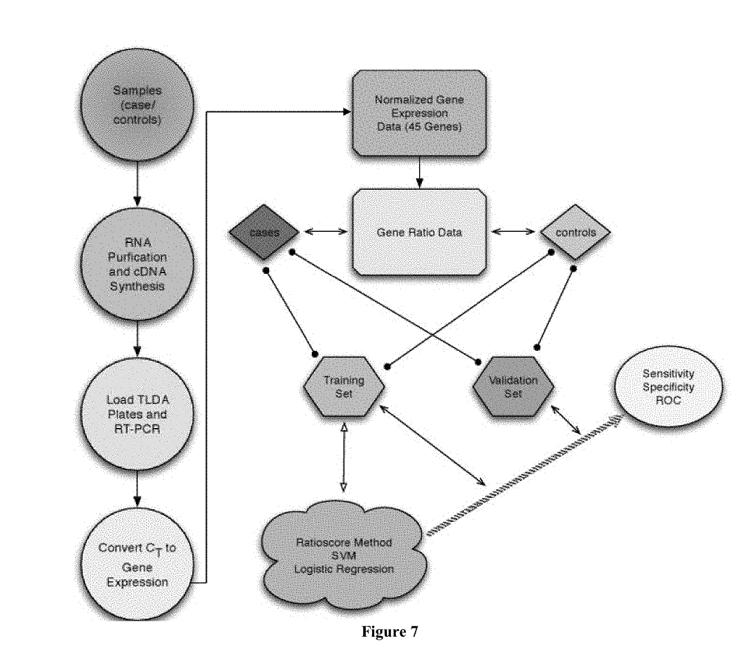

IBS and IBD can exhibit overlapping clinical symptoms making diagnosis difficult without invasive procedures [4,12,34]. Therapy and medication for IBS and IBD are vastly different and incorrect diagnosis and treatment plans have significant consequences. Differentiation between UC and CD can also be difficult, having important implications when considering medical and operative treatment options. For example, ASCA and p-ANCA have clinical utility in diagnosing IBD. ASCA IgA is found in 35-50% of patients with CD but <1% of patients with UC. ASCA IgG is found in 50-80% of patients with CD but only 20% of patients with UC. In contrast, atypical p-ANCA is found in 70% of UC patients but only 20% of CD patients [19]. Here, we describe a relatively non-invasive procedure capable of accurately discriminating between (a) IBS and IBD, and (b) the two forms of IBD, UC and CD, using three independent methods based upon transcript levels in blood of a discrete set of genes. Each method employs the same input, which are multiple ratios of expression levels of two genes. The analytic methods, ratioscore, two SVM methods, and logistic regression, produce similar levels of overall accuracy determined by ROC curves which exceed 95%. We have summarized the overall process of going from the raw samples to classification in FIG. 7.

In contrast, biomarkers for IBS are non-existent and diagnosis largely depends upon the absence of pathological findings in the colon. Previously identified experimental biomarkers to distinguish UC and CD clearly do not perform with the same degree of accuracy as experimental approaches described here. Thus, we propose these gene expression ratio tests using the Ratioscore Method, SVM, or logistic regression for analysis represent simple non-invasive tests that could accurately classify patients to IBS or IBD categories and IBD patients to UC or CD categories even without colonoscopy or sigmoidoscopy and tissue biopsy.

UC and CD are chronic inflammatory autoimmune diseases. Using various strategies, numerous studies have identified unique gene-expression signatures in blood or peripheral blood mononuclear cells (PBMC) associated with different autoimmune diseases [22]. Some are unique to a single autoimmune disease, some discriminate between two autoimmune diseases and some are shared among multiple autoimmune diseases. Thus perhaps it is not too surprising that we could employ a similar strategy to identify gene-expression signatures capable of discriminating the two forms of IBD, UC and CD, or IBD from CTRL or IBD from IBS.

Somewhat surprising is that IBS can be readily distinguished from CTRL. IBS is a disorder whose etiology and pathogenic mechanisms are incompletely understood [4]. Our results clearly demonstrate that IBS possesses an underlying gene-expression signature. One possibility is that IBS possesses an unrecognized mucosal pathology sensed by the immune system and expressed by changes in transcript levels of specific genes. Another possibility is that IBS generates expression of cytokines, chemokines, adhesion molecules, neurotransmitters or other mediators read by the immune system. In support of this notion, over-expression of PGK1 is associated with IBS, CeD, CD, and UC and PGK1 is known to be induced by hypoxia and may be induced by other forms of stress, inflammation or generalized mucosal irritation [35]. Further, ABR, ACTR1A, EXT2, HRAS, and KRAS are over-expressed in both IBS and CeD but not CD and UC. In contrast, APOBEC3F, ASL and SPIB are under-expressed in CD and UC, but not IBS and CeD. Thus, the IBS gene-expression signature is more similar to the CeD gene-expression signature and the UC signature is more similar to the CD signature. It is uncertain if this suggests that IBS may bear additional relationships to CeD. An improved understanding of mechanisms producing differences in levels of specific gene transcripts in IBS may further our understanding of the pathogenesis of IBS.

### Conclusions

Limitations to our study include selection of patients with pre-existing diagnoses of IBS and IBD, as this may not completely represent patients in the general population in whom these tests may be performed. However, in other studies we have shown that subjects with clinically isolated syndrome, a precursor of multiple sclerosis, who progress to a diagnosis of multiple sclerosis score positive in ratioscore- or SVM-based analyses, similar to those described here. This may suggest that subjects with initial clinical symptoms associated with IBD or IBS, CD or UC, may be discriminated by this approach. Future longitudinal approaches are planned to evaluate utility of these tests. Additional methods, such as analysis of gene-expression ratios in multi-dimensional space rather than binary space may improve the diagnostic capabilities of these tests. We employed three independent approaches to evaluate the ability of gene-expression ratios to discriminate subjects with gastro-intestinal diseases with overlapping clinical symptoms and each produced high degrees of specificity and sensitivity. Thus, these minimally invasive tests may assist in excluding or establishing a diagnosis of IBS or IBD, CD or UC.

Throughout this document, various references are mentioned. All such references are incorporated herein by reference, including the references set forth in the following list:

### REFERENCES

1. Vasiliauskas E: Recent advances in the diagnosis and classification of inflammatory bowel disease. Curr Gastroenterol Rep 2003, 5:493-500.
2. Loftus E V, Sandborn E J: Epidemiology of inflammatory bowel disease. Gastroenterol Clin North Am 2002, 31:1-20.
3. Ray S, Britschgi M, Herbert C, et al: Classification and prediction of clinical Alzheimer's diagnosis based on plasma signaling proteins. Nature Med 2007, 13:1359-1362.
4. Torpy J M, Golub R M: JAMA patient page. Irritable bowel syndrome. JAMA 2011, 306:1501.
5. Schoepfer A M, Trummler M, Seeholzer P, et al: Discriminating IBD from IBS: comparison of the test performance of fecal markers, blood leukocytes, CRP, and IBD antibodies. Inflamm Bowel Dis 2008, 14:32-39.
6. Hammerle C W, Crowe S E: When to reconsider the diagnosis of irritable bowel syndrome. Gastroenterol Clin North Am 2011, 40:291-307. vii.
7. Geboes K, Colombel J F, Greenstein A, et al: Indeterminate colitis: a review of the concept—what's in a name? Inflamm Bowel Dis 2008, 14:850-857.
8. Tekkis P P, Heriot A G, Smith O, et al: Long-term outcomes of restorative proctocolectomy for Crohn's disease and indeterminate colitis. Colorectal Dis 2005, 7:218-223.
9. Landers C J, Cohavy O, Misra R, et al: Selected loss of tolerance evidenced by Crohn's disease-associated immune responses to auto- and microbial antigens. Gastroenterology 2002, 123:689-699.
10. Targan S R, Landers C J, Yang H, et al: Antibodies to CBir1 flagellin define a unique response that is associated independently with complicated Crohn's disease. Gastroenterology 2005, 128:2020-2028.
11. Hui T, Landers C, Vasiliauskas E, et al: Serologic responses in indeterminate colitis patients before ileal pouch-anal anastomosis may determine those at risk for continuous pouch inflammation. Dis Colon Rectum 2005, 48:1254-1262.
12. Tamboli C P, Doman D B, Patel A: Current and future role of biomarkers in Crohn's disease risk assessment and treatment. Clin Exp Gastroenterol 2001, 4:127-140.
13. Barrett J C, Hansoul S, Nicolae D, et al: Genome-wide association defines more than 30 distinct susceptibility loci for Crohn's disease. Nat Genet 2008, 40:955-962.
14. Burczynski M E, Dorner A J: Transcriptional profiling of peripheral blood cells in clinical pharmacogenomic studies. Pharmacogenomics 2006, 7:187-202.
15. Burczynski M E, Peterson R L, Twine N C, et al: Molecular classification of Crohn's disease and ulcerative colitis patients using transcriptional profiles in peripheral blood mononuclear cells. J Mol Diagn 2006, 8:51-61.
16. Franke A, Balschun T, Sina C, et al: Genome-wide association study for ulcerative colitis identifies risk loci at 7q22 and 22q13 (IL17REL). Nat Genet 2010, 42:292-294.
17. Harris V K, Sadiq S A: Disease biomarkers: Potential for use in therapeutic decision making. Mol Diagn Ther 2009, 13:225-244.
18. Hugot J P, Chamaillard M, Zouali H, et al: Association of NOD2 leucine-rich repeat variants with susceptibility to Crohn's disease. Nature 2001, 411:599-603.
19. Jaskowski T D, Litwin C M, Hill H R: Analysis of serum antibodies in patients suspected of having inflammatory bowel disease. Clin Vaccine Immunol 2006, 13:655-660.
20. Maas K, Chan S, Parker J, et al: Cutting edge: molecular portrait of human autoimmune disease. J Immunol 2002, 169:5-9.
21. Mannick E E, Bonomolo J C, Horswell R, et al: Gene expression in mononuclear cells from patients with inflammatory bowel disease. Clin Immunol 2004,112:247-257.
22. Pascual V, Chaussabel D, Banchereau J: A genomic approach to human autoimmune diseases. Annu Rev Immunol 2010, 28:535-571.
23. Quackenbush J: Microarray Analysis and Tumor Classification. N Engl J Med 2006, 354:2463-2472.
24. Quintana F J, Farez M F, Viglietta V, et al: Antigen microarrays identify unique serum autoantibody signatures in clinical and pathologic subtypes of multiple sclerosis. Proc Natl Acad Sci USA 2008, 105:18889-18894.
25. Fossey S C, Vnencak-Jones C L, Olsen N J, et al: Identification of molecular biomarkers for multiple sclerosis. J Mol Diagn 2007, 9:197-204.
26. Tossberg J T, Crooke P S, Henderson M A, et al: Gene-expression signatures: biomarkers toward diagnosing multiple sclerosis. Genes Immun 2012, 13:146-154.
27. Abdi H: The Bonferonni and Sidak corrections for multiple comparisons. Sage; 2007:1-9.
28. Ochs R L, Stein T W, Chan E K, et al: cDNA cloning and characterization of a novel nucleolar protein. Mol Biol Cell 1996, 7:1015-1024.
29. Pines J: Cubism and the cell cycle: the many faces of the APC/C. Nat Rev Mol Cell Biol 2011, 12:427-438.
30. Moshe Y, Bar-On O, Ganoth D, et al: Regulation of the action of early mitotic inhibitor 1 on the anaphase-promoting complex/cyclosome by cyclin-dependent kinases. J Biol Chem 2011, 286:16647-16657.
31. Arnaoutov A, Dasso M: The Ran GTPase regulates kinetochore function. Dev Cell 2003, 5:99-111.
32. Qiao X, Pham D N, Luo H, et al: Ran overexpression leads to diminished T cell responses and selectively modulates nuclear levels of c-Jun and c-Fos. J Biol Chem 2010, 285:5488-5496.
33. Quimby B B, Dasso M: The small GTPase Ran: interpreting the signs. Curr Opin Cell Biol 2003, 15:338-344.
34. Spiller R C: Irritable bowel syndrome: gender, infection, lifestyle or what else. Dig Dis 2011, 29:215-221.
35. Lam W, Leung C-H, Bussom S, et al: The impact of hypoxic treatment on the expression of phosphogycerate kinase and the cytotoxicity of troxacitabine and gemcitabine. Mol Pharm 2007, 72:536-544.

It will be understood that various details of the presently disclosed subject matter can be changed without departing from the scope of the subject matter disclosed herein. Furthermore, the foregoing description is for the purpose of illustration only, and not for the purpose of limitation.

### SEQUENCES

The following are complementary DNA (cDNA) sequences of genes-of-interest identified in Table A. The portion of the sequences bolded and underlined are Applied BioSystems context sequences, the region of that can be amplified in some embodiments of the presently-disclosed subject matter. ABI assay numbers for the sequences are provided in Table A.

SEQ ID NO: 1
Homo sapiens active BCR-related gene (ABR), transcript variant 3, mRNA
GGACTGCAGAGGGAACTTGCCTTGAAGAGGCCTGGTCCTTAAAGAGACACAGCACACACGGCCCGACCGG
CAGCCCCAGAGCAGAGGCTCCACTGATGGCAGGCGCCCCTGGCTAGGCTCTGAGGTTCCTTTGCCCTCGC
CTTGCTGAATGGTGAGCCGCTGCCTCTCGGAGCCCGTCTCCTTGACAGCCTGCCCTCGGCTCCTGCAGCC
ACTCCTGGGCCTGATGGGGACAGGGCCAGCCTGGTGGGTGGTGTCAGAGGTCCTGGCAGAGCAGCGTAGG
CCTGGGATGCGTCTGCAGAATTCTGGCTGAACGAGCGAGGAGCACGGCCAGCTTCGGGGCCGTCGTGACC
ACAGGAGGGCAGAGGGCCAGCCCGTGAGCTCTGACCCCAGCTGGACGTGCTCTTGTTTCCCTTGGGGCTA
AGGAGATTGGAGCCACTGAACTGAATCTCTGGGTTTTGGAGACTTAGAGAATCCATTGGACTCTTCTGCT
GGCGTCTTTCTGAATGCTGATGGGGACTTGGTGACTTCAGCTACGGGACGGACGAGTACGACGGAGAGGG
GAATGAGGAGCAGAAGGGGCCCCCGGAGGGCTCAGAGACCATGCCGTACATCGATGAGTCGCCCACCATG
TCCCCGCAGCTCAGCGCCCGCAGCCAGGGCGGGGGGGATGGCGTCTCCCCGACTCCACCTGAGGGACTGG
CTCCTGGGGTGGAAGCAGGGAAAGGCCTGGAGATGAGGAAGCTGGTTCTCTCGGGGTTCTTGGCCAGCGA
AGAGATCTACATTAACCAGCTGGAAGCCCTGTTGCTGCCCATGAAACCCCTGAAGGCCACCGCCACCACC
TCCCAGCCCGTGCTCACCATCCAGCAGATCGAGACCATCTTCTACAAGATCCAGGACATCTATGAGATCC
ACAAGGAGTTCTATGACAACCTGTGCCCCAAGGTGCAACAGTGGGACAGCCAGGTCACCATGGGCCACCT
CTTCCAGAAGCTGGCCAGCCAGCTCGGTGTGTACAAAGCGTTTGTCGATAACTATAAAGTCGCTCTGGAG
ACAGCTGAGAAGTGCAGCCAGTCCAACAACCAGTTCCAGAAGATCTCAGAGGAACTCAAAGTGAAAGGTC
CCAAGGACTCCAAGGACAGCCACACGTCTGTCACCATGGAAGCTCTGCTCTACAAGCCCATTGACCGGGT
CACTCGGAGCACCCTAGTCCTACACGACCTGCTGAAGCACACACCTGTGGACCACCCCGACTACCCGCTG
CTGCAGGATGCCCTCCGCATCTCCCAGAACTTCCTGTCCAGCATCAACGAGGACATCGACCCCCGCCGGA
CTGCAGTGACAACGCCCAAGGGGGAGACGCGACAGCTGGTGAAGGACGGCTTCCTGGTGGAAGTGTCAGA
GAGCTCCCGGAAGCTGCGGCACGTCTTCCTCTTTACAGATGTCCTACTGTGTGCCAAGCTGAAGAAGACC
TCTGCAGGGAAGCACCAGCAGTATGACTGTAAGTGGTACATCCCCCTGGCCGACCTGGTGTTTCCATCCC
CCGAGGAGTCTGAGGCCAGCCCCCAGGTGCACCCCTTCCCAGACCATGAGCTGGAGGACATGAAGATGAA
GATCTCTGCCCTCAAGAGTGAAATCCAGAAGGAGAAAGCCAACAAAGGCCAGAGCCGGGCCATCGAGCGC
CTGAAGAAGAAGATGTTTGAGAATGAGTTCCTGCTGCTGCTCAACTCCCCCACAATCCCGTTCAGGATCC
ACAATCGGAATGGAAAGAGTTACCTGTTCCTACTGTCCTCGGACTACGAGAGGTCAGAGTGGAGAGAAGC
AATTCAGAAACTACAGAAGAAGGATCTCCAGGCCTTTGTCCTGAGCTCAGTGGAGCTCCAGGTGCTCACA
GGATCCTGTTTCAAGCTTAGGACTGTACACAACATTCCTGTCACCAGCAATAAAGACGACGATGAGTCTC
CAGGACTCTATGGCTTCCTTCATGTCATCGTCCACTCTGCCAAGGGATTTAAGCAATCAGCCAACCTGTA
CTGTACCCTGGAGGTGGATTCCTTCGGCTATTTTGTCAGCAAAGCCAAAACCAGGGTGTTCCGGGACACA
GCGGAGCCCAAGTGGGATGAGGAGTTTGAGATCGAGCTGGAGGGCTCCCAGTCCCTGAGGATCCTGTGCT
ATGAGAAGTGCTATGACAAGACCAAGGTCAACAAGGACAACAATGAGATCGTGGACAAGATCATGGGCAA
AGGACAGATCCAGCTGGACCCACAAACCGTGGAGACCAAGAACTGGCACACGGACGTGATTGAGATGAAC
GGGATCAAAGTGGAATTTTCCATGAAATTCACCAGCCGAGATATGAGCCTGAAGAGGACCCCGTCCAAAA
AGCAGACCGGCGTCTTCGGTGTGAAGATCAGCGTGGTGACGAAGCGGGAGCGCTCCAAGGTGCCCTACAT
CGTCCGGCAGTGTGTGGAGGAGGTGGAGAAGAGGGGTATCGAGGAGGTTGGCATCTACAGGATATCGGGC
GTGGCCACGGACATCCAGGCGCTCAAGGCCGTCTTCGATGCCAATAACAAGGACATCCTGCTGATGCTGA
GTGACATGGACATCAACGCCATCGCCGGGACGCTCAAGCTGTACTTCCGGGAACTGCCCGAGCCGCTCCT
CACGGACCGACTCTACCCAGCCTTCATGGAGGGCATCGCCCTGTCAGACCCTGCTGCCAAGGAAAACTGC
ATGATGCACCTGCTCCGCTCCCTGCCCGACCCCAACCTCATCACCTTCCTCTTCCTGCTGGAACACTTGA
AAAGGGTTGCCGAGAAGGAGCCCATCAACAAAATGTCACTTCACAACCTGGCTACCGTGTTTGGACCCAC
GTTACTGAGACCCTCAGAAGTGGAGAGCAAAGCACACCTCACCTCGGCTGCGGACATCTGGTCCCATGAC
GTCATGGCGCAGGTCCAGGTCCTCCTCTACTACCTGCAGCACCCCCCCATTTCCTTCGCAGAACTCAAGC
GGAACACACTGTACTTCTCCACCGACGTGTAGCCCGAGGCAGGGTGGCTGCGGGCGGGTGGTGGAACCAG
CCCCTCCAGCCTGGGGTCCAACTCAGACTTGAAAGACTGCAATAGAAAACTCCCAAACCCAGCACTCCAG
ACTCGAGGGAAGCCAGCTTCCAAGAACTGGAATGCGTACGTCTTTTGTGCCACCTTGTACAAAGCCGGCT
GCCCAGCCCCAGCCTCACCACCGCATCCCACCTCCTGCCCTCCATACCTCTAGTTGTGTCTGATGCTCCG
TGCTGTTCGGGAATTGTTTTATGTACACTTGTCAGGCAGAAAAGGTAGTGACCGGCCCGGCGTGGGCACA
CAGACAGCCCGCTTTGTTCTTTCATTTCCTCCAGCACTTTCTTTCCGCCTGAGTCCAGCCCAAGGCCTTT
TATTTTGCGCTGTGTAACTGCTGCCAGCTTCTCTCTTGGCCCTGCTCCCAGATGGCGGTCTCCTGGCAGC
CTCCCCTCAGTCTTCCTCCACCCGCTCTTCCTTCCCAGCCTGCCTGCATGCATGTGCACCCTTGGTCTTC
GCTCCATCGCCTTGAAAGCTCTGAAGAGGCCCTGGGTTGCCGCGGCAGCAGTGGTCTGTTTGATGCTGCC
GTTTGCCGCTGCCGGCCCCTCCTCAGACTCCGCCTTTGGGAGCACACCTGCTTTGCCTTGCTGCCTGTGC
AAATGTTGGACAAGCAGACACACTCACACTCGTCCCCAGCTTAGCACAGAGCTGGAGCGCCCATTTCTGG
AATTTTCCGTTTGGGAATCTCCACTTCTGGGGTTTACCTGTTCGGCCTCCTGTCTATCAGTGAGGCATCT
CTGACTGTTTCTTCTACTGCTTTTCAGTTCCCTTCCCTGCTGTTCTATTTCCTTTGAGTGTAAAGACTCA
CAGGTGACCTGCTATCGAGATAGCCAGAGGGTCAGGAGAGAATGGGGGAGGAGGCGGTCAGGCTGCTGAG
GAAACACCACAGGCTGAACGGGGGAGGAATGCACATGCCACGCTGGGTGTCCCGGGTCGCGGGGAGGCAG
CTCAGCTCTTAGGAGCAAGTTGTGGGGGCTTTTCAAGAGGGGCCAGGCTTCCTGGAGGGTGACTGATGTG
GCCGAAGCAGGTGTCCAGGCAGGTAGGCTGCAGCCAGGAGCTCCCTGGCACCGCAGGACCTCGTGGTACT
CTTGCCTTAGATTTTACACACACTCCACAGCCAAGCACTGCCACGGTCCTCCAGGACCTGGGAAGCAAAG
GCACAGGCCCACGGTGGCCAGCCATTGTGGTGCCGCCCCAGCTTCTGGATACAGCCTTTTGGGTAAACAC
TGGGAACTCCAGAAGTTGTGGGGAGAGTGGGGAATCAGACAGCCGCCTCTAGGGGCTGGGTTCTGCTGGG
GCCTCCTTGTTGGTGCTGTAGGCACCCGCCAGGGAGCAGGGACCCGACTTGCAGACGCATTGCCCGGTAC
TAGGAAGGAGTGAGGTGTGTTCCCACCGTACACTTCCCACACGAGCTGCGGCTGCCAGCCTCGGGCCATC
AGCCTAGGAGAGCAGATGCAGCTCCAGGGGCTCGACTTATAGCCAGTTACAGCTCCCCGGCTCTTCTGTG
TGGCAGAGCGTCGTTTCCGGGCCCTCAGGGCTGGGGAGCTCAGTTCCCATTGCTTGTGCTCAGGGCTGAG
TCTTAAAGAAGGGTTTGCCGGCCCTAACGCTGCAGCGCGTGCGCGGTGAGAGGCCCTTTTTGAGCCTGTT
TACTCCTGTGGCCTTGGGCAGAACAGTAAATACTCTGTGCACGGAGGAAAGACATGCCCAAGAGGAAGGA
AGTACTGACCATCGGCTGCCTGTGAGCAGCTTAGCAAGGAGCCCTTGCTCCCTGGGAAAGGCGGTGAACT
TGAGTCTAAAGATGCAGTGCCTGGCCCTTCCTAAGGTCCCTGCCTGGCATCCGAGTGTCGGTGTGTGGCA
CAGAAGGCTCCTGCTTGCTTCCAAAGTGATGGACAGGAAGGGGCAGAGTGAGTCACGGCCCAGACTGGGC
ACCTTCGCGTCTCAGCCTCAGGGAGCCCCACAGCCCCAAGCTCGCTGAGGCAACGTGAGAACAGGCTATG
GGAAGGCTGCAAAGGCTGAGAAATGCAAAGGCTCATATTTATAAATCCCACCCCCAGAGTGGGGAGGGTC
AGGTGCCAGACCTGGACTAAACTGCACCAAGGAAACACCCAGCAGGGTCTCCTGTGAGCCGGGGACCATG
CAGCCCGAAACCTCCAGTCACTGCGCCCGGCAGGAGTCAGGAGCCAGGGACTGTGCAGCCTGGAACCTCC
AGTCACTGTGCCCAGCAGGGTGGGCTGTGCCCAGCAGGAGTCAGGCTAAGAAACGCCAGGTCTGCCTGTT
CTTGCTGGGCAATGGCTGATGGCTGCCAGTTTCTGCTGATACACAGGTAGGATGGGACCCTTCATGAATA
TCTGACTTTAATAAGTTGGTAAGGATATATTTTTTTGTCTATGTTCTGTTTCAACTTATGTAGATTATTA
TAAATTGATGTAAACCACGTGAGAGGAAAATGTTAATAAAAAATGCAAAGCCCCATCATTTGCACAAAAC
TCA
SEQ ID NO: 2
Homo sapiens actin, beta (ACTB), mRNA
ACCGCCGAGACCGCGTCCGCCCCGCGAGCACAGAGCCTCGCCTTTGCCGATCCGCCGCCCGTCCACACCC
GCCGCCAGCTCACCATGGATGATGATATCGCCGCGCTCGTCGTCGACAACGGCTCCGGCATGTGCAAGGC
CGGCTTCGCGGGCGACGATGCCCCCCGGGCCGTCTTCCCCTCCATCGTGGGGCGCCCCAGGCACCAGGGC
GTGATGGTGGGCATGGGTCAGAAGGATTCCTATGTGGGCGACGAGGCCCAGAGCAAGAGAGGCATCCTCA
CCCTGAAGTACCCCATCGAGCACGGCATCGTCACCAACTGGGACGACATGGAGAAAATCTGGCACCACAC
CTTCTACAATGAGCTGCGTGTGGCTCCCGAGGAGCACCCCGTGCTGCTGACCGAGGCCCCCCTGAACCCC
AAGGCCAACCGCGAGAAGATGACCCAGATCATGTTTGAGACCTTCAACACCCCAGCCATGTACGTTGCTA
TCCAGGCTGTGCTATCCCTGTACGCCTCTGGCCGTACCACTGGCATCGTGATGGACTCCGGTGACGGGGT
CACCCACACTGTGCCCATCTACGAGGGGTATGCCCTCCCCCATGCCATCCTGCGTCTGGACCTGGCTGGC
CGGGACCTGACTGACTACCTCATGAAGATCCTCACCGAGCGCGGCTACAGCTTCACCACCACGGCCGAGC
GGGAAATCGTGCGTGACATTAAGGAGAAGCTGTGCTACGTCGCCCTGGACTTCGAGCAAGAGATGGCCAC
GGCTGCTTCCAGCTCCTCCCTGGAGAAGAGCTACGAGCTGCCTGACGGCCAGGTCATCACCATTGGCAAT
GAGCGGTTCCGCTGCCCTGAGGCACTCTTCCAGCCTTCCTTCCTGGGCATGGAGTCCTGTGGCATCCACG
AAACTACCTTCAACTCCATCATGAAGTGTGACGTGGACATCCGCAAAGACCTGTACGCCAACACAGTGCT
GTCTGGCGGCACCACCATGTACCCTGGCATTGCCGACAGGATGCAGAAGGAGATCACTGCCCTGGCACCC
AGCACAATGAAGATCAAGATCATTGCTCCTCCTGAGCGCAAGTACTCCGTGTGGATCGGCGGCTCCATCC
TGGCCTCGCTGTCCACCTTCCAGCAGATGTGGATCAGCAAGCAGGAGTATGACGAGTCCGGCCCCTCCAT
CGTCCACCGCAAATGCTTCTAGGCGGACTATGACTTAGTTGCGTTACACCCTTTCTTGACAAAACCTAAC
TTGCGCAGAAAACAAGATGAGATTGGCATGGCTTTATTTGTTTTTTTTGTTTTGTTTTGGTTTTTTTTTT
TTTTTTGGCTTGACTCAGGATTTAAAAACTGGAACGGTGAAGGTGACAGCAGTCGGTTGGAGCGAGCATC
CCCCAAAGTTCACAATGTGGCCGAGGACTTTGATTGCACATTGTTGTTTTTTTAATAGTCATTCCAAATA
TGAGATGCGTTGTTACAGGAAGTCCCTTGCCATCCTAAAAGCCACCCCACTTCTCTCTAAGGAGAATGGC
CCAGTCCTCTCCCAAGTCCACACAGGGGAGGTGATAGCATTGCTTTCGTGTAAATTATGTAATGCAAAAT
TTTTTTAATCTTCGCCTTAATACTTTTTTATTTTGTTTTATTTTGAATGATGAGCCTTCGTGCCCCCCCT
TCCCCCTTTTTTGTCCCCCAACTTGAGATGTATGAAGGCTTTTGGTCTCCCTGGGAGTGGGTGGAGGCAG
CCAGGGCTTACCTGTACACTGACTTGAGACCAGTTGAATAAAAGTGCACACCTTAAAAATGAAAAAAAAA
AAAAAAAAAAAAAAAAAAAAAAAAAAAAAAAA
SEQ ID NO: 3
Homo sapiens ARP1 actin-related protein 1 homolog A, centractin alpha
(yeast) (ACTR1A), mRNA
GCTCCCTCGCCGCCCTGAACCGGCGGCTAGACTGCGCATGCGTGTCAGTGGCGCTAGCGGCGGACCCGGC
TGGGCAGTTCCTTCCCCAGAAGGAGAGATTCCTCTGCCATGGAGTCCTACGATGTGATCGCCAACCAGCC
TGTCGTGATCGACAACGGATCCGGTGTGATTAAAGCTGGTTTTGCTGGTGATCAGATCCCCAAATACTGC
TTTCCAAACTATGTGGGCCGACCCAAGCACGTTCGTGTCATGGCAGGAGCCCTTGAAGGCGACATCTTCA
TTGGCCCCAAAGCTGAGGAGCACCGAGGGCTGCTTTCAATCCGCTATCCCATGGAGCATGGCATCGTCAA
GGATTGGAACGACATGGAACGCATTTGGCAATATGTCTATTCTAAGGACCAGCTGCAGACTTTCTCAGAG
GAGCATCCTGTGCTCCTGACTGAGGCGCCTTTAAACCCACGAAAAAACCGGGAACGAGCTGCCGAAGTTT
TCTTCGAGACCTTCAATGTGCCCGCTCTTTTCATCTCCATGCAAGCTGTACTCAGCCTTTACGCTACAGG
CAGGACCACAGGGGTGGTGCTGGATTCTGGGGATGGAGTCACCCATGCTGTGCCCATCTATGAGGGCTTT
GCCATGCCCCACTCCATCATGCGCATCGACATCGCGGGCCGGGACGTCTCTCGCTTCCTGCGCCTCTACC
TGCGTAAGGAGGGCTACGACTTCCACTCATCCTCTGAGTTTGAGATTGTCAAGGCCATAAAAGAAAGAGC
CTGTTACCTATCCATAAACCCCCAAAAGGATGAGACGCTAGAGACAGAGAAAGCTCAGTACTACCTGCCT
GATGGCAGCACCATTGAGATTGGTCCTTCCCGATTCCGGGCCCCTGAGTTGCTCTTCAGGCCAGATTTGA
TTGGAGAGGAGAGTGAAGGCATCCACGAGGTCCTGGTGTTCGCCATTCAGAAGTCAGACATGGACCTGCG
GCGCACGCTTTTCTCTAACATTGTCCTCTCAGGAGGCTCTACCCTGTTCAAAGGTTTTGGTGACAGGCTC
CTGAGTGAAGTGAAGAAACTAGCTCCAAAAGATGTGAAGATCAGGATATCTGCACCTCAGGAGAGACTGT
ATTCCACGTGGATTGGGGGCTCCATCCTTGCCTCCCTGGACACCTTTAAGAAGATGTGGGTCTCCAAAAA
GGAATATGAGGAAGACGGTGCCCGATCCATCCACAGAAAAACCTTCTAATGTCGGGACATCATCTTCACC
TCTCTCTGAAGTTAACTCCACTTTAAAACTCGCTTTCTTGAGTCGGAGTGTTTGCGAGGAACTGCCTGTG
TGTGAGTGCGTGTGTGGATATGAGTGTGTGTGCACATGCGAGTGCCGTGTGGCCCTGGGACCCTGGGCCC
AGAAAGGACGATGAACTACCTGCAGTGGTGATGGCCTGAGGCCTGGGGTTGACCACTAACTGGCTCCTGA
CAGGGAAGAGCGCTGGCAGAGGCTGTGCTCCCTCCTCAGGTGGCCTCTGGCTGGCTGTGGGGGACTCCGT
TTACTACCACAGGGAGACAGAGGGAGGTAAGCCATCCCCCGGGAGACCTTGCTGCTGACCATCCTAGGCT
GGGCTGGCCCCACCCTCACCCCCACCCCCAGGGTGCCCTGAGGCCCCAGGCAGCTGCTGCCTCCACTATC
GATGCCTCCTGACTGCACACTGAGGACTGGGACTGGGGTTGAGTTCTGTCTGGTTTTGTTGCCATTTTGG
TTTGGGAGGCTGGAAAAGCACCCCAAGAGCTATTACAGAGACTGGAGTCAGGAGAGAGCAGGAGGCCCTC
ATGTTCACCAGGGAACAGGACCACACCGGCCACTGGAGGAGGGCAGGAGCAGTCCTCACTCTGAATGGCT
GCAGAGTTAATGTTCCCAGCCCAGTCCCCTTTCGGGGGCCTTGGGAGAGTTTAAGGCACCTGCTGGTTCC
AGGACCTCGCTTTCCATCTGTTCTTGTTGCAATGCCATCTTCAAACCGTTTTATTTATTGAAGTGTTTGT
TCAGTTAGGGGCTGGAGAGAGGGAGCTTGCTGCCTCCTGCCTTGCTACACTAATGTTTACAGCACCTAAG
CTTAGCCTCCAGGGCCCCACCTCTCCCAGCTGATGGTGAGCTGACAGTGTCCACAGGTTCCAGGACCATT
TGAGATTGGAAGCTACACTCAAAGACACTCCCACCAGGCTCTTTCTCCCTTTTCCTCTTGCTCACTGCCC
TGGAATCAACAGGCTGGTTGCTGGTTAGATTTTCTGAAACAGGAGGTAAAATTTTTCTTTGGCAGAGGCC
CCTAAGCAAGGGAGGGGTGTTGGAGAGCCAGTGCCCTTAAGACTGGAGAAAGCTGCAATTTACCAAGTTG
CCTTTTGCCACTGTAGCTGACCAGGGGACTAGGTTGTAGAGGTGGGAAGGCCCCCTCTGGGCTGATCTTG
TGCCATTCTTGACCTTGGACCTGCTTGGTTAAGGAGGGAGTGGGCCAGACCAGAGTGCCAGGAGCTAATG
GAGCCAGGCCTGACTCCTAGGAGTGGTCCAAAGGCCTTCAGCCTAGATGGTGCAAAGCTGGGGCCAGCCT
GTCTTCACCGGCACCCTCACCTGTGACACCAAGACCCACCCCAATCCCAGACTTCACACAGTATTCTCCC
CCACGCCGTCCTATGACCAAAGGCCCCTGCCAGGTGTGGGCCACAGCAGCAGGTATGTGTGAAAGCAACG
TAGCGCCCCGCGGACTGCAGTGCGCTTAACCAACTCACCTCCCTTCTCTTAGCCCAAGCCTGTCCCTCGC
ACAGCCTCGCACAAACCACATTGCCTGGTGGGGCCCAGTGTACTGAAATAAAGTCGTTCCGATAGACACG
TCAAAAAAAAAAAAAAAAAAA
SEQ ID NO: 4
Homo sapiens ADAMTS-like 4 (ADAMTSL4), transcript variant 1, mRNA
CCGCCGCGGAGCGAGGTTGCCTGGAGAGAGCGCCTGGGCGCAGAAGGGTTAACGGGCCACCGGGGGCTCG
CAGAGCAGGAGGGTGCTCTCGGACGGTGTGTCCCCCACTGCACTCCTGAACTTGGAGGACAGGGTCGCCG
CGAGGGACGCAGAGAGCACCCTCCACGCCCAGATGCCTGCGTAGTTTTTGTGACCAGTCCGCTCCTGCCT
CCCCCTGGGGCAGTAGAGGGGGAGCGATGGAGAACTGGACTGGCAGGCCCTGGCTGTATCTGCTGCTGCT
TCTGTCCCTCCCTCAGCTCTGCTTGGATCAGGAGGTGTTGTCCGGACACTCTCTTCAGACACCTACAGAG
GAGGGCCAGGGCCCCGAAGGTGTCTGGGGACCTTGGGTCCAGTGGGCCTCTTGCTCCCAGCCCTGCGGGG
TGGGGGTGCAGCGCAGGAGCCGGACATGTCAGCTCCCTACAGTGCAGCTCCACCCGAGTCTGCCCCTCCC
TCCCCGGCCCCCAAGACATCCAGAAGCCCTCCTCCCCCGGGGCCAGGGTCCCAGACCCCAGACTTCTCCA
GAAACCCTCCCCTTGTACAGGACACAGTCTCGGGGAAGGGGTGGCCCACTTCGAGGTCCCGCTTCCCACC
TAGGGAGAGAGGAGACCCAGGAGATTCGAGCGGCCAGGAGGTCCCGGCTTCGAGACCCCATCAAGCCAGG
AATGTTCGGTTATGGGAGAGTGCCCTTTGCATTGCCACTGCACCGGAACCGCAGGCACCCTCGGAGCCCA
CCCAGATCTGAGCTGTCCCTGATCTCTTCTAGAGGGGAAGAGGCTATTCCGTCCCCTACTCCAAGAGCAG
AGCCATTCTCCGCAAACGGCAGCCCCCAAACTGAGCTCCCTCCCACAGAACTGTCTGTCCACACCCCATC
CCCCCAAGCAGAACCTCTAAGCCCTGAAACTGCTCAGACAGAGGTGGCCCCCAGAACCAGGCCTGCCCCC
CTACGGCATCACCCCAGAGCCCAGGCCTCTGGCACAGAGCCCCCCTCACCCACGCACTCCTTAGGAGAAG
GTGGCTTCTTCCGTGCATCCCCTCAGCCACGAAGGCCAAGTTCCCAGGGTTGGGCCAGTCCCCAGGTAGC
AGGGAGACGCCCTGATCCTTTTCCTTCGGTCCCTCGGGGCCGAGGCCAGCAGGGCCAAGGGCCTTGGGGA
ACGGGGGGGACTCCTCACGGGCCCCGCCTGGAGCCTGACCCTCAGCACCCGGGCGCCTGGCTGCCCCTGC
TGAGCAACGGCCCCCATGCCAGCTCCCTCTGGAGCCTCTTTGCTCCCAGTAGCCCTATTCCAAGATGTTC
TGGGGAGAGTGAACAGCTAAGAGCCTGCAGCCAAGCGCCCTGCCCCCCTGAGCAGCCAGACCCCCGGGCC
CTGCAGTGCGCAGCCTTTAACTCCCAGGAATTCATGGGCCAGCTGTATCAGTGGGAGCCCTTCACTGAAG
TCCAGGGCTCCCAGCGCTGTGAACTGAACTGCCGGCCCCGTGGCTTCCGCTTCTATGTCCGTCACACTGA
AAAGGTCCAGGATGGGACCCTGTGTCAGCCTGGAGCCCCTGACATCTGTGTGGCTGGACGCTGTCTGAGC
CCCGGCTGTGATGGGATCCTTGGCTCTGGCAGGCGTCCTGATGGCTGTGGAGTCTGTGGGGGTGATGATT
CTACCTGTCGCCTTGTTTCGGGGAACCTCACTGACCGAGGGGGCCCCCTGGGCTATCAGAAGATCTTGTG
GATTCCAGCGGGAGCCTTGCGGCTCCAGATTGCCCAGCTCCGGCCTAGCTCCAACTACCTGGCACTTCGT
GGCCCTGGGGGCCGGTCCATCATCAATGGGAACTGGGCTGTGGATCCCCCTGGGTCCTACAGGGCCGGCG
GGACCGTCTTTCGATATAACCGTCCTCCCAGGGAGGAGGGCAAAGGGGAGAGTCTGTCGGCTGAAGGCCC
CACCACCCAGCCTGTGGATGTCTATATGATCTTTCAGGAGGAAAACCCAGGCGTTTTTTATCAGTATGTC
ATCTCTTCACCTCCTCCAATCCTTGAGAACCCCACCCCAGAGCCCCCTGTCCCCCAGCTTCAGCCGGAGA
TTCTGAGGGTGGAGCCCCCACTTGCTCCGGCACCCCGCCCAGCCCGGACCCCAGGCACCCTCCAGCGTCA
GGTGCGGATCCCCCAGATGCCCGCCCCGCCCCATCCCAGGACACCCCTGGGGTCTCCAGCTGCGTACTGG
AAACGAGTGGGACACTCTGCATGCTCAGCGTCCTGCGGGAAAGGTGTCTGGCGCCCCATTTTCCTCTGCA
TCTCCCGTGAGTCGGGAGAGGAACTGGATGAACGCAGCTGTGCCGCGGGTGCCAGGCCCCCAGCCTCCCC
TGAACCCTGCCACGGCACCCCATGCCCCCCATACTGGGAGGCTGGCGAGTGGACATCCTGCAGCCGCTCC
TGTGGCCCCGGCACCCAGCACCGCCAGCTGCAGTGCCGGCAGGAATTTGGGGGGGGTGGCTCCTCGGTGC
CCCCGGAGCGCTGTGGACATCTCCCCCGGCCCAACATCACCCAGTCTTGCCAGCTGCGCCTCTGTGGCCA
TTGGGAAGTTGGCTCTCCTTGGAGCCAGTGCTCCGTGCGGTGCGGCCGGGGCCAGAGAAGCCGGCAGGTT
CGCTGTGTTGGGAACAATGGTGATGAAGTGAGCGAGCAGGAGTGTGCGTCAGGCCCCCCGCAGCCCCCCA
GCAGAGAGGCCTGTGACATGGGGCCCTGTACTACTGCCTGGTTCCACAGCGACTGGAGCTCCAAGTGCTC
AGCCGAGTGTGGGACGGGAATCCAGCGGCGCTCTGTGGTCTGCCTTGGGAGTGGGGCAGCCCTCGGGCCA
GGCCAGGGGGAAGCAGGAGCAGGAACTGGGCAGAGCTGTCCAACAGGAAGCCGGCCCCCTGACATGCGCG
CCTGCAGCCTGGGGCCCTGTGAGAGAACTTGGCGCTGGTACACAGGGCCCTGGGGTGAGTGCTCCTCCGA
ATGTGGCTCTGGCACACAGCGTAGAGACATCATCTGTGTATCCAAACTGGGGACGGAGTTCAACGTGACT
TCTCCGAGCAACTGTTCTCACCTCCCCAGGCCCCCTGCCCTGCAGCCCTGTCAAGGGCAGGCCTGCCAGG
ACCGATGGTTTTCCACGCCCTGGAGCCCATGTTCTCGCTCCTGCCAAGGGGGAACGCAGACACGGGAGGT
CCAGTGCCTGAGCACCAACCAGACCCTCAGCACCCGATGCCCTCCTCAACTGCGGCCCTCCAGGAAGCGC
CCCTGTAACAGCCAACCCTGCAGCCAGCGCCCTGATGATCAATGCAAGGACAGCTCTCCACATTGCCCCC
TGGTGGTACAGGCCCGGCTCTGCGTCTACCCCTACTACACAGCCACCTGTTGCCGCTCTTGCGCACATGT
CCTGGAGCGGTCTCCCCAGGATCCCTCCTGAAAGGGGTCCGGGGCACCTTCACGGTTTTCTGTGCCACCA
TCGGTCACCCATTGATCGGCCCACTCTGAACCCCCTGGCTCTCCAGCCTGTCCCAGTCTCAGCAGGGATG
TCCTCCAGGTGACAGAGGGTGGCAAGGTGACTGACACAAAGTGACTTTCAGGGCTGTGGTCAGGCCCATG
TGGTGGTGTGATGGGTGTGTGCACATATGCCTCAGGTGTGCTTTTGGGACTGCATGGATATGTGTGTGCT
CAAACGTGTATCACTTTTCAAAAAGAGGTTACACAGACTGAGAAGGACAAGACCTGTTTCCTTGAGACTT
TCCTAGGTGGAAAGGAAAGCAAGTCTGCAGTTCCTTGCTAATCTGAGCTACTTAGAGTGTGGTCTCCCCA
CCAACTCCAGTTTTGTGCCCTAAGCCTCATTTCTCATGTTCAGACCTCACATCTTCTAAGCCGCCCTGTG
TCTCTGACCCCTTCTCATTTGCCTAGTATCTCTGCCCCTGCCTCCCTAATTAGCTAGGGCTGGGGTCAGC
CACTGCCAATCCTGCCTTACTCAGGAAGGCAGGAGGAAAGAGACTGCCTCTCCAGAGCAAGGCCCAGCTG
GGCAGAGGGTGAAAAAGAGAAATGTGAGCATCCGCTCCCCCACCACCCCGCCCAGCCCCTAGCCCCACTC
CCTGCCTCCTGAAATGGTTCCCACCCAGAACTAATTTATTTTTTATTAAAGATGGTCATGACAAATGAGA
AAAAAAAAA
SEQ ID NO: 5
Homo sapiens anaphase promoting complex subunit 1 (ANAPC1), mRNA
CGCGTCCATTTGAACGTCTCGCACGCCTTCCTGCCATTAGCACTCGAGCCCGCTGCTGTTGCCCGTTCTT
CCTCCAGAATAGGGGAGGGAGAGGGAATGAGAAGCTGCTGCGGCCCAAGAGTCACTGTGAAGGACCCCGC
CGCTGCCCTCGGGCCTCCTCGGCCCCTGCGCCTCCGGGGAGCAGCCGGGGCTCGCCGCGCCTGACGCGTC
CCGAGTTATACAGAAATAATGTTGATATTTGGAACCCATGTCGAACTTCTATGAAGAAAGGACAACGATG
ATTGCAGCAAGGGATTTGCAGGAATTTGTTCCTTTTGGTCGAGACCACTGCAAGCACCACCCTAATGCTT
TGAACCTTCAACTTCGCCAGCTGCAGCCAGCTTCTGAATTATGGTCTTCTGATGGTGCTGCTGGCTTGGT
GGGATCCCTTCAGGAGGTTACAATCCACGAGAAACAGAAGGAAAGCTGGCAGTTAAGGAAAGGAGTAAGT
GAAATTGGAGAAGATGTGGACTATGATGAGGAACTCTATGTTGCTGGAAATATGGTGATATGGAGCAAAG
GAAGTAAAAGCCAGGCATTGGCAGTTTATAAAGCATTTACAGTTGACAGTCCTGTTCAGCAGGCATTGTG
GTGTGACTTCATTATATCACAGGATAAGTCTGAAAAGGCCTACAGTAGCAATGAAGTAGAAAAATGCATA
TGTATATTGCAAAGCTCATGTATTAACATGCATAGCATAGAAGGAAAGGATTACATAGCTTCATTACCAT
TTCAGGTTGCAAATGTTTGGCCCACTAAATATGGATTGCTGTTTGAACGAAGCGCTTCTTCACATGAAGT
ACCTCCAGGTTCACCCAGAGAACCTTTACCTACTATGTTCAGCATGCTGCACCCACTAGATGAAATAACT
CCACTTGTTTGTAAATCTGGAAGTCTTTTTGGTTCATCACGGGTGCAATATGTTGTAGATCATGCAATGA
AAATTGTTTTCCTCAATACTGACCCCTCTATTGTAATGACTTATGATGCTGTTCAAAATGTGCATTCTGT
GTGGACTCTCCGGAGAGTCAAATCAGAGGAAGAGAATGTTGTTTTAAAGTTCTCTGAACAGGGGGGAACC
CCACAGAATGTGGCCACTAGCAGCTCCCTCACAGCACATCTCAGAAGCCTCTCCAAAGGAGATTCCCCTG
TGACTTCACCTTTCCAGAATTACTCCTCCATTCACAGCCAGAGTCGCTCAACCTCATCACCCAGTCTACA
TTCTCGCTCACCTTCTATTTCCAACATGGCAGCTCTAAGTCGTGCTCATTCTCCTGCGTTAGGAGTGCAC
TCTTTTTCAGGGGTGCAAAGGTTCAACATTTCAAGCCATAATCAGTCTCCAAAGAGACATAGTATTTCTC
ATTCTCCAAATAGTAATTCTAATGGCTCCTTTCTTGCACCAGAAACGGAGCCAATTGTTCCTGAACTGTG
TATTGACCATTTGTGGACAGAAACGATTACTAATATAAGAGAGAAAAATTCACAAGCCTCAAAAGTGTTT
ATTACATCTGACCTATGTGGGCAAAAGTTCCTGTGCTTTTTAGTAGAGTCCCAGCTCCAGTTACGCTGTG
TAAAGTTTCAAGAGAGTAATGATAAAACCCAGCTCATCTTTGGTTCAGTGACCAACATACCAGCAAAGGA
TGCAGCACCAGTGGAGAAAATAGACACCATGCTGGTCTTGGAAGGCAGTGGAAACCTGGTGCTATACACA
GGAGTGGTTCGGGTGGGAAAGGTTTTTATTCCTGGACTGCCAGCTCCCTCTCTGACGATGTCCAACACAA
TGCCTCGGCCCAGTACTCCACTAGATGGCGTTAGTACTCCAAAGCCTCTTAGTAAACTCCTTGGATCATT
GGACGAGGTTGTTCTGTTGTCCCCAGTTCCAGAACTGAGGGATTCTTCAAAACTTCATGATTCTCTCTAT
AATGAGGATTGTACTTTCCAACAGCTTGGAACTTACATTCATTCTATCAGAGATCCTGTCCATAACAGAG
TCACCCTGGAACTGAGTAATGGCTCCATGGTTAGGATCACTATTCCTGAAATTGCCACCTCTGAGTTAGT
ACAAACGTGTTTGCAAGCAATTAAGTTTATCCTGCCAAAAGAAATAGCAGTTCAGATGCTTGTCAAGTGG
TACAATGTCCACAGTGCTCCAGGAGGACCCAGTTATCACTCAGAGTGGAATTTATTTGTGACTTGTCTCA
TGAACATGATGGGTTATAACACAGACCGCTTAGCATGGACTAGAAATTTTGACTTTGAAGGATCACTTTC
TCCTGTCATTGCGCCCAAAAAAGCAAGGCCTTCCGAGACTGGATCTGATGATGACTGGGAATATTTACTA
AATTCAGACTACCACCAGAATGTTGAGTCTCATCTTTTGAACAGATCTTTATGTCTGAGTCCTTCAGAAG
CTTCACAGATGAAGGATGAGGATTTTTCACAGAATCTCAGTCTGGATTCTTCTACACTTCTCTTTACTCA
CATACCTGCAATTTTTTTCGTTCTTCACCTTGTGTATGAGGAGCTTAAGTTGAATACTCTAATGGGAGAA
GGAATTTGTTCACTTGTTGAACTTCTCGTTCAGTTGGCAAGGGACTTAAAATTGGGGCCTTATGTAGATC
ATTACTATAGAGACTACCCAACGCTTGTCAGAACTACTGGACAAGTGTGCACAATTGATCCAGGTCAAAC
AGGATTTATGCATCATCCATCATTTTTTACGTCTGAGCCACCAAGTATTTATCAGTGGGTGAGTTCTTGT
CTGAAGGGTGAAGGAATGCCACCTTATCCTTACCTCCCTGGAATCTGTGAAAGAAGCAGACTTGTAGTCT
TGAGTATTGCACTGTACATACTTGGTGATGAGAGCTTGGTTTCTGATGAATCCTCACAGTATTTAACCAG
AATAACTATAGCCCCCCAGAAGTTGCAAGTAGAACAAGAGGAAAACAGGTTTAGTTTCAGGCATTCTACA
TCTGTTTCTAGTCTAGCTGAAAGATTGGTTGTCTGGATGACTAATGTAGGATTCACTTTAAGAGATTTGG
AAACTCTTCCCTTTGGAATTGCTCTTCCCATCAGAGATGCAATTTATCACTGTCGTGAGCAGCCTGCCTC
AGACTGGCCAGAAGCTGTCTGTCTCTTGATTGGACGTCAGGATCTTTCCAAGCAGGCCTGCGAAGGAAAC
TTACCCAAAGGGAAGTCTGTGCTCTCATCAGATGTTCCTTCAGGAACAGAAACTGAGGAGGAAGATGACG
GCATGAATGACATGAATCACGAGGTCATGTCATTAATATGGAGTGAAGATTTAAGGGTGCAGGATGTGCG
AAGGCTTCTTCAGAGTGCGCATCCTGTCCGTGTCAACGTAGTGCAGTACCCAGAGCTCAGTGACCACGAG
TTCATCGAGGAAAAGGAAAACAGATTGCTCCAATTGTGTCAGCGAACTATGGCTCTTCCTGTAGGACGAG
GAATGTTTACCTTGTTTTCGTACCATCCTGTTCCAACAGAGCCATTGCCTATTCCTAAATTGAATCTGAC
TGGGCGTGCCCCTCCTCGGAACACAACAGTAGACCTTAATAGTGGAAACATCGATGTGCCTCCCAACATG
ACAAGCTGGGCCAGCTTTCATAATGGTGTGGCTGCTGGCCTGAAGATAGCTCCTGCCTCCCAGATCGACT
CAGCTTGGATTGTTTACAATAAGCCCAAGCATGCTGAGTTGGCCAATGAGTATGCTGGCTTTCTCATGGC
TCTGGGTTTGAATGGGCACCTTACCAAGCTGGCGACTCTCAATATCCATGACTACTTGACCAAGGGCCAT
GAAATGACAAGCATTGGACTGCTACTTGGTGTTTCTGCTGCAAAACTAGGCACCATGGATATGTCTATTA
CTCGGCTTCTTAGCATTCACATTCCTGCTCTCTTACCCCCAACGTCCACAGAGCTGGATGTTCCTCACAA
TGTCCAAGTGGCTGCAGTGGTTGGCATTGGCCTTGTATATCAAGGGACAGCTCACAGACATACTGCAGAA
GTCCTGTTGGCTGAGATAGGACGGCCTCCTGGTCCTGAAATGGAATACTGCACTGACAGAGAGTCATACT
CCTTAGCTGCTGGCTTGGCCCTGGGCATGGTCTGCTTGGGGCATGGCAGCAATTTGATAGGTATGTCTGA
TCTCAATGTGCCTGAGCAGCTCTATCAGTACATGGTTGGAGGACATAGGCGCTTTCAAACAGGAATGCAT
AGGGAGAAACATAAATCACCAAGTTATCAAATCAAAGAAGGAGATACCATAAATGTGGATGTGACTTGTC
CAGGTGCTACTCTAGCTTTGGCTATGATCTACTTAAAAACCAATAACAGATCTATTGCAGATTGGCTCCG
AGCCCCTGACACCATGTATTTGTTGGACTTTGTGAAGCCAGAATTTCTCTTGCTTAGGACACTTGCTCGA
TGCCTGATTTTGTGGGATGATATTTTACCAAATTCCAAGTGGGTTGACAGCAATGTTCCTCAAATTATAA
GAGAAAATAGTATCTCTCTCAGTGAAATCGAATTGCCGTGCTCAGAGGATTTGAATTTGGAAACTTTGTC
CCAAGCACATGTCTACATAATTGCAGGAGCCTGCTTGTCTCTGGGTTTTCGATTTGCTGGCTCAGAAAAC
TTATCAGCATTTAACTGTTTGCATAAATTTGCCAAAGATTTTATGACTTATTTGTCCGCACCTAATGCTT
CTGTTACAGGTCCTCATAACCTAGAAACTTGTCTGAGCGTGGTGCTGCTGTCTCTCGCCATGGTCATGGC
TGGCTCAGGAAACCTAAAGGTTTTGCAGCTTTGTCGCTTCTTACACATGAAAACGGGTGGTGAAATGAAC
TATGGTTTTCACTTAGCCCACCACATGGCCCTTGGACTTCTATTTTTGGGAGGAGGAAGGTACTCTTTGA
GCACATCAAATTCTTCCATTGCCGCTCTTCTCTGTGCCCTTTATCCGCACTTCCCAGCTCACAGCACTGA
CAACCGGTATCATCTCCAGGCTCTCCGGCACCTCTATGTGCTGGCCGCGGAGCCCAGGCTTCTAGTGCCT
GTGGATGTGGACACAAACACGCCCTGCTATGCCCTCTTAGAAGTTACCTACAAGGGCACTCAGTGGTATG
AACAAACCAAAGAAGAATTGATGGCTCCTACCCTTCTTCCAGAACTCCATCTTTTAAAGCAGATTAAAGT
AAAAGGCCCAAGATACTGGGAACTGCTCATAGATTTAAGCAAAGGAACACAACACTTGAAGTCCATCCTT
TCCAAGGATGGGGTTTTATATGTTAAACTCCGGGCGGGTCAGCTCTCCTACAAAGAAGATCCAATGGGAT
GGCAAAGTTTGTTGGCTCAGACTGTTGCTAACAGGAACTCTGAAGCCCGGGCTTTCAAGCCAGAAACAAT
CTCAGCATTCACTTCTGATCCAGCACTTCTGTCATTTGCTGAATATTTCTGCAAGCCAACTGTGAACATG
GGTCAGAAACAGGAAATTCTGGATCTCTTTTCTTCAGTACTCTATGAATGTGTTACCCAGGAGACCCCAG
AGATGTTGCCTGCATACATAGCAATGGATCAGGCTATAAGAAGACTTGGGAGAAGAGAAATGTCTGAGAC
TTCTGAACTTTGGCAGATAAAGTTGGTGTTAGAGTTTTTCAGCTCCCGAAGCCATCAGGAGCGGCTGCAG
AACCACCCTAAGCGGGGGCTCTTTATGAACTCGGAATTCCTCCCTGTTGTGAAGTGCACCATTGATAATA
CCCTGGACCAGTGGCTACAAGTCGGGGGTGATATGTGTGTGCACGCCTACCTCAGCGGGCAGCCCTTGGA
GGAATCACAGCTGAGCATGCTGGCCTGCTTCCTCGTCTACCACTCTGTGCCAGCTCCACAGCACCTGCCA
CCTATAGGACTAGAAGGGAGCACAAGCTTTGCTGAACTGCTCTTCAAATTTAAGCAGCTAAAAATGCCAG
TGCGAGCTTTGCTGAGATTGGCTCCTTTGCTTCTTGGAAATCCACAGCCAATGGTGATGTGACCGTGTCT
GGCGGTGAACCTACCCTGAAACGTGACTTCTGCACAACAAACGTGACCAAACATCAAAGCTAAAGCAATG
TTTATAAAGTTTTATGGTATAACTAGGGGGAAATGAGCTGCACAAACCTCAATGTATTTTAAATCTGTTG
CTGTCATCATTAACGGTATATGACATATAAAAGCAAGTTAAAATTTACTTTTGTAAATAAAGTTTTTGGT
TTGTTTCCAAAAAAAAAAAAAAAAAAAAA
SEQ ID NO: 6
Homo sapiens apolipoprotein B mRNA editing enzyme, catalytic polypeptide-
like 3F (APOBEC3F), transcript variant 1, mRNA
TTCCCTTTGCAATTGCCTTGGGTCCTGCCGCACAGAGCGGCCTGTCTTTATCAGAGGTCCCTCTGCCAGG
GGGAGGGCCCCAGAGAAAACCAGAAAGAGGGTGAGAGACTGAGGAAGATAAAGCGTCCCAGGGCCTCCTA
CACCAGCGCCTGAGCAGGAAGGGGGAGGGGCCATGACTACGAGGCCCTGGGAGGTCACTTTAGGGAGGGC
TGTCCTGAAACCTGGAGCCTGGAGCAGAAAGTGAAACCCTGGTGCTCCAGACAAAGATCTTAGTCGGGAC
TAGCCGGCCAAGGATGAAGCCTCACTTCAGAAACACAGTGGAGCGAATGTATCGAGACACATTCTCCTAC
AACTTTTATAATAGACCCATCCTTTCTCGTCGGAATACCGTCTGGCTGTGCTACGAAGTGAAAACAAAGG
GTCCCTCAAGGCCCCGTTTGGACGCAAAGATCTTTCGAGGCCAGGTGTATTCCCAGCCTGAGCACCACGC
AGAAATGTGCTTCCTCTCTTGGTTCTGTGGCAACCAGCTGCCTGCTTACAAGTGTTTCCAGATCACCTGG
TTTGTATCCTGGACCCCCTGCCCGGACTGTGTGGCGAAGCTGGCCGAATTCCTGGCTGAGCACCCCAATG
TCACCCTGACCATCTCCGCCGCCCGCCTCTACTACTACTGGGAAAGAGATTACCGAAGGGCGCTCTGCAG
GCTGAGTCAGGCAGGGGCCCGCGTGAAGATTATGGACGATGAAGAATTTGCATACTGCTGGGAAAACTTT
GTGTACAGTGAAGGTCAGCCATTCATGCCTTGGTACAAATTCGATGACAATTATGCATTCCTGCACCGCA
CGCTAAAGGAGATTCTCAGAAACCCGATGGAGGCAATGTATCCACACATATTCTACTTCCACTTTAAAAA
CCTACGCAAAGCCTATGGTCGGAACGAAAGCTGGCTGTGCTTCACCATGGAAGTTGTAAAGCACCACTCA
CCTGTCTCCTGGAAGAGGGGCGTCTTCCGAAACCAGGTGGATCCTGAGACCCATTGTCATGCAGAAAGGT
GCTTCCTCTCTTGGTTCTGTGACGACATACTGTCTCCTAACACAAACTACGAGGTCACCTGGTACACATC
TTGGAGCCCTTGCCCAGAGTGTGCAGGGGAGGTGGCCGAGTTCCTGGCCAGGCACAGCAACGTGAATCTC
ACCATCTTCACCGCCCGCCTCTACTACTTCTGGGATACAGATTACCAGGAGGGGCTCCGCAGCCTGAGTC
AGGAAGGGGCCTCCGTGGAGATCATGGGCTACAAAGATTTTAAATATTGTTGGGAAAACTTTGTGTACAA
TGATGATGAGCCATTCAAGCCTTGGAAAGGACTAAAATACAACTTTCTATTCCTGGACAGCAAGCTGCAG
GAGATTCTCGAGTGAGGGGTCTCCCCGGGCCTCATGGTCTGTCTCCTCTAGCCTCCTGCTCATGTTGTGC
AGGCCTCCCCTCCATCCTGGACCAGCTGTGCTTTTGCCTGGTCATCCTGAGCCCCTCCTGGCCTCAGGGC
CATTCCATAGTGCTCCCCTGCCTCACCACCTCCTCTCCGCTCTCCCAGGCTCTTCCTGCAGAGGCCTCTT
TCTGCCTCCATGGCTATCCATCCACCCACCAAGACCCTGTTCCCTGAGCCTGCATGCCCCTAACCTGCCT
TTTCCCATCTCCCCAGCATAACCTAATATTTTTTTTTTTTTTTTGAGACGGAATTTCGCTCTGTCACCCA
GACTGGAGTGCAATGGCTTGATCTTGGCTCACTGCAAACTCTGCCTACCAGGTTCAAGCGATTCTCCTGC
CTCCGCCTCCCGAGTAGCTGGAATTACAGACGCCTGCCACCACGCACAGCTAACTTTTTTTTTTTTTGTA
TTTTTAGTAGTGACTGGGTTTCACCATGTTGGCCAGGCTGGTCTTGAACTCCTGACCTCAGGTGATCCGC
CTATCTCAGCCTCCCAAAGTGCTGGGATTACAGGCGTGAGCCACTGGCCCGGCGGCACAACCAAATCTTA
TTAAACTCACCCTAGGCTGGCCGCGGTGACTCATGCCTATAATCCCCCAGCAATTTGGGAGGCAGAGGTG
AGAGAATCGCTTGAGCCCAGGAATTCGAGACCAGCCTGGGCCACATGACAAAGCCCCATCTCTACAAAAA
AATTACAAAAAAAAAAAAAACAGGTGTGGTGGCATGCACCTGTAGTTGAAGCTACTTGGAAGGATGAAGT
GGGAGGATTGCTTGAGCCGGGGAGGTGGAGGCTGCAGTGAACTGAGATCACGTCACTGAACTCCAGTCTG
AGCAACAGATCGAGACCCTGCCTGAAAATAAATCAATAAATAAACTCAACCGAAATGGGTATGAAAGTTG
AAATGGGTATGTAAGTTGAAAACCAGAAGTTTTGAGAAACATCCTTTGTTAACTTTCATCCTACAAATTG
GGTCATTCATGTCCTACGCAGCTAAAACAGAGCCCAGGAGCCAGGGAGGAAAAGCAGTCAGGCCACACAC
CATTGCTCCCAAAATGGACTTCTCTGCAAGCCTGACTCCTGAAACTGTGCATTGTACCCTGAAACCAGCT
TTATCCATAGCTTCTGCAATAAATGGCTGTAAGTCTTGGACTCCTTGCTATAATCGCAGCTATTCAGCAA
TGGAACCTCCCAGTTCCCAACCCTTCCTAGTGCCCATGGGCTTTCCCATAGGACAAGAGAACATTTCTCC
TTTTCTTTTTTTTTTTCTTTGAAATGGAGTCTCGCCCTGTCACCCAGGCTGGAGTGCAATGGTGCGGTCT
CGGCTCACTGCAACCTCTGCCTCCCTTGTTCAAGTGATTCTCCTGTCTCAGCCTCCCGAGTAGCTGGGAT
TACAGGCGTCCACCACCAAACCAGGCTAATTTTTGTATTTTTCATAAAAACGGGTTTCATCATGTTTCCC
AGGCTGGTCTTATTTTTATTTTATTTTTTGAGATGGAGTCTTGCTCTGTTGCCCAGGCTGGGGTGCAGTG
GTGCAATCTGGGTTCACTGCAGCCTCTGCCGCCTGAGTTCAAGCTATTTTCCTACCTAAGCCTCCCAAGT
AGCTGGGATTACATGCGCGTGCCACCACGCCTAGCTAATTTTTGTGTTTTTAGTAGAGACGGGGTTTCAA
CATCTTGACCAGGCTGGTCTTGAACTCCTGACCTCGTGATCCACCCGTCTCGGCCTCCCAAAGTGCTGGG
ATTACAGGCGTGAGCCACCTGGCCAGGCTTAGGCTGGTCTTAAACTCCTGACCTCAAGTGATCCAACCTC
CTTGGCCTCCCAAATTGCTGGGATTGCTGGTGTGAGCCACAGCGCCTAGCCCATTTCTCCTTTTAATAGG
ACCTGTTGCTGTCTCTGTTCTCCCAACATGGTGAACACCACCCGGACTGCGTGTATGTCCCAAATTACAA
TTCTTTCTTTGCAAATGAAATGTGAAATTTAGAGGCCCTTCTCCACACTTTAAATTTGACTTGACATTTT
CTAGGCAGATATAAGTTATTAGAGAATGAGATTCTCTATAAAAATGATCCCTTCATGCTGTGGCCTCCAC
AGAAGATGCCCTGGGCCAGGTGCCCACATGAATAATGCGGGCCACAGGCAGGCATTTATTTTCTCACAGA
TATGGAGGCTACAAGTCCAAGGTGGAGGGGTCGGCGGGGTTGTTTGCTCTGAGGCCGCTCCTCCTGGATG
GCAGGGATCCCTTCTGGCTGTGTCCTCTGTGGCCTTTCCTCTATGAACCTGTACTGTACCTCTGGGGTCT
CTCTGCTTCCAAATATCTTTTTTTTTTTTTTCAGACAGTTTTGCTCTTGTTTTCTAGGCTGGAGTGCAAT
GGCACAATCTCAGCTCACTGCAACCTCTGCCTTCCGAGTTCAAGCGATTCTCGTGCCTCAGCCTCCTGAG
TAGCTGGGACTACAGGCGTGTGCCACCACGCCTGGCTAATTTTGTAGTTTTAGTAGAGACGGGGTTTCTC
CATGTTGCTCAGGCTGGTCTTGAACTCATGAGCTCAGGCGATCCACTCTCCTCAGCCTCCCAAAGTGCTG
GGATTACAGATATAAGCCACCATACACAACTTTTTTTTTTTTTTGAGATGGAGTTTCACTCTGTTGCCCA
GGCTGGAGTGCTAAATAGCAGAATCACTGCTCACTGCAACCTCTGCCTGCTGGGTTCAAGCAATTCTCCC
ACCTCAGCCTCCTGAGTAGCTGGGATTACAGATGCCCAGAACCAATCTCTGCTAATTTTTCTATTTTTTA
GTAGAGATGGGGTTTCACTGAGGAAGGAGACCACCTCTCTCATTGTCTCCTATTTCAGAAGGAAGCAAAA
AGTTAGAAAGATGCAGAAGTAAGATCAATGGCCAGACTGTTTGGCGCTGCTACCTGGGCCTGGTAGTTAA
AGATCAACTCCTGACCTGACCGCTTGTTTTATCTAAAGATTCCAGACATTGTATGAGGAAGCATTGTGAA
ACTTTCTGGTCTGTTCTGCTAGCCCCCACCACTGATGCATGTAGCCCCCCAGTCACGTAGCCCACGCTTG
CACAATCTATCACGACCCTTTCACGTGGACCCCTTAGAATTGTAAGCCCTTAAAAGGGCCAGGGACTTCT
TCAGGGAGCTCCAATCTTCAGATGCAAGTCTGTCAACGCTCCCAGCTGATTAAAGCCTCTTCCTTCCTAA
AAAAAAAAAAAAAAAA
SEQ ID NO: 7
Homo sapiens argininosuccinate lyase (ASL), transcript variant 1, mRNA
CCAGGCGGAGGTGAGTGCGCGGCGGCCGGATGGGCGGGACGGGCGTGGAGGACGCCGAGCACCGTGGCGC
GCGCTCACGTCCGCGTCCCCAAGGGCTGCGCTCCCTCAAGCGCAGTGCCCAGAACTCGGAGCCAGCCCGG
CCCGGGGGACCCTGCTGGCCAAGGAGGTCGTCAGTCCGGTCTTGTCTTCCAGACCCGGAGGACCGAAGCT
TCCGGACGACGAGGAACCGCCCAACATGGCCTCGGAGAGTGGGAAGCTTTGGGGTGGCCGGTTTGTGGGT
GCAGTGGACCCCATCATGGAGAAGTTCAACGCGTCCATTGCCTACGACCGGCACCTTTGGGAGGTGGATG
TTCAAGGCAGCAAAGCCTACAGCAGGGGCCTGGAGAAGGCAGGGCTCCTCACCAAGGCCGAGATGGACCA
GATACTCCATGGCCTAGACAAGGTGGCTGAGGAGTGGGCCCAGGGCACCTTCAAACTGAACTCCAATGAT
GAGGACATCCACACAGCCAATGAGCGCCGCCTGAAGGAGCTCATTGGTGCAACGGCAGGGAAGCTGCACA
CGGGACGGAGCCGGAATGACCAGGTGGTCACAGACCTCAGGCTGTGGATGCGGCAGACCTGCTCCACGCT
CTCGGGCCTCCTCTGGGAGCTCATTAGGACCATGGTGGATCGGGCAGAGGCGGAACGTGATGTTCTCTTC
CCGGGGTACACCCATTTGCAGAGGGCCCAGCCCATCCGCTGGAGCCACTGGATTCTGAGCCACGCCGTGG
CACTGACCCGAGACTCTGAGCGGCTGCTGGAGGTGCGGAAGCGGATCAATGTCCTGCCCCTGGGGAGTGG
GGCCATTGCAGGCAATCCCCTGGGTGTGGACCGAGAGCTGCTCCGAGCAGAACTCAACTTTGGGGCCATC
ACTCTCAACAGCATGGATGCCACTAGTGAGCGGGACTTTGTGGCCGAGTTCCTGTTCTGGGCTTCGCTGT
GCATGACCCATCTCAGCAGGATGGCCGAGGACCTCATCCTCTACTGCACCAAGGAATTCAGCTTCGTGCA
GCTCTCAGATGCCTACAGCACGGGAAGCAGCCTGATGCCCCAGAAGAAAAACCCCGACAGTTTGGAGCTG
ATCCGGAGCAAGGCTGGGCGTGTGTTTGGGCGGTGTGCCGGGCTCCTGATGACCCTCAAGGGACTTCCCA
GCACCTACAACAAAGACTTACAGGAGGACAAGGAAGCTGTGTTTGAAGTGTCAGACACTATGAGTGCCGT
GCTCCAGGTGGCCACTGGCGTCATCTCTACGCTGCAGATTCACCAAGAGAACATGGGACAGGCTCTCAGC
CCCGACATGCTGGCCACTGACCTTGCCTATTACCTGGTCCGCAAAGGGATGCCATTCCGCCAGGCCCACG
AGGCCTCCGGGAAAGCTGTGTTCATGGCCGAGACCAAGGGGGTCGCCCTCAACCAGCTGTCACTGCAGGA
GCTGCAGACCATCAGCCCCCTGTTCTCGGGCGACGTGATCTGCGTGTGGGACTACGGGCACAGTGTGGAG
CAGTATGGTGCCCTGGGCGGCACTGCGCGCTCCAGCGTCGACTGGCAGATCCGCCAGGTGCGGGCGCTAC
TGCAGGCACAGCAGGCCTAGGTCCTCCCACACCTGCCCCCTAATAAAGTGGGCGCGAGAGGAGGCTGCTG
TGTGTTTCCTGCCCCAGCCTGGCTCCCTCGTTGCTGGGCTTTCGGGGCTGGCCAGTGGGGACAGTCAGGG
ACTGGAGAGGCAGGGCAGGGTGGCCTGTAATCCCAGCACTTTGGAAGGGCAAGGTGCGAGGATGCTTGAG
GCCAGGAGTTTGACACAGCCTGGGCAACACAGGGAGACCCCCATCTCTACTCAATAATAAAACAAATAGC
CTGGCGTGGTGGCCCATGCATATAGTCCCAGCTACTTGTAAGGCTGAGGTGAGAGGACACTTGTGCCCAG
GAGTGGAGGCTGCAGTGAGCTATGATCACGCCACTGCATTCCAGCCTGGATAACAGAGTGAGAACCTATC
TCTAAAAATAAATAAATAAACGAAAAATAAA
SEQ ID NO: 8
Homo sapiens beta-2-microglobulin (B2M), mRNA
AATATAAGTGGAGGCGTCGCGCTGGCGGGCATTCCTGAAGCTGACAGCATTCGGGCCGAGATGTCTCGCT
CCGTGGCCTTAGCTGTGCTCGCGCTACTCTCTCTTTCTGGCCTGGAGGCTATCCAGCGTACTCCAAAGAT
TCAGGTTTACTCACGTCATCCAGCAGAGAATGGAAAGTCAAATTTCCTGAATTGCTATGTGTCTGGGTTT
CATCCATCCGACATTGAAGTTGACTTACTGAAGAATGGAGAGAGAATTGAAAAAGTGGAGCATTCAGACT
TGTCTTTCAGCAAGGACTGGTCTTTCTATCTCTTGTACTACACTGAATTCACCCCCACTGAAAAAGATGA
GTATGCCTGCCGTGTGAACCATGTGACTTTGTCACAGCCCAAGATAGTTAAGTGGGATCGAGACATGTAA
GCAGCATCATGGAGGTTTGAAGATGCCGCATTTGGATTGGATGAATTCCAAATTCTGCTTGCTTGCTTTT
TAATATTGATATGCTTATACACTTACACTTTATGCACAAAATGTAGGGTTATAATAATGTTAACATGGAC
ATGATCTTCTTTATAATTCTACTTTGAGTGCTGTCTCCATGTTTGATGTATCTGAGCAGGTTGCTCCACA
GGTAGCTCTAGGAGGGCTGGCAACTTAGAGGTGGGGAGCAGAGAATTCTCTTATCCAACATCAACATCTT
GGTCAGATTTGAACTCTTCAATCTCTTGCACTCAAAGCTTGTTAAGATAGTTAAGCGTGCATAAGTTAAC
TTCCAATTTACATACTCTGCTTAGAATTTGGGGGAAAATTTAGAAATATAATTGACAGGATTATTGGAAA
TTTGTTATAATGAATGAAACATTTTGTCATATAAGATTCATATTTACTTCTTATACATTTGATAAAGTAA
GGCATGGTTGTGGTTAATCTGGTTTATTTTTGTTCCACAAGTTAAATAAATCATAAAACTTGATGTGTTA
TCTCTTA
SEQ ID NO: 9
Homo sapiens breast cancer 1, early onset (BRCA1), transcript variant
6, non-coding RNA
AGATAACTGGGCCCCTGCGCTCAGGAGGCCTTCACCCTCTGCTCTGGGTAAAGGTAGTAGAGTCCCGGGA
AAGGGACAGGGGGCCCAAGTGATGCTCTGGGGTACTGGCGTGGGAGAGTGGATTTCCGAAGCTGACAGAT
GGTTCATTGGAACAGAAAGAAATGGATTTATCTGCTCTTCGCGTTGAAGAAGTACAAAATGTCATTAATG
CTATGCAGAAAATCTTAGAGTGTCCCATCTGTCTGGAGTTGATCAAGGAACCTGTCTCCACAAAGTGTGA
CCACATATTTTGCAAATTTTGCATGCTGAAACTTCTCAACCAGAAGAAAGGGCCTTCACAGTGTCCTTTA
TGAGCCTACAAGAAAGTACGAGATTTAGTCAACTTGTTGAAGAGCTATTGAAAATCATTTGTGCTTTTCA
GCTTGACACAGGTTTGGAGTATGCAAACAGCTATAATTTTGCAAAAAAGGAAAATAACTCTCCTGAACAT
CTAAAAGATGAAGTTTCTATCATCCAAAGTATGGGCTACAGAAACCGTGCCAAAAGACTTCTACAGAGTG
AACCCGAAAATCCTTCCTTGGAAACCAGTCTCAGTGTCCAACTCTCTAACCTTGGAACTGTGAGAACTCT
GAGGACAAAGCAGCGGATACAACCTCAAAAGACGTCTGTCTACATTGAATTGGGATCTGATTCTTCTGAA
GATACCGTTAATAAGGCAACTTATTGCAGTGTGGGAGATCAAGAATTGTTACAAATCACCCCTCAAGGAA
CCAGGGATGAAATCAGTTTGGATTCTGCAAAAAAGGCTGCTTGTGAATTTTCTGAGACGGATGTAACAAA
TACTGAACATCATCAACCCAGTAATAATGATTTGAACACCACTGAGAAGCGTGCAGCTGAGAGGCATCCA
GAAAAGTATCAGGGTAGTTCTGTTTCAAACTTGCATGTGGAGCCATGTGGCACAAATACTCATGCCAGCT
CATTACAGCATGAGAACAGCAGTTTATTACTCACTAAAGACAGAATGAATGTAGAAAAGGCTGAATTCTG
TAATAAAAGCAAACAGCCTGGCTTAGCAAGGAGCCAACATAACAGATGGGCTGGAAGTAAGGAAACATGT
AATGATAGGCGGACTCCCAGCACAGAAAAAAAGGTAGATCTGAATGCTGATCCCCTGTGTGAGAGAAAAG
AATGGAATAAGCAGAAACTGCCATGCTCAGAGAATCCTAGAGATACTGAAGATGTTCCTTGGATAACACT
AAATAGCAGCATTCAGAAAGTTAATGAGTGGTTTTCCAGAAGTGATGAACTGTTAGGTTCTGATGACTCA
CATGATGGGGAGTCTGAATCAAATGCCAAAGTAGCTGATGTATTGGACGTTCTAAATGAGGTAGATGAAT
ATTCTGGTTCTTCAGAGAAAATAGACTTACTGGCCAGTGATCCTCATGAGGCTTTAATATGTAAAAGTGA
AAGAGTTCACTCCAAATCAGTAGAGAGTAATATTGAAGACAAAATATTTGGGAAAACCTATCGGAAGAAG
GCAAGCCTCCCCAACTTAAGCCATGTAACTGAAAATCTAATTATAGGAGCATTTGTTACTGAGCCACAGA
TAATACAAGAGCGTCCCCTCACAAATAAATTAAAGCGTAAAAGGAGACCTACATCAGGCCTTCATCCTGA
GGATTTTATCAAGAAAGCAGATTTGGCAGTTCAAAAGACTCCTGAAATGATAAATCAGGGAACTAACCAA
ACGGAGCAGAATGGTCAAGTGATGAATATTACTAATAGTGGTCATGAGAATAAAACAAAAGGTGATTCTA
TTCAGAATGAGAAAAATCCTAACCCAATAGAATCACTCGAAAAAGAATCTGCTTTCAAAACGAAAGCTGA
ACCTATAAGCAGCAGTATAAGCAATATGGAACTCGAATTAAATATCCACAATTCAAAAGCACCTAAAAAG
AATAGGCTGAGGAGGAAGTCTTCTACCAGGCATATTCATGCGCTTGAACTAGTAGTCAGTAGAAATCTAA
GCCCACCTAATTGTACTGAATTGCAAATTGATAGTTGTTCTAGCAGTGAAGAGATAAAGAAAAAAAAGTA
CAACCAAATGCCAGTCAGGCACAGCAGAAACCTACAACTCATGGAAGGTAAAGAACCTGCAACTGGAGCC
AAGAAGAGTAACAAGCCAAATGAACAGACAAGTAAAAGACATGACAGCGATACTTTCCCAGAGCTGAAGT
TAACAAATGCACCTGGTTCTTTTACTAAGTGTTCAAATACCAGTGAACTTAAAGAATTTGTCAATCCTAG
CCTTCCAAGAGAAGAAAAAGAAGAGAAACTAGAAACAGTTAAAGTGTCTAATAATGCTGAAGACCCCAAA
GATCTCATGTTAAGTGGAGAAAGGGTTTTGCAAACTGAAAGATCTGTAGAGAGTAGCAGTATTTCATTGG
TACCTGGTACTGATTATGGCACTCAGGAAAGTATCTCGTTACTGGAAGTTAGCACTCTAGGGAAGGCAAA
AACAGAACCAAATAAATGTGTGAGTCAGTGTGCAGCATTTGAAAACCCCAAGGGACTAATTCATGGTTGT
TCCAAAGATAATAGAAATGACACAGAAGGCTTTAAGTATCCATTGGGACATGAAGTTAACCACAGTCGGG
AAACAAGCATAGAAATGGAAGAAAGTGAACTTGATGCTCAGTATTTGCAGAATACATTCAAGGTTTCAAA
GCGCCAGTCATTTGCTCCGTTTTCAAATCCAGGAAATGCAGAAGAGGAATGTGCAACATTCTCTGCCCAC
TCTGGGTCCTTAAAGAAACAAAGTCCAAAAGTCACTTTTGAATGTGAACAAAAGGAAGAAAATCAAGGAA
AGAATGAGTCTAATATCAAGCCTGTACAGACAGTTAATATCACTGCAGGCTTTCCTGTGGTTGGTCAGAA
AGATAAGCCAGTTGATAATGCCAAATGTAGTATCAAAGGAGGCTCTAGGTTTTGTCTATCATCTCAGTTC
AGAGGCAACGAAACTGGACTCATTACTCCAAATAAACATGGACTTTTACAAAACCCATATCGTATACCAC
CACTTTTTCCCATCAAGTCATTTGTTAAAACTAAATGTAAGAAAAATCTGCTAGAGGAAAACTTTGAGGA
ACATTCAATGTCACCTGAAAGAGAAATGGGAAATGAGAACATTCCAAGTACAGTGAGCACAATTAGCCGT
AATAACATTAGAGAAAATGTTTTTAAAGAAGCCAGCTCAAGCAATATTAATGAAGTAGGTTCCAGTACTA
ATGAAGTGGGCTCCAGTATTAATGAAATAGGTTCCAGTGATGAAAACATTCAAGCAGAACTAGGTAGAAA
CAGAGGGCCAAAATTGAATGCTATGCTTAGATTAGGGGTTTTGCAACCTGAGGTCTATAAACAAAGTCTT
CCTGGAAGTAATTGTAAGCATCCTGAAATAAAAAAGCAAGAATATGAAGAAGTAGTTCAGACTGTTAATA
CAGATTTCTCTCCATATCTGATTTCAGATAACTTAGAACAGCCTATGGGAAGTAGTCATGCATCTCAGGT
TTGTTCTGAGACACCTGATGACCTGTTAGATGATGGTGAAATAAAGGAAGATACTAGTTTTGCTGAAAAT
GACATTAAGGAAAGTTCTGCTGTTTTTAGCAAAAGCGTCCAGAAAGGAGAGCTTAGCAGGAGTCCTAGCC
CTTTCACCCATACACATTTGGCTCAGGGTTACCGAAGAGGGGCCAAGAAATTAGAGTCCTCAGAAGAGAA
CTTATCTAGTGAGGATGAAGAGCTTCCCTGCTTCCAACACTTGTTATTTGGTAAAGTAAACAATATACCT
TCTCAGTCTACTAGGCATAGCACCGTTGCTACCGAGTGTCTGTCTAAGAACACAGAGGAGAATTTATTAT
CATTGAAGAATAGCTTAAATGACTGCAGTAACCAGGTAATATTGGCAAAGGCATCTCAGGAACATCACCT
TAGTGAGGAAACAAAATGTTCTGCTAGCTTGTTTTCTTCACAGTGCAGTGAATTGGAAGACTTGACTGCA
AATACAAACACCCAGGATCCTTTCTTGATTGGTTCTTCCAAACAAATGAGGCATCAGTCTGAAAGCCAGG
GAGTTGGTCTGAGTGACAAGGAATTGGTTTCAGATGATGAAGAAAGAGGAACGGGCTTGGAAGAAAATAA
TCAAGAAGAGCAAAGCATGGATTCAAACTTAGGTGAAGCAGCATCTGGGTGTGAGAGTGAAACAAGCGTC
TCTGAAGACTGCTCAGGGCTATCCTCTCAGAGTGACATTTTAACCACTCAGCAGAGGGATACCATGCAAC
ATAACCTGATAAAGCTCCAGCAGGAAATGGCTGAACTAGAAGCTGTGTTAGAACAGCATGGGAGCCAGCC
TTCTAACAGCTACCCTTCCATCATAAGTGACTCTTCTGCCCTTGAGGACCTGCGAAATCCAGAACAAAGC
ACATCAGAAAAAGCAGTATTAACTTCACAGAAAAGTAGTGAATACCCTATAAGCCAGAATCCAGAAGGCC
TTTCTGCTGACAAGTTTGAGGTGTCTGCAGATAGTTCTACCAGTAAAAATAAAGAACCAGGAGTGGAAAG
GTCATCCCCTTCTAAATGCCCATCATTAGATGATAGGTGGTACATGCACAGTTGCTCTGGGAGTCTTCAG
AATAGAAACTACCCATCTCAAGAGGAGCTCATTAAGGTTGTTGATGTGGAGGAGCAACAGCTGGAAGAGT
CTGGGCCACACGATTTGACGGAAACATCTTACTTGCCAAGGCAAGATCTAGAGGGAACCCCTTACCTGGA
ATCTGGAATCAGCCTCTTCTCTGATGACCCTGAATCTGATCCTTCTGAAGACAGAGCCCCAGAGTCAGCT
CGTGTTGGCAACATACCATCTTCAACCTCTGCATTGAAAGTTCCCCAATTGAAAGTTGCAGAATCTGCCC
AGAGTCCAGCTGCTGCTCATACTACTGATACTGCTGGGTATAATGCAATGGAAGAAAGTGTGAGCAGGGA
GAAGCCAGAATTGACAGCTTCAACAGAAAGGGTCAACAAAAGAATGTCCATGGTGGTGTCTGGCCTGACC
CCAGAAGAATTTATGCTCGTGTACAAGTTTGCCAGAAAACACCACATCACTTTAACTAATCTAATTACTG
AAGAGACTACTCATGTTGTTATGAAAACAGATGCTGAGTTTGTGTGTGAACGGACACTGAAATATTTTCT
AGGAATTGCGGGAGGAAAATGGGTAGTTAGCTATTTCTGGGTGACCCAGTCTATTAAAGAAAGAAAAATG
CTGAATGAGCATGATTTTGAAGTCAGAGGAGATGTGGTCAATGGAAGAAACCACCAAGGTCCAAAGCGAG
CAAGAGAATCCCAGGACAGAAAGATCTTCAGGGGGCTAGAAATCTGTTGCTATGGGCCCTTCACCAACAT
GCCCACAGATCAACTGGAATGGATGGTACAGCTGTGTGGTGCTTCTGTGGTGAAGGAGCTTTCATCATTC
ACCCTTGGCACAGGTGTCCACCCAATTGTGGTTGTGCAGCCAGATGCCTGGACAGAGGACAATGGCTTCC
ATGCAATTGGGCAGATGTGTGAGGCACCTGTGGTGACCCGAGAGTGGGTGTTGGACAGTGTAGCACTCTA
CCAGTGCCAGGAGCTGGACACCTACCTGATACCCCAGATCCCCCACAGCCACTACTGACTGCAGCCAGCC
ACAGGTACAGAGCCACAGGACCCCAAGAATGAGCTTACAAAGTGGCCTTTCCAGGCCCTGGGAGCTCCTC
TCACTCTTCAGTCCTTCTACTGTCCTGGCTACTAAATATTTTATGTACATCAGCCTGAAAAGGACTTCTG
GCTATGCAAGGGTCCCTTAAAGATTTTCTGCTTGAAGTCTCCCTTGGAAATCTGCCATGAGCACAAAATT
ATGGTAATTTTTCACCTGAGAAGATTTTAAAACCATTTAAACGCCACCAATTGAGCAAGATGCTGATTCA
TTATTTATCAGCCCTATTCTTTCTATTCAGGCTGTTGTTGGCTTAGGGCTGGAAGCACAGAGTGGCTTGG
CCTCAAGAGAATAGCTGGTTTCCCTAAGTTTACTTCTCTAAAACCCTGTGTTCACAAAGGCAGAGAGTCA
GACCCTTCAATGGAAGGAGAGTGCTTGGGATCGATTATGTGACTTAAAGTCAGAATAGTCCTTGGGCAGT
TCTCAAATGTTGGAGTGGAACATTGGGGAGGAAATTCTGAGGCAGGTATTAGAAATGAAAAGGAAACTTG
AAACCTGGGCATGGTGGCTCACGCCTGTAATCCCAGCACTTTGGGAGGCCAAGGTGGGCAGATCACTGGA
GGTCAGGAGTTCGAAACCAGCCTGGCCAACATGGTGAAACCCCATCTCTACTAAAAATACAGAAATTAGC
CGGTCATGGTGGTGGACACCTGTAATCCCAGCTACTCAGGTGGCTAAGGCAGGAGAATCACTTCAGCCCG
GGAGGTGGAGGTTGCAGTGAGCCAAGATCATACCACGGCACTCCAGCCTGGGTGACAGTGAGACTGTGGC
TCAAAAAAAAAAAAAAAAAAAAAAAAATGAAACTAGAAGAGATTTCTAAAAGTCTGAGATATATTTGCTA
GATTTCTAAAGAATGTGTTCTAAAACAGCAGAAGATTTTCAAGAACCGGTTTCCAAAGACAGTCTTCTAA
TTCCTCATTAGTAATAAGTAAAATGTTTATTGTTGTAGCTCTGGTATATAATCCATTCCTCTTAAAATAT
AAGACCTCTGGCATGAATATTTCATATCTATAAAATGACAGATCCCACCAGGAAGGAAGCTGTTGCTTTC
TTTGAGGTGATTTTTTTCCTTTGCTCCCTGTTGCTGAAACCATACAGCTTCATAAATAATTTTGCTTGCT
GAAGGAAGAAAAAGTGTTTTTCATAAACCCATTATCCAGGACTGTTTATAGCTGTTGGAAGGACTAGGTC
TTCCCTAGCCCCCCCAGTGTGCAAGGGCAGTGAAGACTTGATTGTACAAAATACGTTTTGTAAATGTTGT
GCTGTTAACACTGCAAATAAACTTGGTAGCAAACACTTCCAAAAAAAAAAAAAAAAAA
SEQ ID NO: 10
Homo sapiens CD55 molecule, decay accelerating factor for complement
(Cromer blood group)(CD55), transcript variant 1, mRNA
AGCGAGCTCCTCCTCCTTCCCCTCCCCACTCTCCCCGAGTCTAGGGCCCCCGGGGCGTATGACGCCGGAG
CCCTCTGACCGCACCTCTGACCACAACAAACCCCTACTCCACCCGTCTTGTTTGTCCCACCCTTGGTGAC
GCAGAGCCCCAGCCCAGACCCCGCCCAAAGCACTCATTTAACTGGTATTGCGGAGCCACGAGGCTTCTGC
TTACTGCAACTCGCTCCGGCCGCTGGGCGTAGCTGCGACTCGGCGGAGTCCCGGCGGCGCGTCCTTGTTC
TAACCCGGCGCGCCATGACCGTCGCGCGGCCGAGCGTGCCCGCGGCGCTGCCCCTCCTCGGGGAGCTGCC
CCGGCTGCTGCTGCTGGTGCTGTTGTGCCTGCCGGCCGTGTGGGGTGACTGTGGCCTTCCCCCAGATGTA
CCTAATGCCCAGCCAGCTTTGGAAGGCCGTACAAGTTTTCCCGAGGATACTGTAATAACGTACAAATGTG
AAGAAAGCTTTGTGAAAATTCCTGGCGAGAAGGACTCAGTGATCTGCCTTAAGGGCAGTCAATGGTCAGA
TATTGAAGAGTTCTGCAATCGTAGCTGCGAGGTGCCAACAAGGCTAAATTCTGCATCCCTCAAACAGCCT
TATATCACTCAGAATTATTTTCCAGTCGGTACTGTTGTGGAATATGAGTGCCGTCCAGGTTACAGAAGAG
AACCTTCTCTATCACCAAAACTAACTTGCCTTCAGAATTTAAAATGGTCCACAGCAGTCGAATTTTGTAA
AAAGAAATCATGCCCTAATCCGGGAGAAATACGAAATGGTCAGATTGATGTACCAGGTGGCATATTATTT
GGTGCAACCATCTCCTTCTCATGTAACACAGGGTACAAATTATTTGGCTCGACTTCTAGTTTTTGTCTTA
TTTCAGGCAGCTCTGTCCAGTGGAGTGACCCGTTGCCAGAGTGCAGAGAAATTTATTGTCCAGCACCACC
ACAAATTGACAATGGAATAATTCAAGGGGAACGTGACCATTATGGATATAGACAGTCTGTAACGTATGCA
TGTAATAAAGGATTCACCATGATTGGAGAGCACTCTATTTATTGTACTGTGAATAATGATGAAGGAGAGT
GGAGTGGCCCACCACCTGAATGCAGAGGAAAATCTCTAACTTCCAAGGTCCCACCAACAGTTCAGAAACC
TACCACAGTAAATGTTCCAACTACAGAAGTCTCACCAACTTCTCAGAAAACCACCACAAAAACCACCACA
CCAAATGCTCAAGCAACACGGAGTACACCTGTTTCCAGGACAACCAAGCATTTTCATGAAACAACCCCAA
ATAAAGGAAGTGGAACCACTTCAGGTACTACCCGTCTTCTATCTGGGCACACGTGTTTCACGTTGACAGG
TTTGCTTGGGACGCTAGTAACCATGGGCTTGCTGACTTAGCCAAAGAAGAGTTAAGAAGAAAATACACAC
AAGTATACAGACTGTTCCTAGTTTCTTAGACTTATCTGCATATTGGATAAAATAAATGCAATTGTGCTCT
TCATTTAGGATGCTTTCATTGTCTTTAAGATGTGTTAGGAATGTCAACAGAGCAAGGAGAAAAAAGGCAG
TCCTGGAATCACATTCTTAGCACACCTACACCTCTTGAAAATAGAACAACTTGCAGAATTGAGAGTGATT
CCTTTCCTAAAAGTGTAAGAAAGCATAGAGATTTGTTCGTATTTAGAATGGGATCACGAGGAAAAGAGAA
GGAAAGTGATTTTTTTCCACAAGATCTGTAATGTTATTTCCACTTATAAAGGAAATAAAAAATGAAAAAC
ATTATTTGGATATCAAAAGCAAATAAAAACCCAATTCAGTCTCTTCTAAGCAAAATTGCTAAAGAGAGAT
GAACCACATTATAAAGTAATCTTTGGCTGTAAGGCATTTTCATCTTTCCTTCGGGTTGGCAAAATATTTT
AAAGGTAAAACATGCTGGTGAACCAGGGGTGTTGATGGTGATAAGGGAGGAATATAGAATGAAAGACTGA
ATCTTCCTTTGTTGCACAAATAGAGTTTGGAAAAAGCCTGTGAAAGGTGTCTTCTTTGACTTAATGTCTT
TAAAAGTATCCAGAGATACTACAATATTAACATAAGAAAAGATTATATATTATTTCTGAATCGAGATGTC
CATAGTCAAATTTGTAAATCTTATTCTTTTGTAATATTTATTTATATTTATTTATGACAGTGAACATTCT
GATTTTACATGTAAAACAAGAAAAGTTGAAGAAGATATGTGAAGAAAAATGTATTTTTCCTAAATAGAAA
TAAATGATCCCATTTTTTGGTATCATGTAGTATGTGAAATTTATTCTTAAACGTGACTACTTTATTTCTA
AATAAGAAATTCCCTACCTGCTTCCTACAAGCAGTTCAGAATGCCATGCCTTGGTTGTCCTAGTGTGAAT
AATTTTCAGCTACTTTAAAATTATATTGTACTTTCTCAAGCATGTCATATCCTTTCCTATTAGAGTATCT
ATATTACTTGTTACTGATTTACCTGAAGGCAATCTGATTAATTTCTAGGTTTTTACCATATTCTTGTCAT
CTTGCCAATTACATTTTAAGTGTTAGACTAGACTAAGATGTACTAGTTGTATAGAATATAACTAGATTTA
TTATGGCAATGTTTATTTTGTCATTTTGCTTCATCTGTTTTGTTGTTGAAGTACTTTAAATTTCATACGT
TCATGGCATTTCACTGTAAAGACTTTAATGTGTATTTCTTAAAATAAAACTTTTTTTCCTCCTTAAAAAA
AAAAAAAAAAAA
SEQ ID NO: 11
Homo sapiens cadherin 1, type 1, E-cadherin (epithelial)(CDH1), mRNA
AGTGGCGTCGGAACTGCAAAGCACCTGTGAGCTTGCGGAAGTCAGTTCAGACTCCAGCCCGCTCCAGCCC
GGCCCGACCCGACCGCACCCGGCGCCTGCCCTCGCTCGGCGTCCCCGGCCAGCCATGGGCCCTTGGAGCC
GCAGCCTCTCGGCGCTGCTGCTGCTGCTGCAGGTCTCCTCTTGGCTCTGCCAGGAGCCGGAGCCCTGCCA
CCCTGGCTTTGACGCCGAGAGCTACACGTTCACGGTGCCCCGGCGCCACCTGGAGAGAGGCCGCGTCCTG
GGCAGAGTGAATTTTGAAGATTGCACCGGTCGACAAAGGACAGCCTATTTTTCCCTCGACACCCGATTCA
AAGTGGGCACAGATGGTGTGATTACAGTCAAAAGGCCTCTACGGTTTCATAACCCACAGATCCATTTCTT
GGTCTACGCCTGGGACTCCACCTACAGAAAGTTTTCCACCAAAGTCACGCTGAATACAGTGGGGCACCAC
CACCGCCCCCCGCCCCATCAGGCCTCCGTTTCTGGAATCCAAGCAGAATTGCTCACATTTCCCAACTCCT
CTCCTGGCCTCAGAAGACAGAAGAGAGACTGGGTTATTCCTCCCATCAGCTGCCCAGAAAATGAAAAAGG
CCCATTTCCTAAAAACCTGGTTCAGATCAAATCCAACAAAGACAAAGAAGGCAAGGTTTTCTACAGCATC
ACTGGCCAAGGAGCTGACACACCCCCTGTTGGTGTCTTTATTATTGAAAGAGAAACAGGATGGCTGAAGG
TGACAGAGCCTCTGGATAGAGAACGCATTGCCACATACACTCTCTTCTCTCACGCTGTGTCATCCAACGG
GAATGCAGTTGAGGATCCAATGGAGATTTTGATCACGGTAACCGATCAGAATGACAACAAGCCCGAATTC
ACCCAGGAGGTCTTTAAGGGGTCTGTCATGGAAGGTGCTCTTCCAGGAACCTCTGTGATGGAGGTCACAG
CCACAGACGCGGACGATGATGTGAACACCTACAATGCCGCCATCGCTTACACCATCCTCAGCCAAGATCC
TGAGCTCCCTGACAAAAATATGTTCACCATTAACAGGAACACAGGAGTCATCAGTGTGGTCACCACTGGG
CTGGACCGAGAGAGTTTCCCTACGTATACCCTGGTGGTTCAAGCTGCTGACCTTCAAGGTGAGGGGTTAA
GCACAACAGCAACAGCTGTGATCACAGTCACTGACACCAACGATAATCCTCCGATCTTCAATCCCACCAC
GTACAAGGGTCAGGTGCCTGAGAACGAGGCTAACGTCGTAATCACCACACTGAAAGTGACTGATGCTGAT
GCCCCCAATACCCCAGCGTGGGAGGCTGTATACACCATATTGAATGATGATGGTGGACAATTTGTCGTCA
CCACAAATCCAGTGAACAACGATGGCATTTTGAAAACAGCAAAGGGCTTGGATTTTGAGGCCAAGCAGCA
GTACATTCTACACGTAGCAGTGACGAATGTGGTACCTTTTGAGGTCTCTCTCACCACCTCCACAGCCACC
GTCACCGTGGATGTGCTGGATGTGAATGAAGCCCCCATCTTTGTGCCTCCTGAAAAGAGAGTGGAAGTGT
CCGAGGACTTTGGCGTGGGCCAGGAAATCACATCCTACACTGCCCAGGAGCCAGACACATTTATGGAACA
GAAAATAACATATCGGATTTGGAGAGACACTGCCAACTGGCTGGAGATTAATCCGGACACTGGTGCCATT
TCCACTCGGGCTGAGCTGGACAGGGAGGATTTTGAGCACGTGAAGAACAGCACGTACACAGCCCTAATCA
TAGCTACAGACAATGGTTCTCCAGTTGCTACTGGAACAGGGACACTTCTGCTGATCCTGTCTGATGTGAA
TGACAACGCCCCCATACCAGAACCTCGAACTATATTCTTCTGTGAGAGGAATCCAAAGCCTCAGGTCATA
AACATCATTGATGCAGACCTTCCTCCCAATACATCTCCCTTCACAGCAGAACTAACACACGGGGCGAGTG
CCAACTGGACCATTCAGTACAACGACCCAACCCAAGAATCTATCATTTTGAAGCCAAAGATGGCCTTAGA
GGTGGGTGACTACAAAATCAATCTCAAGCTCATGGATAACCAGAATAAAGACCAAGTGACCACCTTAGAG
GTCAGCGTGTGTGACTGTGAAGGGGCCGCTGGCGTCTGTAGGAAGGCACAGCCTGTCGAAGCAGGATTGC
AAATTCCTGCCATTCTGGGGATTCTTGGAGGAATTCTTGCTTTGCTAATTCTGATTCTGCTGCTCTTGCT
GTTTCTTCGGAGGAGAGCGGTGGTCAAAGAGCCCTTACTGCCCCCAGAGGATGACACCCGGGACAACGTT
TATTACTATGATGAAGAAGGAGGCGGAGAAGAGGACCAGGACTTTGACTTGAGCCAGCTGCACAGGGGCC
TGGACGCTCGGCCTGAAGTGACTCGTAACGACGTTGCACCAACCCTCATGAGTGTCCCCCGGTATCTTCC
CCGCCCTGCCAATCCCGATGAAATTGGAAATTTTATTGATGAAAATCTGAAAGCGGCTGATACTGACCCC
ACAGCCCCGCCTTATGATTCTCTGCTCGTGTTTGACTATGAAGGAAGCGGTTCCGAAGCTGCTAGTCTGA
GCTCCCTGAACTCCTCAGAGTCAGACAAAGACCAGGACTATGACTACTTGAACGAATGGGGCAATCGCTT
CAAGAAGCTGGCTGACATGTACGGAGGCGGCGAGGACGACTAGGGGACTCGAGAGAGGCGGGCCCCAGAC
CCATGTGCTGGGAAATGCAGAAATCACGTTGCTGGTGGTTTTTCAGCTCCCTTCCCTTGAGATGAGTTTC
TGGGGAAAAAAAAGAGACTGGTTAGTGATGCAGTTAGTATAGCTTTATACTCTCTCCACTTTATAGCTCT
AATAAGTTTGTGTTAGAAAAGTTTCGACTTATTTCTTAAAGCTTTTTTTTTTTTCCCATCACTCTTTACA
TGGTGGTGATGTCCAAAAGATACCCAAATTTTAATATTCCAGAAGAACAACTTTAGCATCAGAAGGTTCA
CCCAGCACCTTGCAGATTTTCTTAAGGAATTTTGTCTCACTTTTAAAAAGAAGGGGAGAAGTCAGCTACT
CTAGTTCTGTTGTTTTGTGTATATAATTTTTTAAAAAAAATTTGTGTGCTTCTGCTCATTACTACACTGG
TGTGTCCCTCTGCCTTTTTTTTTTTTTTAAGACAGGGTCTCATTCTATCGGCCAGGCTGGAGTGCAGTGG
TGCAATCACAGCTCACTGCAGCCTTGTCCTCCCAGGCTCAAGCTATCCTTGCACCTCAGCCTCCCAAGTA
GCTGGGACCACAGGCATGCACCACTACGCATGACTAATTTTTTAAATATTTGAGACGGGGTCTCCCTGTG
TTACCCAGGCTGGTCTCAAACTCCTGGGCTCAAGTGATCCTCCCATCTTGGCCTCCCAGAGTATTGGGAT
TACAGACATGAGCCACTGCACCTGCCCAGCTCCCCAACTCCCTGCCATTTTTTAAGAGACAGTTTCGCTC
CATCGCCCAGGCCTGGGATGCAGTGATGTGATCATAGCTCACTGTAACCTCAAACTCTGGGGCTCAAGCA
GTTCTCCCACCAGCCTCCTTTTTATTTTTTTGTACAGATGGGGTCTTGCTATGTTGCCCAAGCTGGTCTT
AAACTCCTGGCCTCAAGCAATCCTTCTGCCTTGGCCCCCCAAAGTGCTGGGATTGTGGGCATGAGCTGCT
GTGCCCAGCCTCCATGTTTTAATATCAACTCTCACTCCTGAATTCAGTTGCTTTGCCCAAGATAGGAGTT
CTCTGATGCAGAAATTATTGGGCTCTTTTAGGGTAAGAAGTTTGTGTCTTTGTCTGGCCACATCTTGACT
AGGTATTGTCTACTCTGAAGACCTTTAATGGCTTCCCTCTTTCATCTCCTGAGTATGTAACTTGCAATGG
GCAGCTATCCAGTGACTTGTTCTGAGTAAGTGTGTTCATTAATGTTTATTTAGCTCTGAAGCAAGAGTGA
TATACTCCAGGACTTAGAATAGTGCCTAAAGTGCTGCAGCCAAAGACAGAGCGGAACTATGAAAAGTGGG
CTTGGAGATGGCAGGAGAGCTTGTCATTGAGCCTGGCAATTTAGCAAACTGATGCTGAGGATGATTGAGG
TGGGTCTACCTCATCTCTGAAAATTCTGGAAGGAATGGAGGAGTCTCAACATGTGTTTCTGACACAAGAT
CCGTGGTTTGTACTCAAAGCCCAGAATCCCCAAGTGCCTGCTTTTGATGATGTCTACAGAAAATGCTGGC
TGAGCTGAACACATTTGCCCAATTCCAGGTGTGCACAGAAAACCGAGAATATTCAAAATTCCAAATTTTT
TTCTTAGGAGCAAGAAGAAAATGTGGCCCTAAAGGGGGTTAGTTGAGGGGTAGGGGGTAGTGAGGATCTT
GATTTGGATCTCTTTTTATTTAAATGTGAATTTCAACTTTTGACAATCAAAGAAAAGACTTTTGTTGAAA
TAGCTTTACTGTTTCTCAAGTGTTTTGGAGAAAAAAATCAACCCTGCAATCACTTTTTGGAATTGTCTTG
ATTTTTCGGCAGTTCAAGCTATATCGAATATAGTTCTGTGTAGAGAATGTCACTGTAGTTTTGAGTGTAT
ACATGTGTGGGTGCTGATAATTGTGTATTTTCTTTGGGGGTGGAAAAGGAAAACAATTCAAGCTGAGAAA
AGTATTCTCAAAGATGCATTTTTATAAATTTTATTAAACAATTTTGTTAAACCAT
SEQ ID NO: 12
Homo sapiens cyclin-dependent kinase inhibitor 1B (p27, Kip1)(CDKN1B),
mRNA
CTTCTTCGTCAGCCTCCCTTCCACCGCCATATTGGGCCACTAAAAAAAGGGGGCTCGTCTTTTCGGGGTG
TTTTTCTCCCCCTCCCCTGTCCCCGCTTGCTCACGGCTCTGCGACTCCGACGCCGGCAAGGTTTGGAGAG
CGGCTGGGTTCGCGGGACCCGCGGGCTTGCACCCGCCCAGACTCGGACGGGCTTTGCCACCCTCTCCGCT
TGCCTGGTCCCCTCTCCTCTCCGCCCTCCCGCTCGCCAGTCCATTTGATCAGCGGAGACTCGGCGGCCGG
GCCGGGGCTTCCCCGCAGCCCCTGCGCGCTCCTAGAGCTCGGGCCGTGGCTCGTCGGGGTCTGTGTCTTT
TGGCTCCGAGGGCAGTCGCTGGGCTTCCGAGAGGGGTTCGGGCTGCGTAGGGGCGCTTTGTTTTGTTCGG
TTTTGTTTTTTTGAGAGTGCGAGAGAGGCGGTCGTGCAGACCCGGGAGAAAGATGTCAAACGTGCGAGTG
TCTAACGGGAGCCCTAGCCTGGAGCGGATGGACGCCAGGCAGGCGGAGCACCCCAAGCCCTCGGCCTGCA
GGAACCTCTTCGGCCCGGTGGACCACGAAGAGTTAACCCGGGACTTGGAGAAGCACTGCAGAGACATGGA
AGAGGCGAGCCAGCGCAAGTGGAATTTCGATTTTCAGAATCACAAACCCCTAGAGGGCAAGTACGAGTGG
CAAGAGGTGGAGAAGGGCAGCTTGCCCGAGTTCTACTACAGACCCCCGCGGCCCCCCAAAGGTGCCTGCA
AGGTGCCGGCGCAGGAGAGCCAGGATGTCAGCGGGAGCCGCCCGGCGGCGCCTTTAATTGGGGCTCCGGC
TAACTCTGAGGACACGCATTTGGTGGACCCAAAGACTGATCCGTCGGACAGCCAGACGGGGTTAGCGGAG
CAATGCGCAGGAATAAGGAAGCGACCTGCAACCGACGATTCTTCTACTCAAAACAAAAGAGCCAACAGAA
CAGAAGAAAATGTTTCAGACGGTTCCCCAAATGCCGGTTCTGTGGAGCAGACGCCCAAGAAGCCTGGCCT
CAGAAGACGTCAAACGTAAACAGCTCGAATTAAGAATATGTTTCCTTGTTTATCAGATACATCACTGCTT
GATGAAGCAAGGAAGATATACATGAAAATTTTAAAAATACATATCGCTGACTTCATGGAATGGACATCCT
GTATAAGCACTGAAAAACAACAACACAATAACACTAAAATTTTAGGCACTCTTAAATGATCTGCCTCTAA
AAGCGTTGGATGTAGCATTATGCAATTAGGTTTTTCCTTATTTGCTTCATTGTACTACCTGTGTATATAG
TTTTTACCTTTTATGTAGCACATAAACTTTGGGGAAGGGAGGGCAGGGTGGGGCTGAGGAACTGACGTGG
AGCGGGGTATGAAGAGCTTGCTTTGATTTACAGCAAGTAGATAAATATTTGACTTGCATGAAGAGAAGCA
ATTTTGGGGAAGGGTTTGAATTGTTTTCTTTAAAGATGTAATGTCCCTTTCAGAGACAGCTGATACTTCA
TTTAAAAAAATCACAAAAATTTGAACACTGGCTAAAGATAATTGCTATTTATTTTTACAAGAAGTTTATT
CTCATTTGGGAGATCTGGTGATCTCCCAAGCTATCTAAAGTTTGTTAGATAGCTGCATGTGGCTTTTTTA
AAAAAGCAACAGAAACCTATCCTCACTGCCCTCCCCAGTCTCTCTTAAAGTTGGAATTTACCAGTTAATT
ACTCAGCAGAATGGTGATCACTCCAGGTAGTTTGGGGCAAAAATCCGAGGTGCTTGGGAGTTTTGAATGT
TAAGAATTGACCATCTGCTTTTATTAAATTTGTTGACAAAATTTTCTCATTTTCTTTTCACTTCGGGCTG
TGTAAACACAGTCAAAATAATTCTAAATCCCTCGATATTTTTAAAGATCTGTAAGTAACTTCACATTAAA
AAATGAAATATTTTTTAATTTAAAGCTTACTCTGTCCATTTATCCACAGGAAAGTGTTATTTTTCAAGGA
AGGTTCATGTAGAGAAAAGCACACTTGTAGGATAAGTGAAATGGATACTACATCTTTAAACAGTATTTCA
TTGCCTGTGTATGGAAAAACCATTTGAAGTGTACCTGTGTACATAACTCTGTAAAAACACTGAAAAATTA
TACTAACTTATTTATGTTAAAAGATTTTTTTTAATCTAGACAATATACAAGCCAAAGTGGCATGTTTTGT
GCATTTGTAAATGCTGTGTTGGGTAGAATAGGTTTTCCCCTCTTTTGTTAAATAATATGGCTATGCTTAA
AAGGTTGCATACTGAGCCAAGTATAATTTTTTGTAATGTGTGAAAAAGATGCCAATTATTGTTACACATT
AAGTAATCAATAAAGAAAACTTCCATAGCTATT
SEQ ID NO: 13
Homo sapiens checkpoint kinase 2 (CHEK2), transcript variant 3, mRNA
GCAGGTTTAGCGCCACTCTGCTGGCTGAGGCTGCGGAGAGTGTGCGGCTCCAGGTGGGCTCACGCGGTCG
TGATGTCTCGGGAGTCGGATGTTGAGGCTCAGCAGTCTCATGGCAGCAGTGCCTGTTCACAGCCCCATGG
CAGCGTTACCCAGTCCCAAGGCTCCTCCTCACAGTCCCAGGGCATATCCAGCTCCTCTACCAGCACGATG
CCAAACTCCAGCCAGTCCTCTCACTCCAGCTCTGGGACACTGAGCTCCTTAGAGACAGTGTCCACTCAGG
AACTCTATTCTATTCCTGAGGACCAAGAACCTGAGGACCAAGAACCTGAGGAGCCTACCCCTGCCCCCTG
GGCTCGATTATGGGCCCTTCAGGATGGATTTGCCAATCTTGAGACAGAGTCTGGCCATGTTACCCAATCT
GATCTTGAACTCCTGCTGTCATCTGATCCTCCTGCCTCAGCCTCCCAAAGTGCTGGGATAAGAGGTGTGA
GGCACCATCCCCGGCCAGTTTGCAGTCTAAAATGTGTGAATGACAACTACTGGTTTGGGAGGGACAAAAG
CTGTGAATATTGCTTTGATGAACCACTGCTGAAAAGAACAGATAAATACCGAACATACAGCAAGAAACAC
TTTCGGATTTTCAGGGAAGTGGGTCCTAAAAACTCTTACATTGCATACATAGAAGATCACAGTGGCAATG
GAACCTTTGTAAATACAGAGCTTGTAGGGAAAGGAAAACGCCGTCCTTTGAATAACAATTCTGAAATTGC
ACTGTCACTAAGCAGAAATAAAGTTTTTGTCTTTTTTGATCTGACTGTAGATGATCAGTCAGTTTATCCT
AAGGCATTAAGAGATGAATACATCATGTCAAAAACTCTTGGAAGTGGTGCCTGTGGAGAGGTAAAGCTGG
CTTTCGAGAGGAAAACATGTAAGAAAGTAGCCATAAAGATCATCAGCAAAAGGAAGTTTGCTATTGGTTC
AGCAAGAGAGGCAGACCCAGCTCTCAATGTTGAAACAGAAATAGAAATTTTGAAAAAGCTAAATCATCCT
TGCATCATCAAGATTAAAAACTTTTTTGATGCAGAAGATTATTATATTGTTTTGGAATTGATGGAAGGGG
GAGAGCTGTTTGACAAAGTGGTGGGGAATAAACGCCTGAAAGAAGCTACCTGCAAGCTCTATTTTTACCA
GATGCTCTTGGCTGTGCAGTACCTTCATGAAAACGGTATTATACACCGTGACTTAAAGCCAGAGAATGTT
TTACTGTCATCTCAAGAAGAGGACTGTCTTATAAAGATTACTGATTTTGGGCACTCCAAGATTTTGGGAG
AGACCTCTCTCATGAGAACCTTATGTGGAACCCCCACCTACTTGGCGCCTGAAGTTCTTGTTTCTGTTGG
GACTGCTGGGTATAACCGTGCTGTGGACTGCTGGAGTTTAGGAGTTATTCTTTTTATCTGCCTTAGTGGG
TATCCACCTTTCTCTGAGCATAGGACTCAAGTGTCACTGAAGGATCAGATCACCAGTGGAAAATACAACT
TCATTCCTGAAGTCTGGGCAGAAGTCTCAGAGAAAGCTCTGGACCTTGTCAAGAAGTTGTTGGTAGTGGA
TCCAAAGGCACGTTTTACGACAGAAGAAGCCTTAAGACACCCGTGGCTTCAGGATGAAGACATGAAGAGA
AAGTTTCAAGATCTTCTGTCTGAGGAAAATGAATCCACAGCTCTACCCCAGGTTCTAGCCCAGCCTTCTA
CTAGTCGAAAGCGGCCCCGTGAAGGGGAAGCCGAGGGTGCCGAGACCACAAAGCGCCCAGCTGTGTGTGC
TGCTGTGTTGTGAACTCCGTGGTTTGAACACGAAAGAAATGTACCTTCTTTCACTCTGTCATCTTTCTTT
TCTTTGAGTCTGTTTTTTTATAGTTTGTATTTTAATTATGGGAATAATTGCTTTTTCACAGTCACTGATG
TACAATTAAAAACCTGATGGAACCTGGAAAA
SEQ ID NO: 14
Homo sapiens colony stimulating factor 3 receptor (granulocyte)(CSF3R),
transcript variant 3, mRNA
GAGTACTGTGAAGATGTGGTCCCCAAGGCTAGAGCTGAAAAGAGGCTTAGGGCCGGGTGAGCCTTCCAGC
CAGGGCCTGCCTCCAAGTGATGCTCCCCCAGGGCAGGGGGCATAAGGATGGCACCCAGCCAGGTGGGAGC
CTGGGCCCTGCCCAGCCTCAAAGCTTTGAGCTCAGGAAATCCGGAGGCAGGGGAGGGGGACATCGTTGCC
ACATTCCCCAGCCCTTTAAGACCCCCAAGGCAGGAAGGCTGCCCGGGCCTCACCAGCTTCCCTCACAGGC
TCCTTCCTGGGAGGAAGGGGCTGCCTGTGCCCTCGAAGGCGCAAGGGAGGGCAGGAGGGAGGCTCGGAAG
GTGTTGCAATCCCCAGCCCCCGGGCCTGTCAGAGGCTGAGCCATTAACGACAGAGCTCGGGGAGAGAAGC
TGGACTGCAGCTGGTTTCAGGAACTTCTCTTGACGAGAAGAGAGACCAAGGAGGCCAAGCAGGGGCTGGG
CCAGAGGTGCCAACATGGGGAAACTGAGGCTCGGCTCGGAAAGGTGAAGTAACTTGTCCAAGATCACAAA
GCTGGTGAACATCAAGTTGGTGCTATGGCAAGGCTGGGAAACTGCAGCCTGACTTGGGCTGCCCTGATCA
TCCTGCTGCTCCCCGGAAGTCTGGAGGAGTGCGGGCACATCAGTGTCTCAGCCCCCATCGTCCACCTGGG
GGATCCCATCACAGCCTCCTGCATCATCAAGCAGAACTGCAGCCATCTGGACCCGGAGCCACAGATTCTG
TGGAGACTGGGAGCAGAGCTTCAGCCCGGGGGCAGGCAGCAGCGTCTGTCTGATGGGACCCAGGAATCTA
TCATCACCCTGCCCCACCTCAACCACACTCAGGCCTTTCTCTCCTGCTGCCTGAACTGGGGCAACAGCCT
GCAGATCCTGGACCAGGTTGAGCTGCGCGCAGGCTACCCTCCAGCCATACCCCACAACCTCTCCTGCCTC
ATGAACCTCACAACCAGCAGCCTCATCTGCCAGTGGGAGCCAGGACCTGAGACCCACCTACCCACCAGCT
TCACTCTGAAGAGTTTCAAGAGCCGGGGCAACTGTCAGACCCAAGGGGACTCCATCCTGGACTGCGTGCC
CAAGGACGGGCAGAGCCACTGCTGCATCCCACGCAAACACCTGCTGTTGTACCAGAATATGGGCATCTGG
GTGCAGGCAGAGAATGCGCTGGGGACCAGCATGTCCCCACAACTGTGTCTTGATCCCATGGATGTTGTGA
AACTGGAGCCCCCCATGCTGCGGACCATGGACCCCAGCCCTGAAGCGGCCCCTCCCCAGGCAGGCTGCCT
ACAGCTGTGCTGGGAGCCATGGCAGCCAGGCCTGCACATAAATCAGAAGTGTGAGCTGCGCCACAAGCCG
CAGCGTGGAGAAGCCAGCTGGGCACTGGTGGGCCCCCTCCCCTTGGAGGCCCTTCAGTATGAGCTCTGCG
GGCTCCTCCCAGCCACGGCCTACACCCTGCAGATACGCTGCATCCGCTGGCCCCTGCCTGGCCACTGGAG
CGACTGGAGCCCCAGCCTGGAGCTGAGAACTACCGAACGGGCCCCCACTGTCAGACTGGACACATGGTGG
CGGCAGAGGCAGCTGGACCCCAGGACAGTGCAGCTGTTCTGGAAGCCAGTGCCCCTGGAGGAAGACAGCG
GACGGATCCAAGGTTATGTGGTTTCTTGGAGACCCTCAGGCCAGGCTGGGGCCATCCTGCCCCTCTGCAA
CACCACAGAGCTCAGCTGCACCTTCCACCTGCCTTCAGAAGCCCAGGAGGTGGCCCTTGTGGCCTATAAC
TCAGCCGGGACCTCTCGTCCCACTCCGGTGGTCTTCTCAGAAAGCAGAGGCCCAGCTCTGACCAGACTCC
ATGCCATGGCCCGAGACCCTCACAGCCTCTGGGTAGGCTGGGAGCCCCCCAATCCATGGCCTCAGGGCTA
TGTGATTGAGTGGGGCCTGGGCCCCCCCAGCGCGAGCAATAGCAACAAGACCTGGAGGATGGAACAGAAT
GGGAGAGCCACGGGGTTTCTGCTGAAGGAGAACATCAGGCCCTTTCAGCTCTATGAGATCATCGTGACTC
CCTTGTACCAGGACACCATGGGACCCTCCCAGCATGTCTATGCCTACTCTCAAGAAATGGCTCCCTCCCA
TGCCCCAGAGCTGCATCTAAAGCACATTGGCAAGACCTGGGCACAGCTGGAGTGGGTGCCTGAGCCCCCT
GAGCTGGGGAAGAGCCCCCTTACCCACTACACCATCTTCTGGACCAACGCTCAGAACCAGTCCTTCTCCG
CCATCCTGAATGCCTCCTCCCGTGGCTTTGTCCTCCATGGCCTGGAGCCCGCCAGTCTGTATCACATCCA
CCTCATGGCTGCCAGCCAGGCTGGGGCCACCAACAGTACAGTCCTCACCCTGATGACCTTGACCCCAGAG
GGGTCGGAGCTACACATCATCCTGGGCCTGTTCGGCCTCCTGCTGTTGCTCACCTGCCTCTGTGGAACTG
CCTGGCTCTGTTGCAGCCCCAACAGGAAGAATCCCCTCTGGCCAAGTGTCCCAGACCCAGCTCACAGCAG
CCTGGGCTCCTGGGTGCCCACAATCATGGAGGAGCTGCCCGGACCCAGACAGGGACAGTGGCTGGGGCAG
ACATCTGAAATGAGCCGTGCTCTCACCCCACATCCTTGTGTGCAGGATGCCTTCCAGCTGCCCGGCCTTG
GCACGCCACCCATCACCAAGCTCACAGTGCTGGAGGAGGATGAAAAGAAGCCGGTGCCCTGGGAGTCCCA
TAACAGCTCAGAGACCTGTGGCCTCCCCACTCTGGTCCAGACCTATGTGCTCCAGGGGGACCCAAGAGCA
GTTTCCACCCAGCCCCAATCCCAGTCTGGCACCAGCGATCAGGTCCTTTATGGGCAGCTGCTGGGCAGCC
CCACAAGCCCAGGGCCAGGGCACTATCTCCGCTGTGACTCCACTCAGCCCCTCTTGGCGGGCCTCACCCC
CAGCCCCAAGTCCTATGAGAACCTCTGGTTCCAGGCCAGCCCCTTGGGGACCCTGGTAACCCCAGCCCCA
AGCCAGGAGGACGACTGTGTCTTTGGGCCACTGCTCAACTTCCCCCTCCTGCAGGGGATCCGGGTCCATG
GGATGGAGGCGCTGGGGAGCTTCTAGGGCTTCCTGGGGTTCCCTTCTTGGGCCTGCCTCTTAAAGGCCTG
AGCTAGCTGGAGAAGAGGGGAGGGTCCATAAGCCCATGACTAAAAACTACCCCAGCCCAGGCTCTCACCA
TCTCCAGTCACCAGCATCTCCCTCTCCTCCCAATCTCCATAGGCTGGGCCTCCCAGGCGATCTGCATACT
TTAAGGACCAGATCATGCTCCATCCAGCCCCACCCAATGGCCTTTTGTGCTTGTTTCCTATAACTTCAGT
ATTGTAAACTAGTTTTTGGTTTGCAGTTTTTGTTGTTGTTTATAGACACTCTTGGGTGTAAAAAAAAAAA
SEQ ID NO: 15
CYHomo sapiens cathepsin S (CTSS), transcript variant 1, mRNA
GACAAGGGCTCTTCTTGATGGCTTACTGTATCCACTTTGTCCCCAAGACCATAGGGAAATGACTAGAGGT
GACTGTACTAGCTAGATTTTAAATGAAACTGAAATGAAAGTTCACTTCCTCATTTTGAGTACCTCATGTG
ACAAGTTCCAATTTCTTTTCAAGTCAATTGAACTGAAATCTCCTTGTTGCTTTGAAATCTTAGAAGAGAG
CCCACTAATTCAAGGACTCTTACTGTGGGAGCAACTGCTGGTTCTATCACAATGAAACGGCTGGTTTGTG
TGCTCTTGGTGTGCTCCTCTGCAGTGGCACAGTTGCATAAAGATCCTACCCTGGATCACCACTGGCATCT
CTGGAAGAAAACCTATGGCAAACAATACAAGGAAAAGAATGAAGAAGCAGTACGACGTCTCATCTGGGAA
AAGAATCTAAAGTTTGTGATGCTTCACAACCTGGAGCATTCAATGGGAATGCACTCATACGATCTGGGCA
TGAACCACCTGGGAGACATGACCAGTGAAGAAGTGATGTCTTTGATGAGTTCCCTGAGAGTTCCCAGCCA
GTGGCAGAGAAATATCACATATAAGTCAAACCCTAATCGGATATTGCCTGATTCTGTGGACTGGAGAGAG
AAAGGGTGTGTTACTGAAGTGAAATATCAAGGTTCTTGTGGTGCTTGCTGGGCTTTCAGTGCTGTGGGGG
CCCTGGAAGCACAGCTGAAGCTGAAAACAGGAAAGCTGGTGTCTCTCAGTGCCCAGAACCTGGTGGATTG
CTCAACTGAAAAATATGGAAACAAAGGCTGCAATGGTGGCTTCATGACAACGGCTTTCCAGTACATCATT
GATAACAAGGGCATCGACTCAGACGCTTCCTATCCCTACAAAGCCATGGATCAGAAATGTCAATATGACT
CAAAATATCGTGCTGCCACATGTTCAAAGTACACTGAACTTCCTTATGGCAGAGAAGATGTCCTGAAAGA
AGCTGTGGCCAATAAAGGCCCAGTGTCTGTTGGTGTAGATGCGCGTCATCCTTCTTTCTTCCTCTACAGA
AGTGGTGTCTACTATGAACCATCCTGTACTCAGAATGTGAATCATGGTGTACTTGTGGTTGGCTATGGTG
ATCTTAATGGGAAAGAATACTGGCTTGTGAAAAACAGCTGGGGCCACAACTTTGGTGAAGAAGGATATAT
TCGGATGGCAAGAAATAAAGGAAATCATTGTGGGATTGCTAGCTTTCCCTCTTACCCAGAAATCTAGAGG
ATCTCTCCTTTTTATAACAAATCAAGAAATATGAAGCACTTTCTCTTAACTTAATTTTTCCTGCTGTATC
CAGAAGAAATAATTGTGTCATGATTAATGTGTATTTACTGTACTAATTAGAAAATATAGTTTGAGGCCGG
GCACGGTGGCTCACGCCTGTAATCCCAGTACTTGGGAGGCCAAGGCAGGCATATCAACTTGAGGCCAGGA
GTTAAAGAGCAGCCTGGCTAACATGGTGAAACCCCATCTCTACTAAAAATACAAAAAATTAGCCGAGCAC
GGTGGTGCATGCCTGTAATCCCAGCTACTTGGGAGGCTGAGGCACGAGATTCCTTGAACCCAAGAGGTTG
AGGCTATGTTGAGCTGAGATCACACCACTGTACTCCAGCCTGGATGACAGAGTGGAGACTCTGTTTCAAA
AAAACAGAAAAGAAAATATAGTTTGATTCTTCATTTTTTTAAATTTGCAAATCTCAGGATAAAGTTTGCT
AAGTAAATTAGTAATGTACTATAGATATAACTGTACAAAAATTGTTCAACCTAAAACAATCTGTAATTGC
TTATTGTTTTATTGTATACTCTTTGTCTTTTTAAGACCCCTAATAGCCTTTTGTAACTTGATGGCTTAAA
AATACTTAATAAATCTGCCATTTCAAATTTCTATCATTGCCACATACCATTCTTATTCCTAGGCAACTAT
TAATAATCTATCCTGAGAATATTAATTGTGGTATTCTGGTGATGGGGTTTAGCAACTTTGATGGAAGAAA
ATATTAGGCTATAAATGTCCTAAGGACTCAGATTGTATCTTTGTACAGAAGAGGATTCAAAACGCCACGT
GTAGTGGCTCATGCCTGTAATCCCAACACTTTGGGAGGCTGAAGTAGGAGGATCGTCTTGAGCCCAGGAG
TTCAAGACCAGCCTGGACAACATAGTGAGACCTTGTCTCCACAAAAATAAAAAAGAAACTATCCAGGAGT
GGTGGTGTGTGCCTGTGGTCCCTGCTATGCAGATGTCTAAGACAGGAGGATCACAAGAGCCCAGGAGGTT
GAGAATGCAGTGAGCTTGTAATTGCACCACTGCACTCCAGCCTGGGTGACAGAGCAAGACCCTGTCTTAA
AAAAAGAGGATTCAACACATATTTTTATATTATGTTAAAGTAAAGAAATGCATAAAAGACAAGCACTTTG
GAAGAATTATTTTAATGATCAACAATTTAATGTATTAGTCCAAATTATTTTTACGTAGTCATCAACAATT
TGACCAGGGCCTTTATTTGGCAAATAACTGAGCCAACCAGAATAAAATAACCAATACTCCACTGCTCATA
TTTTTATCTAATTCAGATGGATCTTCCTTACAACTGCTCTAGATTAGTAGATGCATCTAAGCAGGCAGCA
GGAACTTTAAATTTTTTAAGTTCATGTCTATGACATGAACAATGTGTGGGATAATGTCATTAATATATCC
TAAATTAACCTAAACGTATTTCACTAACTCTGGCTCCTTCTCCATAAAGCACATTTTAAGGAACAAGAAT
TGCTAAATATAAAAACATAAATAATACCATAATACATGGCTATCATCAAAAGTGTATAGAATATTATAGT
TTAAAAGTATTTAGTTGATTACTTTTCAGTTTTGTTTTGTTTTTTGAGACGGAGTCTCACTCTGTTGCCC
AGGCTGGAGTGCAGTGGCACCATCTCAGTTCACTGCAACTTCTGCCTCCCGAGTTCAAGCGATTCTCCTG
CCTCAGCCTCCCGAGTAGCTGGAATTATAGGCGTGCACCACCACGCCCAGCTAATTTTTGTATTTTTAGT
AAAGACAGGGTTTTGCCACATTAGCCAGGCTGGTCTCAAACTCCTGACCTCAGGTGATCCACCCACCCCA
GCCTCCCAAAGTGCTAAGATTACAGGCGTGAGCCACTGAGCCCAGCCTACTTTTCAGTTTTTAACATAAT
TTTTGTTTTATCCACAACTTTTCAAGTATTGAAAGTAGAATAAAAACATGGGTTCTTAGTCTTTAGCTAT
CTGTTAAAGCCTATGAATGCCTTCTTAAAATCATGTTTTTAAATGCATAAAATATATAGGATTACAAAGG
AATCTAATTATATCGAAATACAGTTATTAAAATGTTAAAAGATAAGTTTGTTATATATTAATATGCATGC
TTCTTTATAAATGCATTAAATAAGAGTTAATAGCTATCCTAAATTTGAAATAGTGATAAGCATAATGAAA
ATAGATGCAAAAAACTAATGTGATATGAAAATATCTGGGTTTTTCTTTTGATGATGAAGTATTGCTAATA
TTACCGTGGTTTATGAACTATGTTCAGAATTGAAGAAAATCCTAACTTTCAGTTAGAGGTTAGTGACGGG
GTTCAGGACACCCTACACAAAATACAGCACTTTGACATATTGAATATTTTAAGCTGAAGGCATTTGAGGA
AATTGCAGAAGCAGGAAGGTGACTCTGACCTTCTGCCTGCTGTTCTCCCCAGAAGCAGCCATAAAACCTG
GGAAGGATTTTCTGACCTTCCCCTGAAGTAGATCATAAGACTGTCATGTAAGAGGTGCTCTCCTGGCACC
CAGAGAAAAGGAGCATCCTTACCTCCAAAAGCACAGGGACACAAAGAGGAATCTAAACAAACAGGCCTCT
CAGTTTCCCCCAGTTTATTACATTTAGCTTGTTCACACTTTGCCCTATGACATTTCTACATCACTGGCTG
CTCTTCATCAAACCTACTATAAAAAACATTCAAGTTCAACTGTTTCTTTGGGCCTTTATTTCCTTATGGA
GCCCCTCGTGTCGTGTAAAACTTATATTAAATAAATGTGCATGCTTT
SEQ ID NO: 16
Homo sapiens epoxide hydrolase 2, cytoplasmic (EPHX2), mRNA
CTGGGCGGGTCATGCGCCCTGGCCTTCGCGCATCTCCCAGGTTAGCTGCGTGTCCGGGTGCTAGGCTGCA
GACCCGCCGCCATGACGCTGCGCGCGGCCGTCTTCGACCTTGACGGGGTGCTGGCGCTGCCAGCGGTGTT
CGGCGTCCTCGGCCGCACGGAGGAGGCCCTGGCGCTGCCCAGAGGACTTCTGAATGATGCTTTCCAGAAA
GGGGGACCAGAGGGTGCCACTACCCGGCTTATGAAAGGAGAGATCACACTTTCCCAGTGGATACCACTCA
TGGAAGAAAACTGCAGGAAGTGCTCCGAGACCGCTAAAGTCTGCCTCCCCAAGAATTTCTCCATAAAAGA
AATCTTTGACAAGGCGATTTCAGCCAGAAAGATCAACCGCCCCATGCTCCAGGCAGCTCTCATGCTCAGG
AAGAAAGGATTCACTACTGCCATCCTCACCAACACCTGGCTGGACGACCGTGCTGAGAGAGATGGCCTGG
CCCAGCTGATGTGTGAGCTGAAGATGCACTTTGACTTCCTGATAGAGTCGTGTCAGGTGGGAATGGTCAA
ACCTGAACCTCAGATCTACAAGTTTCTGCTGGACACCCTGAAGGCCAGCCCCAGTGAGGTCGTTTTTTTG
GATGACATCGGGGCTAATCTGAAGCCAGCCCGTGACTTGGGAATGGTCACCATCCTGGTCCAGGACACTG
ACACGGCCCTGAAAGAACTGGAGAAAGTGACCGGAATCCAGCTTCTCAATACCCCGGCCCCTCTGCCGAC
CTCTTGCAATCCAAGTGACATGAGCCATGGGTACGTGACAGTAAAGCCCAGGGTCCGTCTGCATTTTGTG
GAGCTGGGCTCCGGCCCTGCTGTGTGCCTCTGCCATGGATTTCCCGAGAGTTGGTATTCTTGGAGGTACC
AGATCCCTGCTCTGGCCCAGGCAGGTTACCGGGTCCTAGCTATGGACATGAAAGGCTATGGAGAGTCATC
TGCTCCTCCCGAAATAGAAGAATATTGCATGGAAGTGTTATGTAAGGAGATGGTAACCTTCCTGGATAAA
CTGGGCCTCTCTCAAGCAGTGTTCATTGGCCATGACTGGGGTGGCATGCTGGTGTGGTACATGGCTCTCT
TCTACCCCGAGAGAGTGAGGGCGGTGGCCAGTTTGAATACTCCCTTCATACCAGCAAATCCCAACATGTC
CCCTTTGGAGAGTATCAAAGCCAACCCAGTATTTGATTACCAGCTCTACTTCCAAGAACCAGGAGTGGCT
GAGGCTGAACTGGAACAGAACCTGAGTCGGACTTTCAAAAGCCTCTTCAGAGCAAGCGATGAGAGTGTTT
TATCCATGCATAAAGTCTGTGAAGCGGGAGGACTTTTTGTAAATAGCCCAGAAGAGCCCAGCCTCAGCAG
GATGGTCACTGAGGAGGAAATCCAGTTCTATGTGCAGCAGTTCAAGAAGTCTGGTTTCAGAGGTCCTCTA
AACTGGTACCGAAACATGGAAAGGAACTGGAAGTGGGCTTGCAAAAGCTTGGGACGGAAGATCCTGATTC
CGGCCCTGATGGTCACGGCGGAGAAGGACTTCGTGCTCGTTCCTCAGATGTCCCAGCACATGGAGGACTG
GATTCCCCACCTGAAAAGGGGACACATTGAGGACTGTGGGCACTGGACACAGATGGACAAGCCAACCGAG
GTGAATCAGATCCTCATTAAGTGGCTGGATTCTGATGCCCGGAACCCACCGGTGGTCTCAAAGATGTAGA
ACGCAGCGTGTGCCCACGCTCAGCAGGTGTGCCATCCTTCCACCTGCTGGGGCACCATTCTTAGTATACA
GAGGTGGCCTTACACACATCTTGCATGGATGGCAGCATTGTTCTGAAGGGGTTTGCAGAAAAAAAAGATT
TTCTTTACATAAAGTGAATCAAATTTGACATTATTTTAGATCCCAGAGAAATCAGGTGTGATTAGTTCTC
CAGGCATGAATGCATCGTCCCTTTATCTGTAAGAACCCTTAGTGTCCTGTAGGGGGACAGAATGGGGTGG
CCAGGTGGTGATTTCTCTTTGACCAATGCATAGTTTGGCAGAAAAATCAGCCGTTCATTTAGAAGAATCT
TAGCAGAGATTGGGATGCCTTACTCAATAAAGCTAAGATGACTATGCTGCTGGCTGTCTTTGTTCTTGGA
GAGGTGGAGTGACTGTTCACGGAGAA
SEQ ID NO: 17
Homo sapiens exostosin 2 (EXT2), transcript variant 2, mRNA
CTGTCTGAGCATTTCACTGCGGAGCCTGAGCGCGCCTGCCTGGGAAAACACTGCAGCGGTGCTCGGACTC
CTCCTGTCCAGCAGGAGGCGCGGCCCGGCAGCTCCCGCATGCGCAGTGCGCTCGGTGTCAGACGGCCCGG
ATCCCGGTTACCGGCCCCTCGCTCGCTGCTCGCCAGCCCAGACTCGGCCCTGGCAGTGGCGGCTGGCGAT
TCGGACCGATCCGACCTGGGCGGAGGTGGCCCGCGCCCCGCGGCATGAGCCGGTGACCAAGCTCGGGGCC
GAGCGGGAGGCAGCCGTGGCCGAGGAGTGTGAGGAAGAGGCTGTCTGTGTCATTATGTGTGCGTCGGTCA
AGTATAATATCCGGGGTCCTGCCCTCATCCCAAGAATGAAGACCAAGCACCGAATCTACTATATCACCCT
CTTCTCCATTGTCCTCCTGGGCCTCATTGCCACTGGCATGTTTCAGTTTTGGCCCCATTCTATCGAGTCC
TCAAATGACTGGAATGTAGAGAAGCGCAGCATCCGTGATGTGCCGGTTGTTAGGCTGCCAGCCGACAGTC
CCATCCCAGAGCGGGGGGATCTCAGTTGCAGAATGCACACGTGTTTTGATGTCTATCGCTGTGGCTTCAA
CCCAAAGAACAAAATCAAGGTGTATATCTATGCTCTGAAAAAGTACGTGGATGACTTTGGCGTCTCTGTC
AGCAACACCATCTCCCGGGAGTATAATGAACTGCTCATGGCCATCTCAGACAGTGACTACTACACTGATG
ACATCAACCGGGCCTGTCTGTTTGTTCCCTCCATCGATGTGCTTAACCAGAACACACTGCGCATCAAGGA
GACAGCACAAGCGATGGCCCAGCTCTCTAGGTGGGATCGAGGTACGAATCACCTGTTGTTCAACATGTTG
CCTGGAGGTCCCCCAGATTATAACACAGCCCTGGATGTCCCCAGAGACAGGGCCCTGTTGGCTGGTGGCG
GCTTTTCTACGTGGACTTACCGGCAAGGCTACGATGTCAGCATTCCTGTCTATAGTCCACTGTCAGCTGA
GGTGGATCTTCCAGAGAAAGGACCAGGTCCACGGCAATACTTCCTCCTGTCATCTCAGGTGGGTCTCCAT
CCTGAGTACAGAGAGGACCTAGAAGCCCTCCAGGTCAAACATGGAGAGTCAGTGTTAGTACTCGATAAAT
GCACCAACCTCTCAGAGGGTGTCCTTTCTGTCCGTAAGCGCTGCCACAAGCACCAGGTCTTCGATTACCC
ACAGGTGCTACAGGAGGCTACTTTCTGTGTGGTTCTTCGTGGAGCTCGGCTGGGCCAGGCAGTATTGAGC
GATGTGTTACAAGCTGGCTGTGTCCCGGTTGTCATTGCAGACTCCTATATTTTGCCTTTCTCTGAAGTTC
TTGACTGGAAGAGAGCATCTGTGGTTGTACCAGAAGAAAAGATGTCAGATGTGTACAGTATTTTGCAGAG
CATCCCCCAAAGACAGATTGAAGAAATGCAGAGACAGGCCCGGTGGTTCTGGGAAGCGTACTTCCAGTCA
ATTAAAGCCATTGCCCTGGCCACCCTGCAGATTATCAATGACCGGATCTATCCATATGCTGCCATCTCCT
ATGAAGAATGGAATGACCCTCCTGCTGTGAAGTGGGGCAGCGTGAGCAATCCACTCTTCCTCCCGCTGAT
CCCACCACAGTCTCAAGGGTTCACCGCCATAGTCCTCACCTACGACCGAGTAGAGAGCCTCTTCCGGGTC
ATCACTGAAGTGTCCAAGGTGCCCAGTCTATCCAAACTACTTGTCGTCTGGAATAATCAGAATAAAAACC
CTCCAGAAGATTCTCTCTGGCCCAAAATCCGGGTTCCATTAAAAGTTGTGAGGACTGCTGAAAACAAGTT
AAGTAACCGTTTCTTCCCTTATGATGAAATCGAGACAGAAGCTGTTCTGGCCATTGATGATGATATCATT
ATGCTGACCTCTGACGAGCTGCAATTTGGTTATGAGGTCTGGCGGGAATTTCCTGACCGGTTGGTGGGTT
ACCCGGGTCGTCTGCATCTCTGGGACCATGAGATGAATAAGTGGAAGTATGAGTCTGAGTGGACGAATGA
AGTGTCCATGGTGCTCACTGGGGCAGCTTTTTATCACAAGTATTTTAATTACCTGTATACCTACAAAATG
CCTGGGGATATCAAGAACTGGGTAGATGCTCATATGAACTGTGAAGATATTGCCATGAACTTCCTGGTGG
CCAACGTCACGGGAAAAGCAGTTATCAAGGTAACCCCACGAAAGAAATTCAAGTGTCCTGAGTGCACAGC
CATAGATGGGCTTTCACTAGACCAAACACACATGGTGGAGAGGTCAGAGTGCATCAACAAGTTTGCTTCA
GTCTTCGGGACCATGCCTCTCAAGGTGGTGGAACACCGAGCTGACCCTGTCCTGTACAAAGATGACTTTC
CTGAGAAGCTGAAGAGCTTCCCCAACATTGGCAGCTTATGAAACGTGTCATTGGTGGAGGTCTGAATGTG
AGGCTGGGACAGAGGGAGAGAACAAGGCCTCCCAGCACTCTGATGTCAGAGTAGTAGGTTAAGGGTGGAA
GGTTGACCTACTTGGATCTTGGCATGCACCCACCTAACCCACTTTCTCAAGAACAAGAACCTAGAATGAA
TATCCAAGCACCTCGAGCTATGCAACCTCTGTTCTTGTATTTCTTATGATCTCTGATGGGTTCTTCTCGA
AAATGCCAAGTGGAAGACTTTGTGGCATGCTCCAGATTTAAATCCAGCTGAGGCTCCCTTTGTTTTCAGT
TCCATGTAACAATCTGGAAGGAAACTTCACGGACAGGAAGACTGCTGGAGAAGAGAAGCGTGTTAGCCCA
TTTGAGGTCTGGGGAATCATGTAAAGGGTACCCAGACCTCACTTTTAGTTATTTACATCAATGAGTTCTT
TCAGGGAACCAAACCCAGAATTCGGTGCAAAAGCCAAACATCTTGGTGGGATTTGATAAATGCCTTGGGA
CCTGGAGTGCTGGGCTTGTGCACAGGAAGAGCACCAGCCGCTGAGTCAGGATCCTGTCAGTTCCATGAGC
TATTCCTCTTTGGTTTGGCTTTTTGATATGATTAAAATTATTTTTTATTCCTTTTTCTACTGTGTCTTAA
ACACCAATTCCTGATAGTCCAAGGAACCACCTTTCTCCCTTGATATATTTAACTCCGTCTTTGGCCTGAC
AACAGTCTTCTGCCCATGTCTGGGAACACACGCCAGGAGGAATGTCTGATACCCTCTGCATCAAGCGTAA
GAAGGTCCCAAATCATAACCATTTTAAGAACAGATGACTCAGAAACCTCCAGAGGAATCTGTTTGCTTCC
TGATTAGATCCAGTCAATGTTTTAAAGGTATTGTCAGAGAAAAACAGAGGGTCTGTACTAGCCATGCAAG
GAGTCGCTCTAGCTGGTACCCGTAAAAGTTGTGGGAATTGTGACCCCCATCCCAAGGGGATGCCAAAATT
TCTCTCATTCTTTTGGTATAAACTTAACATTAGCCAGGGAGGTTCTGGCTAACGTTAAATGCTGCTATAC
AACTGCTTTGCAACAGTTGCTGGTATATTTAAATCATTAAATTTCAGCATTTACTAATACTGCAAAAAAA
AAAAAAAAAAA
SEQ ID NO: 18
Homo sapiens FBJ murine osteosarcoma viral oncogene homolog (FOS), mRNA
ATTCATAAAACGCTTGTTATAAAAGCAGTGGCTGCGGCGCCTCGTACTCCAACCGCATCTGCAGCGAGCA
TCTGAGAAGCCAAGACTGAGCCGGCGGCCGCGGCGCAGCGAACGAGCAGTGACCGTGCTCCTACCCAGCT
CTGCTCCACAGCGCCCACCTGTCTCCGCCCCTCGGCCCCTCGCCCGGCTTTGCCTAACCGCCACGATGAT
GTTCTCGGGCTTCAACGCAGACTACGAGGCGTCATCCTCCCGCTGCAGCAGCGCGTCCCCGGCCGGGGAT
AGCCTCTCTTACTACCACTCACCCGCAGACTCCTTCTCCAGCATGGGCTCGCCTGTCAACGCGCAGGACT
TCTGCACGGACCTGGCCGTCTCCAGTGCCAACTTCATTCCCACGGTCACTGCCATCTCGACCAGTCCGGA
CCTGCAGTGGCTGGTGCAGCCCGCCCTCGTCTCCTCCGTGGCCCCATCGCAGACCAGAGCCCCTCACCCT
TTCGGAGTCCCCGCCCCCTCCGCTGGGGCTTACTCCAGGGCTGGCGTTGTGAAGACCATGACAGGAGGCC
GAGCGCAGAGCATTGGCAGGAGGGGCAAGGTGGAACAGTTATCTCCAGAAGAAGAAGAGAAAAGGAGAAT
CCGAAGGGAAAGGAATAAGATGGCTGCAGCCAAATGCCGCAACCGGAGGAGGGAGCTGACTGATACACTC
CAAGCGGAGACAGACCAACTAGAAGATGAGAAGTCTGCTTTGCAGACCGAGATTGCCAACCTGCTGAAGG
AGAAGGAAAAACTAGAGTTCATCCTGGCAGCTCACCGACCTGCCTGCAAGATCCCTGATGACCTGGGCTT
CCCAGAAGAGATGTCTGTGGCTTCCCTTGATCTGACTGGGGGCCTGCCAGAGGTTGCCACCCCGGAGTCT
GAGGAGGCCTTCACCCTGCCTCTCCTCAATGACCCTGAGCCCAAGCCCTCAGTGGAACCTGTCAAGAGCA
TCAGCAGCATGGAGCTGAAGACCGAGCCCTTTGATGACTTCCTGTTCCCAGCATCATCCAGGCCCAGTGG
CTCTGAGACAGCCCGCTCCGTGCCAGACATGGACCTATCTGGGTCCTTCTATGCAGCAGACTGGGAGCCT
CTGCACAGTGGCTCCCTGGGGATGGGGCCCATGGCCACAGAGCTGGAGCCCCTGTGCACTCCGGTGGTCA
CCTGTACTCCCAGCTGCACTGCTTACACGTCTTCCTTCGTCTTCACCTACCCCGAGGCTGACTCCTTCCC
CAGCTGTGCAGCTGCCCACCGCAAGGGCAGCAGCAGCAATGAGCCTTCCTCTGACTCGCTCAGCTCACCC
ACGCTGCTGGCCCTGTGAGGGGGCAGGGAAGGGGAGGCAGCCGGCACCCACAAGTGCCACTGCCCGAGCT
GGTGCATTACAGAGAGGAGAAACACATCTTCCCTAGAGGGTTCCTGTAGACCTAGGGAGGACCTTATCTG
TGCGTGAAACACACCAGGCTGTGGGCCTCAAGGACTTGAAAGCATCCATGTGTGGACTCAAGTCCTTACC
TCTTCCGGAGATGTAGCAAAACGCATGGAGTGTGTATTGTTCCCAGTGACACTTCAGAGAGCTGGTAGTT
AGTAGCATGTTGAGCCAGGCCTGGGTCTGTGTCTCTTTTCTCTTTCTCCTTAGTCTTCTCATAGCATTAA
CTAATCTATTGGGTTCATTATTGGAATTAACCTGGTGCTGGATATTTTCAAATTGTATCTAGTGCAGCTG
ATTTTAACAATAACTACTGTGTTCCTGGCAATAGTGTGTTCTGATTAGAAATGACCAATATTATACTAAG
AAAAGATACGACTTTATTTTCTGGTAGATAGAAATAAATAGCTATATCCATGTACTGTAGTTTTTCTTCA
ACATCAATGTTCATTGTAATGTTACTGATCATGCATTGTTGAGGTGGTCTGAATGTTCTGACATTAACAG
TTTTCCATGAAAACGTTTTATTGTGTTTTTAATTTATTTATTAAGATGGATTCTCAGATATTTATATTTT
TATTTTATTTTTTTCTACCTTGAGGTCTTTTGACATGTGGAAAGTGAATTTGAATGAAAAATTTAAGCAT
TGTTTGCTTATTGTTCCAAGACATTGTCAATAAAAGCATTTAAGTTGAATGCGACCAA
SEQ ID NO: 19
Homo sapiens FOS-like antigen 1 (FOSL1), mRNA
ACGGGCCAAGGCGGCGCGTCTCGGGGGTGGAGCCTGGAGGTGACCGCGCCGCTGCAACGCCCCCACCCCC
CGCGGTCGCAGTGGTTCAGCCCGAGAACTTTTCATTCATAAAAAGAAAAGACTCCGCACGGCGCGGGTGA
GTCAGAACCCAGCAGCCGTGTACCCCGCAGAGCCGCCAGCCCCGGGCATGTTCCGAGACTTCGGGGAACC
CGGCCCGAGCTCCGGGAACGGCGGCGGGTACGGCGGCCCCGCGCAGCCCCCGGCCGCAGCGCAGGCAGCC
CAGCAGAAGTTCCACCTGGTGCCAAGCATCAACACCATGAGTGGCAGTCAGGAGCTGCAGTGGATGGTAC
AGCCTCATTTCCTGGGGCCCAGCAGTTACCCCAGGCCTCTGACCTACCCTCAGTACAGCCCCCCACAACC
CCGGCCAGGAGTCATCCGGGCCCTGGGGCCGCCTCCAGGGGTACGTCGAAGGCCTTGTGAACAGATCAGC
CCGGAGGAAGAGGAGCGCCGCCGAGTAAGGCGCGAGCGGAACAAGCTGGCTGCGGCCAAGTGCAGGAACC
GGAGGAAGGAACTGACCGACTTCCTGCAGGCGGAGACTGACAAACTGGAAGATGAGAAATCTGGGCTGCA
GCGAGAGATTGAGGAGCTGCAGAAGCAGAAGGAGCGCCTAGAGCTGGTGCTGGAAGCCCACCGACCCATC
TGCAAAATCCCGGAAGGAGCCAAGGAGGGGGACACAGGCAGTACCAGTGGCACCAGCAGCCCACCAGCCC
CCTGCCGCCCTGTACCTTGTATCTCCCTTTCCCCAGGGCCTGTGCTTGAACCTGAGGCACTGCACACCCC
CACACTCATGACCACACCCTCCCTAACTCCTTTCACCCCCAGCCTGGTCTTCACCTACCCCAGCACTCCT
GAGCCTTGTGCCTCAGCTCATCGCAAGAGTAGCAGCAGCAGCGGAGACCCATCCTCTGACCCCCTTGGCT
CTCCAACCCTCCTCGCTTTGTGAGGCGCCTGAGCCCTACTCCCTGCAGATGCCACCCTAGCCAATGTCTC
CTCCCCTTCCCCCACCGGTCCAGCTGGCCTGGACAGTATCCCACATCCAACTCCAGCAACTTCTTCTCCA
TCCCTCTAATGAGACTGACCATATTGTGCTTCACAGTAGAGCCAGCTTGGGGCCACCAAAGCTGCCCACT
GTTTCTCTTGAGCTGGCCTCTCTAGCACAATTTGCACTAAATCAGAGACAAAATATTTCCCATTTGTGCC
AGAGGAATCCTGGCAGCCCAGAGACTTTGTAGATCCTTAGAGGTCCTCTGGAGCCCTAACCCCTTCCAGA
TCACTGCCACACTCTCCATCACCCTCTTCCTGTGATCCACCCAACCCTATCTCCTGACAGAAGGTGCCAC
TTTACCCACCTAGAACACTAACTCACCAGCCCCACTGCCAGCAGCAGCAGGTGATTGGACCAGGCCATTC
TGCCGCCCCCTCCTGAACCGCACAGCTCAGGAGGCGCCCTTGGCTTCTGTGATGAGCTGATCTGCGGATC
TCAGCTTTGAGAAGCCTTCAGCTCCAGGGAATCCAAGCCTCCACAGCGAGGGCAGCTGCTATTTATTTTC
CTAAAGAGAGTATTTTTATACAAACCTACCAAAATGGAATAAAAGGCTTGAAGCTGTG
SEQ ID NO: 20
Homo sapiens forkhead box N3 (FOXN3), transcript variant 1, mRNA
CGCGATCTGCTGCAGCTCGGCCGGGAGACGGCGCGACCCGGCGGCGGGGCCACCCGCGAGTCCAGCGTCG
CCGCAGCCCCCCAATGCGGCCGCGAGAAGCAGCGGGGGGGCAGGCGATCGAAGGAGCCTTCACGTAAATG
GGTCCAGTCATGCCTCCCAGTAAGAAGCCAGAAAGCTCAGGAATTAGTGTCTCCAGTGGACTGAGTCAGT
GTTACGGGGGCAGCGGTTTCTCCAAGGCCCTTCAGGAAGACGATGACCTCGACTTTTCTCTGCCTGACAT
CCGATTAGAAGAGGGGGCCATGGAAGATGAAGAGCTGACCAACCTGAACTGGCTGCACGAGAGCAAGAAC
TTGCTGAAGAGCTTTGGGGAGTCGGTCCTCAGGAGTGTCAGCCCCGTCCAGGACCTGGACGATGACACCC
CCCCATCCCCTGCCCACTCTGACATGCCCTACGATGCCAGGCAGAACCCCAACTGCAAACCCCCCTACTC
CTTCAGCTGCCTCATATTTATGGCCATCGAGGACTCTCCAACCAAGCGCCTGCCAGTGAAGGATATCTAC
AACTGGATCTTGGAACATTTTCCGTATTTTGCAAATGCACCTACTGGGTGGAAAAACTCAGTGAGACACA
ATTTATCATTGAATAAGTGTTTTAAGAAAGTGGACAAAGAGAGGAGTCAGAGTATTGGGAAAGGGTCGTT
GTGGTGCATAGACCCAGAGTATAGACAAAATCTAATTCAGGCTTTGAAAAAGACACCTTATCACCCACAC
CCACACGTGTTCAATACACCTCCCACCTGTCCTCAGGCATATCAAAGCACATCAGGTCCACCCATCTGGC
CGGGCAGTACCTTCTTCAAGAGAAATGGAGCCCTTCTCCAAGATCCTGACATTGATGCTGCCAGTGCCAT
GATGCTTTTGAATACTCCCCCTGAGATACAAGCAGGTTTTCCTCCAGGAGTGATCCAAAATGGAGCGCGG
GTCCTGAGCCGAGGGCTGTTTCCTGGCGTGCGGCCGCTGCCAATCACTCCCATTGGGGTGACAGCGGCCA
TGAGGAATGGCATCACCAGCTGCCGGATGCGGACTGAGAGTGAGCCATCTTGTGGCTCCCCAGTGGTCAG
CGGAGACCCCAAGGAGGATCACAACTACAGCAGTGCCAAGTCCTCCAACGCCCGGAGCACCTCGCCCACC
AGCGACTCCATCTCCTCCTCCTCCTCCTCAGCCGACGACCACTATGAGTTTGCCACCAAGGGGAGCCAGG
AGGGCAGCGAGGGCAGCGAGGGGAGCTTCCGGAGCCACGAGAGCCCCAGCGACACGGAAGAGGACGACAG
GAAGCACAGCCAGAAGGAGCCCAAGGATTCTCTGGGGGACAGCGGGTACGCATCCCAGCACAAGAAGCGC
CAGCACTTCGCCAAGGCCAGGAAGGTCCCCAGCGACACACTGCCCCTCAAAAAGAGACGCACCGAAAAGC
CCCCCGAGAGCGATGATGAGGAGATGAAAGAAGCGGCAGGGTCCCTCCTGCACTTAGCAGGGATCCGGTC
CTGTTTGAATAACATCACCAATCGGACGGCAAAGGGGCAGAAAGAGCAAAAGGAAACCACAAAAAATTAA
AAACAAGTCACTGATTTGTTTTGAACTTACGACCATTTGGTTTCAGCATGTCAGGAGATTTCTAATGATT
TGTGGCAATATCAGCAATTTTTTTTCTTTTTTCTTGTTTTTGGTTTGGTTTTCTTTCTTTTCTTTTCCTT
TTATTTTGTTTTAATTTGCCCCCTCTTCTTTGTTTTGGACCCTTAAGAATTTTATTTTTAAAGGAGATTG
AAGCCATAGAACTCATATTGACACTCAGCTGTTTTACAAAAGCTTTTCATTATCTGAAGACAAAACCGAA
AAAGCCAAAATTACCATTGCTTCCTCCAGCTTGTCAGAAACCTGTGGCTGAATCCGCAGGGATGTCAACG
TCAATATCACAGGAACACACATTCGGCACCTAGAAGGCACGTGGGCAAAGTAATCATCGTTCAGGCCCAA
CCCTTAGGTTTAAAAAGTCAGGTTGTCCATCCCATTGGGTTCACTGAGTGAAGGCACATAAAGCAATTGA
GGAGGAGGAGGAACCCCTCGTCCCCCTAGGAGCAGACCCAAGCTTGTGGCACCAGGCATCTGATGGTGCC
AGGAAAGCCACTGGAATTGTCACACGGCGAGCACAGAGGGCCGGCCACCAGTCCTCGATGCTTCTGAACC
CTGAAGCCCGATGACATCTTACGAGGTGGACGTTGGACTGTTCATGCGCATCGGGTGTCAGTGACTCATG
GAGAAGAAATGGGGTAAATTTTTAGTGATGTTGCTAATCATTGAATTCTGTTCTCTATTAAATTAAGAAA
ATGTTCCAAAAGCCATAAGCCTGAAGATTGGCCCTGTGCACGCACGCACACACACACACACACACACACA
CACACACACACACACACGAAGGAGAGAGAGAGAAAACTGATGGGGAAAACAAGCTGTGTCTTCTTAACTG
CCCAAGTGAAAAGCAACCAAGTCCAGGAAATTACAATAGCTGTTAAGGAAAGGAAATAATGGTACAGATC
TTTTTCTGTCTATCAAAACTATTTGATCCAAGTGAAAAAAAAAAAAAAACTAGAAAGCTACGGAACCTGC
CATTAGTATTGTGGTGTATTTTTAAGATTAAAGGTACACTGATGGACAAAAAAAAAAAGTAAAACATGGC
AAAAAATAAAATAACTCCTATACTGCCCTCAAAATGGAGTTTGCAATTAATATCAGGATTTATCTTTGCA
AAAATCAGTGATTTCCACATTCAGCCAGTATAGCCAGCAGAAATTTCTGATCCACAATGCATGGATTCCT
TTGAGAAAAAAAAAGAAAAAGAGAAAAAAATCACAAAAACAAACTTTTTTTATTCAAAAGTAACAAAGTT
CTTGTAAGGTAAATAATGTATTTAGCATGAAGCATGAATTATTTTCATATAAATATAGAAAATAGAGAAA
AGGCTATGCCTGTAATTTTTAAGCCCTTAGGCTTAGAGTTTCTTTTGGTTTTCTTCTTTTTTCTTTCCTT
TTCTTTGCTTTCTTTTTTTCCTTTTTGTTTTTGTTTTTGTTTTTTGTTTTTGTTTTTTTTTCGGGTTATT
TTGTTTTGGTTTTTTGAAGCAGGTGTTTAAGGTTTAACCTTCTTCAGGGACAAATTCTGACTGTTGGGGA
ACTTACTCTGCAATATAAAAATATCTTCATGCTCTGGTAGGGCTTGGATGGTTGAACTCTGTACTGCCTT
GTGTGCACTTCAGCCCCGACCCCCTCTGATTCTCTGTTGAAAAGTGTGTCCTTTCTCTCTGTCTGTACAT
GTTTAACATGACGCAATAATTTGAGGGCAAACTTAGTAGTGAGTGTGTATGATAGAATCAAGAGAATTAT
GGGACGCTTACTTGAGAAAATCATTACCATGATTTGGTTCTAGGAAAAAGGCAGTGAATAATTATGCAAA
TTAGCCAGAAGAAGGGGAACCGTGCTAATGGGCCTTATTGGGTGAGGGGACGAGATGGGGTTCATGTGAA
GGAGGAAGCGATGCCGAGGTAGGAAAGGCCAGCCCCAGACATCCTATCGCCACAATGCCATGTCGCAATA
GGAAGCAGGGGCCGGCCATCGCTACCTTCAGCACACTGACCAACCTGGAATTAAGACCACCTAGATTGCG
AGAGCTGAATTTAGAAACCAGACAACGTCATGCAGCCCAGAAACTCCTGTTGTTACCTTTGCCTAAGAAA
TTTTCTTTAATGGCGGGGGCGGGGGGCGGGGGTACAAAGAGAAATCTCTAAAAGAATATGATCTTCCATC
CAAGTGGAGGGAAACTTTAAAACAAAAACACCCAGTACTGTGGCTCAGGATATGATGCGTGAGGAGAGGG
AGGGAACAGAGATGACCTTAACTTTTAAAAAAGGGACTGCTGTGGGCCAAAGCCAAGCCCATCTGCCAGG
ACGAGGTAATGTCAGAGCTCCATCAGCCCGGACAGTGGGAACTAACTGGTGCATTCCCCACACTTACCTT
CCGGTGGGTTGCTGATGAGAGAACCTGAAAAAACCTACACCTCTACAGCAGGTCGAATTCATGACCTGAA
GCTGAATACTTCCAGCATATTTATTCAGGGTGTAGGTGGGAATAAAGTATCTTCGCAGTGCTCTGTTCCC
TCCGTCTCCCCAGACATCTGACACCCTAAAAGCCATCCACAGCTATGGAACCTGAGCGACACCTTGATTT
GTGTTGTCACCTGACCAAGCCTAAAGACCTCCAGCTCAGTCCCCCACCTTCATCCCACCCCACAGATGAT
AAAATTCAGACCTCTCTCCTGAAAGGCAGAGGTTCAACATTCAGGACTGTTTCTGGCCGAGGACTTCTTC
CAATTAAAACCCCCACCGTGGGCTGTCTCCCCTCATTTCATTTTTCTAAAGGGGCAGAGGCCTCTTTTAG
AAAATAATAAAATGCAATGTGTGTGATTTACTTTTCTGATCTCTTTGAGAAATAGAGAAATATAAAAGTG
TGTTCTTAACTCCAGAACCACTCTTTTTGCATAAATACCTCATCGGGCAGCTTTCTAAGTGTGATTTTCC
TGAGTCTCCCTTCGTTGGATCTGCCGGAAGACTTGTCGGGGAACCTTTAGTGAGGGTACTTCTTCCTATT
TTTCTTCTGTTTTTGGAGGCATACACATTATGCATAACCAAAACAATGGCTCAATTGTGTTTAACTTTGT
ATTTTGATTGTTGAGAACAAAAACAAAAAGTATCAATGTGTATGTGGCTGTTTGTAGTGAATTTATTGGA
GAATGAGGTTGTCCGTGTCCTTAACAAGCCAAGGGGCAGGAGGCACCCTCTCTTATCCCCTCCTCCAAGA
GCAGTAGAGAATTTAAGCACAAGCCTATTTGTGAAAGAATATTTTGCTTAAGTGTCATTCACTTTAGTCT
TGGAATTCCTTCCCAAACGTCAGGTGTTCTTTTAGCTTCCAAACTAGCATATGTATCCATTAGTCTGACA
GATCGCCTGAACACCATTAAGAGGTGTGGCGTTTTTGCTTTCATTTCTCCTGCTGGGAGAAGTGGCGGTT
CATGTGTCATTCCAGTATCTCACATACTCACACGGGGCAGGGGGGAGGGGGAAACGGGGAACTATAGCAA
TATTTAAAGATGCTTTGGAAACCAACCGTGAACACATCAACACCACGACGTCTACGATTACTTGCTATTG
GCCCTCGGATACATTTAAGAGAAAGAGACAGTCACTCTTTTTTTTCTTAAATGATATACATATAAACAGT
TATTTTTATCCTATTATAATTGTCTTTTGTCTTTATCTAGTACTATGTGGAAAGGGTTTGCATCATAGAT
TTTTCCCAGCCTTATAATATACCATAAGCTCCTACTTCCCTGCCCCTCCCTAATCAGTATTCTTTCAAGA
GTTCTTTGGTGAAGCCATCTATCTGAAACTAAAATGAACCAAACCCATATTTCACTGGTGGTTGGAGAAA
ACCATGGCCAAAACGATTGTGGCAGGTCTCAATCTTGGGAGTTTTTAAGAAGGAATGTGCCAGAGGCCGA
TTCCCAAGAACAGAGTTTTCTTTTGTTTTGCAGAGGCATTCAATGTGTCTAGTGCTTGCTGGCCACAGCA
GTTACTACCACAGAGCCTTCTGGGAGGGGCCGTTGTGTTGAAGGAGGCTCCTGCCTGAGGGACAGCATCA
GGCAGTGGGCTCTGTAGAGTGAGAACCAGGTGGAGGCCTTCTGTGCCCAGCTCAGAGTTCTGCACCACGC
CAGGACTGCCCAGGCCAAGGGCTACTGACGCAAGTTCCACTCATTCCACTCTGTGGGGGGCGCCTTGGGC
CTCTCCTGGAAGGGCTCTTGGAGAAGGAATTGGAGTTACGTACAAGTGACCTAAATGGGAAGCTTTTCTA
GATGAGATTGGATTAAATTCCATGTGATTTCTCTTTCCCTTTAATCCAGGTTGGGACTCGTTTCTTTCTG
GTGGATCACAGCTGCCCAGATGTTGCAATTGATTTTTATGTTTCTGTAGAGAAGTATTTTTCTTTCATCT
TCAGGATTTTTTTTGCCACCAAAAGAAAACATTGGAACTCTGTGTTTCCTCTTGATTGTGACTTCCCAGT
GTTGACAGTTAAGTCCTTAGTGTCGTAGGTCCCAGCCCACCAATACTATATCAAACACTGTTATGCACAT
AATGCAGCACTGTGATCTAATTTAAATAATACTTTTTTATTATTTATACTACTATATATAATATACATCA
ACACTTTTGCTATATAACCTAAGTGATAACCCTCTTTTAGTTACCTGCCAAACTCTGGACTTGGTTTATA
TTGCAGTTAACACAGTTACAAAGCTGTAATGGTGTCTTTTTTTCCTTTGTAACGGAATGTGTAAATCAAA
GTATATACATTGTGTGGTGTTCCTGTTTCTGGAGTTTCATGAGGATTTACACATGGCATTCAGTGTTCTG
TATAGATCTGCCTACCTTTGTGAATTCATCTGTTAACCCCTCTTCCTTTGAGAGAGCACCGGCGATGGTG
GTTAACTCCTTGTGTTTTCTCTCTCTCCTACTGGTTATTCTTGAATTAAGCACAGACTCGTCAGCTCGGT
TGCTTTATCATGAATAATGTGTGTGACCTTGCAGTTCTTCCACAGTTCAGCAAACAAGTGCTAGCTTCAC
TGACCAAAAATTAAGGAAGGAAAACACAGTTTTTAAAACGATCCATCTTTTAACAGCCGAAACCGATGTG
TCTATGGTGCTGCACCTTGCTGTTGTACTTCTGAAATCAGACGTGTGTGAACGATCATTTCTGACTTAAC
CGTGAGATGCTCACGAGTACCCTTCCTGTTGTTTTGTTAGCATTGAAATCGAGACTATTTATTTGGAATA
TATACAACAGTGTTTTTCCACTGTATTTCATTTGCAAAAGTTGAGAACTGCTTTCTCTACCTTTTGCAAA
ATAATTGATATTCCATATTGGATTCTCAAAGACTTCGATATGGTGAACCTATTAAACCTAGAAATTGTAT
TCATCCTTTCATGACTGTGGCCTGAGTTCCCCAGCCCCTCTCCTCCTTTTTTTTAGATGAGATTTAGCAC
ACTCTCAGTTATTTAAACATGCAACATTTCTTGAGTATGTATGTTGAGGCCATCTGAGCTCATAGCTGAT
TCAGTAACCAGTTTCATGCTGTGTCATTCACACTCACTACTTAATACTGCCATGGTGAAAATGTGGAGGA
AAAATGTATCCATGTGTGTCTGGGAAGCATATACACTTGTACATTTTTTAATACTCTGATTCTGTAACAT
TTCTGAGTTTTGTTTTGTTTTACAGAAAAAAAAAAAAAGTGATAAAGCAATCAGAAGACCAAGAGGTTTA
CTATTGATGCTTAGGGTCGTCTGACCTTGGCTGGCCAATAGACCTACACGGCCAAATTAATTTACGAGAG
TAATAATTTTTCAAAAGCCAATTTTTTTTCTGTATTTTCTGTATGAAACTGCCAATATCATGAATAGAAA
GGGAGAACCATAAAGGAGAAAGAACGTGATGTTCTGTTATGTTCATGTAAACCTAAAGAAACAGTGTGGA
GGCAGGCGCGATCAGCCGAACTCTAGGGACTTGGTGTTGCTTGGAAGGCATCCATACCTGCATTTTGCAT
TCTTCGTATGTAATCATATTGCCAAAGACAAACTATTTCATCATTTATTGTAAATAACACTTTTCCCCAG
ACCTACCATAAAGTTTCTGTGATGTATTGTCTTCCAGTTGCAATAAAAATTACTGAGTTGCATCAATTGA
AGAAAAACACCAAAAA
SEQ ID NO: 21
Homo sapiens glyceraldehyde-3-phosphate dehydrogenase (GAPDH), mRNA
AAATTGAGCCCGCAGCCTCCCGCTTCGCTCTCTGCTCCTCCTGTTCGACAGTCAGCCGCATCTTCTTTTG
CGTCGCCAGCCGAGCCACATCGCTCAGACACCATGGGGAAGGTGAAGGTCGGAGTCAACGGATTTGGTCG
TATTGGGCGCCTGGTCACCAGGGCTGCTTTTAACTCTGGTAAAGTGGATATTGTTGCCATCAATGACCCC
TTCATTGACCTCAACTACATGGTTTACATGTTCCAATATGATTCCACCCATGGCAAATTCCATGGCACCG
TCAAGGCTGAGAACGGGAAGCTTGTCATCAATGGAAATCCCATCACCATCTTCCAGGAGCGAGATCCCTC
CAAAATCAAGTGGGGCGATGCTGGCGCTGAGTACGTCGTGGAGTCCACTGGCGTCTTCACCACCATGGAG
AAGGCTGGGGCTCATTTGCAGGGGGGAGCCAAAAGGGTCATCATCTCTGCCCCCTCTGCTGATGCCCCCA
TGTTCGTCATGGGTGTGAACCATGAGAAGTATGACAACAGCCTCAAGATCATCAGCAATGCCTCCTGCAC
CACCAACTGCTTAGCACCCCTGGCCAAGGTCATCCATGACAACTTTGGTATCGTGGAAGGACTCATGACC
ACAGTCCATGCCATCACTGCCACCCAGAAGACTGTGGATGGCCCCTCCGGGAAACTGTGGCGTGATGGCC
GCGGGGCTCTCCAGAACATCATCCCTGCCTCTACTGGCGCTGCCAAGGCTGTGGGCAAGGTCATCCCTGA
GCTGAACGGGAAGCTCACTGGCATGGCCTTCCGTGTCCCCACTGCCAACGTGTCAGTGGTGGACCTGACC
TGCCGTCTAGAAAAACCTGCCAAATATGATGACATCAAGAAGGTGGTGAAGCAGGCGTCGGAGGGCCCCC
TCAAGGGCATCCTGGGCTACACTGAGCACCAGGTGGTCTCCTCTGACTTCAACAGCGACACCCACTCCTC
CACCTTTGACGCTGGGGCTGGCATTGCCCTCAACGACCACTTTGTCAAGCTCATTTCCTGGTATGACAAC
GAATTTGGCTACAGCAACAGGGTGGTGGACCTCATGGCCCACATGGCCTCCAAGGAGTAAGACCCCTGGA
CCACCAGCCCCAGCAAGAGCACAAGAGGAAGAGAGAGACCCTCACTGCTGGGGAGTCCCTGCCACACTCA
GTCCCCCACCACACTGAATCTCCCCTCCTCACAGTTGCCATGTAGACCCCTTGAAGAGGGGAGGGGCCTA
GGGAGCCGCACCTTGTCATGTACCATCAATAAAGTACCCTGTGCTCAACC
SEQ ID NO: 22
Homo sapiens glyceraldehyde-3-phosphate dehydrogenase (GAPDH), mRNA
AAATTGAGCCCGCAGCCTCCCGCTTCGCTCTCTGCTCCTCCTGTTCGACAGTCAGCCGCATCTTCTTTTG
CGTCGCCAGCCGAGCCACATCGCTCAGACACCATGGGGAAGGTGAAGGTCGGAGTCAACGGATTTGGTCG
TATTGGGCGCCTGGTCACCAGGGCTGCTTTTAACTCTGGTAAAGTGGATATTGTTGCCATCAATGACCCC
TTCATTGACCTCAACTACATGGTTTACATGTTCCAATATGATTCCACCCATGGCAAATTCCATGGCACCG
TCAAGGCTGAGAACGGGAAGCTTGTCATCAATGGAAATCCCATCACCATCTTCCAGGAGCGAGATCCCTC
CAAAATCAAGTGGGGCGATGCTGGCGCTGAGTACGTCGTGGAGTCCACTGGCGTCTTCACCACCATGGAG
AAGGCTGGGGCTCATTTGCAGGGGGGAGCCAAAAGGGTCATCATCTCTGCCCCCTCTGCTGATGCCCCCA
TGTTCGTCATGGGTGTGAACCATGAGAAGTATGACAACAGCCTCAAGATCATCAGCAATGCCTCCTGCAC
CACCAACTGCTTAGCACCCCTGGCCAAGGTCATCCATGACAACTTTGGTATCGTGGAAGGACTCATGACC
ACAGTCCATGCCATCACTGCCACCCAGAAGACTGTGGATGGCCCCTCCGGGAAACTGTGGCGTGATGGCC
GCGGGGCTCTCCAGAACATCATCCCTGCCTCTACTGGCGCTGCCAAGGCTGTGGGCAAGGTCATCCCTGA
GCTGAACGGGAAGCTCACTGGCATGGCCTTCCGTGTCCCCACTGCCAACGTGTCAGTGGTGGACCTGACC
TGCCGTCTAGAAAAACCTGCCAAATATGATGACATCAAGAAGGTGGTGAAGCAGGCGTCGGAGGGCCCCC
TCAAGGGCATCCTGGGCTACACTGAGCACCAGGTGGTCTCCTCTGACTTCAACAGCGACACCCACTCCTC
CACCTTTGACGCTGGGGCTGGCATTGCCCTCAACGACCACTTTGTCAAGCTCATTTCCTGGTATGACAAC
GAATTTGGCTACAGCAACAGGGTGGTGGACCTCATGGCCCACATGGCCTCCAAGGAGTAAGACCCCTGGA
CCACCAGCCCCAGCAAGAGCACAAGAGGAAGAGAGAGACCCTCACTGCTGGGGAGTCCCTGCCACACTCA
GTCCCCCACCACACTGAATCTCCCCTCCTCACAGTTGCCATGTAGACCCCTTGAAGAGGGGAGGGGCCTA
GGGAGCCGCACCTTGTCATGTACCATCAATAAAGTACCCTGTGCTCAACC
SEQ ID NO: 23
Homo sapiens GATA binding protein 3 (GATA3), transcript variant 1, mRNA
GGCGCCGTCTTGATACTTTCAGAGATGCATTCCCTGTAAAAAAAAAAAAAAAAAAATACTGAGAGAGGGA
GAGAGAGAGAGAAGAAGAGAGAGAGACGGAGGGAGAGCGAGACAGAGCGAGCAACGCAATCTGACCGAGC
AGGTCGTACGCCGCCGCCTCCTCCTCCTCTCTGCTCTTCGCTACCCAGGTGACCCGAGGAGGGACTCCGC
CTCCGAGCGGCTGAGGACCCCGGTGCAGAGGAGCCTGGCTCGCAGAATTGCAGAGTCGTCGCCCCTTTTT
ACAACCTGGTCCCGTTTTATTCTGCCGTACCCAGTTTTTGGATTTTTGTCTTCCCCTTCTTCTCTTTGCT
AAACGACCCCTCCAAGATAATTTTTAAAAAACCTTCTCCTTTGCTCACCTTTGCTTCCCAGCCTTCCCAT
CCCCCCACCGAAAGCAAATCATTCAACGACCCCCGACCCTCCGACGGCAGGAGCCCCCCGACCTCCCAGG
CGGACCGCCCTCCCTCCCCGCGCGCGGGTTCCGGGCCCGGCGAGAGGGCGCGAGCACAGCCGAGGCCATG
GAGGTGACGGCGGACCAGCCGCGCTGGGTGAGCCACCACCACCCCGCCGTGCTCAACGGGCAGCACCCGG
ACACGCACCACCCGGGCCTCAGCCACTCCTACATGGACGCGGCGCAGTACCCGCTGCCGGAGGAGGTGGA
TGTGCTTTTTAACATCGACGGTCAAGGCAACCACGTCCCGCCCTACTACGGAAACTCGGTCAGGGCCACG
GTGCAGAGGTACCCTCCGACCCACCACGGGAGCCAGGTGTGCCGCCCGCCTCTGCTTCATGGATCCCTAC
CCTGGCTGGACGGCGGCAAAGCCCTGGGCAGCCACCACACCGCCTCCCCCTGGAATCTCAGCCCCTTCTC
CAAGACGTCCATCCACCACGGCTCCCCGGGGCCCCTCTCCGTCTACCCCCCGGCCTCGTCCTCCTCCTTG
TCGGGGGGCCACGCCAGCCCGCACCTCTTCACCTTCCCGCCCACCCCGCCGAAGGACGTCTCCCCGGACC
CATCGCTGTCCACCCCAGGCTCGGCCGGCTCGGCCCGGCAGGACGAGAAAGAGTGCCTCAAGTACCAGGT
GCCCCTGCCCGACAGCATGAAGCTGGAGTCGTCCCACTCCCGTGGCAGCATGACCGCCCTGGGTGGAGCC
TCCTCGTCGACCCACCACCCCATCACCACCTACCCGCCCTACGTGCCCGAGTACAGCTCCGGACTCTTCC
CCCCCAGCAGCCTGCTGGGCGGCTCCCCCACCGGCTTCGGATGCAAGTCCAGGCCCAAGGCCCGGTCCAG
CACAGAAGGCAGGGAGTGTGTGAACTGTGGGGCAACCTCGACCCCACTGTGGCGGCGAGATGGCACGGGA
CACTACCTGTGCAACGCCTGCGGGCTCTATCACAAAATGAACGGACAGAACCGGCCCCTCATTAAGCCCA
AGCGAAGGCTGTCTGCAGCCAGGAGAGCAGGGACGTCCTGTGCGAACTGTCAGACCACCACAACCACACT
CTGGAGGAGGAATGCCAATGGGGACCCTGTCTGCAATGCCTGTGGGCTCTACTACAAGCTTCACAATATT
AACAGACCCCTGACTATGAAGAAGGAAGGCATCCAGACCAGAAACCGAAAAATGTCTAGCAAATCCAAAA
AGTGCAAAAAAGTGCATGACTCACTGGAGGACTTCCCCAAGAACAGCTCGTTTAACCCGGCCGCCCTCTC
CAGACACATGTCCTCCCTGAGCCACATCTCGCCCTTCAGCCACTCCAGCCACATGCTGACCACGCCCACG
CCGATGCACCCGCCATCCAGCCTGTCCTTTGGACCACACCACCCCTCCAGCATGGTCACCGCCATGGGTT
AGAGCCCTGCTCGATGCTCACAGGGCCCCCAGCGAGAGTCCCTGCAGTCCCTTTCGACTTGCATTTTTGC
AGGAGCAGTATCATGAAGCCTAAACGCGATGGATATATGTTTTTGAAGGCAGAAAGCAAAATTATGTTTG
CCACTTTGCAAAGGAGCTCACTGTGGTGTCTGTGTTCCAACCACTGAATCTGGACCCCATCTGTGAATAA
GCCATTCTGACTCATATCCCCTATTTAACAGGGTCTCTAGTGCTGTGAAAAAAAAAATGCTGAACATTGC
ATATAACTTATATTGTAAGAAATACTGTACAATGACTTTATTGCATCTGGGTAGCTGTAAGGCATGAAGG
ATGCCAAGAAGTTTAAGGAATATGGGAGAAATAGTGTGGAAATTAAGAAGAAACTAGGTCTGATATTCAA
ATGGACAAACTGCCAGTTTTGTTTCCTTTCACTGGCCACAGTTGTTTGATGCATTAAAAGAAAATAAAAA
AAAGAAAAAAGAGAAAAGAAAAAAAAAGAAAAAAGTTGTAGGCGAATCATTTGTTCAAAGCTGTTGGCCT
CTGCAAAGGAAATACCAGTTCTGGGCAATCAGTGTTACCGTTCACCAGTTGCCGTTGAGGGTTTCAGAGA
GCCTTTTTCTAGGCCTACATGCTTTGTGAACAAGTCCCTGTAATTGTTGTTTGTATGTATAATTCAAAGC
ACCAAAATAAGAAAAGATGTAGATTTATTTCATCATATTATACAGACCGAACTGTTGTATAAATTTATTT
ACTGCTAGTCTTAAGAACTGCTTTCTTTCGTTTGTTTGTTTCAATATTTTCCTTCTCTCTCAATTTTTGG
TTGAATAAACTAGATTACATTCAGTTGGCCTAAGGTGGTTGTGCTCGGAGGGTTTCTTGTTTCTTTTCCA
TTTTGTTTTTGGATGATATTTATTAAATAGCTTCTAAGAGTCCGGCGGCATCTGTCTTGTCCCTATTCCT
GCAGCCTGTGCTGAGGGTAGCAGTGTATGAGCTACCAGCGTGCATGTCAGCGACCCTGGCCCGACAGGCC
ACGTCCTGCAATCGGCCCGGCTGCCTCTTCGCCCTGTCGTGTTCTGTGTTAGTGATCACTGCCTTTAATA
CAGTCTGTTGGAATAATATTATAAGCATAATAATAAAGTGAAAATATTTTAAAACTACAA
SEQ ID NO: 24
Homo sapiens guanine nucleotide binding protein (G protein), beta 5
(GNB5), transcript variant 1, mRNA
CCGGGGACGGCTGCTGGAGCGGCGCCCGCCGCGGCTCAGCGCATTCCCGCTCTCCGCTTCCCTCTCCGCT
GCGTCCCCGCGCGAAGATGGCAACCGAGGGGCTGCACGAGAACGAGACGCTGGCGTCGCTGAAGAGCGAG
GCCGAGAGCCTCAAGGGCAAGCTGGAGGAGGAGCGAGCCAAGCTGCACGATGTGGAGCTGCACCAGGTGG
CGGAGCGGGTGGAGGCCCTGGGGCAGTTTGTCATGAAGACCAGAAGGACCCTCAAAGGCCACGGGAACAA
AGTCCTGTGCATGGACTGGTGCAAAGATAAGAGGAGGATCGTGAGCTCGTCACAGGATGGGAAGGTGATC
GTGTGGGATTCCTTCACCACAAACAAGGAGCACGCGGTCACCATGCCCTGCACGTGGGTGATGGCATGTG
CTTATGCCCCATCGGGATGTGCCATTGCTTGTGGTGGTTTGGATAATAAGTGTTCTGTGTACCCCTTGAC
GTTTGACAAAAATGAAAACATGGCTGCCAAAAAGAAGTCTGTTGCTATGCACACCAACTACCTGTCGGCC
TGCAGCTTCACCAACTCTGACATGCAGATCCTGACAGCGAGCGGCGATGGCACATGTGCCCTGTGGGACG
TGGAGAGCGGGCAGCTGCTGCAGAGCTTCCACGGACATGGGGCTGACGTCCTCTGCTTGGACCTGGCCCC
CTCAGAAACTGGAAACACCTTCGTGTCTGGGGGATGTGACAAGAAAGCCATGGTGTGGGACATGCGCTCC
GGCCAGTGCGTGCAGGCCTTTGAAACACATGAATCTGACATCAACAGTGTCCGGTACTACCCCAGTGGAG
ATGCCTTTGCTTCAGGGTCAGATGACGCTACGTGTCGCCTCTATGACCTGCGGGCAGATAGGGAGGTTGC
CATCTATTCCAAAGAAAGCATCATATTTGGAGCATCCAGCGTGGACTTCTCCCTCAGTGGTCGCCTGCTG
TTTGCTGGATACAATGATTACACTATCAACGTCTGGGATGTTCTCAAAGGGTCCCGGGTCTCCATCCTGT
TTGGACATGAAAACCGCGTTAGCACTCTACGAGTTTCCCCCGATGGGACTGCTTTCTGCTCTGGATCATG
GGATCATACCCTCAGAGTCTGGGCCTAATCATCTTCTGACAGTGCACTCATGTATACCTGAGAATTTGAA
ATCTTCACATGTAAATAGATATTACTTCTAGAGGAGCTTAGAGTTTATTGCAGTGTAGCTTAGGGGAGCA
ACCCATGGCTCACAGGTCACTAAGCGTCTCCAATATGACTATTAAAACTGTCACCTCTGGAAATACACTA
GTGTGAGCCTTCAGCACTGCGAGAATACCTTCAAGTACAGTATTTTTCTTTTGGAACACTTTTTAAAATG
TATCTGTTTTTAAGGTTATTCTAAATTATAGTAGCCTCAACTCATTCTGTCACCAGTAGAATTCAGCAGT
TAATATATTCCATATTATTTCTTTGAATCAATTCATTTTCAGAGCACTTTAAAGTCTGATATTTCTCGAT
GTGCACTGTGATGCCTGGAACCTTCCTCTGGAAGTGCTGATTTTATGGACTGAGGACTGGTGACTGGTCT
GTGATAGAAGCAAATTCCAATTCCAAATGTAATTAGACAAAAATCATTTTTTTAGAATGTGTTTTTATTG
TAAAAGTATCTTTTTCAGCTTCCTGTTCTATTGTCTTTTTTCAGATACAACATTTTTGTCTATGGTGAAC
TGCTGTAAATGACGCAGAGAAATGCCTAAAAAGGACAGGTGGTTTGACTCATGGATGATGATGATGTCAC
TGTGCCACTTGGACAGGGCGTTTTCTCTGAATTGAAGGGAAAGCCAATGGTGTTTGTAAACAAATGCTTC
TGAGAGCAAAGAAAAGTCTTCTGTGTGGGAACACAAGATAGTAAACTTATTTAAAAACCTATTAGTAGAA
TTAGTGGAAACACTTAGGTTAAAGTGAATCTTGTCCATATAAATTATATTCATGGCCGGGCGCGGTGGCT
CACGCTTGTAATCCCAGCACTTTGGGAGGCCGAGGCGGGCGGATCACGAGGTCAGGAGTTCGAGACCACG
GTGAAACCCTGTCTCTACTAAAAAATACAAAAAATTAGCCGGGCGTGGTGGCGGGCGCCTGTAGTCCCAG
CTACTCGGAGAGGCTGAGGCAGGAGAATGGCGTGAACCCGGGAGGTGGAGCTTGCAGTGAGCCGAGGTCG
AGCCACTGCAGCCTGGGTGACAGCGAGACTCCGTCTCAAAAAAAAAAAAAAAAATTATATTCATATGTAT
TGCATTGCAATTATAATTACATATGCAGATTGATTGATAGTCATGAATAATAACGTCTGCTCCTCTTACA
TAGAAAAACGATATTAAAAGAAGATCTTCTCTTTATTTGAGACTCAGAATTCCTTCTAGAAGAAGGAAGT
GCTTTTTGTTATAGGATCCCTTCTTTTCCTTTTTTTGTTTTTTTGTAAGATGTAGATGCTTATTCTTTGC
TTTAGAAAACTTCTCACTTAAAAAGATGGCATGCACCTAGGGGAATAAAAGGTCACCTCAGACACCAGGT
GTCATTCCTGGTGAGGCCTGCCTCGTCGGTGGCCTGGGGTCTGCCGGCAGGTTCTGGCTGCACCTGAAGG
CTGCGTGCACCTTGTCCCCTGGACAGGTCTCCTTTCCTGGCCCTGCTCCAGCCCAGCCCTTCTTCTAGTG
GTAGCTCTGGCTTTGCAGGCCCAGCTCCAGGCCCTGCTCCTCAGAGAGACTCTTCCAGAGCTGGAGCTGG
GCACAGCCATAAGACAGGACTGGACCAGATGCTCCTGTAAACATCCAGGGGTGTGCCAGGCCCACCCTCA
CAACTGCTTGTTCAGGTATCGTGATGGGCCACTCGGTCCAAAATCAGCCAGGCCATCTTTTCCATCATCT
CACTTCAAATAAACATAATAATTATATTTGATCATTTGC
SEQ ID NO: 25
Homo sapiens glutathione S-transferase mu 4 (GSTM4), transcript variant
2, mRNA
AAGCTGGCGAGGCCGAGCCCCTCCTAGTGCTTCCGGACCTTGCTCCCTGAACACTCGGAGGTGGCGGTGG
ATCTTACTCCTTCCAGCCAGTGAGGATCCAGCAACCTGCTCCGTGCCTCCCGCGCCTGTTGGTTGGAAGT
GACGACCTTGAAGATCGGCCGGTTGGAAGTGACGACCTTGAAGATCGGCGGGCGCAGCGGGGCCGAGGGG
GCGGGTCTGGCGCTAGGTCCAGCCCCTGCGTGCCGGGAACCCCAGAGGAGGTCGCAGTTCAGCCCAGCTG
AGGCCTGTCTGCAGAATCGACACCAACCAGCATCATGTCCATGACACTGGGGTACTGGGACATCCGCGGG
CTGGCCCACGCCATCCGCCTGCTCCTGGAATACACAGACTCAAGCTACGAGGAAAAGAAGTATACGATGG
GGGACGCTCCTGACTATGACAGAAGCCAGTGGCTGAATGAAAAATTCAAGCTGGGCCTGGACTTTCCCAA
TCTGCCCTACTTGATTGATGGGGCTCACAAGATCACCCAGAGCAACGCCATCCTGTGCTACATTGCCCGC
AAGCACAACCTGTGTGGGGAGACAGAAGAGGAGAAGATTCGTGTGGACATTTTGGAGAACCAGGCTATGG
ACGTCTCCAATCAGCTGGCCAGAGTCTGCTACAGCCCTGACTTTGAGAAACTGAAGCCAGAATACTTGGA
GGAACTTCCTACAATGATGCAGCACTTCTCACAGTTCCTGGGGAAGAGGCCATGGTTTGTTGGAGACAAG
ATCACCTTTGTAGATTTCCTCGCCTATGATGTCCTTGACCTCCACCGTATATTTGAGCCCAACTGCTTGG
ACGCCTTTCCAAATCTGAAGGACTTCATCTCCCGCTTTGAGGTTTCCTGTGGCATAATGTGATGGTCAAT
TTTCTGCATCAACTTGACTGGGCTAAGGGATGCTCAGATGGCAGGTAAAATCATTGTGCTTGTGAGGGTG
TTTCCAGAAGAGATTTGCCTTTGAATCAGAAGACAGCAAAGATTTCCTTCAGCAATGAAGGAGGCATCCA
CCAAACTGTCAGGGCCCAGAGAGAAGAAAAAGACAGGAAGGGTGAATTTGACCTCTCTGACTGGGACATC
CATCTCTGCCTATCCTGGGACCTCCACACTCCTGGTTCTCTGGCCTTCAGACTTGATCAGGGACTAACAC
CATCGCCTCCCACCCCCACCTTTGTTCTGAGGCCTTTAGCCTCTGAATGATACCACTGGCTTTCCTGCTT
CTCTATCCTGCAGTCGGCAGATCATGGGACTTCTTCACTCCAAAATTGTGTGAGCCAATTCCCATAACAG
ATAGATAAATTTATAAATAAACACACAAATTTCCTACAGCCT
SEQ ID NO: 26
Homo sapiens major histocompatibility complex, class II, DR alpha
(HLA-DRA), mRNA
TTTTAATGGTCAGACTCTATTACACCCCACATTCTCTTTTCTTTTATTCTTGTCTGTTCTGCCTCACTCC
CGAGCTCTACTGACTCCCAACAGAGCGCCCAAGAAGAAAATGGCCATAAGTGGAGTCCCTGTGCTAGGAT
TTTTCATCATAGCTGTGCTGATGAGCGCTCAGGAATCATGGGCTATCAAAGAAGAACATGTGATCATCCA
GGCCGAGTTCTATCTGAATCCTGACCAATCAGGCGAGTTTATGTTTGACTTTGATGGTGATGAGATTTTC
CATGTGGATATGGCAAAGAAGGAGACGGTCTGGCGGCTTGAAGAATTTGGACGATTTGCCAGCTTTGAGG
CTCAAGGTGCATTGGCCAACATAGCTGTGGACAAAGCCAACCTGGAAATCATGACAAAGCGCTCCAACTA
TACTCCGATCACCAATGTACCTCCAGAGGTAACTGTGCTCACAAACAGCCCTGTGGAACTGAGAGAGCCC
AACGTCCTCATCTGTTTCATAGACAAGTTCACCCCACCAGTGGTCAATGTCACGTGGCTTCGAAATGGAA
AACCTGTCACCACAGGAGTGTCAGAGACAGTCTTCCTGCCCAGGGAAGACCACCTTTTCCGCAAGTTCCA
CTATCTCCCCTTCCTGCCCTCAACTGAGGACGTTTACGACTGCAGGGTGGAGCACTGGGGCTTGGATGAG
CCTCTTCTCAAGCACTGGGAGTTTGATGCTCCAAGCCCTCTCCCAGAGACTACAGAGAACGTGGTGTGTG
CCCTGGGCCTGACTGTGGGTCTGGTGGGCATCATTATTGGGACCATCTTCATCATCAAGGGATTGCGCAA
AAGCAATGCAGCAGAACGCAGGGGGCCTCTGTAAGGCACATGGAGGTGATGGTGTTTCTTAGAGAGAAGA
TCACTGAAGAAACTTCTGCTTTAATGGCTTTACAAAGCTGGCAATATTACAATCCTTGACCTCAGTGAAA
GCAGTCATCTTCAGCATTTTCCAGCCCTATAGCCACCCCAAGTGTGGATATGCCTCTTCGATTGCTCCGT
ACTCTAACATCTAGCTGGCTTCCCTGTCTATTGCCTTTTCCTGTATCTATTTTCCTCTATTTCCTATCAT
TTTATTATCACCATGCAATGCCTCTGGAATAAAACATACAGGAGTCTGTCTCTGCTATGGAATGCCCCAT
GGGGCATCTCTTGTGTACTTATTGTTTAAGGTTTCCTCAAACTGTGATTTTTCTGAACACAATAAACTAT
TTTGATGATCTTGGGTGGAAAAAAAAAAAAAAAAAAAAAAAAAAAAAAAAAA
SEQ ID NO: 27
Homo sapiens v-Ha-ras Harvey rat sarcoma viral oncogene homolog (HRAS),
transcript variant 3, mRNA
TGCCCTGCGCCCGCAACCCGAGCCGCACCCGCCGCGGACGGAGCCCATGCGCGGGGCGAACCGCGCGCCC
CCGCCCCCGCCCCGCCCCGGCCTCGGCCCCGGCCCTGGCCCCGGGGGCAGTCGCGCCTGTGAACGGTGGG
GCAGGAGACCCTGTAGGAGGACCCCGGGCCGCAGGCCCCTGAGGAGCGATGACGGAATATAAGCTGGTGG
TGGTGGGCGCCGGCGGTGTGGGCAAGAGTGCGCTGACCATCCAGCTGATCCAGAACCATTTTGTGGACGA
ATACGACCCCACTATAGAGGATTCCTACCGGAAGCAGGTGGTCATTGATGGGGAGACGTGCCTGTTGGAC
ATCCTGGATACCGCCGGCCAGGAGGAGTACAGCGCCATGCGGGACCAGTACATGCGCACCGGGGAGGGCT
TCCTGTGTGTGTTTGCCATCAACAACACCAAGTCTTTTGAGGACATCCACCAGTACAGGGAGCAGATCAA
ACGGGTGAAGGACTCGGATGACGTGCCCATGGTGCTGGTGGGGAACAAGTGTGACCTGGCTGCACGCACT
GTGGAATCTCGGCAGGCTCAGGACCTCGCCCGAAGCTACGGCATCCCCTACATCGAGACCTCGGCCAAGA
CCCGGCAGGGAGTGGAGGATGCCTTCTACACGTTGGTGCGTGAGATCCGGCAGCACAAGCTGCGGAAGCT
GAACCCTCCTGATGAGAGTGGCCCCGGCTGCATGAGCTGCAAGTGTGTGCTCTCCTGACGCAGGTGAGGG
GGACTCCCAGGGCGGCCGCCACGCCCACCGGATGACCCCGGCTCCCCGCCCCTGCCGGTCTCCTGGCCTG
CGGTCAGCAGCCTCCCTTGTGCCCCGCCCAGCACAAGCTCAGGACATGGAGGTGCCGGATGCAGGAAGGA
GGTGCAGACGGAAGGAGGAGGAAGGAAGGACGGAAGCAAGGAAGGAAGGAAGGGCTGCTGGAGCCCAGTC
ACCCCGGGACCGTGGGCCGAGGTGACTGCAGACCCTCCCAGGGAGGCTGTGCACAGACTGTCTTGAACAT
CCCAAATGCCACCGGAACCCCAGCCCTTAGCTCCCCTCCCAGGCCTCTGTGGGCCCTTGTCGGGCACAGA
TGGGATCACAGTAATTATTGGATGGTCTTGAAAAAAAAAAAAAAAAAAA
SEQ ID NO: 28
Homo sapiens interferon, alpha-inducible protein 27 (TF127), transcript
variant 1, mRNA
GGGAACACATCCAAGCTTAAGACGGTGAGGTCAGCTTCACATTCTCAGGAACTCTCCTTCTTTGGGTCTG
GCTGAAGTTGAGGATCTCTTACTCTCTAGGCCACGGAATTAACCCGAGCAGGCATGGAGGCCTCTGCTCT
CACCTCATCAGCAGTGACCAGTGTGGCCAAAGTGGTCAGGGTGGCCTCTGGCTCTGCCGTAGTTTTGCCC
CTGGCCAGGATTGCTACAGTTGTGATTGGAGGAGTTGTGGCCATGGCGGCTGTGCCCATGGTGCTCAGTG
CCATGGGCTTCACTGCGGCGGGAATCGCCTCGTCCTCCATAGCAGCCAAGATGATGTCCGCGGCGGCCAT
TGCCAATGGGGGTGGAGTTGCCTCGGGCAGCCTTGTGGCTACTCTGCAGTCACTGGGAGCAACTGGACTC
TCCGGATTGACCAAGTTCATCCTGGGCTCCATTGGGTCTGCCATTGCGGCTGTCATTGCGAGGTTCTACT
AGCTCCCTGCCCCTCGCCCTGCAGAGAAGAGAACCATGCCAGGGGAGAAGGCACCCAGCCATCCTGACCC
AGCGAGGAGCCAACTATCCCAAATATACCTGGGGTGAAATATACCAAATTCTGCATCTCCAGAGGAAAAT
AAGAAATAAAGATGAATTGTTGCAACTCTTCAAAA
SEQ ID NO: 29
Homo sapiens interleukin 11 receptor, alpha (IL11RA), transcript variant
3, mRNA
AGAGGGCGAGGGCGAGGGCAGAGGGCGCTGGCGGCAGCGGCCGCGGAAGATGAGCAGCAGCTGCTCAGGG
CTGAGCAGGGTCCTGGTGGCCGTGGCTACAGCCCTGGTGTCTGCCTCCTCCCCCTGCCCCCAGGCCTGGG
GCCCCCCAGGGGTCCAGTATGGGCAGCCAGGCAGGTCCGTGAAGCTGTGTTGTCCTGGAGTGACTGCCGG
GGACCCAGTGTCCTGGTTTCGGGATGGGGAGCCAAAGCTGCTCCAGGGACCTGACTCTGGGCTAGGGCAT
GAACTGGTCCTGGCCCAGGCAGACAGCACTGATGAGGGCACCTACATCTGCCAGACCCTGGATGGTGCAC
TTGGGGGCACAGTGACCCTGCAGCTGGGCTACCCTCCAGCCCGCCCTGTTGTCTCCTGCCAAGCAGCCGA
CTATGAGAACTTCTCTTGCACTTGGAGTCCCAGCCAGATCAGCGGTTTACCCACCCGCTACCTCACCTCC
TACAGGAAGAAGACAGTCCTAGGAGCTGATAGCCAGAGGAGGAGTCCATCCACAGGGCCCTGGCCATGCC
CACAGGATCCCCTAGGGGCTGCCCGCTGTGTTGTCCACGGGGCTGAGTTCTGGAGCCAGTACCGGATTAA
TGTGACTGAGGTGAACCCACTGGGTGCCAGCACACGCCTGCTGGATGTGAGCTTGCAGAGCATCTTGCGC
CCTGACCCACCCCAGGGCCTGCGGGTAGAGTCAGTACCAGGTTACCCCCGACGCCTGCGAGCCAGCTGGA
CATACCCTGCCTCCTGGCCGTGCCAGCCCCACTTCCTGCTCAAGTTCCGTTTGCAGTACCGTCCGGCGCA
GCATCCAGCCTGGTCCACGGTGGAGCCAGCTGGACTGGAGGAGGTGATCACAGATGCTGTGGCTGGGCTG
CCCCATGCTGTACGAGTCAGTGCCCGGGACTTTCTAGATGCTGGCACCTGGAGCACCTGGAGCCCGGAGG
CCTGGGGAACTCCGAGCACTGGGACCATACCAAAGGAGATACCAGCATGGGGCCAGCTACACACGCAGCC
AGAGGTGGAGCCTCAGGTGGACAGCCCTGCTCCTCCAAGGCCCTCCCTCCAACCACACCCTCGGCTACTT
GATCACAGGGACTCTGTGGAGCAGGTAGCTGTGCTGGCGTCTTTGGGAATCCTTTCTTTCCTGGGACTGG
TGGCTGGGGCCCTGGCACTGGGGCTCTGGCTGAGGCTGAGACGGGGTGGGAAGGATGGATCCCCAAAGCC
TGGGTTCTTGGCCTCAGTGATTCCAGTGGACAGGCGTCCAGGAGCTCCAAACCTGTAGAGGACCCAGGAG
GGCTTCGGCAGATTCCACCTATAATTCTGTCTTGCTGGTGTGGATAGAAACCAGGCAGGACAGTAGATCC
CTATGGTTGGATCTCAGCTGGAAGTTCTGTTTGGAGCCCATTTCTGTGAGACCCTGTATTTCAAATTTGC
AGCTGAAAGGTGCTTGTACCTCTGATTTCACCCCAGAGTTGGAGTTCTGCTCAAGGAACGTGTGTAATGT
GTACATCTGTGTCCATGTGTGACCATGTGTCTGTGAGGCAGGGAACATGTATTCTCTGCATGCATGTATG
TAGGTGCCTGGGGAGTGTGTGTGGGTCCTTGGCTCTTGGCCTTTCCCCTTGCAGGGGTTGTGCAGGTGTG
AATAAAGAGAATAAGGAAGTTCTTGGAAAAAAAAAAAAAAAAAAAAAAAAAAAAAAAAAAAAAAAAAAAA
AAAAAAAAAA
SEQ ID NO: 30
Homo sapiens jun proto-oncogene (JUN), mRNA
GACATCATGGGCTATTTTTAGGGGTTGACTGGTAGCAGATAAGTGTTGAGCTCGGGCTGGATAAGGGCTC
AGAGTTGCACTGAGTGTGGCTGAAGCAGCGAGGCGGGAGTGGAGGTGCGCGGAGTCAGGCAGACAGACAG
ACACAGCCAGCCAGCCAGGTCGGCAGTATAGTCCGAACTGCAAATCTTATTTTCTTTTCACCTTCTCTCT
AACTGCCCAGAGCTAGCGCCTGTGGCTCCCGGGCTGGTGTTTCGGGAGTGTCCAGAGAGCCTGGTCTCCA
GCCGCCCCCGGGAGGAGAGCCCTGCTGCCCAGGCGCTGTTGACAGCGGCGGAAAGCAGCGGTACCCACGC
GCCCGCCGGGGGAAGTCGGCGAGCGGCTGCAGCAGCAAAGAACTTTCCCGGCTGGGAGGACCGGAGACAA
GTGGCAGAGTCCCGGAGCGAACTTTTGCAAGCCTTTCCTGCGTCTTAGGCTTCTCCACGGCGGTAAAGAC
CAGAAGGCGGCGGAGAGCCACGCAAGAGAAGAAGGACGTGCGCTCAGCTTCGCTCGCACCGGTTGTTGAA
CTTGGGCGAGCGCGAGCCGCGGCTGCCGGGCGCCCCCTCCCCCTAGCAGCGGAGGAGGGGACAAGTCGTC
GGAGTCCGGGCGGCCAAGACCCGCCGCCGGCCGGCCACTGCAGGGTCCGCACTGATCCGCTCCGCGGGGA
GAGCCGCTGCTCTGGGAAGTGAGTTCGCCTGCGGACTCCGAGGAACCGCTGCGCCCGAAGAGCGCTCAGT
GAGTGACCGCGACTTTTCAAAGCCGGGTAGCGCGCGCGAGTCGACAAGTAAGAGTGCGGGAGGCATCTTA
ATTAACCCTGCGCTCCCTGGAGCGAGCTGGTGAGGAGGGCGCAGCGGGGACGACAGCCAGCGGGTGCGTG
CGCTCTTAGAGAAACTTTCCCTGTCAAAGGCTCCGGGGGGCGCGGGTGTCCCCCGCTTGCCAGAGCCCTG
TTGCGGCCCCGAAACTTGTGCGCGCAGCCCAAACTAACCTCACGTGAAGTGACGGACTGTTCTATGACTG
CAAAGATGGAAACGACCTTCTATGACGATGCCCTCAACGCCTCGTTCCTCCCGTCCGAGAGCGGACCTTA
TGGCTACAGTAACCCCAAGATCCTGAAACAGAGCATGACCCTGAACCTGGCCGACCCAGTGGGGAGCCTG
AAGCCGCACCTCCGCGCCAAGAACTCGGACCTCCTCACCTCGCCCGACGTGGGGCTGCTCAAGCTGGCGT
CGCCCGAGCTGGAGCGCCTGATAATCCAGTCCAGCAACGGGCACATCACCACCACGCCGACCCCCACCCA
GTTCCTGTGCCCCAAGAACGTGACAGATGAGCAGGAGGGCTTCGCCGAGGGCTTCGTGCGCGCCCTGGCC
GAACTGCACAGCCAGAACACGCTGCCCAGCGTCACGTCGGCGGCGCAGCCGGTCAACGGGGCAGGCATGG
TGGCTCCCGCGGTAGCCTCGGTGGCAGGGGGCAGCGGCAGCGGCGGCTTCAGCGCCAGCCTGCACAGCGA
GCCGCCGGTCTACGCAAACCTCAGCAACTTCAACCCAGGCGCGCTGAGCAGCGGCGGCGGGGCGCCCTCC
TACGGCGCGGCCGGCCTGGCCTTTCCCGCGCAACCCCAGCAGCAGCAGCAGCCGCCGCACCACCTGCCCC
AGCAGATGCCCGTGCAGCACCCGCGGCTGCAGGCCCTGAAGGAGGAGCCTCAGACAGTGCCCGAGATGCC
CGGCGAGACACCGCCCCTGTCCCCCATCGACATGGAGTCCCAGGAGCGGATCAAGGCGGAGAGGAAGCGC
ATGAGGAACCGCATCGCTGCCTCCAAGTGCCGAAAAAGGAAGCTGGAGAGAATCGCCCGGCTGGAGGAAA
AAGTGAAAACCTTGAAAGCTCAGAACTCGGAGCTGGCGTCCACGGCCAACATGCTCAGGGAACAGGTGGC
ACAGCTTAAACAGAAAGTCATGAACCACGTTAACAGTGGGTGCCAACTCATGCTAACGCAGCAGTTGCAA
ACATTTTGAAGAGAGACCGTCGGGGGCTGAGGGGCAACGAAGAAAAAAAATAACACAGAGAGACAGACTT
GAGAACTTGACAAGTTGCGACGGAGAGAAAAAAGAAGTGTCCGAGAACTAAAGCCAAGGGTATCCAAGTT
GGACTGGGTTGCGTCCTGACGGCGCCCCCAGTGTGCACGAGTGGGAAGGACTTGGCGCGCCCTCCCTTGG
CGTGGAGCCAGGGAGCGGCCGCCTGCGGGCTGCCCCGCTTTGCGGACGGGCTGTCCCCGCGCGAACGGAA
CGTTGGACTTTTCGTTAACATTGACCAAGAACTGCATGGACCTAACATTCGATCTCATTCAGTATTAAAG
GGGGGAGGGGGAGGGGGTTACAAACTGCAATAGAGACTGTAGATTGCTTCTGTAGTACTCCTTAAGAACA
CAAAGCGGGGGGAGGGTTGGGGAGGGGCGGCAGGAGGGAGGTTTGTGAGAGCGAGGCTGAGCCTACAGAT
GAACTCTTTCTGGCCTGCCTTCGTTAACTGTGTATGTACATATATATATTTTTTAATTTGATGAAAGCTG
ATTACTGTCAATAAACAGCTTCATGCCTTTGTAAGTTATTTCTTGTTTGTTTGTTTGGGTATCCTGCCCA
GTGTTGTTTGTAAATAAGAGATTTGGAGCACTCTGAGTTTACCATTTGTAATAAAGTATATAATTTTTTT
ATGTTTTGTTTCTGAAAATTCCAGAAAGGATATTTAAGAAAATACAATAAACTATTGGAAAGTACTCCCC
TAACCTCTTTTCTGCATCATCTGTAGATACTAGCTATCTAGGTGGAGTTGAAAGAGTTAAGAATGTCGAT
TAAAATCACTCTCAGTGCTTCTTACTATTAAGCAGTAAAAACTGTTCTCTATTAGACTTTAGAAATAAAT
GTACCTGATGTACCTGATGCTATGGTCAGGTTATACTCCTCCTCCCCCAGCTATCTATATGGAATTGCTT
ACCAAAGGATAGTGCGATGTTTCAGGAGGCTGGAGGAAGGGGGGTTGCAGTGGAGAGGGACAGCCCACTG
AGAAGTCAAACATTTCAAAGTTTGGATTGTATCAAGTGGCATGTGCTGTGACCATTTATAATGTTAGTAG
AAATTTTACAATAGGTGCTTATTCTCAAAGCAGGAATTGGTGGCAGATTTTACAAAAGATGTATCCTTCC
AATTTGGAATCTTCTCTTTGACAATTCCTAGATAAAAAGATGGCCTTTGCTTATGAATATTTATAACAGC
ATTCTTGTCACAATAAATGTATTCAAATACCAAAAAAAAAAAAAAAAA
SEQ ID NO: 31
Homo sapiens v-Ki-ras2 Kirsten rat sarcoma viral oncogene homolog (KRAS),
transcript variant b, mRNA
GGCCGCGGCGGCGGAGGCAGCAGCGGCGGCGGCAGTGGCGGCGGCGAAGGTGGCGGCGGCTCGGCCAGTA
CTCCCGGCCCCCGCCATTTCGGACTGGGAGCGAGCGCGGCGCAGGCACTGAAGGCGGCGGCGGGGCCAGA
GGCTCAGCGGCTCCCAGGTGCGGGAGAGAGGCCTGCTGAAAATGACTGAATATAAACTTGTGGTAGTTGG
AGCTGGTGGCGTAGGCAAGAGTGCCTTGACGATACAGCTAATTCAGAATCATTTTGTGGACGAATATGAT
CCAACAATAGAGGATTCCTACAGGAAGCAAGTAGTAATTGATGGAGAAACCTGTCTCTTGGATATTCTCG
ACACAGCAGGTCAAGAGGAGTACAGTGCAATGAGGGACCAGTACATGAGGACTGGGGAGGGCTTTCTTTG
TGTATTTGCCATAAATAATACTAAATCATTTGAAGATATTCACCATTATAGAGAACAAATTAAAAGAGTT
AAGGACTCTGAAGATGTACCTATGGTCCTAGTAGGAAATAAATGTGATTTGCCTTCTAGAACAGTAGACA
CAAAACAGGCTCAGGACTTAGCAAGAAGTTATGGAATTCCTTTTATTGAAACATCAGCAAAGACAAGACA
GGGTGTTGATGATGCCTTCTATACATTAGTTCGAGAAATTCGAAAACATAAAGAAAAGATGAGCAAAGAT
GGTAAAAAGAAGAAAAAGAAGTCAAAGACAAAGTGTGTAATTATGTAAATACAATTTGTACTTTTTTCTT
AAGGCATACTAGTACAAGTGGTAATTTTTGTACATTACACTAAATTATTAGCATTTGTTTTAGCATTACC
TAATTTTTTTCCTGCTCCATGCAGACTGTTAGCTTTTACCTTAAATGCTTATTTTAAAATGACAGTGGAA
GTTTTTTTTTCCTCTAAGTGCCAGTATTCCCAGAGTTTTGGTTTTTGAACTAGCAATGCCTGTGAAAAAG
AAACTGAATACCTAAGATTTCTGTCTTGGGGTTTTTGGTGCATGCAGTTGATTACTTCTTATTTTTCTTA
CCAATTGTGAATGTTGGTGTGAAACAAATTAATGAAGCTTTTGAATCATCCCTATTCTGTGTTTTATCTA
GTCACATAAATGGATTAATTACTAATTTCAGTTGAGACCTTCTAATTGGTTTTTACTGAAACATTGAGGG
AACACAAATTTATGGGCTTCCTGATGATGATTCTTCTAGGCATCATGTCCTATAGTTTGTCATCCCTGAT
GAATGTAAAGTTACACTGTTCACAAAGGTTTTGTCTCCTTTCCACTGCTATTAGTCATGGTCACTCTCCC
CAAAATATTATATTTTTTCTATAAAAAGAAAAAAATGGAAAAAAATTACAAGGCAATGGAAACTATTATA
AGGCCATTTCCTTTTCACATTAGATAAATTACTATAAAGACTCCTAATAGCTTTTCCTGTTAAGGCAGAC
CCAGTATGAAATGGGGATTATTATAGCAACCATTTTGGGGCTATATTTACATGCTACTAAATTTTTATAA
TAATTGAAAAGATTTTAACAAGTATAAAAAATTCTCATAGGAATTAAATGTAGTCTCCCTGTGTCAGACT
GCTCTTTCATAGTATAACTTTAAATCTTTTCTTCAACTTGAGTCTTTGAAGATAGTTTTAATTCTGCTTG
TGACATTAAAAGATTATTTGGGCCAGTTATAGCTTATTAGGTGTTGAAGAGACCAAGGTTGCAAGGCCAG
GCCCTGTGTGAACCTTTGAGCTTTCATAGAGAGTTTCACAGCATGGACTGTGTCCCCACGGTCATCCAGT
GTTGTCATGCATTGGTTAGTCAAAATGGGGAGGGACTAGGGCAGTTTGGATAGCTCAACAAGATACAATC
TCACTCTGTGGTGGTCCTGCTGACAAATCAAGAGCATTGCTTTTGTTTCTTAAGAAAACAAACTCTTTTT
TAAAAATTACTTTTAAATATTAACTCAAAAGTTGAGATTTTGGGGTGGTGGTGTGCCAAGACATTAATTT
TTTTTTTAAACAATGAAGTGAAAAAGTTTTACAATCTCTAGGTTTGGCTAGTTCTCTTAACACTGGTTAA
ATTAACATTGCATAAACACTTTTCAAGTCTGATCCATATTTAATAATGCTTTAAAATAAAAATAAAAACA
ATCCTTTTGATAAATTTAAAATGTTACTTATTTTAAAATAAATGAAGTGAGATGGCATGGTGAGGTGAAA
GTATCACTGGACTAGGAAGAAGGTGACTTAGGTTCTAGATAGGTGTCTTTTAGGACTCTGATTTTGAGGA
CATCACTTACTATCCATTTCTTCATGTTAAAAGAAGTCATCTCAAACTCTTAGTTTTTTTTTTTTACAAC
TATGTAATTTATATTCCATTTACATAAGGATACACTTATTTGTCAAGCTCAGCACAATCTGTAAATTTTT
AACCTATGTTACACCATCTTCAGTGCCAGTCTTGGGCAAAATTGTGCAAGAGGTGAAGTTTATATTTGAA
TATCCATTCTCGTTTTAGGACTCTTCTTCCATATTAGTGTCATCTTGCCTCCCTACCTTCCACATGCCCC
ATGACTTGATGCAGTTTTAATACTTGTAATTCCCCTAACCATAAGATTTACTGCTGCTGTGGATATCTCC
ATGAAGTTTTCCCACTGAGTCACATCAGAAATGCCCTACATCTTATTTCCTCAGGGCTCAAGAGAATCTG
ACAGATACCATAAAGGGATTTGACCTAATCACTAATTTTCAGGTGGTGGCTGATGCTTTGAACATCTCTT
TGCTGCCCAATCCATTAGCGACAGTAGGATTTTTCAAACCTGGTATGAATAGACAGAACCCTATCCAGTG
GAAGGAGAATTTAATAAAGATAGTGCTGAAAGAATTCCTTAGGTAATCTATAACTAGGACTACTCCTGGT
AACAGTAATACATTCCATTGTTTTAGTAACCAGAAATCTTCATGCAATGAAAAATACTTTAATTCATGAA
GCTTACTTTTTTTTTTTGGTGTCAGAGTCTCGCTCTTGTCACCCAGGCTGGAATGCAGTGGCGCCATCTC
AGCTCACTGCAACCTCCATCTCCCAGGTTCAAGCGATTCTCGTGCCTCGGCCTCCTGAGTAGCTGGGATT
ACAGGCGTGTGCCACTACACTCAACTAATTTTTGTATTTTTAGGAGAGACGGGGTTTCACCCTGTTGGCC
AGGCTGGTCTCGAACTCCTGACCTCAAGTGATTCACCCACCTTGGCCTCATAAACCTGTTTTGCAGAACT
CATTTATTCAGCAAATATTTATTGAGTGCCTACCAGATGCCAGTCACCGCACAAGGCACTGGGTATATGG
TATCCCCAAACAAGAGACATAATCCCGGTCCTTAGGTAGTGCTAGTGTGGTCTGTAATATCTTACTAAGG
CCTTTGGTATACGACCCAGAGATAACACGATGCGTATTTTAGTTTTGCAAAGAAGGGGTTTGGTCTCTGT
GCCAGCTCTATAATTGTTTTGCTACGATTCCACTGAAACTCTTCGATCAAGCTACTTTATGTAAATCACT
TCATTGTTTTAAAGGAATAAACTTGATTATATTGTTTTTTTATTTGGCATAACTGTGATTCTTTTAGGAC
AATTACTGTACACATTAAGGTGTATGTCAGATATTCATATTGACCCAAATGTGTAATATTCCAGTTTTCT
CTGCATAAGTAATTAAAATATACTTAAAAATTAATAGTTTTATCTGGGTACAAATAAACAGGTGCCTGAA
CTAGTTCACAGACAAGGAAACTTCTATGTAAAAATCACTATGATTTCTGAATTGCTATGTGAAACTACAG
ATCTTTGGAACACTGTTTAGGTAGGGTGTTAAGACTTACACAGTACCTCGTTTCTACACAGAGAAAGAAA
TGGCCATACTTCAGGAACTGCAGTGCTTATGAGGGGATATTTAGGCCTCTTGAATTTTTGATGTAGATGG
GCATTTTTTTAAGGTAGTGGTTAATTACCTTTATGTGAACTTTGAATGGTTTAACAAAAGATTTGTTTTT
GTAGAGATTTTAAAGGGGGAGAATTCTAGAAATAAATGTTACCTAATTATTACAGCCTTAAAGACAAAAA
TCCTTGTTGAAGTTTTTTTAAAAAAAGCTAAATTACATAGACTTAGGCATTAACATGTTTGTGGAAGAAT
ATAGCAGACGTATATTGTATCATTTGAGTGAATGTTCCCAAGTAGGCATTCTAGGCTCTATTTAACTGAG
TCACACTGCATAGGAATTTAGAACCTAACTTTTATAGGTTATCAAAACTGTTGTCACCATTGCACAATTT
TGTCCTAATATATACATAGAAACTTTGTGGGGCATGTTAAGTTACAGTTTGCACAAGTTCATCTCATTTG
TATTCCATTGATTTTTTTTTTCTTCTAAACATTTTTTCTTCAAACAGTATATAACTTTTTTTAGGGGATT
TTTTTTTAGACAGCAAAAACTATCTGAAGATTTCCATTTGTCAAAAAGTAATGATTTCTTGATAATTGTG
TAGTAATGTTTTTTAGAACCCAGCAGTTACCTTAAAGCTGAATTTATATTTAGTAACTTCTGTGTTAATA
CTGGATAGCATGAATTCTGCATTGAGAAACTGAATAGCTGTCATAAAATGAAACTTTCTTTCTAAAGAAA
GATACTCACATGAGTTCTTGAAGAATAGTCATAACTAGATTAAGATCTGTGTTTTAGTTTAATAGTTTGA
AGTGCCTGTTTGGGATAATGATAGGTAATTTAGATGAATTTAGGGGAAAAAAAAGTTATCTGCAGATATG
TTGAGGGCCCATCTCTCCCCCCACACCCCCACAGAGCTAACTGGGTTACAGTGTTTTATCCGAAAGTTTC
CAATTCCACTGTCTTGTGTTTTCATGTTGAAAATACTTTTGCATTTTTCCTTTGAGTGCCAATTTCTTAC
TAGTACTATTTCTTAATGTAACATGTTTACCTGGAATGTATTTTAACTATTTTTGTATAGTGTAAACTGA
AACATGCACATTTTGTACATTGTGCTTTCTTTTGTGGGACATATGCAGTGTGATCCAGTTGTTTTCCATC
ATTTGGTTGCGCTGACCTAGGAATGTTGGTCATATCAAACATTAAAAATGACCACTCTTTTAATTGAAAT
TAACTTTTAAATGTTTATAGGAGTATGTGCTGTGAAGTGATCTAAAATTTGTAATATTTTTGTCATGAAC
TGTACTACTCCTAATTATTGTAATGTAATAAAAATAGTTACAGTGACAAAAAAAAAAAAAAA
SEQ ID NO: 32
Homo sapiens leprecan-like 4 (LEPREL4), mRNA
GCTTCCTGGGCTTCCCATCTCTGGCGGGAAGCGCTCCCCGACGCATTCTCTACCTAGGGGACACCCCCAA
GGCAGGAGCCCGGGCCGACGGAGAGGACTTAACGACACTATCGGACCCTCTGGGAAAAGAGGGGAGACGT
CGTGACCCAGGCCCCGCCCCACCTTGCCGCCTCGTGCCCGGCGCTAAGACCCAGCGGGCGCGCCGCCCGC
CCGGGGCCCGGCCCTGTCCCCTTCCGTCCGCGGGGCAGCCAGCTCAGCTCCGGAGAGCCGGCGGCGCGGC
GGGCATGGCTCGGGTGGCGTGGGGGCTGCTGTGGTTGCTGCTGGGCAGCGCCGGGGCGCAGTACGAGAAG
TACAGCTTCCGGGGCTTCCCGCCCGAGGACCTGATGCCGCTGGCCGCGGCGTACGGGCACGCTCTGGAGC
AGTACGAGGGAGAGAGCTGGCGCGAGAGCGCGCGCTACCTGGAGGCGGCGCTGCGGCTGCACCGGCTCCT
GCGCGACAGCGAGGCCTTCTGCCACGCCAACTGCAGCGGCCCCGCGCCCGCGGCCAAGCCCGATCCCGAC
GGCGGCCGCGCAGACGAGTGGGCCTGCGAGCTGCGGCTCTTCGGCCGCGTCCTGGAGCGAGCCGCCTGCC
TGCGGCGCTGCAAGCGGACGCTGCCCGCCTTCCAGGTGCCCTACCCGCCGCGGCAGCTGCTGCGTGACTT
CCAGAGCCGCCTGCCCTACCAGTACCTGCACTACGCGCTGTTCAAGGCTAACCGGCTGGAGAAGGCGGTG
GCGGCGGCCTACACCTTCCTCCAGAGGAACCCGAAGCACGAGCTGACCGCCAAGTATCTCAACTACTATC
AGGGGATGCTGGACGTCGCCGACGAGTCCCTCACGGACCTAGAGGCCCAGCCCTACGAGGCCGTGTTCCT
CCGGGCTGTGAAGCTCTACAACAGCGGGGATTTCCGCAGCAGCACGGAGGACATGGAGCGGGCCTTGTCA
GAGTACCTGGCAGTCTTTGCCCGGTGCCTGGCCGGCTGTGAAGGGGCCCATGAGCAGGTGGACTTCAAGG
ACTTCTACCCGGCCATAGCAGATCTCTTTGCAGAGTCCCTGCAGTGCAAGGTGGACTGTGAGGCCAATTT
GACCCCCAATGTGGGTGGCTACTTCGTGGACAAGTTCGTGGCCACCATGTACCACTACCTGCAGTTTGCC
TACTATAAGTTGAATGATGTGCGCCAGGCTGCCCGCAGCGCCGCCAGCTACATGCTCTTCGACCCCAAGG
ACAGCGTCATGCAGCAGAACCTGGTGTATTACCGGTTCCACCGGGCTCGCTGGGGCCTGGAAGAGGAGGA
CTTCCAGCCCCGGGAGGAGGCCATGCTCTACCACAACCAGACCGCCGAGCTGCGGGAGCTGCTGGAGTTC
ACCCACATGTACCTGCAGTCAGATGATGAGATGGAGCTGGAGGAGACAGAACCGCCCCTGGAGCCTGAGG
ATGCCCTATCTGACGCCGAGTTTGAGGGGGAGGGTGACTACGAGGAGGGCATGTATGCTGACTGGTGGCA
GGAGCCGGATGCCAAGGGTGACGAGGCCGAGGCTGAGCCAGAGCCTGAACTCGCATGAGAAGGGGACACC
CCACACCGCTCAAGCTTGGGAAGCCTGGTGCCGATGGCCCCACCCTCACCAGCCTGGGCAGCAGCAAGAA
CTATTTATTAAAAACTTAAGATGGGCCAGGTGCGGTGGCTCACACCTGTAATCCCAGCATTTTGGGAGGC
CAAGGTGGGTGGATCACTTGAGGCCAGGAGTTCAAGACCAGCCTGGCCAACATGATGAGACCTCCGTCTC
TACTAAAATACATAAATTAGCCGGGTGTGGTGGCAGGCGCCTGAAATCCCAGCTACTCAAGAGGCTGAGG
CAGGAGAATCGCTTGAACCTGGGAGGCAAAGGTTGCAGTGAACTGAGATTGCGCCACCGCACTCCAGCCT
GGGCGACAGAGCGAGACTCCATCTTTAAAAAAAAACAAGACGGGCCGGCACGGTGGCTCACGCCTGTAAT
CCCAGCACTGAGAGGCCGATCACTTGAGGTCAGGAGTTCAAGACCAGCCTGGCCAACATGGTGAAACCCC
ATCTCTACTAAAAAATACAAAAATTAGCCAGGCATGGTGGCACACACCTGTAATCGTAGCTGAGGCAGGA
GAATCGCCTGAACCCAGGAGGCGGAGCTTGCAGTGAGCCGAGATCGTGCCACTGCACTCCAGCCTGGGCG
ACAGAGTGAGACTCCATCTCAAAAAAAAAAAAAAAAACTTAAGATGGACACAGCTGACTGGACCCCCATC
CTGCCTCACCCATGGGTGCTGCACCCCAGACCCATCCTGCCACTTCTATGTCTCTGGACCACAGGATGGT
GGTGGCATTGCAGGTTGGCAAGTGGGCTGATGGGGTCCGCCCTCCTCACTGCTGAGCTCCTCACCTGGAC
AGTCTCCTGGACAAGGAGTTTCCAGCTGCTGGCTGGAGTCTCAGGCCAAATTGCAGAGGGTCCTCCAGGG
TCCTGAAGAGCACTGGACTAAGAGTCTAGTGGTTCCAGGGCCCTGACCAGTAGGTGCTCAATAAATGTTT
GTTGTTGAATGAAAAAAAAAAAAAAAAAA
SEQ ID NO: 33
Homo sapiens lethal giant larvae homolog 2 (Drosophila) (LLGL2),
transcript variant 2, mRNA
GGAGGTGAGCAGGAAGGAGACGGCCGCCCAGCAGCCCGTGGGCAGGCGCGGCGGAGCGAGCGGGGCCGGC
GGCGGGCGCCGAGGGACGCCGAGGCCTCGGGCGGGGGCTGGCCCGGGGTTCCAGGTCTCCAGTGGGGGCT
GCAGACTAAGCAAAATGAGGCGGTTCCTGAGGCCAGGGCATGACCCTGTGCGGGAGAGGCTCAAGCGGGA
CCTGTTCCAGTTTAACAAGACGGTGGAGCATGGCTTCCCGCACCAGCCCAGCGCCCTCGGCTACAGCCCG
TCCCTGCGCATCCTGGCCATCGGCACCCGTTCTGGAGCCATCAAGCTCTACGGAGCCCCAGGCGTGGAGT
TCATGGGGCTGCACCAGGAGAACAACGCTGTGACGCAGATCCACCTCCTGCCCGGCCAGTGCCAGCTGGT
CACCCTGCTGGATGACAACAGCCTGCACCTTTGGAGCCTGAAGGTCAAGGGCGGGGCATCGGAGCTGCAG
GAGGATGAGAGCTTCACACTGCGTGGACCCCCAGGGGCTGCCCCCAGTGCCACACAGATCACCGTGGTCC
TGCCACATTCCTCCTGCGAGCTGCTCTACCTGGGCACCGAGAGTGGCAACGTGTTTGTGGTGCAGCTGCC
AGCTTTTCGTGCGCTGGAGGACCGGACCATCAGCTCGGACGCGGTGCTGCAGCGGTTGCCAGAGGAGGCC
CGCCACCGGCGTGTGTTCGAGATGGTGGAGGCACTGCAGGAGCACCCTCGAGACCCCAACCAGATCCTGA
TCGGCTACAGCCGAGGCCTCGTTGTCATCTGGGACCTACAGGGCAGCCGCGTGCTCTACCACTTCCTCAG
CAGCCAGCAACTGGAGAACATCTGGTGGCAGCGGGACGGCCGCCTGCTCGTCAGCTGTCACTCTGACGGC
AGCTACTGCCAGTGGCCCGTGTCCAGCGAAGCCCAGCAACCAGAGCCCCTCCGCAGCCTCGTGCCTTACG
GTCCCTTTCCTTGCAAAGCGATTACCAGAATCCTCTGGCTGACCACTAGGCAGGGGTTGCCCTTCACCAT
CTTCCAGGGTGGCATGCCACGGGCCAGCTACGGGGACCGCCACTGCATCTCAGTGATCCACGATGGCCAG
CAGACGGCCTTCGACTTCACCTCCCGTGTCATCGGCTTCACTGTCCTCACAGAGGCAGACCCTGCAGCCA
GTAGGAGAGCTTCGGGAGTGGGTGCCCAGGGTTAGGTGTGGGAGGCATGGGGCAGGACCATCAGTAAAGA
CAGGGCCAGGTGCAGTGGCTCCTGCCTGTAACCCCAGTGCTGTGGGAGGCCAAGGTGGTAGGATCGCTTG
AACCCAGGAGTTCAAGTCCAGCCTGGACAACGTAGGGAGACCCTTGTCTCTACAAAAAATAAAAAAATTA
GCCAGGAAAAAAAAAAAAAAAAAAAAAAAAAAAAAAAAAA
SEQ ID NO: 34
Homo sapiens neuroblastoma RAS viral (v-ras) oncogene homolog (NRAS),
mRNA
GAAACGTCCCGTGTGGGAGGGGCGGGTCTGGGTGCGGCCTGCCGCATGACTCGTGGTTCGGAGGCCCACG
TGGCCGGGGCGGGGACTCAGGCGCCTGGGGCGCCGACTGATTACGTAGCGGGCGGGGCCGGAAGTGCCGC
TCCTTGGTGGGGGCTGTTCATGGCGGTTCCGGGGTCTCCAACATTTTTCCCGGCTGTGGTCCTAAATCTG
TCCAAAGCAGAGGCAGTGGAGCTTGAGGTTCTTGCTGGTGTGAAATGACTGAGTACAAACTGGTGGTGGT
TGGAGCAGGTGGTGTTGGGAAAAGCGCACTGACAATCCAGCTAATCCAGAACCACTTTGTAGATGAATAT
GATCCCACCATAGAGGATTCTTACAGAAAACAAGTGGTTATAGATGGTGAAACCTGTTTGTTGGACATAC
TGGATACAGCTGGACAAGAAGAGTACAGTGCCATGAGAGACCAATACATGAGGACAGGCGAAGGCTTCCT
CTGTGTATTTGCCATCAATAATAGCAAGTCATTTGCGGATATTAACCTCTACAGGGAGCAGATTAAGCGA
GTAAAAGACTCGGATGATGTACCTATGGTGCTAGTGGGAAACAAGTGTGATTTGCCAACAAGGACAGTTG
ATACAAAACAAGCCCACGAACTGGCCAAGAGTTACGGGATTCCATTCATTGAAACCTCAGCCAAGACCAG
ACAGGGTGTTGAAGATGCTTTTTACACACTGGTAAGAGAAATACGCCAGTACCGAATGAAAAAACTCAAC
AGCAGTGATGATGGGACTCAGGGTTGTATGGGATTGCCATGTGTGGTGATGTAACAAGATACTTTTAAAG
TTTTGTCAGAAAAGAGCCACTTTCAAGCTGCACTGACACCCTGGTCCTGACTTCCCTGGAGGAGAAGTAT
TCCTGTTGCTGTCTTCAGTCTCACAGAGAAGCTCCTGCTACTTCCCCAGCTCTCAGTAGTTTAGTACAAT
AATCTCTATTTGAGAAGTTCTCAGAATAACTACCTCCTCACTTGGCTGTCTGACCAGAGAATGCACCTCT
TGTTACTCCCTGTTATTTTTCTGCCCTGGGTTCTTCCACAGCACAAACACACCTCTGCCACCCCAGGTTT
TTCATCTGAAAAGCAGTTCATGTCTGAAACAGAGAACCAAACCGCAAACGTGAAATTCTATTGAAAACAG
TGTCTTGAGCTCTAAAGTAGCAACTGCTGGTGATTTTTTTTTTCTTTTTACTGTTGAACTTAGAACTATG
CTAATTTTTGGAGAAATGTCATAAATTACTGTTTTGCCAAGAATATAGTTATTATTGCTGTTTGGTTTGT
TTATAATGTTATCGGCTCTATTCTCTAAACTGGCATCTGCTCTAGATTCATAAATACAAAAATGAATACT
GAATTTTGAGTCTATCCTAGTCTTCACAACTTTGACGTAATTAAATCCAACTTTCACAGTGAAGTGCCTT
TTTCCTAGAAGTGGTTTGTAGACTTCCTTTATAATATTTCAGTGGAATAGATGTCTCAAAAATCCTTATG
CATGAAATGAATGTCTGAGATACGTCTGTGACTTATCTACCATTGAAGGAAAGCTATATCTATTTGAGAG
CAGATGCCATTTTGTACATGTATGAAATTGGTTTTCCAGAGGCCTGTTTTGGGGCTTTCCCAGGAGAAAG
ATGAAACTGAAAGCACATGAATAATTTCACTTAATAATTTTTACCTAATCTCCACTTTTTTCATAGGTTA
CTACCTATACAATGTATGTAATTTGTTTCCCCTAGCTTACTGATAAACCTAATATTCAATGAACTTCCAT
TTGTATTCAAATTTGTGTCATACCAGAAAGCTCTACATTTGCAGATGTTCAAATATTGTAAAACTTTGGT
GCATTGTTATTTAATAGCTGTGATCAGTGATTTTCAAACCTCAAATATAGTATATTAACAAATTACATTT
TCACTGTATATCATGGTATCTTAATGATGTATATAATTGCCTTCAATCCCCTTCTCACCCCACCCTCTAC
AGCTTCCCCCACAGCAATAGGGGCTTGATTATTTCAGTTGAGTAAAGCATGGTGCTAATGGACCAGGGTC
ACAGTTTCAAAACTTGAACAATCCAGTTAGCATCACAGAGAAAGAAATTCTTCTGCATTTGCTCATTGCA
CCAGTAACTCCAGCTAGTAATTTTGCTAGGTAGCTGCAGTTAGCCCTGCAAGGAAAGAAGAGGTCAGTTA
GCACAAACCCTTTACCATGACTGGAAAACTCAGTATCACGTATTTAAACATTTTTTTTTCTTTTAGCCAT
GTAGAAACTCTAAATTAAGCCAATATTCTCATTTGAGAATGAGGATGTCTCAGCTGAGAAACGTTTTAAA
TTCTCTTTATTCATAATGTTCTTTGAAGGGTTTAAAACAAGATGTTGATAAATCTAAGCTGATGAGTTTG
CTCAAAACAGGAAGTTGAAATTGTTGAGACAGGAATGGAAAATATAATTAATTGATACCTATGAGGATTT
GGAGGCTTGGCATTTTAATTTGCAGATAATACCCTGGTAATTCTCATGAAAAATAGACTTGGATAACTTT
TGATAAAAGACTAATTCCAAAATGGCCACTTTGTTCCTGTCTTTAATATCTAAATACTTACTGAGGTCCT
CCATCTTCTATATTATGAATTTTCATTTATTAAGCAAATGTCATATTACCTTGAAATTCAGAAGAGAAGA
AACATATACTGTGTCCAGAGTATAATGAACCTGCAGAGTTGTGCTTCTTACTGCTAATTCTGGGAGCTTT
CACAGTACTGTCATCATTTGTAAATGGAAATTCTGCTTTTCTGTTTCTGCTCCTTCTGGAGCAGTGCTAC
TCTGTAATTTTCCTGAGGCTTATCACCTCAGTCATTTCTTTTTTAAATGTCTGTGACTGGCAGTGATTCT
TTTTCTTAAAAATCTATTAAATTTGATGTCAAATTAGGGAGAAAGATAGTTACTCATCTTGGGCTCTTGT
GCCAATAGCCCTTGTATGTATGTACTTAGAGTTTTCCAAGTATGTTCTAAGCACAGAAGTTTCTAAATGG
GGCCAAAATTCAGACTTGAGTATGTTCTTTGAATACCTTAAGAAGTTACAATTAGCCGGGCATGGTGGCC
CGTGCCTGTAGTCCCAGCTACTTGAGAGGCTGAGGCAGGAGAATCACTTCAACCCAGGAGGTGGAGGTTA
CAGTGAGCAGAGATCGTGCCACTGCACTCCAGCCTGGGTGACAAGAGAGACTTGTCTCCAAAAAAAAAGT
TACACCTAGGTGTGAATTTTGGCACAAAGGAGTGACAAACTTATAGTTAAAAGCTGAATAACTTCAGTGT
GGTATAAAACGTGGTTTTTAGGCTATGTTTGTGATTGCTGAAAAGAATTCTAGTTTACCTCAAAATCCTT
CTCTTTCCCCAAATTAAGTGCCTGGCCAGCTGTCATAAATTACATATTCCTTTTGGTTTTTTTAAAGGTT
ACATGTTCAAGAGTGAAAATAAGATGTTCTGTCTGAAGGCTACCATGCCGGATCTGTAAATGAACCTGTT
AAATGCTGTATTTGCTCCAACGGCTTACTATAGAATGTTACTTAATACAATATCATACTTATTACAATTT
TTACTATAGGAGTGTAATAGGTAAAATTAATCTCTATTTTAGTGGGCCCATGTTTAGTCTTTCACCATCC
TTTAAACTGCTGTGAATTTTTTTGTCATGACTTGAAAGCAAGGATAGAGAAACACTTTAGAGATATGTGG
GGTTTTTTTACCATTCCAGAGCTTGTGAGCATAATCATATTTGCTTTATATTTATAGTCATGAACTCCTA
AGTTGGCAGCTACAACCAAGAACCAAAAAATGGTGCGTTCTGCTTCTTGTAATTCATCTCTGCTAATAAA
TTATAAGAAGCAAGGAAAATTAGGGAAAATATTTTATTTGGATGGTTTCTATAAACAAGGGACTATAATT
CTTGTACATTATTTTTCATCTTTGCTGTTTCTTTGAGCAGTCTAATGTGCCACACAATTATCTAAGGTAT
TTGTTTTCTATAAGAATTGTTTTAAAAGTATTCTTGTTACCAGAGTAGTTGTATTATATTTCAAAACGTA
AGATGATTTTTAAAAGCCTGAGTACTGACCTAAGATGGAATTGTATGAACTCTGCTCTGGAGGGAGGGGA
GGATGTCCGTGGAAGTTGTAAGACTTTTATTTTTTTGTGCCATCAAATATAGGTAAAAATAATTGTGCAA
TTCTGCTGTTTAAACAGGAACTATTGGCCTCCTTGGCCCTAAATGGAAGGGCCGATATTTTAAGTTGATT
ATTTTATTGTAAATTAATCCAACCTAGTTCTTTTTAATTTGGTTGAATGTTTTTTCTTGTTAAATGATGT
TTAAAAAATAAAAACTGGAAGTTCTTGGCTTAGTCATAATTCTT
SEQ ID NO: 35
Homo sapiens 2′-5′-oligoadenylate synthetase 1, 40/46 kDa (OAS1),
transcript variant 3, mRNA
TCCCTTCTGAGGAAACGAAACCAACAGCAGTCCAAGCTCAGTCAGCAGAAGAGATAAAAGCAAACAGGTC
TGGGAGGCAGTTCTGTTGCCACTCTCTCTCCTGTCAATGATGGATCTCAGAAATACCCCAGCCAAATCTC
TGGACAAGTTCATTGAAGACTATCTCTTGCCAGACACGTGTTTCCGCATGCAAATCAACCATGCCATTGA
CATCATCTGTGGGTTCCTGAAGGAAAGGTGCTTCCGAGGTAGCTCCTACCCTGTGTGTGTGTCCAAGGTG
GTAAAGGGTGGCTCCTCAGGCAAGGGCACCACCCTCAGAGGCCGATCTGACGCTGACCTGGTTGTCTTCC
TCAGTCCTCTCACCACTTTTCAGGATCAGTTAAATCGCCGGGGAGAGTTCATCCAGGAAATTAGGAGACA
GCTGGAAGCCTGTCAAAGAGAGAGAGCATTTTCCGTGAAGTTTGAGGTCCAGGCTCCACGCTGGGGCAAC
CCCCGTGCGCTCAGCTTCGTACTGAGTTCGCTCCAGCTCGGGGAGGGGGTGGAGTTCGATGTGCTGCCTG
CCTTTGATGCCCTGGGTCAGTTGACTGGCGGCTATAAACCTAACCCCCAAATCTATGTCAAGCTCATCGA
GGAGTGCACCGACCTGCAGAAAGAGGGCGAGTTCTCCACCTGCTTCACAGAACTACAGAGAGACTTCCTG
AAGCAGCGCCCCACCAAGCTCAAGAGCCTCATCCGCCTAGTCAAGCACTGGTACCAAAATTGTAAGAAGA
AGCTTGGGAAGCTGCCACCTCAGTATGCCCTGGAGCTCCTGACGGTCTATGCTTGGGAGCGAGGGAGCAT
GAAAACACATTTCAACACAGCCCAGGGATTTCGGACGGTCTTGGAATTAGTCATAAACTACCAGCAACTC
TGCATCTACTGGACAAAGTATTATGACTTTAAAAACCCCATTATTGAAAAGTACCTGAGAAGGCAGCTCA
CGAAACCCAGGCCTGTGATCCTGGACCCGGCGGACCCTACAGGAAACTTGGGTGGTGGAGACCCAAAGGG
TTGGAGGCAGCTGGCACAAGAGGCTGAGGCCTGGCTGAATTACCCATGCTTTAAGAATTGGGATGGGTCC
CCAGTGAGCTCCTGGATTCTGCTGACCCAGCACACTCCAGGCAGCATCCACCCCACAGGCAGAAGAGGAC
TGGACCTGCACCATCCTCTGAATGCCAGTGCATCTTGGGGGAAAGGGCTCCAGTGTTATCTGGACCAGTT
CCTTCATTTTCAGGTGGGACTCTTGATCCAGAGAGGACAAAGCTCCTCAGTGAGCTGGTGTATAATCCAG
GACAGAACCCAGGTCTCCTGACTCCTGGCCTTCTATGCCCTCTATCCTATCATAGATAACATTCTCCACA
GCCTCACTTCATTCCACCTATTCTCTGAAAATATTCCCTGAGAGAGAACAGAGAGATTTAGATAAGAGAA
TGAAATTCCAGCCTTGACTTTCTTCTGTGCACCTGATGGGAGGGTAATGTCTAATGTATTATCAATAACA
ATAAAAATAAAGCAAATACCATTTAAAAAAAAAAA
SEQ ID NO: 36
Homo sapiens origin recognition complex, subunit 1 (ORC1), transcript
variant 3, mRNA
ACGGTCTGGGGGCGGGGCCACGCCGATTGGCGCGAAGTTTTCTTTTCTCCTTCCACCTTCTTTTCATTTC
TAGTGAGACACACGCTTTGGTCCTGGCTTTCGGCCCGTAGTTGTAGAAGGAGCCCTGCTGGTGCAGGTTA
GAGGTGCCGCATCCCCCGGAGCTCTCGAAGTGGAGGCGGTAGGAAACGGAGGGCTTGCGGCTAGCCGGAG
GAAGCTTTGGAGCCGGAAGCCATGGCACACTACCCCACAAGGCTGAAGACCAGAAAAACTTATTCATGGG
TTGGCAGGCCCTTGTTGGATCGAAAACTGCACTACCAAACCTATAGAGAAATGTGTGTGAAAACAGAAGG
TTGTTCCACCGAGATTCACATCCAGATTGGACAGTTTGTGTTGATTGAAGGGGATGATGATGAAAACCCG
TATGTTGCTAAATTGCTTGAGTTGTTCGAAGATGACTCTGATCCTCCTCCTAAGAAACGTGCTCGAGTAC
AGTGGTTTGTCCGATTCTGTGAAGTCCCTGCCTGTAAACGGCATTTGTTGGGCCGGAAGCCTGGTGCACA
GGAAATATTCTGGTATGATTACCCGGCCTGTGACAGCAACATTAATGCGGAGACCATCATTGGCCTTGTT
CGGGTGATACCTTTAGCCCCAAAGGATGTGGTACCGACGAATCTGAAAAATGAGAAGACACTCTTTGTGA
AACTATCCTGGAATGAGAAGAAATTCAGGCCACTTTCCTCAGAACTATTTGCGGAGTTGAATAAACCACA
AGAGAGTGCAGCCAAGTGCCAGAAACCCGTGAGAGCCAAGAGTAAGAGTGCAGAGAGCCCTTCTTGGACC
CCAGCAGAACATGTGGCCAAAAGGATTGAATCAAGGCACTCCGCCTCCAAATCTCGCCAAACTCCTACCC
ATCCTCTTACCCCAAGAGCCAGAAAGAGGCTGGAGCTTGGCAACTTAGGTAACCCTCAGATGTCCCAGCA
GACTTCATGTGCCTCCTTGGATTCTCCAGGAAGAATAAAACGGAAAGTGGCCTTCTCGGAGATCACCTCA
CCTTCTAAGAGATCTCAGCCTGATAAACTTCAAACCTTGTCTCCAGCTCTGAAAGCCCCAGAGAAAACCA
GAGAGACTGGACTCTCTTATACTGAGGATGACAAGAAGGCTTCACCTGAACATCGCATAATCCTGAGAAC
CCGAATTGCAGCTTCGAAAACCATAGACATTAGAGAGGAGAGAACACTTACCCCTATCAGTGGGGGACAG
AGATCTTCAGTGGTGCCATCCGTGATTCTGAAACCAGAAAACATCAAAAAGAGGGATGCAAAAGAAGCAA
AAGCCCAGAATGAAGCGACCTCTACTCCCCATCGTATCCGCAGAAAGAGTTCTGTCTTGACTATGAATCG
GATTAGGCAGCAGCTTCGGTTTCTAGGTAATAGTAAAAGTGACCAAGAAGAGAAAGAGATTCTGCCAGCA
GCAGAGATTTCAGACTCTAGCAGTGACGAAGAAGAGGCTTCCACACCGCCCCTTCCAAGGAGAGCACCCA
GAACTGTGTCCAGGAACCTGCGATCTTCCTTGAAGTCATCCTTACATACCCTCACGAAGCTCAAGCCTAG
AACGCCACGTTGTGCCGCTCCTCAGATCCGTAGTCGAAGCCTGGCTGCCCAGGAGCCAGCCAGTGTGCTG
GAGGAAGCCCGACTGAGGCTGCATGTTTCTGCTGTACCTGAGTCTCTTCCCTGTCGGGAACAGGAATTCC
AAGACATCTACAATTTTGTGGAAAGCAAACTCCTTGACCATACCGGAGGGTGCATGTACATCTCCGGTGT
CCCTGGGACAGGGAAGACTGCCACTGTTCATGAAGTGATACGCTGCCTGCAGCAGGCAGCCCAAGCCAAT
GATGTTCCTCCCTTTCAATACATTGAGGTCAATGGCATGAAGCTGACGGAGCCCCACCAAGTCTATGTGC
AAATCTTGCAGAAGCTAACAGGCCAAAAAGCAACAGCCAACCATGCGGCAGAACTGCTGGCAAAGCAATT
CTGCACCCGAGGGTCACCTCAGGAAACCACCGTCCTGCTTGTGGATGAGCTCGACCTTCTGTGGACTCAC
AAACAAGACATAATGTACAATCTCTTTGACTGGCCCACTCATAAGGAGGCCCGGCTTGTGGTCCTGGCAA
TTGCCAACACAATGGACCTGCCAGAGCGAATCATGATGAACCGGGTGTCCAGCCGACTGGGTCTTACCAG
GATGTGCTTCCAGCCCTATACATATAGCCAGCTGCAGCAGATCCTAAGGTCCCGGCTCAAGCATCTAAAG
GCCTTTGAAGATGATGCCATCCAGCTGGTAGCCAGGAAGGTAGCAGCACTGTCTGGAGATGCACGACGGT
GCCTGGACATCTGCAGGCGTGCCACAGAGATCTGTGAGTTCTCCCAGCAGAAGCCTGACTCCCCTGGCCT
GGTCACCATAGCCCACTCAATGGAAGCTGTGGATGAGATGTTTTCATCATCATACATCACGGCCATCAAA
AATTCCTCTGTTCTGGAACAGAGCTTCCTGAGAGCCATCCTCGCAGAGTTCCGTCGATCAGGACTGGAGG
AAGCCACGTTTCAACAGATATATAGTCAACATGTGGCACTGTGCAGAATGGAGGGACTGCCGTACCCCAC
CATGTCAGAGACCATGGCCGTGTGTTCTCACCTGGGCTCCTGTCGCCTCCTGCTTGTGGAGCCCAGCAGG
AACGATCTGCTCCTTCGGGTGCGGCTCAACGTCAGCCAGGATGATGTGCTGTATGCGCTGAAAGACGAGT
AAAGGGGCTTCACAAGTTAAAAGACTGGGGTCTTGCTGGGTTTTGTTTTTTGAGACAGGGTCTTGCTCTG
TCGCCCAGGCTGGAGTGCAGTGGCACGATCATGGCTCACTGCAGCCTTGACTTCTCAGGCTTAGGTGACC
CCCCAACCTCATCCTCCCAGGTGGCTGAAACTACAGGCACATGCCACCATGCCCAGCTGATTTTTTGTAG
AGACAGGGCTTCACCATGTTGCCAAGCTAGTCTACAAAGCATCTGATTTTGGAAGTACATGGAATTGTTG
TAACAAAGTATATTGAATGGAAATGGCTCTCATGTATTTTGGAATTTTCCATTAAATAATTTGCTTTTTC
CTGAAAAAAAAAAAAAAAAAAAAAAAA
SEQ ID NO: 37
Homo sapiens phosphoglycerate kinase 1 (PGK1), mRNA
GAGAGCAGCGGCCGGGAAGGGGCGGTGCGGGAGGCGGGGTGTGGGGCGGTAGTGTGGGCCCTGTTCCTGC
CCGCGCGGTGTTCCGCATTCTGCAAGCCTCCGGAGCGCACGTCGGCAGTCGGCTCCCTCGTTGACCGAAT
CACCGACCTCTCTCCCCAGCTGTATTTCCAAAATGTCGCTTTCTAACAAGCTGACGCTGGACAAGCTGGA
CGTTAAAGGGAAGCGGGTCGTTATGAGAGTCGACTTCAATGTTCCTATGAAGAACAACCAGATAACAAAC
AACCAGAGGATTAAGGCTGCTGTCCCAAGCATCAAATTCTGCTTGGACAATGGAGCCAAGTCGGTAGTCC
TTATGAGCCACCTAGGCCGGCCTGATGGTGTGCCCATGCCTGACAAGTACTCCTTAGAGCCAGTTGCTGT
AGAACTCAAATCTCTGCTGGGCAAGGATGTTCTGTTCTTGAAGGACTGTGTAGGCCCAGAAGTGGAGAAA
GCCTGTGCCAACCCAGCTGCTGGGTCTGTCATCCTGCTGGAGAACCTCCGCTTTCATGTGGAGGAAGAAG
GGAAGGGAAAAGATGCTTCTGGGAACAAGGTTAAAGCCGAGCCAGCCAAAATAGAAGCTTTCCGAGCTTC
ACTTTCCAAGCTAGGGGATGTCTATGTCAATGATGCTTTTGGCACTGCTCACAGAGCCCACAGCTCCATG
GTAGGAGTCAATCTGCCACAGAAGGCTGGTGGGTTTTTGATGAAGAAGGAGCTGAACTACTTTGCAAAGG
CCTTGGAGAGCCCAGAGCGACCCTTCCTGGCCATCCTGGGCGGAGCTAAAGTTGCAGACAAGATCCAGCT
CATCAATAATATGCTGGACAAAGTCAATGAGATGATTATTGGTGGTGGAATGGCTTTTACCTTCCTTAAG
GTGCTCAACAACATGGAGATTGGCACTTCTCTGTTTGATGAAGAGGGAGCCAAGATTGTCAAAGACCTAA
TGTCCAAAGCTGAGAAGAATGGTGTGAAGATTACCTTGCCTGTTGACTTTGTCACTGCTGACAAGTTTGA
TGAGAATGCCAAGACTGGCCAAGCCACTGTGGCTTCTGGCATACCTGCTGGCTGGATGGGCTTGGACTGT
GGTCCTGAAAGCAGCAAGAAGTATGCTGAGGCTGTCACTCGGGCTAAGCAGATTGTGTGGAATGGTCCTG
TGGGGGTATTTGAATGGGAAGCTTTTGCCCGGGGAACCAAAGCTCTCATGGATGAGGTGGTGAAAGCCAC
TTCTAGGGGCTGCATCACCATCATAGGTGGTGGAGACACTGCCACTTGCTGTGCCAAATGGAACACGGAG
GATAAAGTCAGCCATGTGAGCACTGGGGGTGGTGCCAGTTTGGAGCTCCTGGAAGGTAAAGTCCTTCCTG
GGGTGGATGCTCTCAGCAATATTTAGTACTTTCCTGCCTTTTAGTTCCTGTGCACAGCCCCTAAGTCAAC
TTAGCATTTTCTGCATCTCCACTTGGCATTAGCTAAAACCTTCCATGTCAAGATTCAGCTAGTGGCCAAG
AGATGCAGTGCCAGGAACCCTTAAACAGTTGCACAGCATCTCAGCTCATCTTCACTGCACCCTGGATTTG
CATACATTCTTCAAGATCCCATTTGAATTTTTTAGTGACTAAACCATTGTGCATTCTAGAGTGCATATAT
TTATATTTTGCCTGTTAAAAAGAAAGTGAGCAGTGTTAGCTTAGTTCTCTTTTGATGTAGGTTATTATGA
TTAGCTTTGTCACTGTTTCACTACTCAGCATGGAAACAAGATGAAATTCCATTTGTAGGTAGTGAGACAA
AATTGATGATCCATTAAGTAAACAATAAAAGTGTCCATTGAAACCGTGATTTTTTTTTTTTTCCTGTCAT
ACTTTGTTAGGAAGGGTGAGAATAGAATCTTGAGGAACGGATCAGATGTCTATATTGCTGAATGCAAGAA
GTGGGGCAGCAGCAGTGGAGAGATGGGACAATTAGATAAATGTCCATTCTTTATCAAGGGCCTACTTTAT
GGCAGACATTGTGCTAGTGCTTTTATTCTAACTTTTATTTTTATCAGTTACACATGATCATAATTTAAAA
AGTCAAGGCTTATAACAAAAAAGCCCCAGCCCATTCCTCCCATTCAAGATTCCCACTCCCCAGAGGTGAC
CACTTTCAACTCTTGAGTTTTTCAGGTATATACCTCCATGTTTCTAAGTAATATGCTTATATTGTTCACT
TCTTTTTTTTTTATTTTTTAAAGAAATCTATTTCATACCATGGAGGAAGGCTCTGTTCCACATATATTTC
CACTTCTTCATTCTCTCGGTATAGTTTTGTCACAATTATAGATTAGATCAAAAGTCTACATAACTAATAC
AGCTGAGCTATGTAGTATGCTATGATTAAATTTACTTATGTAAAAAAAAAAAAAAAAAA
SEQ ID NO: 38
Homo sapiens phorbol-12-myristate-13-acetate-induced protein 1 (PMATP1),
mRNA
ACTGGACAAAAGCGTGGTCTCTGGCGCGGGGATCTCAGAGTTTCCCGGGCACTCACCGTGTGTAGTTGGC
ATCTCCGCGCGTCCGGACACCCGATCCCAGCATCCCTGCCTGCAGGACTGTTCGTGTTCAGCTCGCGTCC
TGCAGCTGTCCGAGGTGCTCCAGTTGGAGGCTGAGGTTCCCGGGCTCTGTAGCTGAGTGGGCGGCGGCAC
CGGCGGAGATGCCTGGGAAGAAGGCGCGCAAGAACGCTCAACCGAGCCCCGCGCGGGCTCCAGCAGAGCT
GGAAGTCGAGTGTGCTACTCAACTCAGGAGATTTGGAGACAAACTGAACTTCCGGCAGAAACTTCTGAAT
CTGATATCCAAACTCTTCTGCTCAGGAACCTGACTGCATCAAAAACTTGCATGAGGGGACTCCTTCAAAA
GAGTTTTCTCAGGAGGTGCACGTTTCATCAATTTGAAGAAAGACTGCATTGTAATTGAGAGGAATGTGAA
GGTGCATTCATGGGTGCCCTTGGAAACGGAAGATGGAATACATCAAAGTGAATTTCTGTTCAAGTTTTCC
CAGATTATCATTCTTTGGGATGAGAGAACATTATAAAACCACTTTGTTTATTTTAAAGCAAGAATGGAAG
ACCCTTGAAAATAAAGAAGTAATTATTGACACATTTCTTTTTTACTTAGAGAATCGTTCTAGTGTTTTTG
CCGAAGATTACCGCTGGCCTACTGTGAAGGGAGATGACCTGTGATTAGACTGGGCGGCTGGGGAGAAACA
GTTCAGTGCATTGTTGTTGTTGCTGTTTTTGGTGTTTTGCTTTTCAGTGCCAACTCAGCACATTGTATAT
GATTCGGTTTATACATATTACCTTGTTATAATGAAAAAACTCATTCTGAGAACACTGAAATGTTATACTC
AGTGTTGATTTCTTCGGTCACTACACAACGTAAAATCATTTGTTTCTTTTGACTCAAATTGTATTGCTTC
TGTTCAGATGATCTTTCATTCAATGTGTTCCTGTTGGGCGTTACTAGAAACTATGGAAAACTGGAAAATA
ACTTTGAAAAAATTGGATAAAGTATAGGAGGGTTACTTGGGGCCAGTAAATCAGTAGACTGAACATTCAA
TATAATAAAAGAACATGGGGATTTTGTATAACCAGGGATAATAAAAAGAAAAAAGAAGTTAATTTTTAAT
TGATGTTTTTGAAACTTAGTAGAACAAATATTCAGAAGTAACTTGATAAGATATGAATGTTTCTAAAGAA
GTTTCTAAAGGTTCGGAAAATGCTCCTTGTCACATTAGTGTGCATCCTACAAAAAGTGATCTCTTAATGT
AAATTAAGAATATTTTCATAATTGGAATATACTTTTCTTAAAAAAAAGGAACAGTTAGTTCTCATCTAGA
ATGAAAGTTCCATATATGCATTGGTGAATATATATGTATACACATACTTACATACTTATATGGGTATCTG
TATAGATAATTTGTATTAGAGTATTATATAGCTTCTTAGTAGGGTCTCAAGTAAGTTTCATTTTTTTTAT
CTGGGCTATATACAGTCCTCAAATAAATAATGTCTTGATTTTATTTCAGCAGGAATAATTTTATTTATTT
TGCCTATTTATAATTAAAGTATTTTTCTTTAGTTTGAAAATGTGTATTAAAGTTACATTTTTGAGTTACA
AGAGTCTTATAACTACTTGAATTTTTAGTTAAAATGTCTTAATGTAGGTTGTAGTCACTTTAGATGGAAA
ATTACCTCACATCTGTTTTCTTCAGTATTACTTAAGATTGTTTATTTAGTGGTAGAGAGTTTTTTTTTTC
AGCCTAGAGGCAGCTATTTTACCATCTGGTATTTATGGTCTAATTTGTATTTAAACATATGCACACATAT
AAAAGTTGATACTGTGGCAGTAAACTATTAAAAGTTTTCACTGTTCAAAAAAAAAAAAAAAAAA
SEQ ID NO: 39
Homo sapiens POU class 6 homeobox 1 (POU6F1), transcript variant 2, non-
coding RNA
AATCGGTGGCCGCCAGACACCCGCGGCGAAGGCGGCTCGGGCTCGGGCTCCGGATGTGCTAGGTGTGGGC
CGGCCCCCACCCGACCCTGACAAGTGACCATGGATCCTGGAGCCGGGTCAGAGACATCTCTGACTGTCAA
TGAGCAGGTCATCGTGATGTCAGGTCATGAGACCATCCGAGTGCTGGAAGTCGGAGTGGATGCCCAACTC
CCTGCTGAGGAAGAGAGCAAAGGACTGGAGGGTGTGGCCGCCGAGGGCTCCCAGAGCGGAGACCCTGCTG
AAGCCAGTCAAGCTGCTGGTGAAGCTGGGCCAGACAACCTGGGCTCCTCTGCAGAGGCAACTGTGAAGTC
ACCCCCGGGGATCCCTCCGAGCCCTGCCCCTGCCATTGCCACCTTCAGCCAAGCCCCAAGCCAGCCTCAG
GCATCGCAGACCCTGACGCCACTGGCTGTACAAGCTGCCCCCCAGTATTGCAGGTCAAGTGGCTGGTCAG
CAGGGGCTGGCCGTGTGGACAATTCCTACAGCAACTGTGGCTGCCCTCCCAGGACTGACCGCTGCTTCTC
CTACGGGGGGAGTGTTCAAGCCACCTTTAGCCGGTCTCCAAGCAGCTGCTGTGCTGAACACCGCTCTTCC
GGCACCGGTACAAGCTGCCGCACCAGTACAGGCCTCCTCGACGGCCCAACCCCGGCCACCAGCCCAGCCC
CAGACGCTGTTCCAGACCCAGCCGCTGCTGCAGACCACACCTGCCATCCTCCCGCAGCCCACTGCTGCCA
CCGCTGCTGCCCCTACCCCCAAGCCAGTGGACACCCCCCCACAGATCACCGTCCAGCCTGCAGGCTTCGC
ATTTAGCCCAGGAATCATCAGTGCTGCTTCCCTCGGGGGACAGACCCAGATCCTGGGGTCCCTCACTACA
GCTCCAGTCATTACCAGCGCCATTCCCAGCATGCCAGGGATCAGCAGTCAGATCCTCACCAATGCTCAGG
GACAGGTTATTGGAACCCTTCCATGGGTAGTGAACTCAGCTAGTGTGGCGGCCCCAGCACCAGCCCAAAG
CCTGCAGGTCCAGGCCGTGACCCCCCAGCTGTTGTTGAACGCCCAGGGCCAGGTGATTGCGACCCTGGCT
AGCAGCCCCCTGCCTCCACCTGTGGCTGTCCGGAAGCCAAGCACACCTGAGTCCCCTGCTAAGAGTGAGG
TGCAGCCCATCCAGCCCACACCAACCGTGCCCCAGCCTGCTGTGGTCATTGCCAGCCCAGCTCCAGCCGC
CAAGCCATCTGCCTCTGCTCCTATCCCAATTACCTGCTCAGAGACCCCCACCGTCAGCCAGTTGGTGTCC
AAGCCACATACTCCAAGTCTGGATGAGGATGGGATCAACTTAGAAGAGATCCGGGAGTTTGCCAAGAACT
TTAAGATCCGGCGGCTCTCGCTGGGCCTTACACAGACCCAGGTGGGTCAGGCTCTGACTGCAACGGAAGG
TCCAGCCTACAGCCAGTCAGCCATCTGCCGGTTCGAGAAGCTAGACATCACACCCAAGAGTGCCCAGAAG
CTAAAGCCGGTGCTGGAAAAGTGGCTAAACGAAGCTGAACTGCGGAACCAGGAAGGCCAGCAGAACCTGA
TGGAGTTTGTGGGAGGCGAGCCCTCCAAGAAACGCAAACGCCGCACCTCCTTCACCCCCCAGGCCATAGA
GGCTCTCAATGCCTATTTTGAGAAGAACCCACTGCCCACAGGCCAGGAGATCACTGAAATTGCTAAGGAG
CTCAACTACGACCGTGAGGTAGTGCGGGTCTGGTTCTGCAATCGGCGCCAGACGCTCAAGAACACCAGCA
AGCTGAACGTCTTTCAGATCCCTTAGGGCTCAGCCCCTGGCCCTGTGTTCTAGCACTTTGTCCATTTCCC
GTGGCATCCGGCTGCAGCCACTGCCATGACAGCACCTGTCATTTTGCCACGTGCAGCTGTGCTCACCCCA
GGTCATCAGACTCCACCGTGTGCATGTGCATCAATGTCCCTCTTTTCTCCCACACATCTCACATCATGGG
GAGGCCAGAGGGGGCCACACGAGAGCTCCAGGCTCTGGGCTGGTCACTCCGAAGAAGAGGATTTGTGACG
TCACTTAGAGAAGCACCTTGCTAGCATGGTTTCTGAAGGGTGAATTCTGGTGGGGAACCAGAAACTCCCT
GTCTTTGGGGCAGGGCTAAAGCAGCTCCTAAGGACCACTGGCCATTAGCTCTTGCTTTTGATGGCATTCT
CTTTCCACCTTGTCTTCTCCTTTGCTCCTCTGTGTTAGTGTGGCAGGTATGACAACTCATCCAGTGGAAA
CACAGCCTCACACTGCCCTTCCGCCCCCCACACTTTGCCTGCAGGTGCACCGAAAGGACCTGGGAGATAA
AATTCAAAAAAGTGTGATGTGCTGCTCAGAAGGTCAGACTCCATGTCTGCCTTGACCTCAAGGTCAGAAG
GTTCCCAAACCCCTGGGGCTGGAACATGGGATCTCCTCTTCCACCTCTTCCTGGTTCCTTTGCGGGGAAA
ATTGCACTAAAACAGAACCTTTTCTTAATCCATGTTGGAAGGAAGCAACAGTGAACTCTACCTGTTCTGG
AGTTCTCCTGGGTCTGCAGAAGGTTGGGAATTTAGAAAATAAGGCTGTTCTTTCATATTTTAATTTAATC
TCTGTCAATGGCCATCCCTCCCACAAAAAAACGTGGGTTAAGAGAACTTGCAGACTGGATATGCAAGCAA
ACGGGCAACTCTGGAGAAAAATAAGGAAAGGAATGCTGACTTTCTCTTTCTTTCTCTTGTCCCCACACCC
ATTCCCAACCCAATACTGGGGCCTTCTCAAAAGGAGCAAATTAAACAATAAACCAGACAGCAAGGCCCTG
GGGGAAAGGACAACATCCTGAAATAAATGATGGAGCCCAGGAAGGTCTCTTGTGGAAGTTGACTTAACTC
TAATTTTCTTTGTAACTTTAAGCCTTGGATACGGGAGGAGAAATCTCATTTTGTCGAGTCTCAGACCATG
TCTGTGTGTAAGCAATCCCCACAGTGTCCTCTGAGCCAAGGACACCCCCAGATCAGATTGAGTTTTGCTT
CTAGACGGGGTAGCTATGGTACCTTGGGGGTTAGCTCTCATCCAAGCTGTTAAGTGAGTTTCCAGCCTCA
CTGTGGCTGGAAAGCCCCTAAAATTCAGTATGTAACTCCAGGAAGTCAGGAGAGAACTGAGATTTGCCTA
GATGACCACAGGCTTGCGGTGTAGATTATCCCTAAAGGGCCCCAAGTCACGGGGGTCAACCACCCCTGTC
TTCAGTACTCTTATCCTTACAGAGGCTGGTCTCTAACAGCTGCCTCCAGTGGACCTCCCATGATCCACCC
TGAGGGAAGGACCGTCAGCTGGGGACACATCACCACCTCTGTCAGTCACTGGTGCAGAGCCACCTCCTAG
CCTAGCTTCCTCTGGTGTCCTGTTTCCTTTCCCACTTACTGTTGGTGCCTCCCAGGCCCTGCAGTGCCAG
CGTGGCCACCCTCTTGGTAGCCTGGCCAGTAAGAGGAGGACAGTTGTGTGCTGAATTAGCACACGCACGT
GCAGCGCGCACAGACGCGCGCACACACACACACATACACGCTCTGCTGCATTTGGACAAACCATGCCTGC
CAGAGTGTAGCAGAGGTGAGGAAGCAGGTGGGCAGCTTGCCTGACCCAGCTTTTCAGGAGAGCGTGTCTC
CAACAGAGAGTCTCCACACTCTAGTTCAGGGTTATCGACCTGCCTCAATGAGATGACAGACTCATTTGGG
AGGGGTGTTGCAAACAAGTTTTCAGTGAGAATAGTTAAGTTCCAGAGCTTGTAAAGGATTCAGTGACTGA
CACTTCAGTAAATTAGGCCAGGCACATTGGCTTATGCCTGTAATTCCAACACTTTGGAAGGCCGAGGTGG
GCGGATCATTTGAGGTCTGGAGTTCGAGACCAGCCTGACCAACATGGTGAAACCCCGTCTCTACTAAAAA
TACAAAAATTAGCCAGGTGTGGTAGTGCACATCTGTAATCCCAGCTACTTGGGAGGTGGAGGCAGGAGAA
TTGCTTGAACCCTGGAGGTTGCAATGAGCTGAGATCACACTACTTCACTCCAGCCTGGGTGACAGAGCAA
GACTCGGTCTCAAACAAACAAAAACTTATGGCGATGCAGGTTTTCATGCTCAGACGCTTGCATTCAGGTA
TGCTTTCTTTTTTGAGAGAGACAAATGGGTCACAGCTGGCACCCTGGGAATAGCACATAATCCAGGGTGT
GTCTGTGGTGGTGGACGTGCAGGGGAACACCATCTGTCCTGTGTCATGATGGGAAAACAATCATGAACCA
CTGGTCTAAATTAGGCCTGGCCATGCTTTCTCAGCCCCTCCCTCATTTAAATTTGTCTTCCCAAAGCTGA
GCTAAAACTAAACCATTTCTCCTCTGCTGGAATGATGGATTGGTCATTCAGAGGAACAATACCAGGGGTG
GGAGGTTTGCAGGCTGAGTTCCCCAGGCATGGGGGTGCAGGGTGTCCCTGAGGTTTACCCAAAGCACAGC
TCGCTGGCCTGTGACCTCTGCCCTTCCTCCCACAGTGTAAGACCCCCCAGGAAGCAGCTGGGGCCTGAAC
CTCTCACCTAGGAGGTAGGTTTATTTTATTTTTTGTTAGCATCAGGCTCTGAAGGAGTTGGTATACATTT
TGTTTTGAAAACATCTTCTGGACTTACACCAGAGCTTAGTGTCGTCTTTACTATGGAAAGAGAGGAGAAT
GGACAGAAATGGTTTAACTGTGTGGAGTTTTGTTTGTTTTGTTTTAAATGGAAGAAAGACCAAAACTTTC
CTGGTGGATCAGCTAGGGCCTTTGACCCTGCATTACCACGGCATTTTATCCAGGTGAAGTCCAGGGAAAG
AACTCAGCCAAATGGACTAAGGAACACACGAGTTTGGAATGCGAGACTCTGACATTTTTGTGTTCTTGGA
AATCCAATTACCTTCCCATGCCCAGATTTCCTTCCTGCCTCTTGGACCAGGCTCTGGCACTGAGGTTCTC
ACTGTTCCCAACACAGACAAAGCTTCCTGAGGGCTGGAGGGGCAGCAAGGGGAGAGGAGAATGGGGAAGA
AGCGCTTGATGTAGTTGTGTGGAATAAACAGTATTTTTTCTTTTGTAAAAAAAAAAAAAAAAA
SEQ ID NO: 40
Homo sapiens Ran GTPase activating protein 1 (RANGAP1), mRNA
AAATCCTCCTCCTCCGCCATCATCCGCCGCGGTGCGGAGAGCAGGTGGTGCTGGAAGCGCGTGAGGCCGG
GAGCTCGAGAGAGCTAACAGACTAGCCGGCTGGACATCTGGACCGCTGGATCCGGAGGTGGCGACCCCGG
CCTGACCCGGACCCTAAATCCGTCCCCGCCCCAGAGGGCGGAGGCGCGCGCTCGATTCCCCCCACGCGGC
GGCGCCGCCTGTTTACGTCTGCAGATCTCCAGGGGAGCCCACCAGCCTAGTCAACATGGCCTCGGAAGAC
ATTGCCAAGCTGGCAGAGACACTTGCCAAGACTCAGGTGGCCGGGGGACAGCTGAGTTTCAAAGGCAAGA
GCCTCAAACTCAACACTGCAGAAGATGCTAAAGATGTGATTAAAGAGATTGAAGACTTTGACAGCTTGGA
GGCTCTGCGTCTGGAAGGCAACACAGTGGGCGTGGAAGCAGCCAGGGTCATCGCCAAGGCCTTAGAGAAG
AAGTCGGAGTTGAAGCGCTGCCACTGGAGTGACATGTTCACGGGAAGGCTGCGGACCGAGATCCCACCAG
CCCTGATCTCACTAGGGGAAGGACTCATCACAGCTGGGGCTCAGCTGGTGGAGCTGGACTTAAGCGACAA
CGCATTCGGGCCCGACGGTGTGCAAGGCTTCGAGGCCCTGCTCAAGAGCTCAGCCTGCTTCACCCTGCAG
GAACTCAAGCTCAACAACTGTGGCATGGGCATTGGCGGCGGCAAGATCCTGGCTGCAGCTCTGACCGAAT
GTCACCGGAAATCCAGTGCCCAAGGCAAGCCTCTGGCCCTGAAGGTCTTTGTGGCTGGCAGAAACCGTCT
GGAGAATGATGGCGCCACTGCCTTGGCAGAAGCTTTTAGGGTCATCGGGACCCTGGAGGAGGTCCACATG
CCACAGAATGGGATCAACCACCCTGGCATCACTGCCCTGGCCCAGGCTTTCGCTGTCAACCCCCTGCTGC
GGGTCATCAACCTGAATGACAACACCTTCACTGAGAAGGGCGCCGTGGCCATGGCCGAGACCTTGAAGAC
CTTGCGGCAGGTGGAGGTGATTAATTTTGGGGACTGCCTGGTGCGCTCCAAGGGTGCAGTTGCCATTGCA
GATGCCATCCGCGGCGGCCTGCCCAAGCTAAAGGAGCTGAACTTGTCATTCTGTGAAATCAAGAGGGATG
CTGCCCTGGCTGTTGCTGAGGCCATGGCAGACAAAGCTGAGCTGGAGAAGCTGGACCTGAATGGCAACAC
CCTGGGAGAAGAAGGCTGTGAACAGCTTCAGGAGGTGCTGGAGGGCTTCAACATGGCCAAGGTGCTGGCG
TCCCTCAGTGATGACGAGGACGAGGAGGAGGAGGAGGAAGGAGAAGAGGAAGAAGAGGAAGCAGAAGAAG
AGGAGGAGGAAGATGAGGAAGAGGAGGAAGAAGAGGAGGAGGAGGAGGAAGAAGAGCCTCAGCAGCGAGG
GCAGGGAGAGAAGTCAGCCACGCCCTCACGGAAGATTCTGGACCCTAACACTGGGGAGCCAGCTCCCGTG
CTGTCCTCCCCACCTCCTGCAGACGTCTCCACCTTCCTGGCTTTTCCCTCTCCAGAGAAGCTGCTGCGCC
TAGGGCCCAAGAGCTCCGTGCTGATAGCCCAGCAGACTGACACGTCTGACCCCGAGAAGGTGGTCTCTGC
CTTCCTAAAGGTGTCATCTGTGTTCAAGGACGAAGCTACTGTGAGGATGGCAGTGCAGGATGCAGTAGAT
GCCCTGATGCAGAAGGCTTTCAACTCCTCGTCCTTCAACTCCAACACCTTCCTCACCAGGCTGCTCGTGC
ACATGGGTCTGCTCAAGAGTGAAGACAAGGTCAAGGCCATTGCCAACCTGTACGGCCCCCTGATGGCGCT
GAACCACATGGTGCAGCAGGACTATTTCCCCAAGGCCCTTGCACCCCTGCTGCTGGCGTTCGTGACCAAG
CCCAACAGCGCCCTGGAATCCTGCTCCTTCGCCCGCCACAGTCTGCTGCAGACGCTGTACAAGGTCTAGA
CTCAAAGCCTCTCCCATCCCTTGGCCTGGACCAGTGAGCTGGGGAGGGACTCGGATGAACTGAGGCGCAG
CCTACGCCATTGCCTTGGACAGGACTCTGGCCACAGGCAGGGCGGGTCTGTGTCCCATGTGTCCTGTCAG
TCCCCTGAGTATGTGTGTGGGTGTGGCGCATGTGCAGGTCTGTGCCTCCTGTCGGGATTTGGGTTTTAAC
GTCTTCTGCTGGCCCAGCCCTGCTCTGTTGTGGGGAGTTGGCCCCCAGGGGAAAGGGCTGTGAGCTGCTC
CGCCATTAAACTCACCTCCACCTGAGGGCGCTCTGCTGATCTCCGCCTGGGCCCTGATGGCCGTCCCCAC
CCACCTGCCTTCCGGCCCGGCTCCCTGGCGGAGCCAGAACCCAGGGAGTTGCCCGCGTGCTGTCCTTCCC
CTCTGTGTTGTGATTGGGTTGTTTCCTGCCCTGCCTGGGGCTGCTTCTCGTCACCAAGCCCTGGTCCTGC
GGCAGCTGTCACCCCTACCATCCATACCACTGTGCTGACCGCTCAGCCTGAAGAGCAGAGAATGCCATGG
GTGGGACTGTGGGGGTCGGATCGTGGGGTTGTTGGCAGAGGGCAACCCTGGGCCCCACACCGTGTGGACA
GGCAGACACCAGATTGTCCAGGAGCAGGAGCTGCTGGGACTGCGCTGGCCCCGGACCTAGTGGGCCTTCT
CCTGGCTGCTGAGATGTCGTCTGTGACTGGCCTGGCTGGAGGGGGAGTGTTGACAACCCAAAGCTGTTCT
CCAGTCTGGGGAGGGAGAGGCAGGGTCCCCAATGTCCGAGCTGCATCTGGACGCTGCTCTTAAAGGACCT
CCTGGGGCAGGGGAGCGGTAGGGTCTGGACTGGGCAGATGCTGTATGACCTCCCTGAGCACCCGTGACTG
CCCCATGCTTTCCCCTTTGTGCTCTGTGTGTGTCTGGGCTGTGCCCGGGGGCTTCACAAATAAAGTCGTG
TGGCAGCTTCAAAAAAAAAAAAAAAAAAAA
SEQ ID NO: 41
Homo sapiens Spi-B transcription factor (Spi-1/PU.1 related) (SPTB),
mRNA
GGCAAACAGCCCGCCCGGCACCACCATGCTCGCCCTGGAGGCTGCACAGCTCGACGGGCCACACTTCAGC
TGTCTGTACCCAGATGGCGTCTTCTATGACCTGGACAGCTGCAAGCATTCCAGCTACCCTGATTCAGAGG
GGGCTCCTGACTCCCTGTGGGACTGGACTGTGGCCCCACCTGTCCCAGCCACCCCCTATGAAGCCTTCGA
CCCGGCAGCAGCCGCTTTTAGCCACCCCCAGGCTGCCCAGCTCTGCTACGAACCCCCCACCTACAGCCCT
GCAGGGAACCTCGAACTGGCCCCCAGCCTGGAGGCCCCGGGGCCTGGCCTCCCCGCATACCCCACGGAGA
ACTTCGCTAGCCAGACCCTGGTTCCCCCGGCATATGCCCCGTACCCCAGCCCTGTGCTATCAGAGGAGGA
AGACTTACCGTTGGACAGCCCTGCCCTGGAGGTCTCGGACAGCGAGTCGGATGAGGCCCTCGTGGCTGGC
CCCGAGGGGAAGGGATCCGAGGCAGGGACTCGCAAGAAGCTGCGCCTGTACCAGTTCCTGCTGGGGCTAC
TGACGCGCGGGGACATGCGTGAGTGCGTGTGGTGGGTGGAGCCAGGCGCCGGCGTCTTCCAGTTCTCCTC
CAAGCACAAGGAACTCCTGGCGCGCCGCTGGGGCCAGCAGAAGGGGAACCGCAAGCGCATGACCTACCAG
AAGCTGGCGCGCGCCCTCCGAAACTACGCCAAGACCGGCGAGATCCGCAAGGTCAAGCGCAAGCTCACCT
ACCAGTTCGACAGCGCGCTGCTGCCTGCAGTCCGCCGGGCCTGAGCACACCCGAGGCTCCCACCTGCGGA
GCCGCTGGGGGACCTCACGTCCCAGCCAGGATCCCCCTGGAAGAAAAAGGGCGTCCCCACACTCTAGGTG
ATAGGACTTACGCATCCCCACCTTTTGGGGTAAGGGGAGTGCTGCCCTGCCATAATCCCCAAGCCCAGCC
CGGGCCTGTCTGGGATTCCCCACTTGTGCCTGGGGTCCCTCTGGGATTTCTTTGTCATGTACAGACTCCC
TGGGATCCTCATGTTTTGGGTGACAGGACCTATGGACCACTATACTCGGGGAGGCAGGGTAGCAGTTCTT
CCAGAATCCCAAGAGCTTCTCTGGGATTTTCTTGTGATATCTGATTCCCCAGTGAGGCCTGGGACGTTTT
TAAGATCGCTGTGTGTCTGTAAACCCTGAATCTCATCTGGGGTGGGGGCCCTGCTGGCAACCCTGAGCCC
TGTCCAAGGTTCCCTCTTGTCAGATCTGAGATTTCCTAGTTATGTCTGGGGCCCTCTGGGAGCTGTTATC
ATCTCAGATCTCTTCGCCCATCTATGGCTGTGTTGTCACATCTGTCCCCTCATTTTTGAGATCCCCCAAT
TCTCTGGAACTATTCTGCTGCCCCTTTTTATGTGTCTGGAGTTCCCCAATCACATCTAGGGCTCCTCCAA
GAAAAAAAAAAAAAAAAAAAAAA
SEQ ID NO: 42
Homo sapiens TAF11 RNA polymerase II, TATA box binding protein (TBP)-
associated factor, 28 kDa (TAF11), mRNA
AAGATCCTGGCCTGTGCAGCTCGGGTTTCCGAGCTTCTGCCTCAGGCATCTCCGCGATCTCCTCTCCCCT
CCAATCCTATCCGTGATGGACGATGCCCACGAGTCGCCCTCCGACAAAGGTGGAGAGACAGGGGAGTCGG
ATGAGACGGCCGCTGTGCCCGGGGACCCGGGGGCTACCGACACCGATGGAATCCCAGAGGAAACTGACGG
AGACGCAGATGTGGACTTGAAAGAAGCTGCAGCGGAGGAAGGCGAGCTCGAGAGTCAGGATGTCTCAGAT
TTAACAACAGTTGAAAGGGAAGACTCATCATTACTTAATCCTGCAGCCAAAAAACTGAAAATAGATACCA
AAGAAAAGAAAGAGAAAAAGCAGAAAGTAGATGAAGATGAGATTCAGAAGATGCAAATCCTGGTTTCTTC
TTTTTCTGAGGAGCAGCTGAACCGTTATGAAATGTATCGCCGCTCAGCTTTCCCTAAGGCAGCCATCAAA
AGGCTGATCCAGTCCATCACTGGCACCTCTGTGTCTCAGAATGTTGTTATTGCTATGTCTGGTATTTCCA
AGGTTTTCGTCGGGGAGGTGGTAGAAGAAGCACTGGATGTGTGTGAGAAGTGGGGAGAAATGCCACCACT
ACAACCCAAACATATGAGGGAAGCCGTTAGAAGGTTAAAGTCAAAAGGACAGATCCCTAACTCGAAGCAC
AAAAAAATCATCTTCTTCTAGACCAAAGTCTAGAAAGGCCTATGTTACTGACGGAAGAAGTATTGGTTCC
AGACTTCCTATAAGACTGTCTGCATTGGTGCTTTAGTATCTCAGGCCTCCAAGGATTCCATGATGATTTT
AATGTCTTTCTCAAAACTCTGATATTTGTCACACCTAGAAAGTATGTAGCCTGATTGATACTTGCCTTGA
CTAAATTTTGGGACCTCTTGGGGCATTTTGAAGTATTTAACTGTCTTGACCAGTTGGAAGAAGATACGTG
GGCCATAAGCATCTTCTGGACAGGGGAACTGCTTTCAGAGAGAAAACCTTTCCAAGAGAGTTTTGTTTTG
TTTTGGTTTCGTTTTGTTTGAGATAGGGTCTTGCTCTATCACCTAGGCTGGAGTGCAGCGGCATGACTGC
AGCCTTGAACTCCTGGGCTTAAGTGACCCTCCCACCTCAGTCTCCTGAGTAGCTAGGACTACAGGCACAC
ACTACTGTGCCCAGCTAACTTATTTTTATTTTTTATGGAGATGGGGTCTTGCTTTGTTGCCCAGGCTGGT
CGTGAACTCCTGGCTTCAAGCAGTCCTCCTGCCTCAGCCTCCTAAAGTGCCGAGGGCTTTAATGGTTTCA
CATTGAAGCCTGAAGTTGCTAAGACTTAGGTTGTTTCTTATATCTGGTTTTAAGTAGATGAAACAACCAG
AAACTTTTACTTGTGATACTCTACCATGAAGGATGCGGTAATGGCAGGAATAGCAGAATAATTGGTGCTT
GTAAACATTTAAGATTCTCCTGTGGATTTTGGTGAGTGATCATTAAACTGTTTTCCAACTTGCAAAAAAA
AAAAAAAAAAAAAAAAAAAAAAAAAAAAAAAAAAAAAAAAAAAAAAAAAAAAAAAAAAA
SEQ ID NO: 43
Homo sapiens TATA box binding protein (TBP), transcript variant 2, mRNA
GGCGGAAGTGACATTATCAACGCGCGCCAGGGGTTCAGTGAGGTCGGGCAGGTTCGCTGTGGCGGGCGCC
TGGGCCGCCGGCTGTTTAACTTCGCTTCCGCTGGCCCATAGTGATCTTTGCAGTGACCCAGGGTGCCATG
ACTCCCGGAATCCCTATCTTTAGTCCAATGATGCCTTATGGCACTGGACTGACCCCACAGCCTATTCAGA
ACACCAATAGTCTGTCTATTTTGGAAGAGCAACAAAGGCAGCAGCAGCAACAACAACAGCAGCAGCAGCA
GCAGCAGCAGCAACAGCAACAGCAGCAGCAGCAGCAGCAGCAGCAGCAGCAGCAGCAGCAGCAGCAGCAG
CAGCAGCAACAGGCAGTGGCAGCTGCAGCCGTTCAGCAGTCAACGTCCCAGCAGGCAACACAGGGAACCT
CAGGCCAGGCACCACAGCTCTTCCACTCACAGACTCTCACAACTGCACCCTTGCCGGGCACCACTCCACT
GTATCCCTCCCCCATGACTCCCATGACCCCCATCACTCCTGCCACGCCAGCTTCGGAGAGTTCTGGGATT
GTACCGCAGCTGCAAAATATTGTATCCACAGTGAATCTTGGTTGTAAACTTGACCTAAAGACCATTGCAC
TTCGTGCCCGAAACGCCGAATATAATCCCAAGCGGTTTGCTGCGGTAATCATGAGGATAAGAGAGCCACG
AACCACGGCACTGATTTTCAGTTCTGGGAAAATGGTGTGCACAGGAGCCAAGAGTGAAGAACAGTCCAGA
CTGGCAGCAAGAAAATATGCTAGAGTTGTACAGAAGTTGGGTTTTCCAGCTAAGTTCTTGGACTTCAAGA
TTCAGAATATGGTGGGGAGCTGTGATGTGAAGTTTCCTATAAGGTTAGAAGGCCTTGTGCTCACCCACCA
ACAATTTAGTAGTTATGAGCCAGAGTTATTTCCTGGTTTAATCTACAGAATGATCAAACCCAGAATTGTT
CTCCTTATTTTTGTTTCTGGAAAAGTTGTATTAACAGGTGCTAAAGTCAGAGCAGAAATTTATGAAGCAT
TTGAAAACATCTACCCTATTCTAAAGGGATTCAGGAAGACGACGTAATGGCTCTCATGTACCCTTGCCTC
CCCCACCCCCTTCTTTTTTTTTTTTTAAACAAATCAGTTTGTTTTGGTACCTTTAAATGGTGGTGTTGTG
AGAAGATGGATGTTGAGTTGCAGGGTGTGGCACCAGGTGATGCCCTTCTGTAAGTGCCCACCGCGGGATG
CCGGGAAGGGGCATTATTTGTGCACTGAGAACACCGCGCAGCGTGACTGTGAGTTGCTCATACCGTGCTG
CTATCTGGGCAGCGCTGCCCATTTATTTATATGTAGATTTTAAACACTGCTGTTGACAAGTTGGTTTGAG
GGAGAAAACTTTAAGTGTTAAAGCCACCTCTATAATTGATTGGACTTTTTAATTTTAATGTTTTTCCCCA
TGAACCACAGTTTTTATATTTCTACCAGAAAAGTAAAAATCTTTTTTAAAAGTGTTGTTTTTCTAATTTA
TAACTCCTAGGGGTTATTTCTGTGCCAGACACATTCCACCTCTCCAGTATTGCAGGACAGAATATATGTG
TTAATGAAAATGAATGGCTGTACATATTTTTTTCTTTCTTCAGAGTACTCTGTACAATAAATGCAGTTTA
TAAAAGTGTTAGATTGTTGTTAAAAAAAAAAAAAA
SEQ ID NO: 44
Homo sapiens transforming growth factor, beta receptor II (70/80 kDa)
(TGFBR2), transcript variant 1, mRNA
GGAGAGGGAGAAGGCTCTCGGGCGGAGAGAGGTCCTGCCCAGCTGTTGGCGAGGAGTTTCCTGTTTCCCC
CGCAGCGCTGAGTTGAAGTTGAGTGAGTCACTCGCGCGCACGGAGCGACGACACCCCCGCGCGTGCACCC
GCTCGGGACAGGAGCCGGACTCCTGTGCAGCTTCCCTCGGCCGCCGGGGGCCTCCCCGCGCCTCGCCGGC
CTCCAGGCCCCCTCCTGGCTGGCGAGCGGGCGCCACATCTGGCCCGCACATCTGCGCTGCCGGCCCGGCG
CGGGGTCCGGAGAGGGCGCGGCGCGGAGGCGCAGCCAGGGGTCCGGGAAGGCGCCGTCCGCTGCGCTGGG
GGCTCGGTCTATGACGAGCAGCGGGGTCTGCCATGGGTCGGGGGCTGCTCAGGGGCCTGTGGCCGCTGCA
CATCGTCCTGTGGACGCGTATCGCCAGCACGATCCCACCGCACGTTCAGAAGTCGGATGTGGAAATGGAG
GCCCAGAAAGATGAAATCATCTGCCCCAGCTGTAATAGGACTGCCCATCCACTGAGACATATTAATAACG
ACATGATAGTCACTGACAACAACGGTGCAGTCAAGTTTCCACAACTGTGTAAATTTTGTGATGTGAGATT
TTCCACCTGTGACAACCAGAAATCCTGCATGAGCAACTGCAGCATCACCTCCATCTGTGAGAAGCCACAG
GAAGTCTGTGTGGCTGTATGGAGAAAGAATGACGAGAACATAACACTAGAGACAGTTTGCCATGACCCCA
AGCTCCCCTACCATGACTTTATTCTGGAAGATGCTGCTTCTCCAAAGTGCATTATGAAGGAAAAAAAAAA
GCCTGGTGAGACTTTCTTCATGTGTTCCTGTAGCTCTGATGAGTGCAATGACAACATCATCTTCTCAGAA
GAATATAACACCAGCAATCCTGACTTGTTGCTAGTCATATTTCAAGTGACAGGCATCAGCCTCCTGCCAC
CACTGGGAGTTGCCATATCTGTCATCATCATCTTCTACTGCTACCGCGTTAACCGGCAGCAGAAGCTGAG
TTCAACCTGGGAAACCGGCAAGACGCGGAAGCTCATGGAGTTCAGCGAGCACTGTGCCATCATCCTGGAA
GATGACCGCTCTGACATCAGCTCCACGTGTGCCAACAACATCAACCACAACACAGAGCTGCTGCCCATTG
AGCTGGACACCCTGGTGGGGAAAGGTCGCTTTGCTGAGGTCTATAAGGCCAAGCTGAAGCAGAACACTTC
AGAGCAGTTTGAGACAGTGGCAGTCAAGATCTTTCCCTATGAGGAGTATGCCTCTTGGAAGACAGAGAAG
GACATCTTCTCAGACATCAATCTGAAGCATGAGAACATACTCCAGTTCCTGACGGCTGAGGAGCGGAAGA
CGGAGTTGGGGAAACAATACTGGCTGATCACCGCCTTCCACGCCAAGGGCAACCTACAGGAGTACCTGAC
GCGGCATGTCATCAGCTGGGAGGACCTGCGCAAGCTGGGCAGCTCCCTCGCCCGGGGGATTGCTCACCTC
CACAGTGATCACACTCCATGTGGGAGGCCCAAGATGCCCATCGTGCACAGGGACCTCAAGAGCTCCAATA
TCCTCGTGAAGAACGACCTAACCTGCTGCCTGTGTGACTTTGGGCTTTCCCTGCGTCTGGACCCTACTCT
GTCTGTGGATGACCTGGCTAACAGTGGGCAGGTGGGAACTGCAAGATACATGGCTCCAGAAGTCCTAGAA
TCCAGGATGAATTTGGAGAATGTTGAGTCCTTCAAGCAGACCGATGTCTACTCCATGGCTCTGGTGCTCT
GGGAAATGACATCTCGCTGTAATGCAGTGGGAGAAGTAAAAGATTATGAGCCTCCATTTGGTTCCAAGGT
GCGGGAGCACCCCTGTGTCGAAAGCATGAAGGACAACGTGTTGAGAGATCGAGGGCGACCAGAAATTCCC
AGCTTCTGGCTCAACCACCAGGGCATCCAGATGGTGTGTGAGACGTTGACTGAGTGCTGGGACCACGACC
CAGAGGCCCGTCTCACAGCCCAGTGTGTGGCAGAACGCTTCAGTGAGCTGGAGCATCTGGACAGGCTCTC
GGGGAGGAGCTGCTCGGAGGAGAAGATTCCTGAAGACGGCTCCCTAAACACTACCAAATAGCTCTTCTGG
GGCAGGCTGGGCCATGTCCAAAGAGGCTGCCCCTCTCACCAAAGAACAGAGGCAGCAGGAAGCTGCCCCT
GAACTGATGCTTCCTGGAAAACCAAGGGGGTCACTCCCCTCCCTGTAAGCTGTGGGGATAAGCAGAAACA
ACAGCAGCAGGGAGTGGGTGACATAGAGCATTCTATGCCTTTGACATTGTCATAGGATAAGCTGTGTTAG
CACTTCCTCAGGAAATGAGATTGATTTTTACAATAGCCAATAACATTTGCACTTTATTAATGCCTGTATA
TAAATATGAATAGCTATGTTTTATATATATATATATATATCTATATATGTCTATAGCTCTATATATATAG
CCATACCTTGAAAAGAGACAAGGAAAAACATCAAATATTCCCAGGAAATTGGTTTTATTGGAGAACTCCA
GAACCAAGCAGAGAAGGAAGGGACCCATGACAGCATTAGCATTTGACAATCACACATGCAGTGGTTCTCT
GACTGTAAAACAGTGAACTTTGCATGAGGAAAGAGGCTCCATGTCTCACAGCCAGCTATGACCACATTGC
ACTTGCTTTTGCAAAATAATCATTCCCTGCCTAGCACTTCTCTTCTGGCCATGGAACTAAGTACAGTGGC
ACTGTTTGAGGACCAGTGTTCCCGGGGTTCCTGTGTGCCCTTATTTCTCCTGGACTTTTCATTTAAGCTC
CAAGCCCCAAATCTGGGGGGCTAGTTTAGAAACTCTCCCTCAACCTAGTTTAGAAACTCTACCCCATCTT
TAATACCTTGAATGTTTTGAACCCCACTTTTTACCTTCATGGGTTGCAGAAAAATCAGAACAGATGTCCC
CATCCATGCGATTGCCCCACCATCTACTAATGAAAAATTGTTCTTTTTTTCATCTTTCCCCTGCACTTAT
GTTACTATTCTCTGCTCCCAGCCTTCATCCTTTTCTAAAAAGGAGCAAATTCTCACTCTAGGCTTTATCG
TGTTTACTTTTTCATTACACTTGACTTGATTTTCTAGTTTTCTATACAAACACCAATGGGTTCCATCTTT
CTGGGCTCCTGATTGCTCAAGCACAGTTTGGCCTGATGAAGAGGATTTCAACTACACAATACTATCATTG
TCAGGACTATGACCTCAGGCACTCTAAACATATGTTTTGTTTGGTCAGCACAGCGTTTCAAAAAGTGAAG
CCACTTTATAAATATTTGGAGATTTTGCAGGAAAATCTGGATCCCCAGGTAAGGATAGCAGATGGTTTTC
AGTTATCTCCAGTCCACGTTCACAAAATGTGAAGGTGTGGAGACACTTACAAAGCTGCCTCACTTCTCAC
TGTAAACATTAGCTCTTTCCACTGCCTACCTGGACCCCAGTCTAGGAATTAAATCTGCACCTAACCAAGG
TCCCTTGTAAGAAATGTCCATTCAAGCAGTCATTCTCTGGGTATATAATATGATTTTGACTACCTTATCT
GGTGTTAAGATTTGAAGTTGGCCTTTTATTGGACTAAAGGGGAACTCCTTTAAGGGTCTCAGTTAGCCCA
AGTTTCTTTTGCTTATATGTTAATAGTTTTACCCTCTGCATTGGAGAGAGGAGTGCTTTACTCCAAGAAG
CTTTCCTCATGGTTACCGTTCTCTCCATCATGCCAGCCTTCTCAACCTTTGCAGAAATTACTAGAGAGGA
TTTGAATGTGGGACACAAAGGTCCCATTTGCAGTTAGAAAATTTGTGTCCACAAGGACAAGAACAAAGTA
TGAGCTTTAAAACTCCATAGGAAACTTGTTAATCAACAAAGAAGTGTTAATGCTGCAAGTAATCTCTTTT
TTAAAACTTTTTGAAGCTACTTATTTTCAGCCAAATAGGAATATTAGAGAGGGACTGGTAGTGAGAATAT
CAGCTCTGTTTGGATGGTGGAAGGTCTCATTTTATTGAGATTTTTAAGATACATGCAAAGGTTTGGAAAT
AGAACCTCTAGGCACCCTCCTCAGTGTGGGTGGGCTGAGAGTTAAAGACAGTGTGGCTGCAGTAGCATAG
AGGCGCCTAGAAATTCCACTTGCACCGTAGGGCATGCTGATACCATCCCAATAGCTGTTGCCCATTGACC
TCTAGTGGTGAGTTTCTAGAATACTGGTCCATTCATGAGATATTCAAGATTCAAGAGTATTCTCACTTCT
GGGTTATCAGCATAAACTGGAATGTAGTGTCAGAGGATACTGTGGCTTGTTTTGTTTATGTTTTTTTTTC
TTATTCAAGAAAAAAGACCAAGGAATAACATTCTGTAGTTCCTAAAAATACTGACTTTTTTCACTACTAT
ACATAAAGGGAAAGTTTTATTCTTTTATGGAACACTTCAGCTGTACTCATGTATTAAAATAGGAATGTGA
ATGCTATATACTCTTTTTATATCAAAAGTCTCAAGCACTTATTTTTATTCTATGCATTGTTTGTCTTTTA
CATAAATAAAATGTTTATTAGATTGAATAAAGCAAAATACTCAGGTGAGCATCCTGCCTCCTGTTCCCAT
TCCTAGTAGCTAAA
SEQ ID NO: 45
Homo sapiens tumor protein p53 (TP53), transcript variant 4, mRNA
GATTGGGGTTTTCCCCTCCCATGTGCTCAAGACTGGCGCTAAAAGTTTTGAGCTTCTCAAAAGTCTAGAG
CCACCGTCCAGGGAGCAGGTAGCTGCTGGGCTCCGGGGACACTTTGCGTTCGGGCTGGGAGCGTGCTTTC
CACGACGGTGACACGCTTCCCTGGATTGGCAGCCAGACTGCCTTCCGGGTCACTGCCATGGAGGAGCCGC
AGTCAGATCCTAGCGTCGAGCCCCCTCTGAGTCAGGAAACATTTTCAGACCTATGGAAACTACTTCCTGA
AAACAACGTTCTGTCCCCCTTGCCGTCCCAAGCAATGGATGATTTGATGCTGTCCCCGGACGATATTGAA
CAATGGTTCACTGAAGACCCAGGTCCAGATGAAGCTCCCAGAATGCCAGAGGCTGCTCCCCCCGTGGCCC
CTGCACCAGCAGCTCCTACACCGGCGGCCCCTGCACCAGCCCCCTCCTGGCCCCTGTCATCTTCTGTCCC
TTCCCAGAAAACCTACCAGGGCAGCTACGGTTTCCGTCTGGGCTTCTTGCATTCTGGGACAGCCAAGTCT
GTGACTTGCACGTACTCCCCTGCCCTCAACAAGATGTTTTGCCAACTGGCCAAGACCTGCCCTGTGCAGC
TGTGGGTTGATTCCACACCCCCGCCCGGCACCCGCGTCCGCGCCATGGCCATCTACAAGCAGTCACAGCA
CATGACGGAGGTTGTGAGGCGCTGCCCCCACCATGAGCGCTGCTCAGATAGCGATGGTCTGGCCCCTCCT
CAGCATCTTATCCGAGTGGAAGGAAATTTGCGTGTGGAGTATTTGGATGACAGAAACACTTTTCGACATA
GTGTGGTGGTGCCCTATGAGCCGCCTGAGGTTGGCTCTGACTGTACCACCATCCACTACAACTACATGTG
TAACAGTTCCTGCATGGGCGGCATGAACCGGAGGCCCATCCTCACCATCATCACACTGGAAGACTCCAGT
GGTAATCTACTGGGACGGAACAGCTTTGAGGTGCGTGTTTGTGCCTGTCCTGGGAGAGACCGGCGCACAG
AGGAAGAGAATCTCCGCAAGAAAGGGGAGCCTCACCACGAGCTGCCCCCAGGGAGCACTAAGCGAGCACT
GCCCAACAACACCAGCTCCTCTCCCCAGCCAAAGAAGAAACCACTGGATGGAGAATATTTCACCCTTCAG
ATGCTACTTGACTTACGATGGTGTTACTTCCTGATAAACTCGTCGTAAGTTGAAAATATTATCCGTGGGC
GTGAGCGCTTCGAGATGTTCCGAGAGCTGAATGAGGCCTTGGAACTCAAGGATGCCCAGGCTGGGAAGGA
GCCAGGGGGGAGCAGGGCTCACTCCAGCCACCTGAAGTCCAAAAAGGGTCAGTCTACCTCCCGCCATAAA
AAACTCATGTTCAAGACAGAAGGGCCTGACTCAGACTGACATTCTCCACTTCTTGTTCCCCACTGACAGC
CTCCCACCCCCATCTCTCCCTCCCCTGCCATTTTGGGTTTTGGGTCTTTGAACCCTTGCTTGCAATAGGT
GTGCGTCAGAAGCACCCAGGACTTCCATTTGCTTTGTCCCGGGGCTCCACTGAACAAGTTGGCCTGCACT
GGTGTTTTGTTGTGGGGAGGAGGATGGGGAGTAGGACATACCAGCTTAGATTTTAAGGTTTTTACTGTGA
GGGATGTTTGGGAGATGTAAGAAATGTTCTTGCAGTTAAGGGTTAGTTTACAATCAGCCACATTCTAGGT
AGGGGCCCACTTCACCGTACTAACCAGGGAAGCTGTCCCTCACTGTTGAATTTTCTCTAACTTCAAGGCC
CATATCTGTGAAATGCTGGCATTTGCACCTACCTCACAGAGTGCATTGTGAGGGTTAATGAAATAATGTA
CATCTGGCCTTGAAACCACCTTTTATTACATGGGGTCTAGAACTTGACCCCCTTGAGGGTGCTTGTTCCC
TCTCCCTGTTGGTCGGTGGGTTGGTAGTTTCTACAGTTGGGCAGCTGGTTAGGTAGAGGGAGTTGTCAAG
TCTCTGCTGGCCCAGCCAAACCCTGTCTGACAACCTCTTGGTGAACCTTAGTACCTAAAAGGAAATCTCA
CCCCATCCCACACCCTGGAGGATTTCATCTCTTGTATATGATGATCTGGATCCACCAAGACTTGTTTTAT
GCTCAGGGTCAATTTCTTTTTTCTTTTTTTTTTTTTTTTTTCTTTTTCTTTGAGACTGGGTCTCGCTTTG
TTGCCCAGGCTGGAGTGGAGTGGCGTGATCTTGGCTTACTGCAGCCTTTGCCTCCCCGGCTCGAGCAGTC
CTGCCTCAGCCTCCGGAGTAGCTGGGACCACAGGTTCATGCCACCATGGCCAGCCAACTTTTGCATGTTT
TGTAGAGATGGGGTCTCACAGTGTTGCCCAGGCTGGTCTCAAACTCCTGGGCTCAGGCGATCCACCTGTC
TCAGCCTCCCAGAGTGCTGGGATTACAATTGTGAGCCACCACGTCCAGCTGGAAGGGTCAACATCTTTTA
CATTCTGCAAGCACATCTGCATTTTCACCCCACCCTTCCCCTCCTTCTCCCTTTTTATATCCCATTTTTA
TATCGATCTCTTATTTTACAATAAAACTTTGCTGCCACCTGTGTGTCTGAGGGGTG
SEQ ID NO: 46
Homo sapiens tumor protein p53 (TP53), transcript variant 2, mRNA
GATTGGGGTTTTCCCCTCCCATGTGCTCAAGACTGGCGCTAAAAGTTTTGAGCTTCTCAAAAGTCTAGAG
CCACCGTCCAGGGAGCAGGTAGCTGCTGGGCTCCGGGGACACTTTGCGTTCGGGCTGGGAGCGTGCTTTC
CACGACGGTGACACGCTTCCCTGGATTGGCCAGACTGCCTTCCGGGTCACTGCCATGGAGGAGCCGCAGT
CAGATCCTAGCGTCGAGCCCCCTCTGAGTCAGGAAACATTTTCAGACCTATGGAAACTACTTCCTGAAAA
CAACGTTCTGTCCCCCTTGCCGTCCCAAGCAATGGATGATTTGATGCTGTCCCCGGACGATATTGAACAA
TGGTTCACTGAAGACCCAGGTCCAGATGAAGCTCCCAGAATGCCAGAGGCTGCTCCCCCCGTGGCCCCTG
CACCAGCAGCTCCTACACCGGCGGCCCCTGCACCAGCCCCCTCCTGGCCCCTGTCATCTTCTGTCCCTTC
CCAGAAAACCTACCAGGGCAGCTACGGTTTCCGTCTGGGCTTCTTGCATTCTGGGACAGCCAAGTCTGTG
ACTTGCACGTACTCCCCTGCCCTCAACAAGATGTTTTGCCAACTGGCCAAGACCTGCCCTGTGCAGCTGT
GGGTTGATTCCACACCCCCGCCCGGCACCCGCGTCCGCGCCATGGCCATCTACAAGCAGTCACAGCACAT
GACGGAGGTTGTGAGGCGCTGCCCCCACCATGAGCGCTGCTCAGATAGCGATGGTCTGGCCCCTCCTCAG
CATCTTATCCGAGTGGAAGGAAATTTGCGTGTGGAGTATTTGGATGACAGAAACACTTTTCGACATAGTG
TGGTGGTGCCCTATGAGCCGCCTGAGGTTGGCTCTGACTGTACCACCATCCACTACAACTACATGTGTAA
CAGTTCCTGCATGGGCGGCATGAACCGGAGGCCCATCCTCACCATCATCACACTGGAAGACTCCAGTGGT
AATCTACTGGGACGGAACAGCTTTGAGGTGCGTGTTTGTGCCTGTCCTGGGAGAGACCGGCGCACAGAGG
AAGAGAATCTCCGCAAGAAAGGGGAGCCTCACCACGAGCTGCCCCCAGGGAGCACTAAGCGAGCACTGCC
CAACAACACCAGCTCCTCTCCCCAGCCAAAGAAGAAACCACTGGATGGAGAATATTTCACCCTTCAGATC
CGTGGGCGTGAGCGCTTCGAGATGTTCCGAGAGCTGAATGAGGCCTTGGAACTCAAGGATGCCCAGGCTG
GGAAGGAGCCAGGGGGGAGCAGGGCTCACTCCAGCCACCTGAAGTCCAAAAAGGGTCAGTCTACCTCCCG
CCATAAAAAACTCATGTTCAAGACAGAAGGGCCTGACTCAGACTGACATTCTCCACTTCTTGTTCCCCAC
TGACAGCCTCCCACCCCCATCTCTCCCTCCCCTGCCATTTTGGGTTTTGGGTCTTTGAACCCTTGCTTGC
AATAGGTGTGCGTCAGAAGCACCCAGGACTTCCATTTGCTTTGTCCCGGGGCTCCACTGAACAAGTTGGC
CTGCACTGGTGTTTTGTTGTGGGGAGGAGGATGGGGAGTAGGACATACCAGCTTAGATTTTAAGGTTTTT
ACTGTGAGGGATGTTTGGGAGATGTAAGAAATGTTCTTGCAGTTAAGGGTTAGTTTACAATCAGCCACAT
TCTAGGTAGGGGCCCACTTCACCGTACTAACCAGGGAAGCTGTCCCTCACTGTTGAATTTTCTCTAACTT
CAAGGCCCATATCTGTGAAATGCTGGCATTTGCACCTACCTCACAGAGTGCATTGTGAGGGTTAATGAAA
TAATGTACATCTGGCCTTGAAACCACCTTTTATTACATGGGGTCTAGAACTTGACCCCCTTGAGGGTGCT
TGTTCCCTCTCCCTGTTGGTCGGTGGGTTGGTAGTTTCTACAGTTGGGCAGCTGGTTAGGTAGAGGGAGT
TGTCAAGTCTCTGCTGGCCCAGCCAAACCCTGTCTGACAACCTCTTGGTGAACCTTAGTACCTAAAAGGA
AATCTCACCCCATCCCACACCCTGGAGGATTTCATCTCTTGTATATGATGATCTGGATCCACCAAGACTT
GTTTTATGCTCAGGGTCAATTTCTTTTTTCTTTTTTTTTTTTTTTTTTCTTTTTCTTTGAGACTGGGTCT
CGCTTTGTTGCCCAGGCTGGAGTGGAGTGGCGTGATCTTGGCTTACTGCAGCCTTTGCCTCCCCGGCTCG
AGCAGTCCTGCCTCAGCCTCCGGAGTAGCTGGGACCACAGGTTCATGCCACCATGGCCAGCCAACTTTTG
CATGTTTTGTAGAGATGGGGTCTCACAGTGTTGCCCAGGCTGGTCTCAAACTCCTGGGCTCAGGCGATCC
ACCTGTCTCAGCCTCCCAGAGTGCTGGGATTACAATTGTGAGCCACCACGTCCAGCTGGAAGGGTCAACA
TCTTTTACATTCTGCAAGCACATCTGCATTTTCACCCCACCCTTCCCCTCCTTCTCCCTTTTTATATCCC
ATTTTTATATCGATCTCTTATTTTACAATAAAACTTTGCTGCCACCTGTGTGTCTGAGGGGTG
SEQ ID NO: 47
Homo sapiens TXK tyrosine kinase (TXK), mRNA
GATTTCAGTTGAAAGATGTGTTTTTGTGAGTAGAGCACCGCAGAAGAACTGAAGACTGTTGTGTGCTCCC
CGCAGAAGGGGCTACCATGATCCTTTCCTCCTATAACACCATCCAGTCGGTTTTCTGTTGCTGCTGTTGC
TGTTCAGTGCAGAAGCGACAAATGAGAACACAGATAAGCCTGAGCACAGATGAAGAGCTTCCAGAAAAAT
ACACCCAGCGTCGCAGGCCGTGGCTCAGCCAATTGTCAAATAAGAAGCAATCCAACACGGGCCGTGTGCA
GCCGTCAAAACGAAAGCCACTGCCTCCCCTCCCACCCTCTGAGGTTGCTGAAGAGAAGATCCAAGTCAAG
GCACTTTATGATTTTCTGCCCAGAGAACCCTGTAATTTAGCCTTAAGGAGAGCAGAAGAATACCTGATAC
TGGAGAAATACAATCCTCACTGGTGGAAGGCAAGAGACCGTTTGGGGAATGAAGGCTTAATCCCAAGCAA
CTATGTGACTGAAAACAAAATAACTAATTTAGAAATATATGAGTGGTACCATAGAAACATTACCAGAAAT
CAGGCAGAACATCTATTGAGACAAGAGTCTAAAGAAGGTGCATTTATTGTCAGAGATTCAAGACATTTAG
GATCCTACACAATTTCCGTATTTATGGGAGCTAGAAGAAGTACGGAGGCTGCCATAAAACATTATCAGAT
AAAAAAGAATGACTCAGGACAGTGGTATGTGGCTGAAAGACACGCCTTTCAATCAATCCCTGAGTTAATC
TGGTATCACCAGCACAATGCAGCCGGTCTCATGACTCGTCTCCGATATCCAGTTGGGCTGATGGGCAGTT
GTTTACCAGCCACAGCTGGGTTTAGCTACGAAAAGTGGGAGATAGATCCATCTGAGTTGGCTTTTATAAA
GGAGATTGGAAGCGGTCAGTTTGGAGTGGTCCATTTAGGTGAATGGCGGTCACATATCCAGGTAGCTATC
AAGGCCATCAATGAAGGCTCCATGTCTGAAGAGGATTTCATTGAAGAGGCCAAAGTGATGATGAAATTAT
CTCATTCAAAGCTAGTGCAACTTTATGGAGTCTGTATACAGCGGAAGCCCCTTTACATTGTGACAGAGTT
CATGGAAAATGGCTGCCTGCTTAACTATCTCAGGGAGAATAAAGGAAAGCTTAGGAAGGAAATGCTACTG
AGTGTATGCCAGGATATATGTGAAGGAATGGAATATCTGGAGAGGAATGGCTATATTCATAGGGATTTGG
CGGCAAGGAATTGTTTGGTCAGTTCAACATGCATAGTAAAAATTTCAGACTTTGGAATGACAAGGTACGT
TTTGGATGATGAGTATGTCAGTTCTTTTGGAGCCAAGTTCCCAATCAAGTGGTCCCCTCCTGAAGTTTTT
CTTTTCAATAAGTACAGCAGTAAATCTGATGTCTGGTCATTTGGAGTTTTAATGTGGGAAGTTTTTACAG
AAGGAAAAATGCCTTTTGAAAATAAGTCAAATTTGCAAGTCGTGGAAGCTATTTCTGAAGGCTTCAGGCT
ATATCGCCCTCACCTGGCACCAATGTCCATATATGAAGTCATGTACAGCTGCTGGCATGAGAAACCTGAA
GGCCGCCCTACATTTGCCGAGCTGCTGCGGGCTGTCACAGAGATTGCGGAAACCTGGTGACCGGAAACAG
AATGCCAACCCAAAGAGTCATCTTGCAAAACTGTCATTTATTGTGAATATCTTCACCATATGGGGTCACT
TATGGTGAATATCTTTCTTCAGAGTTGCTGACTCTTGAAAACAGTGCAAAGATCACAGTTTTTAAAAGTT
TTAAAAATTTAAGAATATTCACACAATCGTTTTTCTATGTGTGAGAGGGATTTGCACACTCTTATTTTTC
TGTAAAATATTTCACATCCCAAATGTGAAGAAGTGAAAAAGACTTCGCAGCAGTCTTCATTGTGGTGCTC
TTCATGATCATAGCCCCAGGAACCCTTGAGGTTCTTCTTCACAAGGCTGAGAGTGCTTCCTTCTTGAAGA
CGAGTGACATTCATCACTTCAGTGATCCATGCATAGAATATGAAAATAAATTCTTCCAACTCATGGGATA
AAGGGGACTCCCTTGAAGAATTTCATGTTTTTGGGCTGTATAGCTCTTTACAGAAAATGCACCTTTATAA
ATCACATGAATGTTAGTATTCTGGAAATGTCTTTTGTTAATATAATCTTCCCATGTTATTTAACAAATTG
TTTTTGCACATATCTGATTATATTGAAAGCAGTTTTTTGCATTCGAGTTTTAAACACTGTTATAAAATGT
AGCCAAAGCTCACCTTTGAACAGATCCCGGTGACATTCTATTTCCAGGAAAATCCGGAACCTGATTTTAG
TTCTGTGATTTTACACTTTTTACATGTGAGATTGGACAGTTTCAGAGGCCTTATTTTGTCATACTAAGTG
TCTCCTGTAATTTTCAGGAAGATGATTTGTTCTTTCCAGAAGAGGAGACAAAAGCAAGATAGCCAAATGT
GACATCAAGCTCCATTGTTTCGGAAATCCAGGATTTTGAATTCGAGATGAAACAACCAGCAATCACAGTT
AAATCTTAACTTTGCCTGCACTCTTTGTAGGAATGATCAGAAATTTATCTTTATCATTCTGAGTGCTTCA
GGAGTACAATAGGAAGAAAGATACTGGAGAAAGCACTAATGTAATCACCATGAAGTCTGACAACAGGAGC
CCATTATTTGCGTACTGTCCCACCCTGTATCATGGTTCTCTGGGAACAAGCTTTATGATTCTCATTAGAG
TTTATTTGTTGATTGTCAGTAGTTGCGACTTTTAAATTATATTTCCCCCACTCAAAGAATGGTATCTTTA
TATATCAATGACATTCAATAAATGTGTATTATTTCTAATGAGAA

What is claimed is:

1. A method of detecting expression levels of biomarkers in a human subject suspected of having IBS, comprising:
(a) providing a blood sample from the human subject;
(b) detecting expression levels of biomarkers in the blood sample by performing a RT-PCR assay on the blood sample to measure the expression level of the biomarkers consisting of: PGK1, POU6F1*, ACTR1A*, TBP, JUN, CD55 molecule, decay accelerating factor for complement (Cromer blood group) (CD55), IL11RA, TAF11 RNA polymerase II, TATA box binding protein (TBP)-associated factor (TAF11), TP53, v-Ha-ras Harvey rat sarcoma viral oncogene homolog (HRAS), origin recognition complex, subunit 1 (ORC1L), KRAS, APOBEC3F, ADAMTS-like 4 (ADAMTSL4), ASL, ANAPC1, GSTM4, ABR, LLGL2, colony stimulating factor 3 receptor (CSF3R), transforming growth factor, beta receptor II (TGFBR2), OAS1, tyrosine kinase (TXK), and NRAS.

2. The method of claim 1, and further comprising providing a series of biological sample obtained from the human subject; and determining a presence of any change in the expression levels of biomarkers in each of the biological samples from the series.

3. The method of claim 1, wherein the detecting is of the expression levels of genes consisting of SEQ ID NOs: 1, 3-7, 10, 14, 25, 27, 29-31, 33-37, 39, 42-44, 45 and 47.

4. A method of detecting expression levels of biomarkers in a human subject suspected of having IBS or IBD, comprising:
(a) providing a blood sample from the human subject;
(b) detecting expression levels of biomarkers in the blood sample by performing a RT-PCR assay on the blood sample to measure the level of the biomarkers consisting of: HRAS, GAPDH, TBP, major histocompatibility complex, class II, DR alpha (HLA-DRA), ORC1L, ABR, OAS1, JUN, cathepsin S, transcript variant 1 (CTSS), CD55, CDH1, PGK1, ACTR1A, TP53, SPIB, EXT2, APOBEC3F, TAF11, ADAMTSL4, cyclin-dependent kinase inhibitor 1B (p27, Kip1) (CDKN1B), phorbol-12-myristate-13-acetate-induced protein 1 (PMAIP1), IL11RA, SC65, CSF3R, ACTB, FOS, ASL, GATA binding protein 3 (GATA3), and guanine nucleotide binding protein (G protein), beta 5, transcript variant 1 (GNB5).

5. The method of claim 4, and further comprising providing a series of biological sample obtained from the human subject; and determining a presence of any change in the expression levels of biomarkers in each of the biological samples from the series.

6. The method of claim 4, wherein the detecting is of the expression levels of genes comprising SEQ ID NOs: 1-4, 6, 7, 10-12, 14, 15, 17, 18, 21-24, 26, 27, 29, 30, 35-38, 41-43 and 45.

7. A method of detecting expression levels of biomarkers in a human subject suspected of having Ulcerative Colitis (UC) or Crohn's Disease (CD), comprising:
(a) providing a blood sample from the human subject;
(b) detecting expression levels of biomarkers in the blood sample by performing a RT-PCR assay on the blood sample to measure the level of the biomarkers consisting of: POU6F1, ANAPC1, GAPDH, TBP, GNB5, ORC1L, TP53, CDH1, NRAS, EXT2, APOBEC3F, GATA3, ASL, LLGL2, JUN, ADAMTSL4, KRAS, CHEK2, IL11RA, PMAIP1, OAS1, and interferon, alpha-inducible protein 27 transcript variant 1 (IFI27).

8. The method of claim 7, and further comprising providing a series of biological sample obtained from the human subject; and determining a presence of any change in the expression levels of biomarkers in each of the biological samples from the series.

9. The method of claim 7, wherein the detecting is of the expression levels of genes comprising SEQ ID NOs: 4-7, 11, 13, 17, 21, 23, 34, 28, 29, 30, 31, 33-36, 38, 39, 43 and 45.


SEQUENCE LISTING (47 sequences)

SEQ ID NO: 1 - DNA, 5603 bp, Artificial Sequence
  cDNA
  misc_feature (699)..(723): Applied BioSystems context sequences, the region of that can be amplified in some embodiments of the presently-disclosed subject matter.
ggactgcagagggaacttgccttgaagaggcctggtccttaaagagacacagcacacacggcccgaccggcagccccagagcagaggctccactgatggcaggcgcccctggctaggctctgaggttcctttgccctcgccttgctgaatggtgagccgctgcctctcggagcccgtctccttgacagcctgccctcggctcctgcagccactcctgggcctgatggggacagggccagcctggtgggtggtgtcagaggtcctggcagagcagcgtaggcctgggatgcgtctgcagaattctggctgaacgagcgaggagcacggccagcttcggggccgtcgtgaccacaggagggcagagggccagcccgtgagctctgaccccagctggacgtgctcttgtttcccttggggctaaggagattggagccactgaactgaatctctgggttttggagacttagagaatccattggactcttctgctggcgtctttctgaatgctgatggggacttggtgacttcagctacgggacggacgagtacgacggagaggggaatgaggagcagaaggggcccccggagggctcagagaccatgccgtacatcgatgagtcgcccaccatgtccccgcagctcagcgcccgcagccagggcgggggggatggcgtctccccgactccacctgagggactggctcctggggtggaagcagggaaaggcctggagatgaggaagctggttctctcggggttcttggccagcgaagagatctacattaaccagctggaagccctgttgctgcccatgaaacccctgaaggccaccgccaccacctcccagcccgtgctcaccatccagcagatcgagaccatcttctacaagatccaggacatctatgagatccacaaggagttctatgacaacctgtgccccaaggtgcaacagtgggacagccaggtcaccatgggccacctcttccagaagctggccagccagctcggtgtgtacaaagcgtttgtcgataactataaagtcgctctggagacagctgagaagtgcagccagtccaacaaccagttccagaagatctcagaggaactcaaagtgaaaggtcccaaggactccaaggacagccacacgtctgtcaccatggaagctctgctctacaagcccattgaccgggtcactcggagcaccctagtcctacacgacctgctgaagcacacacctgtggaccaccccgactacccgctgctgcaggatgccctccgcatctcccagaacttcctgtccagcatcaacgaggacatcgacccccgccggactgcagtgacaacgcccaagggggagacgcgacagctggtgaaggacggcttcctggtggaagtgtcagagagctcccggaagctgcggcacgtcttcctctttacagatgtcctactgtgtgccaagctgaagaagacctctgcagggaagcaccagcagtatgactgtaagtggtacatccccctggccgacctggtgtttccatcccccgaggagtctgaggccagcccccaggtgcaccccttcccagaccatgagctggaggacatgaagatgaagatctctgccctcaagagtgaaatccagaaggagaaagccaacaaaggccagagccgggccatcgagcgcctgaagaagaagatgtttgagaatgagttcctgctgctgctcaactcccccacaatcccgttcaggatccacaatcggaatggaaagagttacctgttcctactgtcctcggactacgagaggtcagagtggagagaagcaattcagaaactacagaagaaggatctccaggcctttgtcctgagctcagtggagctccaggtgctcacaggatcctgtttcaagcttaggactgtacacaacattcctgtcaccagcaataaagacgacgatgagtctccaggactctatggcttccttcatgtcatcgtccactctgccaagggatttaagcaatcagccaacctgtactgtaccctggaggtggattccttcggctattttgtcagcaaagccaaaaccagggtgttccgggacacagcggagcccaagtgggatgaggagtttgagatcgagctggagggctcccagtccctgaggatcctgtgctatgagaagtgctatgacaagaccaaggtcaacaaggacaacaatgagatcgtggacaagatcatgggcaaaggacagatccagctggacccacaaaccgtggagaccaagaactggcacacggacgtgattgagatgaacgggatcaaagtggaattttccatgaaattcaccagccgagatatgagcctgaagaggaccccgtccaaaaagcagaccggcgtcttcggtgtgaagatcagcgtggtgacgaagcgggagcgctccaaggtgccctacatcgtccggcagtgtgtggaggaggtggagaagaggggtatcgaggaggttggcatctacaggatatcgggcgtggccacggacatccaggcgctcaaggccgtcttcgatgccaataacaaggacatcctgctgatgctgagtgacatggacatcaacgccatcgccgggacgctcaagctgtacttccgggaactgcccgagccgctcctcacggaccgactctacccagccttcatggagggcatcgccctgtcagaccctgctgccaaggaaaactgcatgatgcacctgctccgctccctgcccgaccccaacctcatcaccttcctcttcctgctggaacacttgaaaagggttgccgagaaggagcccatcaacaaaatgtcacttcacaacctggctaccgtgtttggacccacgttactgagaccctcagaagtggagagcaaagcacacctcacctcggctgcggacatctggtcccatgacgtcatggcgcaggtccaggtcctcctctactacctgcagcacccccccatttccttcgcagaactcaagcggaacacactgtacttctccaccgacgtgtagcccgaggcagggtggctgcgggcgggtggtggaaccagcccctccagcctggggtccaactcagacttgaaagactgcaatagaaaactcccaaacccagcactccagactcgagggaagccagcttccaagaactggaatgcgtacgtcttttgtgccaccttgtacaaagccggctgcccagccccagcctcaccaccgcatcccacctcctgccctccatacctctagttgtgtctgatgctccgtgctgttcgggaattgttttatgtacacttgtcaggcagaaaaggtagtgaccggcccggcgtgggcacacagacagcccgctttgttctttcatttcctccagcactttctttccgcctgagtccagcccaaggccttttattttgcgctgtgtaactgctgccagcttctctcttggccctgctcccagatggcggtctcctggcagcctcccctcagtcttcctccacccgctcttccttcccagcctgcctgcatgcatgtgcacccttggtcttcgctccatcgccttgaaagctctgaagaggccctgggttgccgcggcagcagtggtctgtttgatgctgccgtttgccgctgccggcccctcctcagactccgcctttgggagcacacctgctttgccttgctgcctgtgcaaatgttggacaagcagacacactcacactcgtccccagcttagcacagagctggagcgcccatttctggaattttccgtttgggaatctccacttctggggtttacctgttcggcctcctgtctatcagtgaggcatctctgactgtttcttctactgcttttcagttcccttccctgctgttctatttcctttgagtgtaaagactcacaggtgacctgctatcgagatagccagagggtcaggagagaatgggggaggaggcggtcaggctgctgaggaaacaccacaggctgaacgggggaggaatgcacatgccacgctgggtgtcccgggtcgcggggaggcagctcagctcttaggagcaagttgtgggggcttttcaagaggggccaggcttcctggagggtgactgatgtggccgaagcaggtgtccaggcaggtaggctgcagccaggagctccctggcaccgcaggacctcgtggtactcttgccttagattttacacacactccacagccaagcactgccacggtcctccaggacctgggaagcaaaggcacaggcccacggtggccagccattgtggtgccgccccagcttctggatacagccttttgggtaaacactgggaactccagaagttgtggggagagtggggaatcagacagccgcctctaggggctgggttctgctggggcctccttgttggtgctgtaggcacccgccagggagcagggacccgacttgcagacgcattgcccggtactaggaaggagtgaggtgtgttcccaccgtacacttcccacacgagctgcggctgccagcctcgggccatcagcctaggagagcagatgcagctccaggggctcgacttatagccagttacagctccccggctcttctgtgtggcagagcgtcgtttccgggccctcagggctggggagctcagttcccattgcttgtgctcagggctgagtcttaaagaagggtttgccggccctaacgctgcagcgcgtgcgcggtgagaggccctttttgagcctgtttactcctgtggccttgggcagaacagtaaatactctgtgcacggaggaaagacatgcccaagaggaaggaagtactgaccatcggctgcctgtgagcagcttagcaaggagcccttgctccctgggaaaggcggtgaacttgagtctaaagatgcagtgcctggcccttcctaaggtccctgcctggcatccgagtgtcggtgtgtggcacagaaggctcctgcttgcttccaaagtgatggacaggaaggggcagagtgagtcacggcccagactgggcaccttcgcgtctcagcctcagggagccccacagccccaagctcgctgaggcaacgtgagaacaggctatgggaaggctgcaaaggctgagaaatgcaaaggctcatatttataaatcccacccccagagtggggagggtcaggtgccagacctggactaaactgcaccaaggaaacacccagcagggtctcctgtgagccggggaccatgcagcccgaaacctccagtcactgcgcccggcaggagtcaggagccagggactgtgcagcctggaacctccagtcactgtgcccagcagggtgggctgtgcccagcaggagtcaggctaagaaacgccaggtctgcctgttcttgctgggcaatggctgatggctgccagtttctgctgatacacaggtaggatgggacccttcatgaatatctgactttaataagttggtaaggatatatttttttgtctatgttctgtttcaacttatgtagattattataaattgatgtaaaccacgtgagaggaaaatgttaataaaaaatgcaaagccccatcatttgcacaaaactca

SEQ ID NO: 2 - DNA, 1852 bp, Artificial Sequence
  cDNA
  misc_feature (50)..(65): Applied BioSystems context sequences, the region of that can be amplified in some embodiments of the presently-disclosed subject matter.
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 3 - DNA, 2891 bp, Artificial Sequence
  cDNA
  misc_feature (752)..(776): Applied BioSystems context sequences, the region of that can be amplified in some embodiments of the presently-disclosed subject matter.
gctccctcgccgccctgaaccggcggctagactgcgcatgcgtgtcagtggcgctagcggcggacccggctgggcagttccttccccagaaggagagattcctctgccatggagtcctacgatgtgatcgccaaccagcctgtcgtgatcgacaacggatccggtgtgattaaagctggttttgctggtgatcagatccccaaatactgctttccaaactatgtgggccgacccaagcacgttcgtgtcatggcaggagcccttgaaggcgacatcttcattggccccaaagctgaggagcaccgagggctgctttcaatccgctatcccatggagcatggcatcgtcaaggattggaacgacatggaacgcatttggcaatatgtctattctaaggaccagctgcagactttctcagaggagcatcctgtgctcctgactgaggcgcctttaaacccacgaaaaaaccgggaacgagctgccgaagttttcttcgagaccttcaatgtgcccgctcttttcatctccatgcaagctgtactcagcctttacgctacaggcaggaccacaggggtggtgctggattctggggatggagtcacccatgctgtgcccatctatgagggctttgccatgccccactccatcatgcgcatcgacatcgcgggccgggacgtctctcgcttcctgcgcctctacctgcgtaaggagggctacgacttccactcatcctctgagtttgagattgtcaaggccataaaagaaagagcctgttacctatccataaacccccaaaaggatgagacgctagagacagagaaagctcagtactacctgcctgatggcagcaccattgagattggtccttcccgattccgggcccctgagttgctcttcaggccagatttgattggagaggagagtgaaggcatccacgaggtcctggtgttcgccattcagaagtcagacatggacctgcggcgcacgcttttctctaacattgtcctctcaggaggctctaccctgttcaaaggttttggtgacaggctcctgagtgaagtgaagaaactagctccaaaagatgtgaagatcaggatatctgcacctcaggagagactgtattccacgtggattgggggctccatccttgcctccctggacacctttaagaagatgtgggtctccaaaaaggaatatgaggaagacggtgcccgatccatccacagaaaaaccttctaatgtcgggacatcatcttcacctctctctgaagttaactccactttaaaactcgctttcttgagtcggagtgtttgcgaggaactgcctgtgtgtgagtgcgtgtgtggatatgagtgtgtgtgcacatgcgagtgccgtgtggccctgggaccctgggcccagaaaggacgatgaactacctgcagtggtgatggcctgaggcctggggttgaccactaactggctcctgacagggaagagcgctggcagaggctgtgctccctcctcaggtggcctctggctggctgtgggggactccgtttactaccacagggagacagagggaggtaagccatcccccgggagaccttgctgctgaccatcctaggctgggctggccccaccctcacccccacccccagggtgccctgaggccccaggcagctgctgcctccactatcgatgcctcctgactgcacactgaggactgggactggggttgagttctgtctggttttgttgccattttggtttgggaggctggaaaagcaccccaagagctattacagagactggagtcaggagagagcaggaggccctcatgttcaccagggaacaggaccacaccggccactggaggagggcaggagcagtcctcactctgaatggctgcagagttaatgttcccagcccagtcccctttcgggggccttgggagagtttaaggcacctgctggttccaggacctcgctttccatctgttcttgttgcaatgccatcttcaaaccgttttatttattgaagtgtttgttcagttaggggctggagagagggagcttgctgcctcctgccttgctacactaatgtttacagcacctaagcttagcctccagggccccacctctcccagctgatggtgagctgacagtgtccacaggttccaggaccatttgagattggaagctacactcaaagacactcccaccaggctctttctcccttttcctcttgctcactgccctggaatcaacaggctggttgctggttagattttctgaaacaggaggtaaaatttttctttggcagaggcccctaagcaagggaggggtgttggagagccagtgcccttaagactggagaaagctgcaatttaccaagttgccttttgccactgtagctgaccaggggactaggttgtagaggtgggaaggccccctctgggctgatcttgtgccattcttgaccttggacctgcttggttaaggagggagtgggccagaccagagtgccaggagctaatggagccaggcctgactcctaggagtggtccaaaggccttcagcctagatggtgcaaagctggggccagcctgtcttcaccggcaccctcacctgtgacaccaagacccaccccaatcccagacttcacacagtattctcccccacgccgtcctatgaccaaaggcccctgccaggtgtgggccacagcagcaggtatgtgtgaaagcaacgtagcgccccgcggactgcagtgcgcttaaccaactcacctcccttctcttagcccaagcctgtccctcgcacagcctcgcacaaaccacattgcctggtggggcccagtgtactgaaataaagtcgttccgatagacacgtcaaaaaaaaaaaaaaaaaaa

SEQ ID NO: 4 - DNA, 4209 bp, Artificial Sequence
  cDNA
  misc_feature (3179)..(3193): Applied BioSystems context sequences, the region of that can be amplified in some embodiments of the presently-disclosed subject matter.
ccgccgcggagcgaggttgcctggagagagcgcctgggcgcagaagggttaacgggccaccgggggctcgcagagcaggagggtgctctcggacggtgtgtcccccactgcactcctgaacttggaggacagggtcgccgcgagggacgcagagagcaccctccacgcccagatgcctgcgtagtttttgtgaccagtccgctcctgcctccccctggggcagtagagggggagcgatggagaactggactggcaggccctggctgtatctgctgctgcttctgtccctccctcagctctgcttggatcaggaggtgttgtccggacactctcttcagacacctacagaggagggccagggccccgaaggtgtctggggaccttgggtccagtgggcctcttgctcccagccctgcggggtgggggtgcagcgcaggagccggacatgtcagctccctacagtgcagctccacccgagtctgcccctccctccccggcccccaagacatccagaagccctcctcccccggggccagggtcccagaccccagacttctccagaaaccctccccttgtacaggacacagtctcggggaaggggtggcccacttcgaggtcccgcttcccacctagggagagaggagacccaggagattcgagcggccaggaggtcccggcttcgagaccccatcaagccaggaatgttcggttatgggagagtgccctttgcattgccactgcaccggaaccgcaggcaccctcggagcccacccagatctgagctgtccctgatctcttctagaggggaagaggctattccgtcccctactccaagagcagagccattctccgcaaacggcagcccccaaactgagctccctcccacagaactgtctgtccacaccccatccccccaagcagaacctctaagccctgaaactgctcagacagaggtggcccccagaaccaggcctgcccccctacggcatcaccccagagcccaggcctctggcacagagcccccctcacccacgcactccttaggagaaggtggcttcttccgtgcatcccctcagccacgaaggccaagttcccagggttgggccagtccccaggtagcagggagacgccctgatccttttccttcggtccctcggggccgaggccagcagggccaagggccttggggaacgggggggactcctcacgggccccgcctggagcctgaccctcagcacccgggcgcctggctgcccctgctgagcaacggcccccatgccagctccctctggagcctctttgctcccagtagccctattccaagatgttctggggagagtgaacagctaagagcctgcagccaagcgccctgcccccctgagcagccagacccccgggccctgcagtgcgcagcctttaactcccaggaattcatgggccagctgtatcagtgggagcccttcactgaagtccagggctcccagcgctgtgaactgaactgccggccccgtggcttccgcttctatgtccgtcacactgaaaaggtccaggatgggaccctgtgtcagcctggagcccctgacatctgtgtggctggacgctgtctgagccccggctgtgatgggatccttggctctggcaggcgtcctgatggctgtggagtctgtgggggtgatgattctacctgtcgccttgtttcggggaacctcactgaccgagggggccccctgggctatcagaagatcttgtggattccagcgggagccttgcggctccagattgcccagctccggcctagctccaactacctggcacttcgtggccctgggggccggtccatcatcaatgggaactgggctgtggatccccctgggtcctacagggccggcgggaccgtctttcgatataaccgtcctcccagggaggagggcaaaggggagagtctgtcggctgaaggccccaccacccagcctgtggatgtctatatgatctttcaggaggaaaacccaggcgttttttatcagtatgtcatctcttcacctcctccaatccttgagaaccccaccccagagccccctgtcccccagcttcagccggagattctgagggtggagcccccacttgctccggcaccccgcccagcccggaccccaggcaccctccagcgtcaggtgcggatcccccagatgcccgccccgccccatcccaggacacccctggggtctccagctgcgtactggaaacgagtgggacactctgcatgctcagcgtcctgcgggaaaggtgtctggcgccccattttcctctgcatctcccgtgagtcgggagaggaactggatgaacgcagctgtgccgcgggtgccaggcccccagcctcccctgaaccctgccacggcaccccatgccccccatactgggaggctggcgagtggacatcctgcagccgctcctgtggccccggcacccagcaccgccagctgcagtgccggcaggaatttggggggggtggctcctcggtgcccccggagcgctgtggacatctcccccggcccaacatcacccagtcttgccagctgcgcctctgtggccattgggaagttggctctccttggagccagtgctccgtgcggtgcggccggggccagagaagccggcaggttcgctgtgttgggaacaatggtgatgaagtgagcgagcaggagtgtgcgtcaggccccccgcagccccccagcagagaggcctgtgacatggggccctgtactactgcctggttccacagcgactggagctccaagtgctcagccgagtgtgggacgggaatccagcggcgctctgtggtctgccttgggagtggggcagccctcgggccaggccagggggaagcaggagcaggaactgggcagagctgtccaacaggaagccggccccctgacatgcgcgcctgcagcctggggccctgtgagagaacttggcgctggtacacagggccctggggtgagtgctcctccgaatgtggctctggcacacagcgtagagacatcatctgtgtatccaaactggggacggagttcaacgtgacttctccgagcaactgttctcacctccccaggccccctgccctgcagccctgtcaagggcaggcctgccaggaccgatggttttccacgccctggagcccatgttctcgctcctgccaagggggaacgcagacacgggaggtccagtgcctgagcaccaaccagaccctcagcacccgatgccctcctcaactgcggccctccaggaagcgcccctgtaacagccaaccctgcagccagcgccctgatgatcaatgcaaggacagctctccacattgccccctggtggtacaggcccggctctgcgtctacccctactacacagccacctgttgccgctcttgcgcacatgtcctggagcggtctccccaggatccctcctgaaaggggtccggggcaccttcacggttttctgtgccaccatcggtcacccattgatcggcccactctgaaccccctggctctccagcctgtcccagtctcagcagggatgtcctccaggtgacagagggtggcaaggtgactgacacaaagtgactttcagggctgtggtcaggcccatgtggtggtgtgatgggtgtgtgcacatatgcctcaggtgtgcttttgggactgcatggatatgtgtgtgctcaaacgtgtatcacttttcaaaaagaggttacacagactgagaaggacaagacctgtttccttgagactttcctaggtggaaaggaaagcaagtctgcagttccttgctaatctgagctacttagagtgtggtctccccaccaactccagttttgtgccctaagcctcatttctcatgttcagacctcacatcttctaagccgccctgtgtctctgaccccttctcatttgcctagtatctctgcccctgcctccctaattagctagggctggggtcagccactgccaatcctgccttactcaggaaggcaggaggaaagagactgcctctccagagcaaggcccagctgggcagagggtgaaaaagagaaatgtgagcatccgctcccccaccaccccgcccagcccctagccccactccctgcctcctgaaatggttcccacccagaactaatttattttttattaaagatggtcatgacaaatgagaaaaaaaaaa

SEQ ID NO: 5 - DNA, 6329 bp, Artificial Sequence
  cDNA
  misc_feature (5039)..(5064): Applied BioSystems context sequences, the region of that can be amplified in some embodiments of the presently-disclosed subject matter.
cgcgtccatttgaacgtctcgcacgccttcctgccattagcactcgagcccgctgctgttgcccgttcttcctccagaataggggagggagagggaatgagaagctgctgcggcccaagagtcactgtgaaggaccccgccgctgccctcgggcctcctcggcccctgcgcctccggggagcagccggggctcgccgcgcctgacgcgtcccgagttatacagaaataatgttgatatttggaacccatgtcgaacttctatgaagaaaggacaacgatgattgcagcaagggatttgcaggaatttgttccttttggtcgagaccactgcaagcaccaccctaatgctttgaaccttcaacttcgccagctgcagccagcttctgaattatggtcttctgatggtgctgctggcttggtgggatcccttcaggaggttacaatccacgagaaacagaaggaaagctggcagttaaggaaaggagtaagtgaaattggagaagatgtggactatgatgaggaactctatgttgctggaaatatggtgatatggagcaaaggaagtaaaagccaggcattggcagtttataaagcatttacagttgacagtcctgttcagcaggcattgtggtgtgacttcattatatcacaggataagtctgaaaaggcctacagtagcaatgaagtagaaaaatgcatatgtatattgcaaagctcatgtattaacatgcatagcatagaaggaaaggattacatagcttcattaccatttcaggttgcaaatgtttggcccactaaatatggattgctgtttgaacgaagcgcttcttcacatgaagtacctccaggttcacccagagaacctttacctactatgttcagcatgctgcacccactagatgaaataactccacttgtttgtaaatctggaagtctttttggttcatcacgggtgcaatatgttgtagatcatgcaatgaaaattgttttcctcaatactgacccctctattgtaatgacttatgatgctgttcaaaatgtgcattctgtgtggactctccggagagtcaaatcagaggaagagaatgttgttttaaagttctctgaacaggggggaaccccacagaatgtggccactagcagctccctcacagcacatctcagaagcctctccaaaggagattcccctgtgacttcacctttccagaattactcctccattcacagccagagtcgctcaacctcatcacccagtctacattctcgctcaccttctatttccaacatggcagctctaagtcgtgctcattctcctgcgttaggagtgcactctttttcaggggtgcaaaggttcaacatttcaagccataatcagtctccaaagagacatagtatttctcattctccaaatagtaattctaatggctcctttcttgcaccagaaacggagccaattgttcctgaactgtgtattgaccatttgtggacagaaacgattactaatataagagagaaaaattcacaagcctcaaaagtgtttattacatctgacctatgtgggcaaaagttcctgtgctttttagtagagtcccagctccagttacgctgtgtaaagtttcaagagagtaatgataaaacccagctcatctttggttcagtgaccaacataccagcaaaggatgcagcaccagtggagaaaatagacaccatgctggtcttggaaggcagtggaaacctggtgctatacacaggagtggttcgggtgggaaaggtttttattcctggactgccagctccctctctgacgatgtccaacacaatgcctcggcccagtactccactagatggcgttagtactccaaagcctcttagtaaactccttggatcattggacgaggttgttctgttgtccccagttccagaactgagggattcttcaaaacttcatgattctctctataatgaggattgtactttccaacagcttggaacttacattcattctatcagagatcctgtccataacagagtcaccctggaactgagtaatggctccatggttaggatcactattcctgaaattgccacctctgagttagtacaaacgtgtttgcaagcaattaagtttatcctgccaaaagaaatagcagttcagatgcttgtcaagtggtacaatgtccacagtgctccaggaggacccagttatcactcagagtggaatttatttgtgacttgtctcatgaacatgatgggttataacacagaccgcttagcatggactagaaattttgactttgaaggatcactttctcctgtcattgcgcccaaaaaagcaaggccttccgagactggatctgatgatgactgggaatatttactaaattcagactaccaccagaatgttgagtctcatcttttgaacagatctttatgtctgagtccttcagaagcttcacagatgaaggatgaggatttttcacagaatctcagtctggattcttctacacttctctttactcacatacctgcaatttttttcgttcttcaccttgtgtatgaggagcttaagttgaatactctaatgggagaaggaatttgttcacttgttgaacttctcgttcagttggcaagggacttaaaattggggccttatgtagatcattactatagagactacccaacgcttgtcagaactactggacaagtgtgcacaattgatccaggtcaaacaggatttatgcatcatccatcattttttacgtctgagccaccaagtatttatcagtgggtgagttcttgtctgaagggtgaaggaatgccaccttatccttacctccctggaatctgtgaaagaagcagacttgtagtcttgagtattgcactgtacatacttggtgatgagagcttggtttctgatgaatcctcacagtatttaaccagaataactatagccccccagaagttgcaagtagaacaagaggaaaacaggtttagtttcaggcattctacatctgtttctagtctagctgaaagattggttgtctggatgactaatgtaggattcactttaagagatttggaaactcttccctttggaattgctcttcccatcagagatgcaatttatcactgtcgtgagcagcctgcctcagactggccagaagctgtctgtctcttgattggacgtcaggatctttccaagcaggcctgcgaaggaaacttacccaaagggaagtctgtgctctcatcagatgttccttcaggaacagaaactgaggaggaagatgacggcatgaatgacatgaatcacgaggtcatgtcattaatatggagtgaagatttaagggtgcaggatgtgcgaaggcttcttcagagtgcgcatcctgtccgtgtcaacgtagtgcagtacccagagctcagtgaccacgagttcatcgaggaaaaggaaaacagattgctccaattgtgtcagcgaactatggctcttcctgtaggacgaggaatgtttaccttgttttcgtaccatcctgttccaacagagccattgcctattcctaaattgaatctgactgggcgtgcccctcctcggaacacaacagtagaccttaatagtggaaacatcgatgtgcctcccaacatgacaagctgggccagctttcataatggtgtggctgctggcctgaagatagctcctgcctcccagatcgactcagcttggattgtttacaataagcccaagcatgctgagttggccaatgagtatgctggctttctcatggctctgggtttgaatgggcaccttaccaagctggcgactctcaatatccatgactacttgaccaagggccatgaaatgacaagcattggactgctacttggtgtttctgctgcaaaactaggcaccatggatatgtctattactcggcttcttagcattcacattcctgctctcttacccccaacgtccacagagctggatgttcctcacaatgtccaagtggctgcagtggttggcattggccttgtatatcaagggacagctcacagacatactgcagaagtcctgttggctgagataggacggcctcctggtcctgaaatggaatactgcactgacagagagtcatactccttagctgctggcttggccctgggcatggtctgcttggggcatggcagcaatttgataggtatgtctgatctcaatgtgcctgagcagctctatcagtacatggttggaggacataggcgctttcaaacaggaatgcatagggagaaacataaatcaccaagttatcaaatcaaagaaggagataccataaatgtggatgtgacttgtccaggtgctactctagctttggctatgatctacttaaaaaccaataacagatctattgcagattggctccgagcccctgacaccatgtatttgttggactttgtgaagccagaatttctcttgcttaggacacttgctcgatgcctgattttgtgggatgatattttaccaaattccaagtgggttgacagcaatgttcctcaaattataagagaaaatagtatctctctcagtgaaatcgaattgccgtgctcagaggatttgaatttggaaactttgtcccaagcacatgtctacataattgcaggagcctgcttgtctctgggttttcgatttgctggctcagaaaacttatcagcatttaactgtttgcataaatttgccaaagattttatgacttatttgtccgcacctaatgcttctgttacaggtcctcataacctagaaacttgtctgagcgtggtgctgctgtctctcgccatggtcatggctggctcaggaaacctaaaggttttgcagctttgtcgcttcttacacatgaaaacgggtggtgaaatgaactatggttttcacttagcccaccacatggcccttggacttctatttttgggaggaggaaggtactctttgagcacatcaaattcttccattgccgctcttctctgtgccctttatccgcacttcccagctcacagcactgacaaccggtatcatctccaggctctccggcacctctatgtgctggccgcggagcccaggcttctagtgcctgtggatgtggacacaaacacgccctgctatgccctcttagaagttacctacaagggcactcagtggtatgaacaaaccaaagaagaattgatggctcctacccttcttccagaactccatcttttaaagcagattaaagtaaaaggcccaagatactgggaactgctcatagatttaagcaaaggaacacaacacttgaagtccatcctttccaaggatggggttttatatgttaaactccgggcgggtcagctctcctacaaagaagatccaatgggatggcaaagtttgttggctcagactgttgctaacaggaactctgaagcccgggctttcaagccagaaacaatctcagcattcacttctgatccagcacttctgtcatttgctgaatatttctgcaagccaactgtgaacatgggtcagaaacaggaaattctggatctcttttcttcagtactctatgaatgtgttacccaggagaccccagagatgttgcctgcatacatagcaatggatcaggctataagaagacttgggagaagagaaatgtctgagacttctgaactttggcagataaagttggtgttagagtttttcagctcccgaagccatcaggagcggctgcagaaccaccctaagcgggggctctttatgaactcggaattcctccctgttgtgaagtgcaccattgataataccctggaccagtggctacaagtcgggggtgatatgtgtgtgcacgcctacctcagcgggcagcccttggaggaatcacagctgagcatgctggcctgcttcctcgtctaccactctgtgccagctccacagcacctgccacctataggactagaagggagcacaagctttgctgaactgctcttcaaatttaagcagctaaaaatgccagtgcgagctttgctgagattggctcctttgcttcttggaaatccacagccaatggtgatgtgaccgtgtctggcggtgaacctaccctgaaacgtgacttctgcacaacaaacgtgaccaaacatcaaagctaaagcaatgtttataaagttttatggtataactagggggaaatgagctgcacaaacctcaatgtattttaaatctgttgctgtcatcattaacggtatatgacatataaaagcaagttaaaatttacttttgtaaataaagtttttggtttgtttccaaaaaaaaaaaaaaaaaaaaa

SEQ ID NO: 6 - DNA, 4706 bp, Artificial Sequence
  cDNA
  misc_feature (849)..(873): Applied BioSystems context sequences, the region of that can be amplified in some embodiments of the presently-disclosed subject matter.
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgtattcccagcctgagcaccacgcagaaatgtgcttcctctcttggttctgtggcaaccagctgcctgcttacaagtgtttccagatcacctggtttgtatcctggaccccctgcccggactgtgtggcgaagctggccgaattcctggctgagcaccccaatgtcaccctgaccatctccgccgcccgcctctactactactgggaaagagattaccgaagggcgctctgcaggctgagtcaggcaggggcccgcgtgaagattatggacgatgaagaatttgcatactgctgggaaaactttgtgtacagtgaaggtcagccattcatgccttggtacaaattcgatgacaattatgcattcctgcaccgcacgctaaaggagattctcagaaacccgatggaggcaatgtatccacacatattctacttccactttaaaaacctacgcaaagcctatggtcggaacgaaagctggctgtgcttcaccatggaagttgtaaagcaccactcacctgtctcctggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctactacttctgggatacagattaccaggaggggctccgcagcctgagtcaggaaggggcctccgtggagatcatgggctacaaagattttaaatattgttgggaaaactttgtgtacaatgatgatgagccattcaagccttggaaaggactaaaatacaactttctattcctggacagcaagctgcaggagattctcgagtgaggggtctccccgggcctcatggtctgtctcctctagcctcctgctcatgttgtgcaggcctcccctccatcctggaccagctgtgcttttgcctggtcatcctgagcccctcctggcctcagggccattccatagtgctcccctgcctcaccacctcctctccgctctcccaggctcttcctgcagaggcctctttctgcctccatggctatccatccacccaccaagaccctgttccctgagcctgcatgcccctaacctgccttttcccatctccccagcataacctaatattttttttttttttttgagacggaatttcgctctgtcacccagactggagtgcaatggcttgatcttggctcactgcaaactctgcctaccaggttcaagcgattctcctgcctccgcctcccgagtagctggaattacagacgcctgccaccacgcacagctaactttttttttttttgtatttttagtagtgactgggtttcaccatgttggccaggctggtcttgaactcctgacctcaggtgatccgcctatctcagcctcccaaagtgctgggattacaggcgtgagccactggcccggcggcacaaccaaatcttattaaactcaccctaggctggccgcggtgactcatgcctataatcccccagcaatttgggaggcagaggtgagagaatcgcttgagcccaggaattcgagaccagcctgggccacatgacaaagccccatctctacaaaaaaattacaaaaaaaaaaaaaacaggtgtggtggcatgcacctgtagttgaagctacttggaaggatgaagtgggaggattgcttgagccggggaggtggaggctgcagtgaactgagatcacgtcactgaactccagtctgagcaacagatcgagaccctgcctgaaaataaatcaataaataaactcaaccgaaatgggtatgaaagttgaaatgggtatgtaagttgaaaaccagaagttttgagaaacatcctttgttaactttcatcctacaaattgggtcattcatgtcctacgcagctaaaacagagcccaggagccagggaggaaaagcagtcaggccacacaccattgctcccaaaatggacttctctgcaagcctgactcctgaaactgtgcattgtaccctgaaaccagctttatccatagcttctgcaataaatggctgtaagtcttggactccttgctataatcgcagctattcagcaatggaacctcccagttcccaacccttcctagtgcccatgggctttcccataggacaagagaacatttctccttttctttttttttttctttgaaatggagtctcgccctgtcacccaggctggagtgcaatggtgcggtctcggctcactgcaacctctgcctcccttgttcaagtgattctcctgtctcagcctcccgagtagctgggattacaggcgtccaccaccaaaccaggctaatttttgtatttttcataaaaacgggtttcatcatgtttcccaggctggtcttatttttattttattttttgagatggagtcttgctctgttgcccaggctggggtgcagtggtgcaatctgggttcactgcagcctctgccgcctgagttcaagctattttcctacctaagcctcccaagtagctgggattacatgcgcgtgccaccacgcctagctaatttttgtgtttttagtagagacggggtttcaacatcttgaccaggctggtcttgaactcctgacctcgtgatccacccgtctcggcctcccaaagtgctgggattacaggcgtgagccacctggccaggcttaggctggtcttaaactcctgacctcaagtgatccaacctccttggcctcccaaattgctgggattgctggtgtgagccacagcgcctagcccatttctccttttaataggacctgttgctgtctctgttctcccaacatggtgaacaccacccggactgcgtgtatgtcccaaattacaattctttctttgcaaatgaaatgtgaaatttagaggcccttctccacactttaaatttgacttgacattttctaggcagatataagttattagagaatgagattctctataaaaatgatcccttcatgctgtggcctccacagaagatgccctgggccaggtgcccacatgaataatgcgggccacaggcaggcatttattttctcacagatatggaggctacaagtccaaggtggaggggtcggcggggttgtttgctctgaggccgctcctcctggatggcagggatcccttctggctgtgtcctctgtggcctttcctctatgaacctgtactgtacctctggggtctctctgcttccaaatatcttttttttttttttcagacagttttgctcttgttttctaggctggagtgcaatggcacaatctcagctcactgcaacctctgccttccgagttcaagcgattctcgtgcctcagcctcctgagtagctgggactacaggcgtgtgccaccacgcctggctaattttgtagttttagtagagacggggtttctccatgttgctcaggctggtcttgaactcatgagctcaggcgatccactctcctcagcctcccaaagtgctgggattacagatataagccaccatacacaacttttttttttttttgagatggagtttcactctgttgcccaggctggagtgctaaatagcagaatcactgctcactgcaacctctgcctgctgggttcaagcaattctcccacctcagcctcctgagtagctgggattacagatgcccagaaccaatctctgctaatttttctattttttagtagagatggggtttcactgaggaaggagaccacctctctcattgtctcctatttcagaaggaagcaaaaagttagaaagatgcagaagtaagatcaatggccagactgtttggcgctgctacctgggcctggtagttaaagatcaactcctgacctgaccgcttgttttatctaaagattccagacattgtatgaggaagcattgtgaaactttctggtctgttctgctagcccccaccactgatgcatgtagccccccagtcacgtagcccacgcttgcacaatctatcacgaccctttcacgtggaccccttagaattgtaagcccttaaaagggccagggacttcttcagggagctccaatcttcagatgcaagtctgtcaacgctcccagctgattaaagcctcttccttcctaaaaaaaaaaaaaaaaaa

SEQ ID NO: 7 - DNA, 2061 bp, Artificial Sequence
  cDNA
  misc_feature (673)..(699): Applied BioSystems context sequences, the region of that can be amplified in some embodiments of the presently-disclosed subject matter.
ccaggcggaggtgagtgcgcggcggccggatgggcgggacgggcgtggaggacgccgagcaccgtggcgcgcgctcacgtccgcgtccccaagggctgcgctccctcaagcgcagtgcccagaactcggagccagcccggcccgggggaccctgctggccaaggaggtcgtcagtccggtcttgtcttccagacccggaggaccgaagcttccggacgacgaggaaccgcccaacatggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa

SEQ ID NO: 8 - DNA, 987 bp, Artificial Sequence
  cDNA
  misc_feature (397)..(421): Applied BioSystems context sequences, the region of that can be amplified in some embodiments of the presently-disclosed subject matter.
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 9 - DNA, 7128 bp, Artificial Sequence
  cDNA
  misc_feature (5108)..(5132): Applied BioSystems context sequences, the region of that can be amplified in some embodiments of the presently-disclosed subject matter.
agataactgggcccctgcgctcaggaggccttcaccctctgctctgggtaaaggtagtagagtcccgggaaagggacagggggcccaagtgatgctctggggtactggcgtgggagagtggatttccgaagctgacagatggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 10 - DNA, 2812 bp, Artificial Sequence
  cDNA
  misc_feature (1369)..(1393): Applied BioSystems context sequences, the region of that can be amplified in some embodiments of the presently-disclosed subject matter.
agcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggtatcatgtagtatgtgaaatttattcttaaacgtgactactttatttctaaataagaaattccctacctgcttcctacaagcagttcagaatgccatgccttggttgtcctagtgtgaataattttcagctactttaaaattatattgtactttctcaagcatgtcatatcctttcctattagagtatctatattacttgttactgatttacctgaaggcaatctgattaatttctaggtttttaccatattcttgtcatcttgccaattacattttaagtgttagactagactaagatgtactagttgtatagaatataactagatttattatggcaatgtttattttgtcattttgcttcatctgttttgttgttgaagtactttaaatttcatacgttcatggcatttcactgtaaagactttaatgtgtatttcttaaaataaaactttttttcctccttaaaaaaaaaaaaaaaaaa

SEQ ID NO: 11 - DNA, 4815 bp, Artificial Sequence
  cDNA
  misc_feature (499)..(523): Applied BioSystems context sequences, the region of that can be amplified in some embodiments of the presently-disclosed subject matter.
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 12 - DNA, 2413 bp, Artificial Sequence
  cDNA
  misc_feature (938)..(962): Applied BioSystems context sequences, the region of that can be amplified in some embodiments of the presently-disclosed subject matter.
cttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggctgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaaacagctcgaattaagaatatgtttccttgtttatcagatacatcactgcttgatgaagcaaggaagatatacatgaaaattttaaaaatacatatcgctgacttcatggaatggacatcctgtataagcactgaaaaacaacaacacaataacactaaaattttaggcactcttaaatgatctgcctctaaaagcgttggatgtagcattatgcaattaggtttttccttatttgcttcattgtactacctgtgtatatagtttttaccttttatgtagcacataaactttggggaagggagggcagggtggggctgaggaactgacgtggagcggggtatgaagagcttgctttgatttacagcaagtagataaatatttgacttgcatgaagagaagcaattttggggaagggtttgaattgttttctttaaagatgtaatgtccctttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaataattctaaatccctcgatatttttaaagatctgtaagtaacttcacattaaaaaatgaaatattttttaatttaaagcttactctgtccatttatccacaggaaagtgttatttttcaaggaaggttcatgtagagaaaagcacacttgtaggataagtgaaatggatactacatctttaaacagtatttcattgcctgtgtatggaaaaaccatttgaagtgtacctgtgtacataactctgtaaaaacactgaaaaattatactaacttatttatgttaaaagattttttttaatctagacaatatacaagccaaagtggcatgttttgtgcatttgtaaatgctgtgttgggtagaataggttttcccctcttttgttaaataatatggctatgcttaaaaggttgcatactgagccaagtataattttttgtaatgtgtgaaaaagatgccaattattgttacacattaagtaatcaataaagaaaacttccatagctatt

SEQ ID NO: 13 - DNA, 1991 bp, Artificial Sequence
  cDNA
  misc_feature (1097)..(1121): Applied BioSystems context sequences, the region of that can be amplified in some embodiments of the presently-disclosed subject matter.
gcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgagacagagtctggccatgttacccaatctgatcttgaactcctgctgtcatctgatcctcctgcctcagcctcccaaagtgctgggataagaggtgtgaggcaccatccccggccagtttgcagtctaaaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctggaaaa

SEQ ID NO: 14 - DNA, 3500 bp, Artificial Sequence
  cDNA
  misc_feature (635)..(659): Applied BioSystems context sequences, the region of that can be amplified in some embodiments of the presently-disclosed subject matter.
gagtactgtgaagatgtggtccccaaggctagagctgaaaagaggcttagggccgggtgagccttccagccagggcctgcctccaagtgatgctcccccagggcagggggcataaggatggcacccagccaggtgggagcctgggccctgcccagcctcaaagctttgagctcaggaaatccggaggcaggggagggggacatcgttgccacattccccagccctttaagacccccaaggcaggaaggctgcccgggcctcaccagcttccctcacaggctccttcctgggaggaaggggctgcctgtgccctcgaaggcgcaagggagggcaggagggaggctcggaaggtgttgcaatccccagcccccgggcctgtcagaggctgagccattaacgacagagctcggggagagaagctggactgcagctggtttcaggaacttctcttgacgagaagagagaccaaggaggccaagcaggggctgggccagaggtgccaacatggggaaactgaggctcggctcggaaaggtgaagtaacttgtccaagatcacaaagctggtgaacatcaagttggtgctatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggagctgcccggacccagacagggacagtggctggggcagacatctgaaatgagccgtgctctcaccccacatccttgtgtgcaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagggcttcctggggttcccttcttgggcctgcctcttaaaggcctgagctagctggagaagaggggagggtccataagcccatgactaaaaactaccccagcccaggctctcaccatctccagtcaccagcatctccctctcctcccaatctccataggctgggcctcccaggcgatctgcatactttaaggaccagatcatgctccatccagccccacccaatggccttttgtgcttgtttcctataacttcagtattgtaaactagtttttggtttgcagtttttgttgttgtttatagacactcttgggtgtaaaaaaaaaaa

SEQ ID NO: 15 - DNA, 4107 bp, Artificial Sequence
  cDNA
  misc_feature (498)..(522): Applied BioSystems context sequences, the region of that can be amplified in some embodiments of the presently-disclosed subject matter.
gacaagggctcttcttgatggcttactgtatccactttgtccccaagaccatagggaaatgactagaggtgactgtactagctagattttaaatgaaactgaaatgaaagttcacttcctcattttgagtacctcatgtgacaagttccaatttcttttcaagtcaattgaactgaaatctccttgttgctttgaaatcttagaagagagcccactaattcaaggactcttactgtgggagcaactgctggttctatcacaatgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaataaaggcccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctagaggatctctcctttttataacaaatcaagaaatatgaagcactttctcttaacttaatttttcctgctgtatccagaagaaataattgtgtcatgattaatgtgtatttactgtactaattagaaaatatagtttgaggccgggcacggtggctcacgcctgtaatcccagtacttgggaggccaaggcaggcatatcaacttgaggccaggagttaaagagcagcctggctaacatggtgaaaccccatctctactaaaaatacaaaaaattagccgagcacggtggtgcatgcctgtaatcccagctacttgggaggctgaggcacgagattccttgaacccaagaggttgaggctatgttgagctgagatcacaccactgtactccagcctggatgacagagtggagactctgtttcaaaaaaacagaaaagaaaatatagtttgattcttcatttttttaaatttgcaaatctcaggataaagtttgctaagtaaattagtaatgtactatagatataactgtacaaaaattgttcaacctaaaacaatctgtaattgcttattgttttattgtatactctttgtctttttaagacccctaatagccttttgtaacttgatggcttaaaaatacttaataaatctgccatttcaaatttctatcattgccacataccattcttattcctaggcaactattaataatctatcctgagaatattaattgtggtattctggtgatggggtttagcaactttgatggaagaaaatattaggctataaatgtcctaaggactcagattgtatctttgtacagaagaggattcaaaacgccacgtgtagtggctcatgcctgtaatcccaacactttgggaggctgaagtaggaggatcgtcttgagcccaggagttcaagaccagcctggacaacatagtgagaccttgtctccacaaaaataaaaaagaaactatccaggagtggtggtgtgtgcctgtggtccctgctatgcagatgtctaagacaggaggatcacaagagcccaggaggttgagaatgcagtgagcttgtaattgcaccactgcactccagcctgggtgacagagcaagaccctgtcttaaaaaaagaggattcaacacatatttttatattatgttaaagtaaagaaatgcataaaagacaagcactttggaagaattattttaatgatcaacaatttaatgtattagtccaaattatttttacgtagtcatcaacaatttgaccagggcctttatttggcaaataactgagccaaccagaataaaataaccaatactccactgctcatatttttatctaattcagatggatcttccttacaactgctctagattagtagatgcatctaagcaggcagcaggaactttaaattttttaagttcatgtctatgacatgaacaatgtgtgggataatgtcattaatatatcctaaattaacctaaacgtatttcactaactctggctccttctccataaagcacattttaaggaacaagaattgctaaatataaaaacataaataataccataatacatggctatcatcaaaagtgtatagaatattatagtttaaaagtatttagttgattacttttcagttttgttttgttttttgagacggagtctcactctgttgcccaggctggagtgcagtggcaccatctcagttcactgcaacttctgcctcccgagttcaagcgattctcctgcctcagcctcccgagtagctggaattataggcgtgcaccaccacgcccagctaatttttgtatttttagtaaagacagggttttgccacattagccaggctggtctcaaactcctgacctcaggtgatccacccaccccagcctcccaaagtgctaagattacaggcgtgagccactgagcccagcctacttttcagtttttaacataatttttgttttatccacaacttttcaagtattgaaagtagaataaaaacatgggttcttagtctttagctatctgttaaagcctatgaatgccttcttaaaatcatgtttttaaatgcataaaatatataggattacaaaggaatctaattatatcgaaatacagttattaaaatgttaaaagataagtttgttatatattaatatgcatgcttctttataaatgcattaaataagagttaatagctatcctaaatttgaaatagtgataagcataatgaaaatagatgcaaaaaactaatgtgatatgaaaatatctgggtttttcttttgatgatgaagtattgctaatattaccgtggtttatgaactatgttcagaattgaagaaaatcctaactttcagttagaggttagtgacggggttcaggacaccctacacaaaatacagcactttgacatattgaatattttaagctgaaggcatttgaggaaattgcagaagcaggaaggtgactctgaccttctgcctgctgttctccccagaagcagccataaaacctgggaaggattttctgaccttcccctgaagtagatcataagactgtcatgtaagaggtgctctcctggcacccagagaaaaggagcatccttacctccaaaagcacagggacacaaagaggaatctaaacaaacaggcctctcagtttcccccagtttattacatttagcttgttcacactttgccctatgacatttctacatcactggctgctcttcatcaaacctactataaaaaacattcaagttcaactgtttctttgggcctttatttccttatggagcccctcgtgtcgtgtaaaacttatattaaataaatgtgcatgcttt

SEQ ID NO: 16 - DNA, 2196 bp, Artificial Sequence
  cDNA
  misc_feature (803)..(827): Applied BioSystems context sequences, the region of that can be amplified in some embodiments of the presently-disclosed subject matter.
ctgggcgggtcatgcgccctggccttcgcgcatctcccaggttagctgcgtgtccgggtgctaggctgcagacccgccgccatgacgctgcgcgcggccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctccggccctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtagaacgcagcgtgtgcccacgctcagcaggtgtgccatccttccacctgctggggcaccattcttagtatacagaggtggccttacacacatcttgcatggatggcagcattgttctgaaggggtttgcagaaaaaaaagattttctttacataaagtgaatcaaatttgacattattttagatcccagagaaatcaggtgtgattagttctccaggcatgaatgcatcgtccctttatctgtaagaacccttagtgtcctgtagggggacagaatggggtggccaggtggtgatttctctttgaccaatgcatagtttggcagaaaaatcagccgttcatttagaagaatcttagcagagattgggatgccttactcaataaagctaagatgactatgctgctggctgtctttgttcttggagaggtggagtgactgttcacggagaa

SEQ ID NO: 17 - DNA, 3651 bp, Artificial Sequence
  cDNA
  misc_feature (1627)..(1651): Applied BioSystems context sequences, the region of that can be amplified in some embodiments of the presently-disclosed subject matter.
ctgtctgagcatttcactgcggagcctgagcgcgcctgcctgggaaaacactgcagcggtgctcggactcctcctgtccagcaggaggcgcggcccggcagctcccgcatgcgcagtgcgctcggtgtcagacggcccggatcccggttaccggcccctcgctcgctgctcgccagcccagactcggccctggcagtggcggctggcgattcggaccgatccgacctgggcggaggtggcccgcgccccgcggcatgagccggtgaccaagctcggggccgagcgggaggcagccgtggccgaggagtgtgaggaagaggctgtctgtgtcattatgtgtgcgtcggtcaagtataatatccggggtcctgccctcatcccaagaatgaagaccaagcaccgaatctactatatcaccctcttctccattgtcctcctgggcctcattgccactggcatgtttcagttttggccccattctatcgagtcctcaaatgactggaatgtagagaagcgcagcatccgtgatgtgccggttgttaggctgccagccgacagtcccatcccagagcggggggatctcagttgcagaatgcacacgtgttttgatgtctatcgctgtggcttcaacccaaagaacaaaatcaaggtgtatatctatgctctgaaaaagtacgtggatgactttggcgtctctgtcagcaacaccatctcccgggagtataatgaactgctcatggccatctcagacagtgactactacactgatgacatcaaccgggcctgtctgtttgttccctccatcgatgtgcttaaccagaacacactgcgcatcaaggagacagcacaagcgatggcccagctctctaggtgggatcgaggtacgaatcacctgttgttcaacatgttgcctggaggtcccccagattataacacagccctggatgtccccagagacagggccctgttggctggtggcggcttttctacgtggacttaccggcaaggctacgatgtcagcattcctgtctatagtccactgtcagctgaggtggatcttccagagaaaggaccaggtccacggcaatacttcctcctgtcatctcaggtgggtctccatcctgagtacagagaggacctagaagccctccaggtcaaacatggagagtcagtgttagtactcgataaatgcaccaacctctcagagggtgtcctttctgtccgtaagcgctgccacaagcaccaggtcttcgattacccacaggtgctacaggaggctactttctgtgtggttcttcgtggagctcggctgggccaggcagtattgagcgatgtgttacaagctggctgtgtcccggttgtcattgcagactcctatattttgcctttctctgaagttcttgactggaagagagcatctgtggttgtaccagaagaaaagatgtcagatgtgtacagtattttgcagagcatcccccaaagacagattgaagaaatgcagagacaggcccggtggttctgggaagcgtacttccagtcaattaaagccattgccctggccaccctgcagattatcaatgaccggatctatccatatgctgccatctcctatgaagaatggaatgaccctcctgctgtgaagtggggcagcgtgagcaatccactcttcctcccgctgatcccaccacagtctcaagggttcaccgccatagtcctcacctacgaccgagtagagagcctcttccgggtcatcactgaagtgtccaaggtgcccagtctatccaaactacttgtcgtctggaataatcagaataaaaaccctccagaagattctctctggcccaaaatccgggttccattaaaagttgtgaggactgctgaaaacaagttaagtaaccgtttcttcccttatgatgaaatcgagacagaagctgttctggccattgatgatgatatcattatgctgacctctgacgagctgcaatttggttatgaggtctggcgggaatttcctgaccggttggtgggttacccgggtcgtctgcatctctgggaccatgagatgaataagtggaagtatgagtctgagtggacgaatgaagtgtccatggtgctcactggggcagctttttatcacaagtattttaattacctgtatacctacaaaatgcctggggatatcaagaactgggtagatgctcatatgaactgtgaagatattgccatgaacttcctggtggccaacgtcacgggaaaagcagttatcaaggtaaccccacgaaagaaattcaagtgtcctgagtgcacagccatagatgggctttcactagaccaaacacacatggtggagaggtcagagtgcatcaacaagtttgcttcagtcttcgggaccatgcctctcaaggtggtggaacaccgagctgaccctgtcctgtacaaagatgactttcctgagaagctgaagagcttccccaacattggcagcttatgaaacgtgtcattggtggaggtctgaatgtgaggctgggacagagggagagaacaaggcctcccagcactctgatgtcagagtagtaggttaagggtggaaggttgacctacttggatcttggcatgcacccacctaacccactttctcaagaacaagaacctagaatgaatatccaagcacctcgagctatgcaacctctgttcttgtatttcttatgatctctgatgggttcttctcgaaaatgccaagtggaagactttgtggcatgctccagatttaaatccagctgaggctccctttgttttcagttccatgtaacaatctggaaggaaacttcacggacaggaagactgctggagaagagaagcgtgttagcccatttgaggtctggggaatcatgtaaagggtacccagacctcacttttagttatttacatcaatgagttctttcagggaaccaaacccagaattcggtgcaaaagccaaacatcttggtgggatttgataaatgccttgggacctggagtgctgggcttgtgcacaggaagagcaccagccgctgagtcaggatcctgtcagttccatgagctattcctctttggtttggctttttgatatgattaaaattattttttattcctttttctactgtgtcttaaacaccaattcctgatagtccaaggaaccacctttctcccttgatatatttaactccgtctttggcctgacaacagtcttctgcccatgtctgggaacacacgccaggaggaatgtctgataccctctgcatcaagcgtaagaaggtcccaaatcataaccattttaagaacagatgactcagaaacctccagaggaatctgtttgcttcctgattagatccagtcaatgttttaaaggtattgtcagagaaaaacagagggtctgtactagccatgcaaggagtcgctctagctggtacccgtaaaagttgtgggaattgtgacccccatcccaaggggatgccaaaatttctctcattcttttggtataaacttaacattagccagggaggttctggctaacgttaaatgctgctatacaactgctttgcaacagttgctggtatatttaaatcattaaatttcagcatttactaatactgcaaaaaaaaaaaaaaaaaa

SEQ ID NO: 18 - DNA, 2158 bp, Artificial Sequence
  cDNA
  misc_feature (340)..(364): Applied BioSystems context sequences, the region of that can be amplified in some embodiments of the presently-disclosed subject matter.
attcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaa

SEQ ID NO: 19 - DNA, 1668 bp, Artificial Sequence
  cDNA
  misc_feature (1097)..(1121): Applied BioSystems context sequences, the region of that can be amplified in some embodiments of the presently-disclosed subject matter.
acgggccaaggcggcgcgtctcgggggtggagcctggaggtgaccgcgccgctgcaacgcccccaccccccgcggtcgcagtggttcagcccgagaacttttcattcataaaaagaaaagactccgcacggcgcgggtgagtcagaacccagcagccgtgtaccccgcagagccgccagccccgggcatgttccgagacttcggggaacccggcccgagctccgggaacggcggcgggtacggcggccccgcgcagcccccggccgcagcgcaggcagcccagcagaagttccacctggtgccaagcatcaacaccatgagtggcagtcaggagctgcagtggatggtacagcctcatttcctggggcccagcagttaccccaggcctctgacctaccctcagtacagccccccacaaccccggccaggagtcatccgggccctggggccgcctccaggggtacgtcgaaggccttgtgaacagatcagcccggaggaagaggagcgccgccgagtaaggcgcgagcggaacaagctggctgcggccaagtgcaggaaccggaggaaggaactgaccgacttcctgcaggcggagactgacaaactggaagatgagaaatctgggctgcagcgagagattgaggagctgcagaagcagaaggagcgcctagagctggtgctggaagcccaccgacccatctgcaaaatcccggaaggagccaaggagggggacacaggcagtaccagtggcaccagcagcccaccagccccctgccgccctgtaccttgtatctccctttccccagggcctgtgcttgaacctgaggcactgcacacccccacactcatgaccacaccctccctaactcctttcacccccagcctggtcttcacctaccccagcactcctgagccttgtgcctcagctcatcgcaagagtagcagcagcagcggagacccatcctctgacccccttggctctccaaccctcctcgctttgtgaggcgcctgagccctactccctgcagatgccaccctagccaatgtctcctccccttcccccaccggtccagctggcctggacagtatcccacatccaactccagcaacttcttctccatccctctaatgagactgaccatattgtgcttcacagtagagccagcttggggccaccaaagctgcccactgtttctcttgagctggcctctctagcacaatttgcactaaatcagagacaaaatatttcccatttgtgccagaggaatcctggcagcccagagactttgtagatccttagaggtcctctggagccctaaccccttccagatcactgccacactctccatcaccctcttcctgtgatccacccaaccctatctcctgacagaaggtgccactttacccacctagaacactaactcaccagccccactgccagcagcagcaggtgattggaccaggccattctgccgccccctcctgaaccgcacagctcaggaggcgcccttggcttctgtgatgagctgatctgcggatctcagctttgagaagccttcagctccagggaatccaagcctccacagcgagggcagctgctatttattttcctaaagagagtatttttatacaaacctaccaaaatggaataaaaggcttgaagctgtg

SEQ ID NO: 20 - DNA, 7856 bp, Artificial Sequence
  cDNA
  misc_feature (113)..(137): Applied BioSystems context sequences, the region of that can be amplified in some embodiments of the presently-disclosed subject matter.
cgcgatctgctgcagctcggccgggagacggcgcgacccggcggcggggccacccgcgagtccagcgtcgccgcagccccccaatgcggccgcgagaagcagcgggggggcaggcgatcgaaggagccttcacgtaaatgggtccagtcatgcctcccagtaagaagccagaaagctcaggaattagtgtctccagtggactgagtcagtgttacgggggcagcggtttctccaaggcccttcaggaagacgatgacctcgacttttctctgcctgacatccgattagaagagggggccatggaagatgaagagctgaccaacctgaactggctgcacgagagcaagaacttgctgaagagctttggggagtcggtcctcaggagtgtcagccccgtccaggacctggacgatgacacccccccatcccctgcccactctgacatgccctacgatgccaggcagaaccccaactgcaaacccccctactccttcagctgcctcatatttatggccatcgaggactctccaaccaagcgcctgccagtgaaggatatctacaactggatcttggaacattttccgtattttgcaaatgcacctactgggtggaaaaactcagtgagacacaatttatcattgaataagtgttttaagaaagtggacaaagagaggagtcagagtattgggaaagggtcgttgtggtgcatagacccagagtatagacaaaatctaattcaggctttgaaaaagacaccttatcacccacacccacacgtgttcaatacacctcccacctgtcctcaggcatatcaaagcacatcaggtccacccatctggccgggcagtaccttcttcaagagaaatggagcccttctccaagatcctgacattgatgctgccagtgccatgatgcttttgaatactccccctgagatacaagcaggttttcctccaggagtgatccaaaatggagcgcgggtcctgagccgagggctgtttcctggcgtgcggccgctgccaatcactcccattggggtgacagcggccatgaggaatggcatcaccagctgccggatgcggactgagagtgagccatcttgtggctccccagtggtcagcggagaccccaaggaggatcacaactacagcagtgccaagtcctccaacgcccggagcacctcgcccaccagcgactccatctcctcctcctcctcctcagccgacgaccactatgagtttgccaccaaggggagccaggagggcagcgagggcagcgaggggagcttccggagccacgagagccccagcgacacggaagaggacgacaggaagcacagccagaaggagcccaaggattctctgggggacagcgggtacgcatcccagcacaagaagcgccagcacttcgccaaggccaggaaggtccccagcgacacactgcccctcaaaaagagacgcaccgaaaagccccccgagagcgatgatgaggagatgaaagaagcggcagggtccctcctgcacttagcagggatccggtcctgtttgaataacatcaccaatcggacggcaaaggggcagaaagagcaaaaggaaaccacaaaaaattaaaaacaagtcactgatttgttttgaacttacgaccatttggtttcagcatgtcaggagatttctaatgatttgtggcaatatcagcaattttttttcttttttcttgtttttggtttggttttctttcttttcttttccttttattttgttttaatttgccccctcttctttgttttggacccttaagaattttatttttaaaggagattgaagccatagaactcatattgacactcagctgttttacaaaagcttttcattatctgaagacaaaaccgaaaaagccaaaattaccattgcttcctccagcttgtcagaaacctgtggctgaatccgcagggatgtcaacgtcaatatcacaggaacacacattcggcacctagaaggcacgtgggcaaagtaatcatcgttcaggcccaacccttaggtttaaaaagtcaggttgtccatcccattgggttcactgagtgaaggcacataaagcaattgaggaggaggaggaacccctcgtccccctaggagcagacccaagcttgtggcaccaggcatctgatggtgccaggaaagccactggaattgtcacacggcgagcacagagggccggccaccagtcctcgatgcttctgaaccctgaagcccgatgacatcttacgaggtggacgttggactgttcatgcgcatcgggtgtcagtgactcatggagaagaaatggggtaaatttttagtgatgttgctaatcattgaattctgttctctattaaattaagaaaatgttccaaaagccataagcctgaagattggccctgtgcacgcacgcacacacacacacacacacacacacacacacacacacacacgaaggagagagagagaaaactgatggggaaaacaagctgtgtcttcttaactgcccaagtgaaaagcaaccaagtccaggaaattacaatagctgttaaggaaaggaaataatggtacagatctttttctgtctatcaaaactatttgatccaagtgaaaaaaaaaaaaaaactagaaagctacggaacctgccattagtattgtggtgtatttttaagattaaaggtacactgatggacaaaaaaaaaaagtaaaacatggcaaaaaataaaataactcctatactgccctcaaaatggagtttgcaattaatatcaggatttatctttgcaaaaatcagtgatttccacattcagccagtatagccagcagaaatttctgatccacaatgcatggattcctttgaagaaaaaaaagaaaaagagaaaaaaatcacaaaaacaaactttttttattcaaaagtaacaaagttcttgtaaggtaaataatgtatttagcatgaagcatgaattattttcatataaatatagaaaatagagaaaaggctatgcctgtaatttttaagcccttaggcttagagtttcttttggttttcttcttttttctttccttttctttgctttctttttttcctttttgtttttgtttttgttttttgtttttgtttttttttcgggttattttgttttggttttttgaagcaggtgtttaaggtttaaccttcttcagggacaaattctgactgttggggaacttactctgcaatataaaaatatcttcatgctctggtagggcttggatggttgaactctgtactgccttgtgtgcacttcagccccgaccccctctgattctctgttgaaaagtgtgtcctttctctctgtctgtacatgtttaacatgacgcaataatttgagggcaaacttagtagtgagtgtgtatgatagaatcaagagaattatgggacgcttacttgagaaaatcattaccatgatttggttctaggaaaaaggcagtgaataattatgcaaattagccagaagaaggggaaccgtgctaatgggccttattgggtgaggggacgagatggggttcatgtgaaggaggaagcgatgccgaggtaggaaaggccagccccagacatcctatcgccacaatgccatgtcgcaataggaagcaggggccggccatcgctaccttcagcacactgaccaacctggaattaagaccacctagattgcgagagctgaatttagaaaccagacaacgtcatgcagcccagaaactcctgttgttacctttgcctaagaaattttctttaatggcgggggcggggggcgggggtacaaagagaaatctctaaaagaatatgatcttccatccaagtggagggaaactttaaaacaaaaacacccagtactgtggctcaggatatgatgcgtgaggagagggagggaacagagatgaccttaacttttaaaaaagggactgctgtgggccaaagccaagcccatctgccaggacgaggtaatgtcagagctccatcagcccggacagtgggaactaactggtgcattccccacacttaccttccggtgggttgctgatgagagaacctgaaaaaacctacacctctacagcaggtcgaattcatgacctgaagctgaatacttccagcatatttattcagggtgtaggtgggaataaagtatcttcgcagtgctctgttccctccgtctccccagacatctgacaccctaaaagccatccacagctatggaacctgagcgacaccttgatttgtgttgtcacctgaccaagcctaaagacctccagctcagtcccccaccttcatcccaccccacagatgataaaattcagacctctctcctgaaaggcagaggttcaacattcaggactgtttctggccgaggacttcttccaattaaaacccccaccgtgggctgtctcccctcatttcatttttctaaaggggcagaggcctcttttagaaaataataaaatgcaatgtgtgtgatttacttttctgatctctttgagaaatagagaaatataaaagtgtgttcttaactccagaaccactctttttgcataaatacctcatcgggcagctttctaagtgtgattttcctgagtctcccttcgttggatctgccggaagacttgtcggggaacctttagtgagggtacttcttcctatttttcttctgtttttggaggcatacacattatgcataaccaaaacaatggctcaattgtgtttaactttgtattttgattgttgagaacaaaaacaaaaagtatcaatgtgtatgtggctgtttgtagtgaatttattggagaatgaggttgtccgtgtccttaacaagccaaggggcaggaggcaccctctcttatcccctcctccaagagcagtagagaatttaagcacaagcctatttgtgaaagaatattttgcttaagtgtcattcactttagtcttggaattccttcccaaacgtcaggtgttcttttagcttccaaactagcatatgtatccattagtctgacagatcgcctgaacaccattaagaggtgtggcgtttttgctttcatttctcctgctgggagaagtggcggttcatgtgtcattccagtatctcacatactcacacggggcaggggggagggggaaacggggaactatagcaatatttaaagatgctttggaaaccaaccgtgaacacatcaacaccacgacgtctacgattacttgctattggccctcggatacatttaagagaaagagacagtcactcttttttttcttaaatgatatacatataaacagttatttttatcctattataattgtcttttgtctttatctagtactatgtggaaagggtttgcatcatagatttttcccagccttataatataccataagctcctacttccctgcccctccctaatcagtattctttcaagagttctttggtgaagccatctatctgaaactaaaatgaaccaaacccatatttcactggtggttggagaaaaccatggccaaaacgattgtggcaggtctcaatcttgggagtttttaagaaggaatgtgccagaggccgattcccaagaacagagttttcttttgttttgcagaggcattcaatgtgtctagtgcttgctggccacagcagttactaccacagagccttctgggaggggccgttgtgttgaaggaggctcctgcctgagggacagcatcaggcagtgggctctgtagagtgagaaccaggtggaggccttctgtgcccagctcagagttctgcaccacgccaggactgcccaggccaagggctactgacgcaagttccactcattccactctgtggggggcgccttgggcctctcctggaagggctcttggagaaggaattggagttacgtacaagtgacctaaatgggaagcttttctagatgagattggattaaattccatgtgatttctctttccctttaatccaggttgggactcgtttctttctggtggatcacagctgcccagatgttgcaattgatttttatgtttctgtagagaagtatttttctttcatcttcaggattttttttgccaccaaaagaaaacattggaactctgtgtttcctcttgattgtgacttcccagtgttgacagttaagtccttagtgtcgtaggtcccagcccaccaatactatatcaaacactgttatgcacataatgcagcactgtgatctaatttaaataatacttttttattatttatactactatatataatatacatcaacacttttgctatataacctaagtgataaccctcttttagttacctgccaaactctggacttggtttatattgcagttaacacagttacaaagctgtaatggtgtctttttttcctttgtaacggaatgtgtaaatcaaagtatatacattgtgtggtgttcctgtttctggagtttcatgaggatttacacatggcattcagtgttctgtatagatctgcctacctttgtgaattcatctgttaacccctcttcctttgagagagcaccggcgatggtggttaactccttgtgttttctctctctcctactggttattcttgaattaagcacagactcgtcagctcggttgctttatcatgaataatgtgtgtgaccttgcagttcttccacagttcagcaaacaagtgctagcttcactgaccaaaaattaaggaaggaaaacacagtttttaaaacgatccatcttttaacagccgaaaccgatgtgtctatggtgctgcaccttgctgttgtacttctgaaatcagacgtgtgtgaacgatcatttctgacttaaccgtgagatgctcacgagtacccttcctgttgttttgttagcattgaaatcgagactatttatttggaatatatacaacagtgtttttccactgtatttcatttgcaaaagttgagaactgctttctctaccttttgcaaaataattgatattccatattggattctcaaagacttcgatatggtgaacctattaaacctagaaattgtattcatcctttcatgactgtggcctgagttccccagcccctctcctccttttttttagatgagatttagcacactctcagttatttaaacatgcaacatttcttgagtatgtatgttgaggccatctgagctcatagctgattcagtaaccagtttcatgctgtgtcattcacactcactacttaatactgccatggtgaaaatgtggaggaaaaatgtatccatgtgtgtctgggaagcatatacacttgtacattttttaatactctgattctgtaacatttctgagttttgttttgttttacagaaaaaaaaaaaaagtgataaagcaatcagaagaccaagaggtttactattgatgcttagggtcgtctgaccttggctggccaatagacctacacggccaaattaatttacgagagtaataatttttcaaaagccaattttttttctgtattttctgtatgaaactgccaatatcatgaatagaaagggagaaccataaaggagaaagaacgtgatgttctgttatgttcatgtaaacctaaagaaacagtgtggaggcaggcgcgatcagccgaactctagggacttggtgttgcttggaaggcatccatacctgcattttgcattcttcgtatgtaatcatattgccaaagacaaactatttcatcatttattgtaaataacacttttccccagacctaccataaagtttctgtgatgtattgtcttccagttgcaataaaaattactgagttgcatcaattgaagaaaaacaccaaaaa

SEQ ID NO: 21 - DNA, 1310 bp, Artificial Sequence
  cDNA
  misc_feature (145)..(169): Applied BioSystems context sequences, the region of that can be amplified in some embodiments of the presently-disclosed subject matter.
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 22 - DNA, 1310 bp, Artificial Sequence; identical to SEQ ID NO: 21
  cDNA
  misc_feature (145)..(169): Applied BioSystems context sequences, the region of that can be amplified in some embodiments of the presently-disclosed subject matter.

SEQ ID NO: 23 - DNA, 3070 bp, Artificial Sequence
  cDNA
  misc_feature (790)..(814): Applied BioSystems context sequences, the region of that can be amplified in some embodiments of the presently-disclosed subject matter.
ggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 24 - DNA, 2979 bp, Artificial Sequence
  cDNA
  misc_feature (186)..(210): Applied BioSystems context sequences, the region of that can be amplified in some embodiments of the presently-disclosed subject matter.
  misc_feature (446)..(470): Applied BioSystems context sequences, the region of that can be amplified in some embodiments of the presently-disclosed subject matter.
ccggggacggctgctggagcggcgcccgccgcggctcagcgcattcccgctctccgcttccctctccgctgcgtccccgcgcgaagatggcaaccgaggggctgcacgagaacgagacgctggcgtcgctgaagagcgaggccgagagcctcaagggcaagctggaggaggagcgagccaagctgcacgatgtggagctgcaccaggtggcggagcgggtggaggccctggggcagtttgtcatgaagaccagaaggaccctcaaaggccacgggaacaaagtcctgtgcatggactggtgcaaagataagaggaggatcgtgagctcgtcacaggatgggaaggtgatcgtgtgggattccttcaccacaaacaaggagcacgcggtcaccatgccctgcacgtgggtgatggcatgtgcttatgccccatcgggatgtgccattgcttgtggtggtttggataataagtgttctgtgtaccccttgacgtttgacaaaaatgaaaacatggctgccaaaaagaagtctgttgctatgcacaccaactacctgtcggcctgcagcttcaccaactctgacatgcagatcctgacagcgagcggcgatggcacatgtgccctgtgggacgtggagagcgggcagctgctgcagagcttccacggacatggggctgacgtcctctgcttggacctggccccctcagaaactggaaacaccttcgtgtctgggggatgtgacaagaaagccatggtgtgggacatgcgctccggccagtgcgtgcaggcctttgaaacacatgaatctgacatcaacagtgtccggtactaccccagtggagatgcctttgcttcagggtcagatgacgctacgtgtcgcctctatgacctgcgggcagatagggaggttgccatctattccaaagaaagcatcatatttggagcatccagcgtggacttctccctcagtggtcgcctgctgtttgctggatacaatgattacactatcaacgtctgggatgttctcaaagggtcccgggtctccatcctgtttggacatgaaaaccgcgttagcactctacgagtttcccccgatgggactgctttctgctctggatcatgggatcataccctcagagtctgggcctaatcatcttctgacagtgcactcatgtatacctgagaatttgaaatcttcacatgtaaatagatattacttctagaggagcttagagtttattgcagtgtagcttaggggagcaacccatggctcacaggtcactaagcgtctccaatatgactattaaaactgtcacctctggaaatacactagtgtgagccttcagcactgcgagaataccttcaagtacagtatttttcttttggaacactttttaaaatgtatctgtttttaaggttattctaaattatagtagcctcaactcattctgtcaccagtagaattcagcagttaatatattccatattatttctttgaatcaattcattttcagagcactttaaagtctgatatttctcgatgtgcactgtgatgcctggaaccttcctctggaagtgctgattttatggactgaggactggtgactggtctgtgatagaagcaaattccaattccaaatgtaattagacaaaaatcatttttttagaatgtgtttttattgtaaaagtatctttttcagcttcctgttctattgtcttttttcagatacaacatttttgtctatggtgaactgctgtaaatgacgcagagaaatgcctaaaaaggacaggtggtttgactcatggatgatgatgatgtcactgtgccacttggacagggcgttttctctgaattgaagggaaagccaatggtgtttgtaaacaaatgcttctgagagcaaagaaaagtcttctgtgtgggaacacaagatagtaaacttatttaaaaacctattagtagaattagtggaaacacttaggttaaagtgaatcttgtccatataaattatattcatggccgggcgcggtggctcacgcttgtaatcccagcactttgggaggccgaggcgggcggatcacgaggtcaggagttcgagaccacggtgaaaccctgtctctactaaaaaatacaaaaaattagccgggcgtggtggcgggcgcctgtagtcccagctactcggagaggctgaggcaggagaatggcgtgaacccgggaggtggagcttgcagtgagccgaggtcgagccactgcagcctgggtgacaaagcgagactccgtctcaaaaaaaaaaaaaaattatattcatatgtattgcattgcaattataattacatatgcagattgattgatagtcatgaataataacgtctgctcctcttacatagaaaaacgatattaaaagaagatcttctctttatttgagactcagaattccttctagaagaaggaagtgctttttgttataggatcccttcttttcctttttttgtttttttgtaagatgtagatgcttattctttgctttagaaaacttctcacttaaaaagatggcatgcacctaggggaataaaaggtcacctcagacaccaggtgtcattcctggtgaggcctgcctcgtcggtggcctggggtctgccggcaggttctggctgcacctgaaggctgcgtgcaccttgtcccctggacaggtctcctttcctggccctgctccagcccagcccttcttctagtggtagctctggctttgcaggcccagctccaggccctgctcctcagagagactcttccagagctggagctgggcacagccataagacaggactggaccagatgctcctgtaaacatccaggggtgtgccaggcccaccctcacaactgcttgttcaggtatcgtgatgggccactcggtccaaaatcagccaggccatcttttccatcatctcacttcaaataaacataataattatatttgatcatttgc

SEQ ID NO: 25 - DNA, 1372 bp, Artificial Sequence
  cDNA
  misc_feature (661)..(686): Applied BioSystems context sequences, the region of that can be amplified in some embodiments of the presently-disclosed subject matter.
aagctggcgaggccgagcccctcctagtgcttccggaccttgctccctgaacactcggaggtggcggtggatcttactccttccagccagtgaggatccagcaacctgctccgtgcctcccgcgcctgttggttggaagtgacgaccttgaagatcggccggttggaagtgacgaccttgaagatcggcgggcgcagcggggccgagggggcgggtctggcgctaggtccagcccctgcgtgccgggaaccccagaggaggtcgcagttcagcccagctgaggcctgtctgcagaatcgacaccaaccagcatcatgtccatgacactggggtactgggacatccgcgggctggcccacgccatccgcctgctcctggaatacacagactcaagctacgaggaaaagaagtatacgatgggggacgctcctgactatgacagaagccagtggctgaatgaaaaattcaagctgggcctggactttcccaatctgccctacttgattgatggggctcacaagatcacccagagcaacgccatcctgtgctacattgcccgcaagcacaacctgtgtggggagacagaagaggagaagattcgtgtggacattttggagaaccaggctatggacgtctccaatcagctggccagagtctgctacagccctgactttgagaaactgaagccagaatacttggaggaacttcctacaatgatgcagcacttctcacagttcctggggaagaggccatggtttgttggagacaagatcacctttgtagatttcctcgcctatgatgtccttgacctccaccgtatatttgagcccaactgcttggacgcctttccaaatctgaaggacttcatctcccgctttgaggtttcctgtggcataatgtgatggtcaattttctgcatcaacttgactgggctaagggatgctcagatggcaggtaaaatcattgtgcttgtgagggtgtttccagaagagatttgcctttgaatcagaagacagcaaagatttccttcagcaatgaaggaggcatccaccaaactgtcagggcccagagagaagaaaaagacaggaagggtgaatttgacctctctgactgggacatccatctctgcctatcctgggacctccacactcctggttctctggccttcagacttgatcagggactaacaccatcgcctcccacccccacctttgttctgaggcctttagcctctgaatgataccactggctttcctgcttctctatcctgcagtcggcagatcatgggacttcttcactccaaaattgtgtgagccaattcccataacagatagataaatttataaataaacacacaaatttcctacagcct

SEQ ID NO: 26 - DNA, 1312 bp, Artificial Sequence
  cDNA
  misc_feature (179)..(203): Applied BioSystems context sequences, the region of that can be amplified in some embodiments of the presently-disclosed subject matter.
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 27 - DNA, 1169 bp, Artificial Sequence
  cDNA
  misc_feature (627)..(651): Applied BioSystems context sequences, the region of that can be amplified in some embodiments of the presently-disclosed subject matter.
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 28 - DNA, 665 bp, Artificial Sequence
  cDNA
  misc_feature (207)..(231): Applied BioSystems context sequences, the region of that can be amplified in some embodiments of the presently-disclosed subject matter.
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 29 - DNA, 1760 bp, Artificial Sequence
  cDNA
  misc_feature (684)..(709): Applied BioSystems context sequences, the region of that can be amplified in some embodiments of the presently-disclosed subject matter.
agagggcgagggcgagggcagagggcgctggcggcagcggccgcggaagatgagcagcagctgctcagggctgagcagggtcctggtggccgtggctacagccctggtgtctgcctcctccccctgcccccaggcctggggccccccaggggtccagtatgggcagccaggcaggtccgtgaagctgtgttgtcctggagtgactgccggggacccagtgtcctggtttcgggatggggagccaaagctgctccagggacctgactctgggctagggcatgaactggtcctggcccaggcagacagcactgatgagggcacctacatctgccagaccctggatggtgcacttgggggcacagtgaccctgcagctgggctaccctccagcccgccctgttgtctcctgccaagcagccgactatgagaacttctcttgcacttggagtcccagccagatcagcggtttacccacccgctacctcacctcctacaggaagaagacagtcctaggagctgatagccagaggaggagtccatccacagggccctggccatgcccacaggatcccctaggggctgcccgctgtgttgtccacggggctgagttctggagccagtaccggattaatgtgactgaggtgaacccactgggtgccagcacacgcctgctggatgtgagcttgcagagcatcttgcgccctgacccaccccagggcctgcgggtagagtcagtaccaggttacccccgacgcctgcgagccagctggacataccctgcctcctggccgtgccagccccacttcctgctcaagttccgtttgcagtaccgtccggcgcagcatccagcctggtccacggtggagccagctggactggaggaggtgatcacagatgctgtggctgggctgccccatgctgtacgagtcagtgcccgggactttctagatgctggcacctggagcacctggagcccggaggcctggggaactccgagcactgggaccataccaaaggagataccagcatggggccagctacacacgcagccagaggtggagcctcaggtggacagccctgctcctccaaggccctccctccaaccacaccctcggctacttgatcacagggactctgtggagcaggtagctgtgctggcgtctttgggaatcctttctttcctgggactggtggctggggccctggcactggggctctggctgaggctgagacggggtgggaaggatggatccccaaagcctgggttcttggcctcagtgattccagtggacaggcgtccaggagctccaaacctgtagaggacccaggagggcttcggcagattccacctataattctgtcttgctggtgtggatagaaaccaggcaggacagtagatccctatggttggatctcagctggaagttctgtttggagcccatttctgtgagaccctgtatttcaaatttgcagctgaaaggtgcttgtacctctgatttcaccccagagttggagttctgctcaaggaacgtgtgtaatgtgtacatctgtgtccatgtgtgaccatgtgtctgtgaggcagggaacatgtattctctgcatgcatgtatgtaggtgcctggggagtgtgtgtgggtccttggctcttggcctttccccttgcaggggttgtgcaggtgtgaataaagagaataaggaagttcttggaaaaaaaaaaaaaaaaaaaaaaaaaaaaaaaaaaaaaaaaaaaaaaaaaaaaaa

SEQ ID NO: 30 - DNA, 3338 bp, Artificial Sequence
  cDNA
  misc_feature (461)..(486): Applied BioSystems context sequences, the region of that can be amplified in some embodiments of the presently-disclosed subject matter.
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 31 - DNA, 5312 bp, Artificial Sequence
  cDNA
  misc_feature (616)..(640): Applied BioSystems context sequences, the region of that can be amplified in some embodiments of the presently-disclosed subject matter.
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 32 - DNA, 2619 bp, Artificial Sequence
  cDNA
  misc_feature (1425)..(1449): Applied BioSystems context sequences, the region of that can be amplified in some embodiments of the presently-disclosed subject matter.
gcttcctgggcttcccatctctggcgggaagcgctccccgacgcattctctacctaggggacacccccaaggcaggagcccgggccgacggagaggacttaacgacactatcggaccctctgggaaaagaggggagacgtcgtgacccaggccccgccccaccttgccgcctcgtgcccggcgctaagacccagcgggcgcgccgcccgcccggggcccggccctgtccccttccgtccgcggggcagccagctcagctccggagagccggcggcgcggcgggcatggctcgggtggcgtgggggctgctgtggttgctgctgggcagcgccggggcgcagtacgagaagtacagcttccggggcttcccgcccgaggacctgatgccgctggccgcggcgtacgggcacgctctggagcagtacgagggagagagctggcgcgagagcgcgcgctacctggaggcggcgctgcggctgcaccggctcctgcgcgacagcgaggccttctgccacgccaactgcagcggccccgcgcccgcggccaagcccgatcccgacggcggccgcgcagacgagtgggcctgcgagctgcggctcttcggccgcgtcctggagcgagccgcctgcctgcggcgctgcaagcggacgctgcccgccttccaggtgccctacccgccgcggcagctgctgcgtgacttccagagccgcctgccctaccagtacctgcactacgcgctgttcaaggctaaccggctggagaaggcggtggcggcggcctacaccttcctccagaggaacccgaagcacgagctgaccgccaagtatctcaactactatcaggggatgctggacgtcgccgacgagtccctcacggacctagaggcccagccctacgaggccgtgttcctccgggctgtgaagctctacaacagcggggatttccgcagcagcacggaggacatggagcgggccttgtcagagtacctggcagtctttgcccggtgcctggccggctgtgaaggggcccatgagcaggtggacttcaaggacttctacccggccatagcagatctctttgcagagtccctgcagtgcaaggtggactgtgaggccaatttgacccccaatgtgggtggctacttcgtggacaagttcgtggccaccatgtaccactacctgcagtttgcctactataagttgaatgatgtgcgccaggctgcccgcagcgccgccagctacatgctcttcgaccccaaggacagcgtcatgcagcagaacctggtgtattaccggttccaccgggctcgctggggcctggaagaggaggacttccagccccgggaggaggccatgctctaccacaaccagaccgccgagctgcgggagctgctggagttcacccacatgtacctgcagtcagatgatgagatggagctggaggagacagaaccgcccctggagcctgaggatgccctatctgacgccgagtttgagggggagggtgactacgaggagggcatgtatgctgactggtggcaggagccggatgccaagggtgacgaggccgaggctgagccagagcctgaactcgcatgagaaggggacaccccacaccgctcaagcttgggaagcctggtgccgatggccccaccctcaccagcctgggcagcagcaagaactatttattaaaaacttaagatgggccaggtgcggtggctcacacctgtaatcccagcattttgggaggccaaggtgggtggatcacttgaggccaggagttcaagaccagcctggccaacatgatgagacctccgtctctactaaaatacataaattagccgggtgtggtggcaggcgcctgaaatcccagctactcaagaggctgaggcaggagaatcgcttgaacctgggaggcaaaggttgcagtgaactgagattgcgccaccgcactccagcctgggcgacagagcgagactccatctttaaaaaaaaacaagacgggccggcacggtggctcacgcctgtaatcccagcactgagaggccgatcacttgaggtcaggagttcaagaccagcctggccaacatggtgaaaccccatctctactaaaaaatacaaaaattagccaggcatggtggcacacacctgtaatcgtagctgaggcaggagaatcgcctgaacccaggaggcggagcttgcagtgagccgagatcgtgccactgcactccagcctgggcgacagagtgagactccatctcaaaaaaaaaaaaaaaaacttaagatggacacagctgactggacccccatcctgcctcacccatgggtgctgcaccccagacccatcctgccacttctatgtctctggaccacaggatggtggtggcattgcaggttggcaagtgggctgatggggtccgccctcctcactgctgagctcctcacctggacagtctcctggacaaggagtttccagctgctggctggagtctcaggccaaattgcagagggtcctccagggtcctgaagagcactggactaagagtctagtggttccagggccctgaccagtaggtgctcaataaatgtttgttgttgaatgaaaaaaaaaaaaaaaaaa

SEQ ID NO: 33 - DNA, 1440 bp, Artificial Sequence
  cDNA
  misc_feature (678)..(702): Applied BioSystems context sequences, the region of that can be amplified in some embodiments of the presently-disclosed subject matter.
ggaggtgagcaggaaggagacggccgcccagcagcccgtgggcaggcgcggcggagcgagcggggccggcggcgggcgccgagggacgccgaggcctcgggcgggggctggcccggggttccaggtctccagtgggggctgcagactaagcaaaatgaggcggttcctgaggccagggcatgaccctgtgcgggagaggctcaagcgggacctgttccagtttaacaagacggtggagcatggcttcccgcaccagcccagcgccctcggctacagcccgtccctgcgcatcctggccatcggcacccgttctggagccatcaagctctacggagccccaggcgtggagttcatggggctgcaccaggagaacaacgctgtgacgcagatccacctcctgcccggccagtgccagctggtcaccctgctggatgacaacagcctgcacctttggagcctgaaggtcaagggcggggcatcggagctgcaggaggatgagagcttcacactgcgtggacccccaggggctgcccccagtgccacacagatcaccgtggtcctgccacattcctcctgcgagctgctctacctgggcaccgagagtggcaacgtgtttgtggtgcagctgccagcttttcgtgcgctggaggaccggaccatcagctcggacgcggtgctgcagcggttgccagaggaggcccgccaccggcgtgtgttcgagatggtggaggcactgcaggagcaccctcgagaccccaaccagatcctgatcggctacagccgaggcctcgttgtcatctgggacctacagggcagccgcgtgctctaccacttcctcagcagccagcaactggagaacatctggtggcagcgggacggccgcctgctcgtcagctgtcactctgacggcagctactgccagtggcccgtgtccagcgaagcccagcaaccagagcccctccgcagcctcgtgccttacggtccctttccttgcaaagcgattaccagaatcctctggctgaccactaggcaggggttgcccttcaccatcttccagggtggcatgccacgggccagctacggggaccgccactgcatctcagtgatccacgatggccagcagacggccttcgacttcacctcccgtgtcatcggcttcactgtcctcacagaggcagaccctgcagccagtaggagagcttcgggagtgggtgcccagggttaggtgtgggaggcatggggcaggaccatcagtaaagacagggccaggtgcagtggctcctgcctgtaaccccagtgctgtgggaggccaaggtggtaggatcgcttgaacccaggagttcaagtccagcctggacaacgtagggagacccttgtctctacaaaaaataaaaaaattagccaggaaaaaaaaaaaaaaaaaaaaaaaaaaaaaaaaaa

SEQ ID NO: 34 - DNA, 4454 bp, Artificial Sequence
  cDNA
  misc_feature (688)..(713): Applied BioSystems context sequences, the region of that can be amplified in some embodiments of the presently-disclosed subject matter.
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 35 - DNA, 1575 bp, Artificial Sequence
  cDNA
  misc_feature (566)..(590): Applied BioSystems context sequences, the region of that can be amplified in some embodiments of the presently-disclosed subject matter.
tcccttctgaggaaacgaaaccaacagcagtccaagctcagtcagcagaagagataaaagcaaacaggtctgggaggcagttctgttgccactctctctcctgtcaatgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctgacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaggacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttaaaaaaaaaaa

SEQ ID NO: 36 - DNA, 3177 bp, Artificial Sequence
  cDNA
  misc_feature (216)..(240): Applied BioSystems context sequences, the region of that can be amplified in some embodiments of the presently-disclosed subject matter.
acggtctgggggcggggccacgccgattggcgcgaagttttcttttctccttccaccttcttttcatttctagtgagacacacgctttggtcctggctttcggcccgtagttgtagaaggagccctgctggtgcaggttagaggtgccgcatcccccggagctctcgaagtggaggcggtaggaaacggagggcttgcggctagccggaggaagctttggagccggaagccatggcacactaccccacaaggctgaagaccagaaaaacttattcatgggttggcaggcccttgttggatcgaaaactgcactaccaaacctatagagaaatgtgtgtgaaaacagaaggttgttccaccgagattcacatccagattggacagtttgtgttgattgaaggggatgatgatgaaaacccgtatgttgctaaattgcttgagttgttcgaagatgactctgatcctcctcctaagaaacgtgctcgagtacagtggtttgtccgattctgtgaagtccctgcctgtaaacggcatttgttgggccggaagcctggtgcacaggaaatattctggtatgattacccggcctgtgacagcaacattaatgcggagaccatcattggccttgttcgggtgatacctttagccccaaaggatgtggtaccgacgaatctgaaaaatgagaagacactctttgtgaaactatcctggaatgagaagaaattcaggccactttcctcagaactatttgcggagttgaataaaccacaagagagtgcagccaagtgccagaaacccgtgagagccaagagtaagagtgcagagagcccttcttggaccccagcagaacatgtggccaaaaggattgaatcaaggcactccgcctccaaatctcgccaaactcctacccatcctcttaccccaagagccagaaagaggctggagcttggcaacttaggtaaccctcagatgtcccagcagacttcatgtgcctccttggattctccaggaagaataaaacggaaagtggccttctcggagatcacctcaccttctaagagatctcagcctgataaacttcaaaccttgtctccagctctgaaagccccagagaaaaccagagagactggactctcttatactgaggatgacaagaaggcttcacctgaacatcgcataatcctgagaacccgaattgcagcttcgaaaaccatagacattagagaggagagaacacttacccctatcagtgggggacagagatcttcagtggtgccatccgtgattctgaaaccagaaaacatcaaaaagagggatgcaaaagaagcaaaagcccagaatgaagcgacctctactccccatcgtatccgcagaaagagttctgtcttgactatgaatcggattaggcagcagcttcggtttctaggtaatagtaaaagtgaccaagaagagaaagagattctgccagcagcagagatttcagactctagcagtgacgaagaagaggcttccacaccgccccttccaaggagagcacccagaactgtgtccaggaacctgcgatcttccttgaagtcatccttacataccctcacgaagctcaagcctagaacgccacgttgtgccgctcctcagatccgtagtcgaagcctggctgcccaggagccagccagtgtgctggaggaagcccgactgaggctgcatgtttctgctgtacctgagtctcttccctgtcgggaacaggaattccaagacatctacaattttgtggaaagcaaactccttgaccataccggagggtgcatgtacatctccggtgtccctgggacagggaagactgccactgttcatgaagtgatacgctgcctgcagcaggcagcccaagccaatgatgttcctccctttcaatacattgaggtcaatggcatgaagctgacggagccccaccaagtctatgtgcaaatcttgcagaagctaacaggccaaaaagcaacagccaaccatgcggcagaactgctggcaaagcaattctgcacccgagggtcacctcaggaaaccaccgtcctgcttgtggatgagctcgaccttctgtggactcacaaacaagacataatgtacaatctctttgactggcccactcataaggaggcccggcttgtggtcctggcaattgccaacacaatggacctgccagagcgaatcatgatgaaccgggtgtccagccgactgggtcttaccaggatgtgcttccagccctatacatatagccagctgcagcagatcctaaggtcccggctcaagcatctaaaggcctttgaagatgatgccatccagctggtagccaggaaggtagcagcactgtctggagatgcacgacggtgcctggacatctgcaggcgtgccacagagatctgtgagttctcccagcagaagcctgactcccctggcctggtcaccatagcccactcaatggaagctgtggatgagatgttttcatcatcatacatcacggccatcaaaaattcctctgttctggaacagagcttcctgagagccatcctcgcagagttccgtcgatcaggactggaggaagccacgtttcaacagatatatagtcaacatgtggcactgtgcagaatggagggactgccgtaccccaccatgtcagagaccatggccgtgtgttctcacctgggctcctgtcgcctcctgcttgtggagcccagcaggaacgatctgctccttcgggtgcggctcaacgtcagccaggatgatgtgctgtatgcgctgaaagacgagtaaaggggcttcacaagttaaaagactggggtcttgctgggttttgttttttgagacagggtcttgctctgtcgcccaggctggagtgcagtggcacgatcatggctcactgcagccttgacttctcaggcttaggtgaccccccaacctcatcctcccaggtggctgaaactacaggcacatgccaccatgcccagctgattttttgtagagacagggcttcaccatgttgccaagctagtctacaaagcatctgattttggaagtacatggaattgttgtaacaaagtatattgaatggaaatggctctcatgtattttggaattttccattaaataatttgctttttcctgaaaaaaaaaaaaaaaaaaaaaaaa

SEQ ID NO: 37 - DNA, 2439 bp, Artificial Sequence
  cDNA
  misc_feature (586)..(670): Applied BioSystems context sequences, the region of that can be amplified in some embodiments of the presently-disclosed subject matter.
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 38 - DNA, 1954 bp, Artificial Sequence
  cDNA
  misc_feature (265)..(289): Applied BioSystems context sequences, the region of that can be amplified in some embodiments of the presently-disclosed subject matter.
actggacaaaagcgtggtctctggcgcggggatctcagagtttcccgggcactcaccgtgtgtagttggcatctccgcgcgtccggacacccgatcccagcatccctgcctgcaggactgttcgtgttcagctcgcgtcctgcagctgtccgaggtgctccagttggaggctgaggttcccgggctctgtagctgagtgggcggcggcaccggcggagatgcctgggaagaaggcgcgcaagaacgctcaaccgagccccgcgcgggctccagcagagctggaagtcgagtgtgctactcaactcaggagatttggagacaaactgaacttccggcagaaacttctgaatctgatatccaaactcttctgctcaggaacctgactgcatcaaaaacttgcatgaggggactccttcaaaagagttttctcaggaggtgcacgtttcatcaatttgaagaaagactgcattgtaattgagaggaatgtgaaggtgcattcatgggtgcccttggaaacggaagatggaatacatcaaagtgaatttctgttcaagttttcccagattatcattctttgggatgagagaacattataaaaccactttgtttattttaaagcaagaatggaagacccttgaaaataaagaagtaattattgacacatttcttttttacttagagaatcgttctagtgtttttgccgaagattaccgctggcctactgtgaagggagatgacctgtgattagactgggcggctggggagaaacagttcagtgcattgttgttgttgctgtttttggtgttttgcttttcagtgccaactcagcacattgtatatgattcggtttatacatattaccttgttataatgaaaaaactcattctgagaacactgaaatgttatactcagtgttgatttcttcggtcactacacaacgtaaaatcatttgtttcttttgactcaaattgtattgcttctgttcagatgatctttcattcaatgtgttcctgttgggcgttactagaaactatggaaaactggaaaataactttgaaaaaattggataaagtataggagggttacttggggccagtaaatcagtagactgaacattcaatataataaaagaacatggggattttgtataaccagggataataaaaagaaaaaagaagttaatttttaattgatgtttttgaaacttagtagaacaaatattcagaagtaacttgataagatatgaatgtttctaaagaagtttctaaaggttcggaaaatgctccttgtcacattagtgtgcatcctacaaaaagtgatctcttaatgtaaattaagaatattttcataattggaatatacttttcttaaaaaaaaggaacagttagttctcatctagaatgaaagttccatatatgcattggtgaatatatatgtatacacatacttacatacttatatgggtatctgtatagataatttgtattagagtattatatagcttcttagtagggtctcaagtaagtttcattttttttatctgggctatatacagtcctcaaataaataatgtcttgattttatttcagcaggaataattttatttattttgcctatttataattaaagtatttttctttagtttgaaaatgtgtattaaagttacatttttgagttacaagagtcttataactacttgaatttttagttaaaatgtcttaatgtaggttgtagtcactttagatggaaaattacctcacatctgttttcttcagtattacttaagattgtttatttagtggtagagagttttttttttcagcctagaggcagctattttaccatctggtatttatggtctaatttgtatttaaacatatgcacacatataaaagttgatactgtggcagtaaactattaaaagttttcactgttcaaaaaaaaaaaaaaaaaa

SEQ ID NO: 39 - DNA, 5173 bp, Artificial Sequence
  cDNA
  misc_feature (1179)..(1203): Applied BioSystems context sequences, the region of that can be amplified in some embodiments of the presently-disclosed subject matter.
aatcggtggccgccagacacccgcggcgaaggcggctcgggctcgggctccggatgtgctaggtgtgggccggcccccacccgaccctgacaagtgaccatggatcctggagccgggtcagagacatctctgactgtcaatgagcaggtcatcgtgatgtcaggtcatgagaccatccgagtgctggaagtcggagtggatgcccaactccctgctgaggaagagagcaaaggactggagggtgtggccgccgagggctcccagagcggagaccctgctgaagccagtcaagctgctggtgaagctgggccagacaacctgggctcctctgcagaggcaactgtgaagtcacccccggggatccctccgagccctgcccctgccattgccaccttcagccaagccccaagccagcctcaggcatcgcagaccctgacgccactggctgtacaagctgccccccagtattgcaggtcaagtggctggtcagcaggggctggccgtgtggacaattcctacagcaactgtggctgccctcccaggactgaccgctgcttctcctacggggggagtgttcaagccacctttagccggtctccaagcagctgctgtgctgaacaccgctcttccggcaccggtacaagctgccgcaccagtacaggcctcctcgacggcccaaccccggccaccagcccagccccagacgctgttccagacccagccgctgctgcagaccacacctgccatcctcccgcagcccactgctgccaccgctgctgcccctacccccaagccagtggacacccccccacagatcaccgtccagcctgcaggcttcgcatttagcccaggaatcatcagtgctgcttccctcgggggacagacccagatcctggggtccctcactacagctccagtcattaccagcgccattcccagcatgccagggatcagcagtcagatcctcaccaatgctcagggacaggttattggaacccttccatgggtagtgaactcagctagtgtggcggccccagcaccagcccaaagcctgcaggtccaggccgtgaccccccagctgttgttgaacgcccagggccaggtgattgcgaccctggctagcagccccctgcctccacctgtggctgtccggaagccaagcacacctgagtcccctgctaagagtgaggtgcagcccatccagcccacaccaaccgtgccccagcctgctgtggtcattgccagcccagctccagccgccaagccatctgcctctgctcctatcccaattacctgctcagagacccccaccgtcagccagttggtgtccaagccacatactccaagtctggatgaggatgggatcaacttagaagagatccgggagtttgccaagaactttaagatccggcggctctcgctgggccttacacagacccaggtgggtcaggctctgactgcaacggaaggtccagcctacagccagtcagccatctgccggttcgagaagctagacatcacacccaagagtgcccagaagctaaagccggtgctggaaaagtggctaaacgaagctgaactgcggaaccaggaaggccagcagaacctgatggagtttgtgggaggcgagccctccaagaaacgcaaacgccgcacctccttcaccccccaggccatagaggctctcaatgcctattttgagaagaacccactgcccacaggccaggagatcactgaaattgctaaggagctcaactacgaccgtgaggtagtgcgggtctggttctgcaatcggcgccagacgctcaagaacaccagcaagctgaacgtctttcagatcccttagggctcagcccctggccctgtgttctagcactttgtccatttcccgtggcatccggctgcagccactgccatgacagcacctgtcattttgccacgtgcagctgtgctcaccccaggtcatcagactccaccgtgtgcatgtgcatcaatgtccctcttttctcccacacatctcacatcatggggaggccagagggggccacacgagagctccaggctctgggctggtcactccgaagaagaggatttgtgacgtcacttagagaagcaccttgctagcatggtttctgaagggtgaattctggtggggaaccagaaactccctgtctttggggcagggctaaagcagctcctaaggaccactggccattagctcttgcttttgatggcattctctttccaccttgtcttctcctttgctcctctgtgttagtgtggcaggtatgacaactcatccagtggaaacacagcctcacactgcccttccgccccccacactttgcctgcaggtgcaccgaaaggacctgggagataaaattcaaaaaagtgtgatgtgctgctcagaaggtcagactccatgtctgccttgacctcaaggtcagaaggttcccaaacccctggggctggaacatgggatctcctcttccacctcttcctggttcctttgcggggaaaattgcactaaaacagaaccttttcttaatccatgttggaaggaagcaacagtgaactctacctgttctggagttctcctgggtctgcagaaggttgggaatttagaaaataaggctgttctttcatattttaatttaatctctgtcaatggccatccctcccacaaaaaaacgtgggttaagagaacttgcagactggatatgcaagcaaacgggcaactctggagaaaaataaggaaaggaatgctgactttctctttctttctcttgtccccacacccattcccaacccaatactggggccttctcaaaaggagcaaattaaacaataaaccagacagcaaggccctgggggaaaggacaacatcctgaaataaatgatggagcccaggaaggtctcttgtggaagttgacttaactctaattttctttgtaactttaagccttggatacgggaggagaaatctcattttgtcgagtctcagaccatgtctgtgtgtaagcaatccccacagtgtcctctgagccaaggacacccccagatcagattgagttttgcttctagacggggtagctatggtaccttgggggttagctctcatccaagctgttaagtgagtttccagcctcactgtggctggaaagcccctaaaattcagtatgtaactccaggaagtcaggagagaactgagatttgcctagatgaccacaggcttgcggtgtagattatccctaaagggccccaagtcacgggggtcaaccacccctgtcttcagtactcttatccttacagaggctggtctctaacagctgcctccagtggacctcccatgatccaccctgagggaaggaccgtcagctggggacacatcaccacctctgtcagtcactggtgcagagccacctcctagcctagcttcctctggtgtcctgtttcctttcccacttactgttggtgcctcccaggccctgcagtgccagcgtggccaccctcttggtagcctggccagtaagaggaggacagttgtgtgctgaattagcacacgcacgtgcagcgcgcacagacgcgcgcacacacacacacatacacgctctgctgcatttggacaaaccatgcctgccagagtgtagcagaggtgaggaagcaggtgggcagcttgcctgacccagcttttcaggagagcgtgtctccaacagagagtctccacactctagttcagggttatcgacctgcctcaatgagatgacagactcatttgggaggggtgttgcaaacaagttttcagtgagaatagttaagttccagagcttgtaaaggattcagtgactgacacttcagtaaattaggccaggcacattggcttatgcctgtaattccaacactttggaaggccgaggtgggcggatcatttgaggtctggagttcgagaccagcctgaccaacatggtgaaaccccgtctctactaaaaatacaaaaattagccaggtgtggtagtgcacatctgtaatcccagctacttgggaggtggaggcaggagaattgcttgaaccctggaggttgcaatgagctgagatcacactacttcactccagcctgggtgacagagcaagactcggtctcaaacaaacaaaaacttatggcgatgcaggttttcatgctcagacgcttgcattcaggtatgctttcttttttgagagagacaaatgggtcacagctggcaccctgggaatagcacataatccagggtgtgtctgtggtggtggacgtgcaggggaacaccatctgtcctgtgtcatgatgggaaaacaatcatgaaccactggtctaaattaggcctggccatgctttctcagcccctccctcatttaaatttgtcttcccaaagctgagctaaaactaaaccatttctcctctgctggaatgatggattggtcattcagaggaacaataccaggggtgggaggtttgcaggctgagttccccaggcatgggggtgcagggtgtccctgaggtttacccaaagcacagctcgctggcctgtgacctctgcccttcctcccacagtgtaagaccccccaggaagcagctggggcctgaacctctcacctaggaggtaggtttattttattttttgttagcatcaggctctgaaggagttggtatacattttgttttgaaaacatcttctggacttacaccagagcttagtgtcgtctttactatggaaagagaggagaatggacagaaatggtttaactgtgtggagttttgtttgttttgttttaaatggaagaaagaccaaaactttcctggtggatcagctagggcctttgaccctgcattaccacggcattttatccaggtgaagtccagggaaagaactcagccaaatggactaaggaacacacgagtttggaatgcgagactctgacatttttgtgttcttggaaatccaattaccttcccatgcccagatttccttcctgcctcttggaccaggctctggcactgaggttctcactgttcccaacacagacaaagcttcctgagggctggaggggcagcaaggggagaggagaatggggaagaagcgcttgatgtagttgtgtggaataaacagtattttttcttttgtaaaaaaaaaaaaaaaaa

SEQ ID NO: 40 - DNA, 3040 bp, Artificial Sequence
  cDNA
  misc_feature (551)..(575): Applied BioSystems context sequences, the region of that can be amplified in some embodiments of the presently-disclosed subject matter.
aaatcctcctcctccgccatcatccgccgcggtgcggagagcaggtggtgctggaagcgcgtgaggccgggagctcgagagagctaacagactagccggctggacatctggaccgctggatccggaggtggcgaccccggcctgacccggaccctaaatccgtccccgccccagagggcggaggcgcgcgctcgattccccccacgcggcggcgccgcctgtttacgtctgcagatctccaggggagcccaccagcctagtcaacatggcctcggaagacattgccaagctggcagagacacttgccaagactcaggtggccgggggacagctgagtttcaaaggcaagagcctcaaactcaacactgcagaagatgctaaagatgtgattaaagagattgaagactttgacagcttggaggctctgcgtctggaaggcaacacagtgggcgtggaagcagccagggtcatcgccaaggccttagagaagaagtcggagttgaagcgctgccactggagtgacatgttcacgggaaggctgcggaccgagatcccaccagccctgatctcactaggggaaggactcatcacagctggggctcagctggtggagctggacttaagcgacaacgcattcgggcccgacggtgtgcaaggcttcgaggccctgctcaagagctcagcctgcttcaccctgcaggaactcaagctcaacaactgtggcatgggcattggcggcggcaagatcctggctgcagctctgaccgaatgtcaccggaaatccagtgcccaaggcaagcctctggccctgaaggtctttgtggctggcagaaaccgtctggagaatgatggcgccactgccttggcagaagcttttagggtcatcgggaccctggaggaggtccacatgccacagaatgggatcaaccaccctggcatcactgccctggcccaggctttcgctgtcaaccccctgctgcgggtcatcaacctgaatgacaacaccttcactgagaagggcgccgtggccatggccgagaccttgaagaccttgcggcaggtggaggtgattaattttggggactgcctggtgcgctccaagggtgcagttgccattgcagatgccatccgcggcggcctgcccaagctaaaggagctgaacttgtcattctgtgaaatcaagagggatgctgccctggctgttgctgaggccatggcagacaaagctgagctggagaagctggacctgaatggcaacaccctgggagaagaaggctgtgaacagcttcaggaggtgctggagggcttcaacatggccaaggtgctggcgtccctcagtgatgacgaggacgaggaggaggaggaggaaggagaagaggaagaagaggaagcagaagaagaggaggaggaagatgaggaagaggaggaagaagaggaggaggaggaggaagaagagcctcagcagcgagggcagggagagaagtcagccacgccctcacggaagattctggaccctaacactggggagccagctcccgtgctgtcctccccacctcctgcagacgtctccaccttcctggcttttccctctccagagaagctgctgcgcctagggcccaagagctccgtgctgatagcccagcagactgacacgtctgaccccgagaaggtggtctctgccttcctaaaggtgtcatctgtgttcaaggacgaagctactgtgaggatggcagtgcaggatgcagtagatgccctgatgcagaaggctttcaactcctcgtccttcaactccaacaccttcctcaccaggctgctcgtgcacatgggtctgctcaagagtgaagacaaggtcaaggccattgccaacctgtacggccccctgatggcgctgaaccacatggtgcagcaggactatttccccaaggcccttgcacccctgctgctggcgttcgtgaccaagcccaacagcgccctggaatcctgctccttcgcccgccacagtctgctgcagacgctgtacaaggtctagactcaaagcctctcccatcccttggcctggaccagtgagctggggagggactcggatgaactgaggcgcagcctacgccattgccttggacaggactctggccacaggcagggcgggtctgtgtcccatgtgtcctgtcagtcccctgagtatgtgtgtgggtgtggcgcatgtgcaggtctgtgcctcctgtcgggatttgggttttaacgtcttctgctggcccagccctgctctgttgtggggagttggcccccaggggaaagggctgtgagctgctccgccattaaactcacctccacctgagggcgctctgctgatctccgcctgggccctgatggccgtccccacccacctgccttccggcccggctccctggcggagccagaacccagggagttgcccgcgtgctgtccttcccctctgtgttgtgattgggttgtttcctgccctgcctggggctgcttctcgtcaccaagccctggtcctgcggcagctgtcacccctaccatccataccactgtgctgaccgctcagcctgaagagcagagaatgccatgggtgggactgtgggggtcggatcgtggggttgttggcagagggcaaccctgggccccacaccgtgtggacaggcagacaccagattgtccaggagcaggagctgctgggactgcgctggccccggacctagtgggccttctcctggctgctgagatgtcgtctgtgactggcctggctggagggggagtgttgacaacccaaagctgttctccagtctggggagggagaggcagggtccccaatgtccgagctgcatctggacgctgctcttaaaggacctcctggggcaggggagcggtagggtctggactgggcagatgctgtatgacctccctgagcacccgtgactgccccatgctttcccctttgtgctctgtgtgtgtctgggctgtgcccgggggcttcacaaataaagtcgtgtggcagcttcaaaaaaaaaaaaaaaaaaaa

SEQ ID NO: 41 - DNA, 1493 bp, Artificial Sequence
  cDNA
  misc_feature (59)..(83): Applied BioSystems context sequences, the region of that can be amplified in some embodiments of the presently-disclosed subject matter.
ggcaaacagcccgcccggcaccaccatgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccccgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggcctgagcacacccgaggctcccacctgcggagccgctgggggacctcacgtcccagccaggatccccctggaagaaaaagggcgtccccacactctaggtgataggacttacgcatccccaccttttggggtaaggggagtgctgccctgccataatccccaagcccagcccgggcctgtctgggattccccacttgtgcctggggtccctctgggatttctttgtcatgtacagactccctgggatcctcatgttttgggtgacaggacctatggaccactatactcggggaggcagggtagcagttcttccagaatcccaagagcttctctgggattttcttgtgatatctgattccccagtgaggcctgggacgtttttaagatcgctgtgtgtctgtaaaccctgaatctcatctggggtgggggccctgctggcaaccctgagccctgtccaaggttccctcttgtcagatctgagatttcctagttatgtctggggccctctgggagctgttatcatctcagatctcttcgcccatctatggctgtgttgtcacatctgtcccctcatttttgagatcccccaattctctggaactattctgctgcccctttttatgtgtctggagttccccaatcacatctagggctcctccaagaaaaaaaaaaaaaaaaaaaaaa

SEQ ID NO: 42 - DNA, 1599 bp, Artificial Sequence
  cDNA
  misc_feature (248)..(272): Applied BioSystems context sequences, the region of that can be amplified in some embodiments of the presently-disclosed subject matter.
aagatcctggcctgtgcagctcgggtttccgagcttctgcctcaggcatctccgcgatctcctctcccctccaatcctatccgtgatggacgatgcccacgagtcgccctccgacaaaggtggagagacaggggagtcggatgagacggccgctgtgcccggggacccgggggctaccgacaccgatggaatcccagaggaaactgacggagacgcagatgtggacttgaaagaagctgcagcggaggaaggcgagctcgagagtcaggatgtctcagatttaacaacagttgaaagggaagactcatcattacttaatcctgcagccaaaaaactgaaaatagataccaaagaaaagaaagagaaaaagcagaaagtagatgaagatgagattcagaagatgcaaatcctggtttcttctttttctgaggagcagctgaaccgttatgaaatgtatcgccgctcagctttccctaaggcagccatcaaaaggctgatccagtccatcactggcacctctgtgtctcagaatgttgttattgctatgtctggtatttccaaggttttcgtcggggaggtggtagaagaagcactggatgtgtgtgagaagtggggagaaatgccaccactacaacccaaacatatgagggaagccgttagaaggttaaagtcaaaaggacagatccctaactcgaagcacaaaaaaatcatcttcttctagaccaaagtctagaaaggcctatgttactgacggaagaagtattggttccagacttcctataagactgtctgcattggtgctttagtatctcaggcctccaaggattccatgatgattttaatgtctttctcaaaactctgatatttgtcacacctagaaagtatgtagcctgattgatacttgccttgactaaattttgggacctcttggggcattttgaagtatttaactgtcttgaccagttggaagaagatacgtgggccataagcatcttctggacaggggaactgctttcagagagaaaacctttccaagagagttttgttttgttttggtttcgttttgtttgagatagggtcttgctctatcacctaggctggagtgcagcggcatgactgcagccttgaactcctgggcttaagtgaccctcccacctcagtctcctgagtagctaggactacaggcacacactactgtgcccagctaacttatttttattttttatggagatggggtcttgctttgttgcccaggctggtcgtgaactcctggcttcaagcagtcctcctgcctcagcctcctaaagtgccgagggctttaatggtttcacattgaagcctgaagttgctaagacttaggttgtttcttatatctggttttaagtagatgaaacaaccagaaacttttacttgtgatactctaccatgaaggatgcggtaatggcaggaatagcagaataattggtgcttgtaaacatttaagattctcctgtggattttggtgagtgatcattaaactgttttccaacttgcaaaaaaaaaaaaaaaaaaaaaaaaaaaaaaaaaaaaaaaaaaaaaaaaaaaaaaaaaaaaaaaaaa

SEQ ID NO: 43 - DNA, 1719 bp, Artificial Sequence
  cDNA
  misc_feature (566)..(590): Applied BioSystems context sequences, the region of that can be amplified in some embodiments of the presently-disclosed subject matter.
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 44 - DNA, 4704 bp, Artificial Sequence
  cDNA
  misc_feature (1970)..(1994): Applied BioSystems context sequences, the region of that can be amplified in some embodiments of the presently-disclosed subject matter.
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 45 - DNA, 2646 bp, Artificial Sequence
  cDNA
  misc_feature (156)..(180): Applied BioSystems context sequences, the region of that can be amplified in some embodiments of the presently-disclosed subject matter.
gattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 46 - DNA, 2583 bp, Artificial Sequence
  cDNA
  misc_feature (555)..(579): Applied BioSystems context sequences, the region of that can be amplified in some embodiments of the presently-disclosed subject matter.
gattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 47 - DNA, 2914 bp, Artificial Sequence
  cDNA
  misc_feature (1253)..(1277): Applied BioSystems context sequences, the region of that can be amplified in some embodiments of the presently-disclosed subject matter.
gatttcagttgaaagatgtgtttttgtgagtagagcaccgcagaagaactgaagactgttgtgtgctccccgcagaaggggctaccatgatcctttcctcctataacaccatccagtcggttttctgttgctgctgttgctgttcagtgcagaagcgacaaatgagaacacagataagcctgagcacagatgaagagcttccagaaaaatacacccagcgtcgcaggccgtggctcagccaattgtcaaataagaagcaatccaacacgggccgtgtgcagccgtcaaaacgaaagccactgcctcccctcccaccctctgaggttgctgaagagaagatccaagtcaaggcactttatgattttctgcccagagaaccctgtaatttagccttaaggagagcagaagaatacctgatactggagaaatacaatcctcactggtggaaggcaagagaccgtttggggaatgaaggcttaatcccaagcaactatgtgactgaaaacaaaataactaatttagaaatatatgagtggtaccatagaaacattaccagaaatcaggcagaacatctattgagacaagagtctaaagaaggtgcatttattgtcagagattcaagacatttaggatcctacacaatttccgtatttatgggagctagaagaagtacggaggctgccataaaacattatcagataaaaaagaatgactcaggacagtggtatgtggctgaaagacacgcctttcaatcaatccctgagttaatctggtatcaccagcacaatgcagccggtctcatgactcgtctccgatatccagttgggctgatgggcagttgtttaccagccacagctgggtttagctacgaaaagtgggagatagatccatctgagttggcttttataaaggagattggaagcggtcagtttggagtggtccatttaggtgaatggcggtcacatatccaggtagctatcaaggccatcaatgaaggctccatgtctgaagaggatttcattgaagaggccaaagtgatgatgaaattatctcattcaaagctagtgcaactttatggagtctgtatacagcggaagcccctttacattgtgacagagttcatggaaaatggctgcctgcttaactatctcagggagaataaaggaaagcttaggaaggaaatgctactgagtgtatgccaggatatatgtgaaggaatggaatatctggagaggaatggctatattcatagggatttggcggcaaggaattgtttggtcagttcaacatgcatagtaaaaatttcagactttggaatgacaaggtacgttttggatgatgagtatgtcagttcttttggagccaagttcccaatcaagtggtcccctcctgaagtttttcttttcaataagtacagcagtaaatctgatgtctggtcatttggagttttaatgtgggaagtttttacagaaggaaaaatgccttttgaaaataagtcaaatttgcaagtcgtggaagctatttctgaaggcttcaggctatatcgccctcacctggcaccaatgtccatatatgaagtcatgtacagctgctggcatgagaaacctgaaggccgccctacatttgccgagctgctgcgggctgtcacagagattgcggaaacctggtgaccggaaacagaatgccaacccaaagagtcatcttgcaaaactgtcatttattgtgaatatcttcaccatatggggtcacttatggtgaatatctttcttcagagttgctgactcttgaaaacagtgcaaagatcacagtttttaaaagttttaaaaatttaagaatattcacacaatcgtttttctatgtgtgagagggatttgcacactcttatttttctgtaaaatatttcacatcccaaatgtgaagaagtgaaaaagacttcgcagcagtcttcattgtggtgctcttcatgatcatagccccaggaacccttgaggttcttcttcacaaggctgagagtgcttccttcttgaagacgagtgacattcatcacttcagtgatccatgcatagaatatgaaaataaattcttccaactcatgggataaaggggactcccttgaagaatttcatgtttttgggctgtatagctctttacagaaaatgcacctttataaatcacatgaatgttagtattctggaaatgtcttttgttaatataatcttcccatgttatttaacaaattgtttttgcacatatctgattatattgaaagcagttttttgcattcgagttttaaacactgttataaaatgtagccaaagctcacctttgaacagatcccggtgacattctatttccaggaaaatccggaacctgattttagttctgtgattttacactttttacatgtgagattggacagtttcagaggccttattttgtcatactaagtgtctcctgtaattttcaggaagatgatttgttctttccagaagaggagacaaaagcaagatagccaaatgtgacatcaagctccattgtttcggaaatccaggattttgaattcgagatgaaacaaccagcaatcacagttaaatcttaactttgcctgcactctttgtaggaatgatcagaaatttatctttatcattctgagtgcttcaggagtacaataggaagaaagatactggagaaagcactaatgtaatcaccatgaagtctgacaacaggagcccattatttgcgtactgtcccaccctgtatcatggttctctgggaacaagctttatgattctcattagagtttatttgttgattgtcagtagttgcgacttttaaattatatttcccccactcaaagaatggtatctttatatatcaatgacattcaataaatgtgtattatttctaatgagaa